(19) United States
(12) Patent Application Publication
(10) Pub. No.: US 2024/0245770 A1
(43) Pub. Date: Jul. 25, 2024

(54) COMPOSITIONS AND METHODS FOR TREATING NEUROMUSCULAR DISORDERS

(71) Applicant: The Regents of the University of California, Oakland, CA (US)

(72) Inventor: Anahid Jewett, Los Angeles, CA (US)

(21) Appl. No.: 17/784,145

(22) PCT Filed: Dec. 16, 2020
(86) PCT No.: PCT/US2020/065355

§ 371 Date: Feb. 1, 2023

Related U.S. Application Data
(60) Provisional application No. 62/948,631, filed on Dec. 16, 2019.

(51) Int. Cl. A61K 39/395; A61K 31/198; A61K 31/4152; A61K 31/428; A61P 25/28

(52) U.S. Cl. CPC A61K 39/3955; A61K 31/198; A61K 31/4152; A61K 31/428; A61P 25/28

(57) ABSTRACT

The present invention relates to compositions and methods that reduce the level of pro-inflammatory cytokines, chemokines, and growth factors for inhibiting motor neuron degeneration and treating neurodegenerative disorders.

### CROSS-REFERENCE TO A RELATED APPLICATION

This application is a national-stage filing under 35 U.S.C. § 371 of PCT Application No. PCT/US2020/065355, filed Dec. 16, 2020, which claims the benefit of U.S. Provisional Application No. 62/948,631, filed on Dec. 16, 2019. The entire contents of both applications are incorporated herein in their entirety by this reference.

### BACKGROUND

Neurodegenerative disorders are incurable and debilitating conditions that result in progressive degeneration and/or death of nerve cells. Amyotrophic Lateral Sclerosis (ALS) is the most common adult-onset motor neuron disease or neuromuscular disorder, caused by the progressive degeneration of motor neurons in the spinal cord, brainstem, and motor cortex, resulting in loss of muscle control (Rowland et al., (2005) N Engl J Med 344:1688-1700). Motor neurons reach from the brain to the spinal cord and from the spinal cord to the muscles throughout the body. The progressive degeneration of the motor neurons in ALS eventually leads to death. When the motor neurons die, the ability of the brain to initiate and control muscle movement is lost, and patients in the later stages of the disease often become paralyzed.

Accordingly, there is a great need for compositions and methods for treating neurodegenerative diseases such as ALS.

### SUMMARY OF THE INVENTION

The present invention is based, at least in part, on the discovery that ALS patients have an increased level of pro-inflammatory cytokines, chemokines, and growth factors that contributes to motor neuron damage and subsequent onset/progression of neurodegenerative diseases such as ALS. It is demonstrated herein that while the overall percentages of CD8+ T cells is lower in peripheral blood of ALS patients as compared to healthy patients, the enhanced function of CD8+ T cells and B cells leads to secretion of excess cytokines, resulting in high serum levels of pro-inflammatory cytokines, chemokines, and growth factors in ALS patients. Accordingly, compositions and methods comprising one or more of agents that reduce the level of pro-inflammatory cytokines, chemokines, and growth factors are important in inhibiting motor neuron degeneration and treating neurodegenerative disorders. Provided herein are compositions and methods for therapy and diagnostic testings for neurodegenerative disorders.

In certain aspects, provided herein is a method of treating a neurodegenerative disease in a subject in need thereof, the method comprising administering to the subject at least one agent that decreases the level of one or more pro-inflammatory cytokines, chemokines, and/or growth factors.

In certain aspects, also provided herein is a method of inhibiting degeneration and/or death of a nerve cell in a subject, the method comprising administering to the subject at least one agent that decreases the level of one or more pro-inflammatory cytokines, chemokines, and/or growth factors.

Numerous embodiments are further provided that can be applied to any aspect encompassed by the present disclosure as described herein. For example, in some embodiments, the neurodegenerative disease is Amyotrophic Lateral Sclerosis (ALS), or the subject is afflicted with ALS.

In some embodiments, the at least one agent comprises at least one of N-Acetyl-Cysteine (NAC), an anti-IL-6 antibody, an anti-TNF-α antibody, an anti-Rantes antibody, and an anti-IFN-g antibody. In some embodiments, the at least one agent comprises NAC. In some embodiments, the at least one agent comprises an anti-IL-6 antibody and an anti-TNF-α antibody. In some embodiments, the at least one agent comprises an anti-IFN-g antibody. In some embodiments, the at least one agent comprises NAC, an anti-TNF-α antibody, and an anti-IFN-g antibody.

In some embodiments, the one or more pro-inflammatory cytokines, chemokines, and/or growth factors are selected from Rantes, EGF, FGF2, Eotaxin, TGF-α, FIT3L, GM-CSF, FRACTALKINE, IFNa2, IFN-g, MCP3, IL-12, MDC, PDGF-AA, PDGF-AB, PDGF-BB, IL-13, IL-15, sCD40L, IL-1Ra, IL-1a, IL-9, IL-1b, IL-3, IL-4, IL-7, IL-8, IP-10, MCP1, TNF-β, VEGF, IL-10, TNF-α, IL-17A, IL-1β, IL-2, IL-21, IL-4, IL-23, IL-5, IL-6, MIP-3α, MIP-1α, and MIP-1β.

In some embodiments, the one or more pro-inflammatory cytokines, chemokines, and/or growth factors are selected from IL-10, IL-12, IFN-g, TNF-α, IL-13, IL-17A, IL-2, IL-21, IL-4, IL-23, IL-5, IL-6, and MIP-3α. In some embodiments, the one or more pro-inflammatory cytokines, chemokines, and/or growth factors are selected from IL-4, IL-10, IL-12, IL-2, IL-13, IL-6, TNF-α, and IFN-g.

In certain embodiments, the at least one agent decreases inflammation in the subject.

In certain embodiments, the at least one agent inhibits the degeneration and/or death of a nerve cell.

In certain embodiments, the at least one agent decreases the likelihood of pulmonary embolism and/or cardiac failure.

In certain embodiments, the method further comprises administering to the subject at least one additional therapy that treats a neurodegenerative disease, or at least one additional therapy that inhibits degeneration and/or cell death of a nerve cell. In some embodiments, the at least one additional therapy is administered before, after, or concurrently with the agent that decreases the level of one or more pro-inflammatory cytokines, chemokines, and/or growth factors. In some embodiments, the at least one additional therapy is edavarone and/or riluzole.

In certain aspects, provided herein is a method of determining whether a subject afflicted with a neurodegenerative disease would likely respond to treatment with at least one agent that decreases the level of one or more pro-inflammatory cytokines, chemokines, and/or growth factors, the method comprising:
a) determining the amount of at least one biomarker in a subject sample;
b) determining the amount of the same biomarker(s) in a control; and
c) comparing the amount of the biomarker(s) in a) and b);
wherein the at least one biomarker is selected from Rantes, IL-10, IL-12, IFN-g, TNF-α, IL-13, IL-17A, IL-2, IL-21, IL-4, IL-23, IL-5, IL-6, and MIP-3α; and
wherein a significant increase in the amount of the biomarker(s) in the subject sample relative to the control indicates that the subject would benefit from treatment with an agent that decreases the level of one or more pro-inflammatory cytokines, chemokines, and/or growth factors.

In some embodiments, the amount of the biomarker is the amount of protein. In some embodiments, the sample comprises serum. In some embodiments, the control is determined from a subject not afflicted with the degenerative disease. In some embodiments, the method further comprises prescribing at least one agent that decreases the level of one or more pro-inflammatory cytokines, chemokines, and/or growth factors, if the amount of the biomarker(s) in the subject sample is increased relative to the control.

In some embodiments, the at least one agent comprises at least one of N-Acetyl-Cysteine (NAC), an anti-IL-6 antibody, an anti-TNF-α antibody, an anti-Rantes antibody, and an anti-IFN-g antibody. In some embodiments, the at least one agent comprises NAC. In some embodiments, the at least one agent comprises an anti-IL-6 antibody and an anti-TNF-α antibody. In some embodiments, the at least one agent comprises an anti-IFN-g antibody. In some embodiments, the at least one agent comprises NAC, an anti-TNF-α antibody, and an anti-IFN-g antibody.

In some embodiments, the one or more pro-inflammatory cytokines, chemokines, and/or growth factors are selected from Rantes, EGF, FGF2, Eotaxin, TGF-α, FIT3L, GM-CSF, FRACTALKINE, IFNa2, IFN-g, MCP3, IL-12, MDC, PDGF-AA, PDGF-AB, PDGF-BB, IL-13, IL-15, sCD40L, IL-1Ra, IL-1a, IL-9, IL-1b, IL-3, IL-4, IL-7, IL-8, IP-10, MCP1, TNF-β, VEGF, IL-10, TNF-α, IL-17A, IL-1β, IL-2, IL-21, IL-4, IL-23, IL-5, IL-6, MIP-3α, MIP-1α, and MIP-1β.

In some embodiments, the one or more pro-inflammatory cytokines, chemokines, and/or growth factors are selected from IL-10, IL-12, IFN-g, TNF-α, IL-13, IL-17A, IL-2, IL-21, IL-4, IL-23, IL-5, IL-6, and MIP-3α. In some embodiments, the one or more pro-inflammatory cytokines, chemokines, and/or growth factors are selected from IL-4, IL-10, IL-12, IL-2, IL-13, IL-6, TNF-α, and IFN-g.

In certain embodiments, the at least one agent decreases inflammation in the subject.

In certain embodiments, the at least one agent inhibits the degeneration and/or death of a nerve cell.

In certain embodiments, the at least one agent decreases the likelihood of pulmonary embolism and/or cardiac failure.

In some embodiments, the subject is a mammal. In some embodiments, the subject is a human.

### BRIEF DESCRIPTION OF FIGURES

*Samples: JC, RS, and WO are ALS patients; TC is a healthy twin of JC; rest of the samples are controls.

FIG. 1A-FIG. 1B show the percentage of immune subsets in PBMCs in lab blood drawn. Flow cytometric dot plots (FIG. 1A). Percentage of the cell types (FIG. 1B).

FIG. 2A-FIG. 2C: FIG. 2A shows purity of NK cells. FIG. 2B shows no significant differences in primary NK cytotoxicity within PBMCs between the donors. FIG. 2C shows no significant differences in NK cytotoxicity by purified NK cells between the donors.

FIG. 3A-FIG. 3G: FIG. 3A shows Day 6 NK Expansion, super-charging the NK cells. FIG. 3B shows Day 9 NK Expansion, increased NKT subsets but no change in CD8+ T cells in ALS. FIG. 3C shows Day 12 NK Expansion, NK cells expand NKT cell subset in all but particularly more in ALS patient. FIG. 3D shows Day 27 NK Expansion, increased contraction of NKT cells after expansion by the NK cells in ALS patient. FIG. 3E shows Day 27 NK Expansion. FIG. 3F shows the fold increase in expansion of super-charged NK cells by day. FIG. 3G shows population Doubling of super-charged NK cells is similar between the donors.

FIG. 4A-FIG. 4B show that during supercharging of the NK cells no significant differences can be seen in secretion of IFN-g from NK cells at different days of expansion.

FIG. 5A-FIG. 5B show that during supercharging of the NK cells no significant differences can be seen in lysis of OSCSCs by the NK cells at day 15 of NK expansion.

FIG. 6A-FIG. 6C: FIG. 6A shows decreased cytotoxicity of Untreated supercharged NK cells by ALS patient. FIG. 6B shows decreased cytotoxicity of IL-2 treated supercharged NK cells by ALS patient. FIG. 6C shows decreased cytotoxicity of IL-2+anti-CD16 treated supercharged NK cells by ALS patient.

FIG. 7 shows decreased cytotoxicity of supercharged NK cells by ALS patient. NK cell-mediated cytotoxicity of OSCSCs.

FIG. 8A-FIG. 8B show no or decreased secretion of IFN-g by supercharged NK cells by ALS patient.

FIG. 9 shows flow cytometry of PBMCs isolated from a patient afflicted with ALS and healthy controls. No therapy is shown.

FIG. 10 shows no differences in the NK cytotoxicity of PBMCs by ALS patient. PBMC 51Cr release.

FIG. 11 shows increased cytotoxicity of NK cells cultured with autologous or allogeneic monocytes by ALS patient. NK with different donor monocytes.

FIG. 12 shows no differences in the cytotoxicity of NK cells cultured with CD4+ T cells by ALS patient. Interaction of NK with CD4+ T cells.

FIG. 13 shows no differences in the cytotoxicity of NK cells cultured with CD8+ T cells by ALS patient. Interaction of NK with CD8+ T cells.

FIG. 14 shows no significant differences in the IFN-g secretion with the exception of IL-2+anti-CD3/CD28 treated PBMCs of ALS patient. PBMC-mediated IFN-g release.

FIG. 15 shows that ALS patient's NK cells increase secretion of IFN-g with both autologous and allogeneic monocytes indicating potential priming of the NK cells. IFN-g release of NK with different donor monocytes.

FIG. 16 shows that the sorted CD8+ T cells from ALS patient secrete significant levels of IFN-g upon activation with IL-2+anti-CD3/CD28 in comparison to CD4+ T cells. NK and/or CD4 and CD8 mediated IFN-g release.

FIG. 17 shows the flow cytometry of PBMCs isolated from a patient afflicted with ALS and healthy controls.

FIG. 18 shows comparable or higher percentages of Tregs within CD4+ T cells between ALS and healthy twin.

FIG. 19 shows comparable or higher percentages of Tregs within naïve CD4+ T cells differentiated to Tregs between ALS and healthy twin.

FIG. 20 shows comparable levels of IL-10 secretion from Tregs obtained from ALS and healthy twin. IL-10 secretion by Tregs.

FIG. 21 shows the flow cytometry of PBMCs isolated from a patient afflicted with ALS and healthy controls.

FIG. 22 shows the CD3/CD28 mediated increase in NK function by ALS patient PBMCs. ELISA of IFN-g.

FIG. 23 shows the CD3/CD28 mediated increase in NK function by ALS patient PBMCs. ELISpot of IFN-g.

FIG. 24 shows the CD3/CD28 mediated increase in NK function by ALS patient PBMCs. The NK cell mediated cytotoxicity was determined by the standard 4-hour chromium release assay using oral squamous carcinoma stem cells (OSCSCs) as targets.

FIG. 25 shows the flow cytometry of PBMCs isolated from a patient afflicted with ALS.

FIG. 26 shows the CD3/CD28 and AJ2 mediated increase in NK function from PBMCs and purified NK cells from ALS patient. PBMC cytotoxicity was determined by the standard 4-hour chromium release assay using oral squamous carcinoma stem cells (OSCSCs) as targets.

FIG. 27 shows the CD3/CD28 and AJ2 mediated increase in NK function from PBMCs and purified NK cells from ALS patient. NK cell cytotoxicity was determined by the standard 4-hour chromium release assay using oral squamous carcinoma stem cells (OSCSCs) as targets.

FIG. 28 shows ELISA of IFN-g secreted by PBMC from an ALS patient.

FIG. 29 shows ELISA of IFN-g secreted by purified NK cells from an ALS patient.

FIG. 30 show ELISpot of IFN-g secreted by PBMC from an ALS patient.

FIG. 31 shows ELISpot of IFN-g secreted by purified NK cells from an ALS patient.

FIG. 32 shows the flow cytometry of PBMCs from patients afflicted with ALS and healthy controls.

FIG. 33 shows the percentages of Treg subset within CD4+ T cells as determined by flow cytometry.

FIG. 34 shows comparable or higher secretion of IFN-g from the PBMCs and purified NK cells between ALS and healthy controls. PBMC ELISpot.

FIG. 35 shows comparable or higher secretion of IFN-g from the PBMCs and purified NK cells between ALS and healthy controls. NK ELISpot.

FIG. 36 shows the flow cytometry of PBMCs isolated from a patient afflicted with ALS.

FIG. 37 shows the CD3/CD28 and AJ2 mediated increase in NK function in ALS patient. NK cell cytotoxicity.

FIG. 38 shows the CD3/CD28 and AJ2 mediated increase in NK function in ALS patient. IFN-g ELISA.

FIG. 39 shows the flow cytometry of PBMCs isolated from patients afflicted with ALS.

FIG. 40 shows the CD3/CD28 mediated increase in IFN-g secretion by CD8 in ALS patient. PBMC ELISpot.

FIG. 41 shows the CD3/CD28 mediated increase in IFN-g secretion by CD8 in ALS patient. NK cell ELISpot.

FIG. 42 shows the CD3/CD28 mediated increase in IFN-g secretion by CD8 in ALS patient. CD8 ELISpot.

FIG. 43 shows the flow cytometry of PBMCs isolated from patients afflicted with ALS.

FIG. 44 shows CD3/CD28 mediated increase in IFN-g spots and secretion by PBMCs of ALS patient. PBMC ELISpot.

FIG. 45 shows comparable or increased IFN-g secretion by the NK cells with and without monocytes in ALS patient as compared to healthy twin. IFN-g ELISA.

FIG. 46 shows comparable or increased IFN-g secretion by the autologous or allogeneic NK cells with and without autologous/allogeneic monocytes in ALS patient as compared to healthy twin, but increased IFN-g in CD8 T cells in ALS vs. healthy twin. NK ELISpot.

FIG. 47 shows comparable or increased IFN-g secretion by the autologous or allogeneic NK cells with and without autologous/allogeneic monocytes in ALS patient as compared to healthy twin, but increased IFN-g in CD8 T cells in ALS vs. healthy twin. CD8 ELISpot.

FIG. 48 shows comparable percentages of immune subsets in ALS patient as compared to healthy twin. The flow cytometry of PBMCs isolated from donors.

FIG. 49 shows increased percentages of CD45RO in CD4+ T cells in ALS patient as compared to healthy twin. Flow cytometry.

FIG. 50 shows increased IFN-g spots and secretion by the PBMCs of ALS patient as compared to healthy twin. PBMC ELISpot.

FIG. 51 shows increased IFN-g secretion by the CD8+ T cells in ALS patient as compared to healthy twin. CD8 ELISpot.

FIG. 52 shows similar proportions of immune subsets in ALS patient as compared to healthy twin. Flow cytometry.

FIG. 53 shows no significant differences in cell death in different immune subsets after activation in ALS patient as compared to healthy twin. Propidium iodide staining for cell death.

FIG. 54 shows increased IFN-g secretion by PBMCs and CD4+ T cells activated with anti-CD3/28 in ALS patient as compared to healthy twin. PBMC ELISpot.

FIG. 55 shows increased IFN-g secretion by PBMCs and CD4+ T cells activated with anti-CD3/28 in ALS patient as compared to healthy twin. CD4+ T cell ELISpot.

FIG. 56 shows the increased IFN-g spots and secretion by CD8+ T cells activated with anti-CD3/28 in ALS patient as compared to healthy twin. ELISpot.

FIG. 57 shows similar proportions of immune subsets in ALS patient as compared to healthy twin. Flow cytometry.

FIG. 58 shows no significant differences in NK cell cytotoxicity and secretion of IFN-g within PBMCs in ALS patient as compared to healthy twin. PBMC ELISpot.

FIG. 59 shows similar proportions of immune subsets with the exception of CD8+ T cells in ALS patient as compared to healthy twin, Decrease in CD8+ T cell percentages in ALS patient.

FIG. 60 shows comparable percentages of immune subsets with the exception of CD8+ T cells and Tregs in ALS patient as compared to healthy twin. Lower CD8+ and Tregs in ALS than control twin. Flow cytometry.

FIG. 61 shows comparable or higher secretion and spots of IFN-g from the PBMCs between ALS and healthy twin. Differences between the two and RS another patient with ALS. PBMC ELISpot.

FIG. 62 shows comparable secretion and spots of IFN-g from the purified NK cells between ALS and healthy controls. NK ELISpot.

FIG. 63 shows increased IFN-g spots and secretion by CD8+ T cells activated with anti-CD3/28 in ALS patients as compared to healthy controls. CD8 ELISpot.

FIG. 64 shows similar proportions of immune subsets with the exception of CD8+T cells in ALS patient as compared to healthy twin, Decrease in CD8+ T cell percentages in ALS patient. PBMC flow cytometry.

FIG. 65 shows testing and selection of the best NAC to be used in ALS treatment. NAC blocks CDDP mediated Cell death in Oral tumors.

FIG. 66 shows testing and selection of the best NAC to be used in ALS treatment. NAC blocks CDDP mediated Cell death in Oral tumors.

FIG. 67 shows testing and selection of the best NAC to be used in ALS treatment. NAC blocks H2O2 mediated Cell death in DPSCs.

FIG. 68 shows Testing and selection of the best NAC to be used in ALS treatment. NAC blocks H2O2 mediated Cell death in OSCSCs.

FIG. 69 shows increased cytokine/chemokine/growth factor/ligands in serum of ALS patient compared to healthy twin and control.

FIG. 70 shows Higher secretion and spots of IFN-g and NK cell mediated cytotoxicity from the PBMCs and CD8+ T cells from ALS patient. Flow cytometry.

FIG. 71 shows Higher secretion and spots of IFN-g and NK cell mediated cytotoxicity from the PBMCs and CD8+ T cells from ALS patient. ELISpot.

FIG. 72 shows Percentages of different immune subsets in ALS patient. Flow cytometry.

FIG. 73 shows Percentages of different immune subsets in ALS patient pre and post NAC infusion compared to controls. Flow cytometry.

FIG. 74 shows IFN-g spots from the PBMCs of ALS patient pre and post NAC infusion.

FIG. 75 shows Similar spots of IFN-g in NK cell s from ALS patient pre and post NAC infusion.

FIG. 76 shows No differences in the percentages of immune subsets between ALS and control twin. Flow cytometry.

FIG. 77 shows IFN-g spots are higher in IL-2 activated PBMCs but not under all other activation in PBMCs.

FIG. 78 shows IFN-g spots and secretion are higher in CD8+ T cells from ALS patient in comparison to twin control.

FIG. 79 shows PBMC flow cytometry.

FIG. 80 shows IFN-g spots are higher in activated CD8s when compared to PBMCs and anti-PD-1 antibody increases CD8+ T cell mediated IFN-g spots and release.

FIG. 81 shows No differences in the percentages of key immune subsets between ALS and control twin, however, lower percentages of Tregs in ALS. Flow cytometry.

FIG. 82 shows No differences in the percentages of PD-1 expression on PBMCs and NK cells with the exception of CD8+ T cells between ALS and control twin. Flow cytometry.

FIG. 83 shows No significant differences in IFN-g spots in PBMCs between ALS and control twin. ELISpot.

FIG. 84 shows Increased IFN-g spots in CD8+ T cells in ALS when compared to control twin. NK ELISpot.

FIG. 85 shows Increased IFN-g spots in CD8+ T cells in ALS when compared to control twin. CD8 ELISpot.

FIG. 86 shows No differences in the percentages of key immune subsets between ALS and control twin. Flow cytometry.

FIG. 87 shows Decreased percentages of Treg subsets between ALS and control twin. Flow cytometry.

FIG. 88 shows No differences in the IFN-g spots in PBMCs between ALS and control twin, and lower NK cytotoxicity in ALS. ELISpot.

FIG. 89 shows No differences in the secretion of IFN-g by PBMCs but significant increases in INF-g spots and secretion in CD8+ T cells in ALS when compared to control twin. CD8 ELISpot.

FIG. 90-FIG. 92 shows Significantly increased cytokines and chemokines in serum from ALS patient as compared to healthy twin before NAC treatment. Serum Luminex.

FIG. 93-FIG. 97 show significantly increased cytokines and chemokines in serum from ALS patient as compared to healthy twin after NAC treatment. Serum Luminex.

FIG. 98 shows PBMC flow cytometry.

FIG. 99 shows Detection of central memory CD8+ T cells and lack or decreased of IFN-g R expression on the CD8+ T cells.

FIG. 100 shows Decreased levels of Tregs in PBMCs from ALS patient.

FIG. 101 shows PBMC Elispot.

FIG. 102 shows NK Elispot.

FIG. 103 shows CD8 Elispot.

FIG. 104-FIG. 105 show Increased cell death of OSCSCs by supernatants from CD8+ T cells from ALS patient as compared to healthy twin and increased CD54 expression on OSCSCs.

FIG. 106 shows increased PD-1 expression on ALS. Flow cytometry.

FIG. 107 shows Detection of central memory CD8+ T cells and lack or decreased of IFN-g R expression on the CD8+ T cells. Flow cytometry.

FIG. 108 shows Increased IFN-g spots and secretion in ALS patients JC and WO when compared to TC. ELISpot.

FIG. 109 shows Increased IFN-g secretion by CD8+ T cells from ALS (JC and WO) when compared to TC.

FIG. 110 shows PBMC flow cytometry.

FIG. 111 shows Decreased CD69 expression in RS when compared to JC ALS patient. RS received mobilization of Bone marrow derived stem cells by GCSF. Had very high levels of peripheral blood white blood cells.

FIG. 112-FIG. 114 show Decreased IFN-g spots in RS when compared to JC ALS patient and JS healthy control. RS received mobilization of Bone marrow derived stem cells by GCSF. RS Had very high levels of peripheral blood white blood cells. Even though no spots could be observed in PBMCs and NK cells of RS we still saw increased spots from CD8+ T cells. JC (ALS) was in general higher than JS healthy control.

FIG. 115 shows JC (ALS) had higher expression of CD69 when compared to RS (ALS) or JS healthy control.

FIG. 116 shows PBMC flow cytometry.

FIG. 117 shows Lower percentages of CD8+ T cells in JC and WO ALS patients when compared to TC. Flow cytometry.

FIG. 118 shows Higher PD-1 expression in JC and WO ALS patients compared to TC healthy control. Higher T regs in PBMCs in JC and WO when compared to TC. No differences in naïve CD4 differentiation.

FIG. 119-FIG. 120 show Increased IFN-g spots in NK and CD8+ T cells from JC and WO when compared to TC. NK ELISpot (FIG. 119). CD8 ELISpot (FIG. 120).

FIG. 121 shows PBMC flow cytometry.

FIG. 122 shows PBMC ELISpot.

FIG. 123 shows Percentages of CD8+ T cells are lower in ALS patients RS, WO and JC when compared to TC healthy control. PBMC flow cytometry.

FIG. 124 shows PBMC flow cytometry after anti-TNF therapy. Decreased or no change in the percentages of CD8+ subsets in ALS patients when compared to control.

FIG. 125 shows PBMC ELISpot.

FIG. 126 shows PBMC ELISpot.

FIG. 127 shows PBMC ELISpot.

FIG. 128 shows PBMC ELISpot.

FIG. 129 shows PBMC flow cytometry pre-stem cell injection in muscle of ALS patient.

FIG. 130 shows PBMC flow cytometry histogram. Increase in the percentages of Treg subset in ALS patient when compared to control.

FIG. 131 shows PBMC ELISpot.

FIG. 132 shows CD8 ELISpot.

FIG. 133 shows PBMC flow cytometry post-stem cell therapy. Decreased in the percentages of CD8+ subsets in ALS patients when compared to control.

FIG. 134 shows PBMC, NK, and CD8 ELISpot. Increased IFN-γ spots in PBMCs, NK and CD8+ T cells in the majority of treatments in ALS when compared to control twin.

FIG. 135 shows PBMC flow cytometry. Decrease in the percentages of CD8+ subsets in ALS patients when compared to control.

FIG. 136 shows PBMC, NK, and CD8 ELISpot. Increased IFN-γ spots in PBMCs, and CD8+ T cells in the majority of treatments but not in NK cells in ALS when compared to control twin.

FIG. 137 shows PBMC flow cytometry histogram. Similar percentages of CD8+ subsets in ALS patients when compared to control.

FIG. 138 shows PBMC flow cytometry.

FIG. 139 shows PBMC ELISpot. Higher NK cytotoxicity, IFN-γ spots, and secretion in PBMCs of ALS patients in the majority of treatments as compared to control.

FIG. 140 shows CD8 ELISpot. Increased IFN-γ spots and higher secretion of IFN-g in CD8+ T cells from ALS patient within all treatments in comparison to twin control.

FIG. 141 shows PBMC flow cytometry. Decreased percentages of CD8+ subsets in ALS patients when compared to control.

FIG. 142 shows PBMC ELISpot. IFN-γ spots are higher in PBMCs of ALS patients with most treatments tested in comparison to twin control FIG. 143 shows CD8 ELISpot. Increased IFN-γ spots with the exception of one treatment, and higher secretion of IFN-g in all treatments from CD8+ T cells from all ALS patients in comparison to twin control.

FIG. 144 shows PBMC flow cytometry.

FIG. 145 shows T reg flow cytometry histogram. Variable levels of Tregs in PBMCs from ALS patients when compared to Twin control or other healthy controls.

FIG. 146 shows T reg flow cytometry.

FIG. 147 shows PBMC flow cytometry.

FIG. 148 shows NK ELISpot. No significant differences in NK cytotoxicity, NK IFN-g spots and IFN-g secretion by the NK cells between ALS patients and control.

FIG. 149 shows CD8 ELISpot. Similar IFN-γ spots and higher secretion of IFN-γ in CD8+ T cells from all ALS patients in IL-2+anti-CD3/CD28 or IL-2+ sAJ2 treated samples in comparison to twin control.

FIG. 150 shows T reg flow cytometry histogram. Similar or decreased levels of Tregs in differentiated nCD4 in JC when compared to RS ALS patient or control twin.

FIG. 151 shows T reg flow cytometry. Decreased levels of Tregs in differentiated nCD4 in JC and RS when compared to control twin at day 20.

FIG. 152 shows T reg flow cytometry histogram.

FIG. 153 shows graph results of Table 218.

FIG. 154 shows PBMC ELISpot.

FIG. 155 shows graphs results of Tables 219.

FIG. 156 shows graph results of Tables 220.

FIG. 157 shows PBMC ELISpot.

FIG. 158 shows graph results of Tables 221.

FIG. 159 shows graph results of Tables 222.

FIG. 160 shows PBMC flow cytometry. No significant change in the percentages of CD8+ subsets in ALS patient when compared to control FIG. 161 shows graph results of Tables 224. Similar or decreased secretion of IFN-γ in PBMCs of ALS patient with different treatments tested in comparison to twin control.

FIG. 162 shows graph results of Tables 225. Similar or decreased secretion of IFN-γ in PBMCs of ALS patient with different treatments tested in comparison to twin control.

FIG. 163 shows graph results of Tables 226. Increased secretion of IFN-γ in CD8+ T cells from ALS patient in all treatments in comparison to twin control.

FIG. 164 shows graph results of Tables 227. Increased secretion of IFN-γ in CD8+ T cells from ALS patient in all treatments in comparison to twin control.

FIG. 165 shows PBMC flow cytometry histogram.

FIG. 166 shows PBMC flow cytometry.

FIG. 167 shows graph results of Tables 229. Higher NK cytotoxicity in ALS patient in all treatments in PBMCs and in sorted NK cells when compared to control.

FIG. 168 shows graph results of Tables 230. Higher NK cytotoxicity in ALS patient in all treatments in PBMCs and in sorted NK cells when compared to control.

FIG. 169 shows PBMC ELISpot. Increased or no difference in IFN-g spots but higher secretion of IFN-γ in PBMCs of ALS patient with the majority of treatments tested in comparison to twin control.

FIG. 170 shows graph results of Tables 231 and 232.

FIG. 171 shows NK ELISpot. No significant differences in IFN-γ spots but higher secretion of IFN-γ by NK cells in ALS patient with the majority of treatments tested in comparison to twin control.

FIG. 172 shows graph results of Table 233.

FIG. 173 shows graph results of Table 234.

FIG. 174 shows CD8 ELISpot. Increased IFN-γ spots and higher secretion of IFN-γ in CD8+ T cells in all treatments from ALS patient in comparison to twin control.

FIG. 175 shows graph results of Table 235.

FIG. 176 shows graph results of Table 236.

FIG. 177 shows PBMC flow cytometry histogram.

FIG. 178 shows PBMC flow cytometry.

FIG. 179 shows graph results of Table 238. Higher NK cytotoxicity in PBMCs and NK cells in all treatments in ALS patient when compared to control twin.

FIG. 180 shows graph results of Table 239. Higher NK cytotoxicity in PBMCs and NK cells in all treatments in ALS patient when compared to control twin.

FIG. 181 shows PBMC ELISpot. Increased IFN-γ spots in all treatments and increased secretion of IFN-γ in some treatments in PBMCs from ALS patient in comparison to twin control.

FIG. 182 shows graph results of Table 240.

FIG. 183 shows graph results of Table 241.

FIG. 184 shows NK ELISpot. Variable results in IFN-γ spots but higher secretion of IFN-γ by the majority of treatments in NK cells in ALS patient in comparison to twin control.

FIG. 185 shows graph results of Table 242.

FIG. 186 shows graph results of Table 243.

FIG. 187 shows CD8 ELISpot. Increased IFN-γ spots and higher secretion of IFN-γ in some treatments in CD8+ T cells from ALS patient in comparison to twin control.

FIG. 188 shows graph results of Table 244.

FIG. 189 shows graph results of Table 245.

### DETAILED DESCRIPTION OF THE INVENTION

The present invention relates, in part, to compositions and methods for therapy and diagnostic testings for neurodegenerative disorders such as ALS. It is discovered herein that enhanced function of CD8+ T cells and B cells in ALS patients results in secretion of excess cytokines, which contributes to increased serum levels of cytokines, chemokines, and growth factors in ALS patients. Neurodegeneration is facilitated by the lack of neurotrophic growth factors and by the persistent production of cytotoxic byproducts of pro-inflammatory response. Thus, inhibition of inflammation using the compositions and methods provided herein prevents neurodegeneration and onset/progression of diseases such as ALS. Accordingly, compositions and methods described herein are useful in the treatment of neurodegenerative and neuromuscular disorders.

For example, it is observed that a number of pro-inflammatory cytokines, chemokines, and or growth factors such as Rantes, EGF, FGF2, Eotaxin, TGF-α, FIT3L, GM-CSF, FRACTALKINE, IFNa2, IFN-g, MCP3, IL-12, MDC, PDGF-AA, PDGF-AB, PDGF-BB, IL-13, IL-15, sCD40L, IL-1Ra, IL-1a, IL-9, IL-1b, IL-3, IL-4, IL-7, IL-8, IP-10, MCP1, TNF-β, VEGF, IL-10, TNF-α, IL-17A, IL-1β, IL-2, IL-21, IL-4, IL-23, IL-5, IL-6, MIP-3α, MIP-1α, and MIP-10 are increased in the blood of a patient afflicted with a neurodegenerative disorder (e.g., ALS).

At least one agent that decreases the level of one or more of pro-inflammatory cytokines, chemokines, and or growth factors such as Rantes, EGF, FGF2, Eotaxin, TGF-α, FIT3L, GM-CSF, FRACTALKINE, IFNa2, IFN-g, MCP3, IL-12, MDC, PDGF-AA, PDGF-AB, PDGF-BB, IL-13, IL-15, sCD40L, IL-1Ra, IL-1a, IL-9, IL-1b, IL-3, IL-4, IL-7, IL-8, IP-10, MCP1, TNF-β, VEGF, IL-10, TNF-α, IL-17A, IL-1β, IL-2, IL-21, IL-4, IL-23, IL-5, IL-6, MIP-3α, MIP-1α, and MIP-10 may prevent neurodegeneration and be an effective treatment for a patient afflicted with a neurodegenerative disorder (e.g., ALS).

In some embodiments, the at least one agent targets and decreases the level of Rantes, IL-6, TNF-α, and/or IFN-g. In some embodiments, the at least one agent comprises N-acetyl-cysteine (NAC) that decreases the level of one or more of pro-inflammatory cytokines, chemokines, and or growth factors, e.g., IL-4, IL-10, IL-12, IL-2, IL-13, Rantes. In some embodiments, the at least one agent comprises an antibody that inhibits Rantes, IL-6, TNF-α, and/or IFN-g. In some embodiments, the at least one agent comprises a combination of anti-IL-6 antibody and anti-TNF-α antibody. In some embodiments, the at least one agent comprises an anti-IFN-g antibody. In some embodiments, the at least one agent comprises NAC, an anti-TNF-α antibody, and an anti-IFN-g antibody.

In certain embodiments, it is beneficial to treat the patients with an additional therapy in addition to the compositions described herein. In certain such embodiments, the conjoint therapy provides a synergistic effect relative to the additional therapy alone. In some embodiments, the additional therapy comprises edavarone and/or riluzole.

In certain embodiments, the at least one agent decreases the likelihood of pulmonary embolism and/or cardiac failure, to which patients afflicted with a neurodegenerative disorder are often susceptible or predisposed. In some such embodiments, the at least one agent decreases the level of PDGF.

Relatedly, the increased levels of cytokines, chemokines, and growth factors in patients afflicted with a neurodegenerative/neuromuscular disorder can be meaningful diagnostic biomarkers indicating that the patients could benefit from a therapy that targets various pro-inflammatory molecules, such as Rantes, IL-10, IL-12, IFN-g, TNF-α, IL-13, IL-17A, IL-2, IL-21, IL-4, IL-23, IL-5, IL-6, and MIP-3α. An increase in the amount of any one of these biomarkers may indicate that a patient would benefit from treatment with an agent that decreases the level of one or more pro-inflammatory cytokines, chemokines, and/or growth factors.

### Definitions

The articles “a” and “an” are used herein to refer to one or to more than one (i.e. to at least one) of the grammatical object of the article. By way of example, “an element” means one element or more than one element.

The term “administering” is intended to include routes of administration which allow a therapy to perform its intended function. Examples of routes of administration include injection (intramuscular, subcutaneous, intravenous, parenterally, intraperitoneally, intrathecal, etc.) routes. The injection can be a bolus injection or can be a continuous infusion. Depending on the route of administration, the agent can be coated with or disposed in a selected material to protect it from natural conditions which may detrimentally affect its ability to perform its intended function.

The terms “conjoint therapy” and “combination therapy,” as used herein, refer to the administration of two or more therapeutic substances. The different agents comprising the combination therapy may be administered concomitant with, prior to, or following the administration of one or more therapeutic agents.

The term “preventing” is art-recognized, and when used in relation to a condition, such as a local recurrence (e.g., loss of muscle control), a disease such as neurodegenerative disorder (e.g., ALS), a syndrome complex such as paralysis or any other medical condition, is well understood in the art, and includes administration of a composition which reduces the frequency of, or delays the onset of, symptoms of a medical condition in a subject relative to a subject which does not receive the composition.

The term “subject” or “patient” refers to any healthy or diseased animal, mammal or human, or any animal, mammal or human. In some embodiments, the subject is afflicted with a neurodegenerative disorder (e.g., ALS). In various embodiments of the methods of the present invention, the subject has not undergone treatment. In other embodiments, the subject has undergone treatment.

A “therapeutically effective amount” of a substance or cells is an amount capable of producing a medically desirable result in a treated patient, e.g., increase muscle control, delay or reduce paralysis, or alleviate any symptom associated with a neurodegenerative disease, with an acceptable benefit: risk ratio, preferably in a human or non-human mammal.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal), then the treatment is prophylactic (i.e., it protects the host against developing the unwanted condition); whereas, if it is administered after manifestation of the unwanted condition, the treatment is therapeutic (i.e., it is intended to diminish, ameliorate, or stabilize the existing unwanted condition or side effects thereof).

### Expression Level/Control

The expression level includes reference to the level of mRNA and/or the level of protein. It is appreciated in the art that an increase in copy number of the DNA results in increased level of mRNA and protein.

The altered level of expression of a biomarker refers to an expression level or copy number of a marker in a test sample e.g., a sample derived from a subject suffering from a neurodegenerative disease, that is greater or less than the standard error of the assay employed to assess expression or copy number, and is higher or lower than the expression level or copy number of the marker or chromosomal region in a control sample (e.g., sample from a healthy subject not having the associated disease) and preferably, the average expression level or copy number of the marker or chromosomal region in several control samples. The altered level of expression is greater or less than the standard error of the assay employed to assess expression or copy number, and higher or lower than the expression level or copy number of the marker in a control sample (e.g., sample from a healthy subject not having the associated disease) and preferably, the average expression level or copy number of the marker in several control samples.

The amount or activity of a biomarker in a subject is significantly higher or lower, or increased or decreased compared with the normal amount or activity of the biomarker, if the amount or activity of the biomarker is greater or less, respectively, than the normal or control level by an amount greater than the standard error of the assay employed to assess amount or activity, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternatively, the amount or activity of the biomarker in the subject can be considered significantly higher or lower, or increased or decreased compared with than the normal and/or control amount or activity if the amount or activity is at least about two, and preferably at least about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, two times, three times, four times, five times, or more, or any range in between, such as 5%-100%, higher or lower, respectively, than the normal and/or control amount or activity of the biomarker. Such modulation values can be applied to any metric described herein, such as altered level of expression, altered activity, degree of inflammation, changes in muscle control, neuronal degeneration, paralysis, and the like. The significant increase or decrease can also be assessed from any desired or known point of comparison, such as a particular post-treatment versus pre-treatment measurement ratio (e.g., 1-fold, 1.1-fold, 1.2-fold, 1.3-fold, 1.4-fold, 1.5-fold, and the like).

As used herein, inhibition, decrease, or grammatical equivalents thereof refer decrease, limiting, and/or blocking a particular action, function, or interaction. In certain embodiments, the term refers to reducing the level of a given output or parameter to a quantity which is at least 10%, 15%, 20%, 25%, 30%, 35%, 40%, 45%, 50%, 55%, 60%, 65%, 70%, 75%, 80%, 85%, 90%, 95%, 99% or less than the quantity in a corresponding control. A reduced level of a given output or parameter need not, although it may, mean an absolute absence of the output or parameter. The invention does not require, and is not limited to, methods that wholly eliminate the output or parameter. The given output or parameter can be determined using methods well-known in the art, including, without limitation, immunohistochemical, molecular biological, cell biological, clinical, and biochemical assays, as discussed herein and in the examples. The opposite terms promotion or decrease, or grammatical equivalents thereof refer to the increase in the level of a given output or parameter that is the reverse of that described for inhibition or decrease.

A control refers to any suitable reference standard, such as a normal patient, blood rom a subject such as a normal subject, cultured primary cells/tissues isolated from a subject such as a normal subject, adjacent normal cells/tissues obtained from the same organ or body location of the patient, a tissue or cell sample isolated from a normal subject, or a primary cells/tissues obtained from a depository. In other preferred embodiments, the control may comprise an expression level (e.g., mRNA or protein level of cytokines, chemokines, and/or growth factors), numbers of a certain cell type, and/or a cellular function of a certain cell type for a set of subject, such as a normal or healthy subject. In some embodiments, a control refers to a sample lacking the test agent (e.g., an agent that decreases the level of one or more of cytokines, chemokines, and/or growth factors).

A control also refers to any reference standard suitable to provide a comparison to the expression products in the test sample. In certain embodiments, the control comprises obtaining a control sample from which expression product levels, e.g., serum, PBMC, blood, are detected and compared to the expression product levels from the test sample.

Such a control sample may comprise any suitable sample, including but not limited to a sample from a control patient afflicted with a neurodegenerative disorder (can be stored sample or previous sample measurement) with a known outcome; normal blood, tissue, or cells isolated from a subject, such as a normal patient or the patient afflicted with a neurodegenerative disorder, cultured primary cells/tissues isolated from a subject such as a normal subject or the patient afflicted with a neurodegenerative disorder, adjacent normal cells/tissues obtained from the same organ or body location of the patient afflicted with a neurodegenerative disorder, a tissue or cell sample isolated from a normal subject, or a primary cells/tissues obtained from a depository. In some embodiments, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care therapy for a neurodegenerative disorder). It will be understood by those of skill in the art that such control samples and reference standard expression product levels can be used in combination as controls in the methods of the present invention.

In some embodiments, the amount of proteins or nucleic acids may be determined within a sample relative to, or as a ratio of, the amount of proteins or nucleic acids of another gene in the same sample. In some embodiments, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In preferred embodiments, the control comprises a control sample which is of the same lineage and/or type as the test sample. In other embodiments, the control may comprise expression product levels grouped as percentiles within or based on a set of patient samples, such as all patients with a neurodegenerative disorder. In some embodiments, a control expression product level is established wherein higher or lower levels of expression product relative to, for instance, a particular percentile, are used as the basis for predicting outcome. In other preferred embodiments, a control expression product level is established using expression product levels from neurodegenerative disorder control patients with a known outcome, and the expression product levels from the test sample are compared to the control expression product level as the basis for predicting outcome. As demonstrated by the data below, the methods of the present invention are not limited to use of a specific cut-point in comparing the level of expression product in the test sample to the control.

In some embodiments, a pre-determined marker amount and/or activity measurement(s) can be any suitable standard. For example, the pre-determined marker amount and/or activity measurement(s) can be obtained from the same or a different human for whom a patient selection is being assessed. In some embodiments, the pre-determined marker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

### Cytokines, Chemokines, Growth Factors

Cytokines are small, non-structural proteins of inflammation and immunology. Cytokines affect nearly every biological process; these include embryonic development, disease pathogenesis, non-specific response to infection, specific response to antigen, changes in cognitive functions and progression of the degenerative processes of aging. In addition, cytokines are part of stem cell differentiation, vaccine efficacy and allograft rejection. Multiple biological properties or pleiotropism is the hallmark of a cytokine, and cytokines encompass interferons, the interleukins, chemokines, lymphokines, mesenchymal growth factors, the tumor necrosis factor family and adipokines.

An inflammatory cytokine or proinflammatory cytokine is a type of signaling molecule (a cytokine) that is secreted from immune cells, e.g., helper T cells (Th) and macrophages, and certain other cell types that promote inflammation. They include interleukin-1 (IL-1), IL-12, and IL-18, tumor necrosis factor alpha (TNF-α), interferon gamma (IFNγ), and granulocyte-macrophage colony stimulating factor (GM-CSF) and play an important role in mediating the innate immune response. Inflammatory cytokines are predominantly produced by and involved in the upregulation of inflammatory reactions.

Excessive chronic production of inflammatory cytokines contribute to inflammatory diseases, that have been linked to different diseases, such as atherosclerosis and cancer. Dysregulation has also been linked to depression and other neurological diseases. A balance between proinflammatory and anti-inflammatory cytokines is necessary to maintain health.

Aging and exercise also play a role in the amount of inflammation from the release of proinflammatory cytokines.

The major proinflammatory cytokines that are responsible for early responses are IL-1-alpha, IL-1-beta, IL-6, and TNF-α. Other proinflammatory mediators include members of the IL-20 family, IL-33 LIF, IFN-gamma, OSM, CNTF, TGF-beta, GM-CSF, IL-11, IL-12, IL-17, IL-18, IL-8, Rantes, and a variety of other chemokines that chemoattract inflammator cells. These cytokines either act as endogenous pyrogens (IL-1, IL-6, TNF-α), upregulate the synthesis of secondary mediators and proinflammatory cytokines by both macrophages and mesenchymal cells (including fibroblasts, epithelial and endothelial cells), stimulate the production of acute phase proteins, or attract inflammatory cells.

IL-6 has been shown to play a central role in the neuronal reaction to nerve injury. Suppression of IL-6R by in vivo application of anti-IL-6R antibodies led to reduced regenerative effects. IL-6 is also involved in microglial and astrocytic activation as well as in regulation of neuronal neuropeptides expression. There is evidence that IL-6 contributes to the development of neuropathic pain behavior following a peripheral nerve injury. For example, sciatic cryoneurolysis, a sympathetically-independent model of neuropathic pain involving repeatedly freezing and thawing a section of the sciatic nerve, results in increased IL-6 immunoreactivity in the spinal cord. In addition, intrathecal infusion of IL-6 induces tactile allodynia and thermal hyperalgesia in intact and nerve-injured rats, respectively.

TNF-α, also known as cachectin, is another inflammatory cytokine that plays a well-established, key role in some pain models. TNF acts on several different signaling pathways through two cell surface receptors, TNFR1 and TNFR2 to regulate apoptotic pathways, NF-kB activation of inflammation, and activate stress-activated protein kinases (SAPKs). TNF-α receptors are present in both neurons and glia. TNF-α has been shown to play important roles in both inflammatory and neuropathic hyperalgesia. Intraplantar injection of complete Freund's adjuvant in adult rats resulted in significant elevation in the levels of TNF-α, IL-1β, and nerve growth factor (NGF) in the inflamed paw. A single injection of anti-TNF-α antiserum before the CFA significantly delayed the onset of the resultant inflammatory hyperalgesia and reduced IL-1β but not NGF levels. Intraplantar injection of TNF-α also produces mechanical and thermal hyperalgesia. It has been found that TNF-α injected into nerves induces Wallerian degeneration and generates the transient display of behaviors and endoneurial pathologies found in experimentally painful nerve injury. TNF binding protein (TNF-BP), an inhibitor of TNF, is a soluble form of a transmembrane TNF-receptor. When TNF-BP is administered systemically, the hyperalgesia normally observed after lipopolysaccharide (LPS) administration is completely eliminated. Intrathecal administration of a combination of TNF-BP and IL-1 antagonist attenuated mechanical allodynia in rats with L5 spinal nerve transection.

Rantes, also known as CCL5, is a chemoattractant for blood monocytes, memory T-helper cells and eosinophils. It causes the release of histamine from basophils and activates eosinophils. It may activate several chemokine receptors including CCR1, CCR3, CCR4 and CCR5. It is one of the major HIV-suppressive factors produced by CD8+ T-cells. Recombinant RANTES protein induces a dose-dependent inhibition of different strains of HIV-1, HIV-2, and simian immunodeficiency virus (SIV). The processed form RANTES (3-68) acts as a natural chemotaxis inhibitor and is a more potent inhibitor of HIV-1-infection. The second processed form RANTES (4-68) exhibits reduced chemotactic and HIV-suppressive activity compared with RANTES (1-68) and RANTES (3-68) and is generated by an unidentified enzyme associated with monocytes and neutrophils. Rantes may also be an agonist of the G protein-coupled receptor GPR75, stimulating inositol trisphosphate production and calcium mobilization through its activation. Together with GPR75, Rantes may play a role in neuron survival through activation of a downstream signaling pathway involving the PI3, Akt and MAP kinases. By activating GPR75 may also play a role in insulin secretion by islet cells

Chemokines are a family of small cytokines, or signaling proteins secreted by cells. Their name is derived from their ability to induce directed chemotaxis in nearby responsive cells; they are chemotactic cytokines. In addition to being known for mediating chemotaxis, chemokines are all approximately 8-10 kilodaltons in mass and have four cysteine residues in conserved locations that are key to forming their 3-dimensional shape.

These proteins have historically been known under several other names including the SIS family of cytokines, SIG family of cytokines, SCY family of cytokines, Platelet factor-4 superfamily or intercrines. Some chemokines are considered pro-inflammatory and can be induced during an immune response to recruit cells of the immune system to a site of infection, while others are considered homeostatic and are involved in controlling the migration of cells during normal processes of tissue maintenance or development.

Chemokines are found in all vertebrates, some viruses and some bacteria, but none have been described for other invertebrates.

Chemokines represent a family of low molecular weight secreted proteins that primarily function in the activation and migration of leukocytes although some of them also possess a variety of other functions. Chemokines have conserved cysteine residues that allow them to be assigned to four groups: C—C chemokines (monocyte chemoattractant protein or MCP-1, monocyte inflammatory protein or MIP-1α, and MIP-1β), C—X—C chemokines (IL-8 also called growth related oncogene or GRO/KC), C chemokines (lymphotactin), and CXXXC chemokines (fractalkine).

A growth factor is a naturally occurring substance capable of stimulating cellular growth, proliferation, healing, and cellular differentiation. Usually it is a protein or a steroid hormone. Growth factors are important for regulating a variety of cellular processes.

Growth factors typically act as signaling molecules between cells. Examples are cytokines and hormones that bind to specific receptors on the surface of their target cells. They often promote cell differentiation and maturation, which varies between growth factors. For example, epidermal growth factor (EGF) enhances osteogenic differentiation, while fibroblast growth factors (FGFs) and vascular endothelial growth factors (VEGFs) stimulate blood vessel differentiation (angiogenesis).

Platelet-derived growth factor (PDGF) is one among numerous growth factors that regulate cell growth and division. In particular, PDGF plays a significant role in blood vessel formation, the growth of blood vessels from already-existing blood vessel tissue, mitogenesis, i.e. proliferation, of mesenchymal cells such as fibroblasts, osteoblasts, tenocytes, vascular smooth muscle cells and mesenchymal stem cells as well as chemotaxis, the directed migration, of mesenchymal cells. Platelet-derived growth factor is a dimeric glycoprotein that can be composed of two A subunits (PDGF-AA), two B subunits (PDGF-BB), or one of each (PDGF-AB).

PDGF is a potent mitogen for cells of mesenchymal origin, including fibroblasts, smooth muscle cells and glial cells. In both mouse and human, the PDGF signalling network consists of five ligands, PDGF-AA through -DD (including -AB), and two receptors, PDGFRalpha and PDGFRbeta. All PDGFs function as secreted, disulphide-linked homodimers, but only PDGFA and B can form functional heterodimers.

Though PDGF is synthesized, stored (in the alpha granules of platelets), and released by platelets upon activation, it is also produced by other cells including smooth muscle cells, activated macrophages, and endothelial cells.

### Neurodegenerative Disorders

The compositions and methods described herein can be used in the treatment of a wide range of conditions related to neurodegeneration, and/or neuromuscular disorders. Representative such conditions are described below.

### Neurodegeneration

Neurodegeneration is the progressive loss of structure or function of neurons, including death of neurons. Many neurodegenerative diseases—including amyotrophic lateral sclerosis, Parkinson's disease, Alzheimer's disease, and Huntington's disease—occur as a result of neurodegenerative processes. Such diseases are incurable, resulting in progressive degeneration and/or death of neuron cells. As research progresses, many similarities appear that relate these diseases to one another on a sub-cellular level. Discovering these similarities offers hope for therapeutic advances that could ameliorate many diseases simultaneously.

Alzheimer's Disease Alzheimer's disease is a chronic neurodegenerative disease that usually starts slowly and gradually worsens over time. It is the cause of 60-70% of cases of dementia. The most common early symptom is difficulty in remembering recent events. As the disease advances, symptoms can include problems with language, disorientation (including easily getting lost), mood swings, loss of motivation, not managing self-care, and behavioural issues. As a person's condition declines, they often withdraw from family and society. Gradually, bodily functions are lost, ultimately leading to death. Although the speed of progression can vary, the typical life expectancy following diagnosis is three to nine years.

The cause of Alzheimer's disease is poorly understood. About 70% of the risk is believed to be inherited from a person's parents with many genes usually involved. Other risk factors include a history of head injuries, depression, and hypertension. The disease process is associated with plaques and neurofibrillary tangles in the brain. A probable diagnosis is based on the history of the illness and cognitive testing with medical imaging and blood tests to rule out other possible causes. Initial symptoms are often mistaken for normal ageing. Examination of brain tissue is needed for a definite diagnosis. There are no medications or supplements that have been shown to decrease risk.

No treatments stop or reverse its progression, though some may temporarily improve symptoms. Affected people increasingly rely on others for assistance, often placing a burden on the caregiver. The pressures can include social, psychological, physical, and economic elements. Exercise programs may be beneficial with respect to activities of daily living and can potentially improve outcomes. Behavioural problems or psychosis due to dementia are often treated with antipsychotics, but this is not usually recommended, as there is little benefit with an increased risk of early death.

In 2015, there were approximately 29.8 million people worldwide with Alzheimer's disease. It most often begins in people over 65 years of age, although 4-5% of cases are early-onset Alzheimer's. In 2015, dementia resulted in about 1.9 million deaths. In developed countries, Alzheimer's disease is one of the most financially costly diseases.

### Parkinson's Disease

Parkinson's disease is a long-term degenerative disorder of the central nervous system that mainly affects the motor system. As the disease worsens, non-motor symptoms become more common. The symptoms usually emerge slowly. Early in the disease, the most obvious symptoms are shaking, rigidity, slowness of movement, and difficulty with walking. Thinking and behavioral problems may also occur. Dementia becomes common in the advanced stages of the disease. Depression and anxiety are also common, occurring in more than a third of people with Parkinson's disease. Other symptoms include sensory, sleep, and emotional problems.

The cause of Parkinson's disease is unknown, but is believed to involve both genetic and environmental factors. Those with a family member affected are more likely to get the disease themselves. There is also an increased risk in people exposed to certain pesticides and among those who have had prior head injuries, while there is a reduced risk in tobacco smokers and those who drink coffee or tea. The motor symptoms of the disease result from the death of cells in the substantia nigra, a region of the midbrain. This results in not enough dopamine in this region of the brain. The cause of this cell death is poorly understood, but it involves the build-up of proteins into Lewy bodies in the neurons.

There is no cure for Parkinson's disease. Treatment aims to improve the symptoms. Initial treatment is typically with the antiparkinson medication levodopa (L-DOPA), followed by dopamine agonists when levodopa becomes less effective. As the disease progresses and neurons continue to be lost, these medications become less effective while at the same time they produce a complication marked by involuntary writhing movements.

Diet and some forms of rehabilitation have shown some effectiveness at improving symptoms. Surgery to place microelectrodes for deep brain stimulation has been used to reduce motor symptoms in severe cases where drugs are ineffective.

In 2015, Parkinson's disease affected 6.2 million people and resulted in about 117,400 deaths globally. Parkinson's disease typically occurs in people over the age of 60, of whom about one percent are affected. Males are more often affected than females at a ratio of around 3:2. When it is seen in people before the age of 50, it is called early-onset PD. The average life expectancy following diagnosis is between 7 and 15 years.

### Huntington's Disease

Huntington's disease, also known as Huntington's chorea, is an inherited disorder that results in the death of brain cells. The earliest symptoms are often subtle problems with mood or mental abilities. A general lack of coordination and an unsteady gait often follow. As the disease advances, uncoordinated, jerky body movements become more apparent. Physical abilities gradually worsen until coordinated movement becomes difficult and the person is unable to talk. Mental abilities generally decline into dementia. The specific symptoms vary somewhat between people. Symptoms usually begin between 30 and 50 years of age, but can start at any age. The disease may develop earlier in life in each successive generation. About eight percent of cases start before the age of 20 years and typically present with symptoms more similar to Parkinson's disease.

Huntington's disease is typically inherited, although up to 10% of cases are due to a new mutation. The disease is caused by an autosomal dominant mutation in either of an individual's two copies of a gene called Huntingtin. This means a child of an affected person typically has a 50% chance of inheriting the disease. The Huntingtin gene provides the genetic information for a protein that is also called “huntingtin.” Expansion of CAG (cytosine-adenine-guanine) triplet repeats in the gene coding for the Huntingtin protein results in an abnormal protein, which gradually damages cells in the brain, through mechanisms that are not fully understood.

There is no cure for Huntington's disease. Treatments can relieve some symptoms and in some improve quality of life. The best evidence for treatment of the movement problems is with tetrabenazine. Huntington's disease affects about 4 to 15 in 100,000 people of European descent. The disease affects men and women equally. Complications such as pneumonia, heart disease, and physical injury from falls reduce life expectancy. Suicide is the cause of death in about 9% of cases. Death typically occurs fifteen to twenty years from when the disease was first detected.

### Batten Disease

Batten disease is a fatal disease of the nervous system that typically begins in childhood. Onset of symptoms is usually between 5 and 10 years of age. Often, it is autosomal recessive. It is the most common form of a group of disorders called the neuronal ceroid lipofuscinoses (NCLs). At least 20 genes have been identified in association with Batten disease, but juvenile NCL, the most prevalent form of Batten disease, has been linked to mutations in the CLN3 gene.

### Muscle Atrophy: Loss of Muscle Function and Muscle Mass Due to Denervation

When the information transfer between the nerve cells and the skeletal muscles is impaired or even disrupted, as it happens with degeneration of motor neurons that gradually leads to denervation, the muscles can no longer function properly and begin to become atrophic.

Denervation is an injury to the peripheral neurons with a partial or completion interruption of the nerve fibers between an organ and the central nervous system, resulting in an interruption of nerve conduction and motoneuron firing which, in turn, prevents the contractability of skeletal muscles. This loss of nerve function can be localized or generalized due to the loss of an entire motor neuron unit. The resulting inability of skeletal muscles to contract leads to muscle atrophy; within only a few week weeks, a major part of the muscle mass can be lost, as evidenced by a decrease in muscle weight as well as muscle function.

Muscle Atrophy Resulting from Traumatic Nerve Injury and Neurodegenerative Motoneuron Diseases

Muscle atrophy severely affects the quality of life, as the concerned individuals are impaired or even incapable of performing tasks that involve lifting, walking or running. In motoneuron diseases, the information transmission from motor neurons in the spinal cord to skeletal muscle fibers via somatic motor nerve fibers is impaired or fully interrupted. Motor neurons and muscle fibers interface at the neuromuscular junction. Upon stimulation in vertebrates, the motor neuron releases neurotransmitters that bind to postsynaptic receptors and trigger an excitatory, i.e. contractile, response in the muscle fiber. Since, thus, the contraction of a skeletal muscle can only be prompted through the firing of motor neurons with the transmission of a nerve impulse, an interruption of that transmission means that the skeletal muscle becomes inactive and atrophic over time. The interruption of nerve function can occur in the brain, spinal cord, or a peripheral nerve.

### Motoneuron Diseases

Motoneuron diseases are neurological disorders that selectively and irreversibly destroy motoneurons, the cells that control voluntary muscle activity such as speaking, walking, breathing, swallowing and general movement of the body. Motoneuron diseases are primarily inherited and occur in children as well as adults; they are classified in accordance to whether they affect upper motor neurons, lower motor neurons or both. Motoneuron diseases are generally progressive in nature, and cause gradually increasing disability and death.

### Amyotrophic Lateral Sclerosis.

Amyotrophic lateral sclerosis (Lou Gehrig's Disease; ALS), also known as motor neurone disease, is considered the most common form of a motoneuron disease with an onset in adult age of, in average, about 50-60 years and an incidence of 1:50,000 per year.

ALS results in the death of neurons controlling voluntary muscles. ALS is characterized by stiff muscles, muscle twitching, and gradually worsening weakness due to muscles decreasing in size. It may begin with weakness in the arms or legs, or with difficulty speaking or swallowing. About half of the people affected develop at least mild difficulties with thinking and behavior and most people experience pain. Most eventually lose the ability to walk, use their hands, speak, swallow, and breathe. ALS is a progressive disease with a fatal outcome due to gradual paralysis of all voluntary muscles throughout the body, whereby the breathing and swallowing muscles become affected early on already.

The cause is not known in 90% to 95% of cases, but is believed to involve both genetic and environmental factors. The remaining 5-10% of cases are inherited from a person's parents. The most common familial forms of ALS in adults are caused by mutations of the superoxide dismutase gene, or SOD1, located on chromosome 21. The underlying mechanism involves damage to both upper and lower motor neurons.

No cure for ALS is known. The goal of current treatment is to improve symptoms. A medication called riluzole may extend life by about two to three months. Non-invasive ventilation may result in both improved quality and length of life. Mechanical ventilation can prolong survival but does not stop disease progression. A feeding tube may help. The disease can affect people of any age, but usually starts around the age of 60 and in inherited cases around the age of 50. The average survival from onset to death is two to four years, though this can vary. About 10% survive longer than 10 years. Most die from respiratory failure.

### Exemplary Inhibitors

### RNAi

An RNA interfering agent as used herein, is any agent which interferes with or inhibits expression of a target biomarker gene (e.g., cytokines, chemokines, and/or growth factors) by RNA interference (RNAi). Such RNA interfering agents include, but are not limited to, nucleic acid molecules including RNA molecules which are homologous to the target biomarker gene of the present invention, or a fragment thereof, short interfering RNA (siRNA), and small molecules which interfere with or inhibit expression of a target biomarker nucleic acid by RNA interference (RNAi).

RNA interference (RNAi) is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target biomarker nucleic acid. In certain embodiments, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs, shRNAs, or other RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

Piwi-interacting RNA (piRNA) is the largest class of small non-coding RNA molecules. piRNAs form RNA-protein complexes through interactions with piwi proteins. These piRNA complexes have been linked to both epigenetic and post-transcriptional gene silencing of retrotransposons and other genetic elements in germ line cells, particularly those in spermatogenesis. They are distinct from microRNA (miRNA) in size (26-31 nt rather than 21-24 nt), lack of sequence conservation, and increased complexity. However, like other small RNAs, piRNAs are thought to be involved in gene silencing, specifically the silencing of transposons. The majority of piRNAs are antisense to transposon sequences, suggesting that transposons are the piRNA target. In mammals it appears that the activity of piRNAs in transposon silencing is most important during the development of the embryo, and in both C. elegans and humans, piRNAs are necessary for spermatogenesis. piRNA has a role in RNA silencing via the formation of an RNA-induced silencing complex (RISC).

Short interfering RNA (siRNA), also referred to herein as small interfering RNA is an agent which functions to inhibit expression of a target biomarker nucleic acid, e.g., by RNAi. An siRNA may be chemically synthesized, may be produced by in vitro transcription, or may be produced within a host cell. In certain embodiments, the siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably, the siRNA is capable of promoting RNA interference through degradation or specific post-transcriptional gene silencing (PTGS) of the target messenger RNA (mRNA).

In other embodiments, an siRNA is a small hairpin (also called stem loop) RNA (shRNA). In some embodiments, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA Apr; 9(4):493-501 incorporated by reference herein).

RNA interfering agents, e.g., siRNA molecules, may be administered to a patient having or at risk for having a neurodegenerative disorder, to inhibit expression of a biomarker gene (e.g., cytokines, chemokines, and/or growth factors) which is overexpressed in a neurodegenerative disease (e.g., ALS) and thereby treat, prevent, or inhibit the neurodegenerative disease in the subject.

Genome Editing by Crispr In addition to RNAi, genome editing can be used to modulate the copy number or genetic sequence of a biomarker of interest, such as constitutive or induced knockout or mutation of a biomarker of interest, such as cytokines/chemokines/growth factors. For example, the CRISPR-Cas system can be used for precise editing of genomic nucleic acids (e.g., for creating non-functional or null mutations). In such embodiments, the CRISPR guide RNA and/or the Cas enzyme may be expressed. For example, a vector containing only the guide RNA can be administered to an animal or cells transgenic for the Cas9 enzyme. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

### Aptamer

Aptamers are oligonucleotide or peptide molecules that bind to a specific target molecule. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. “Peptide aptamers” are artificial proteins selected or engineered to bind specific target molecules. These proteins consist of one or more peptide loops of variable sequence displayed by a protein scaffold. They are typically isolated from combinatorial libraries and often subsequently improved by directed mutation or rounds of variable region mutagenesis and selection. The Affimer protein, an evolution of peptide aptamers, is a small, highly stable protein engineered to display peptide loops which provides a high affinity binding surface for a specific target protein. It is a protein of low molecular weight, 12-14 kDa, derived from the cysteine protease inhibitor family of cystatins. Aptamers are useful in biotechnological and therapeutic applications as they offer molecular recognition properties that rival that of the commonly used biomolecule, antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications.

### Peptidomimetic Inhibitors

Peptidomimetic inhibitors may be used to decrease the function and/or activity of a cytokine, chemokine and/or growth factor by, for example, competing with the binding of its natural binding partners. Peptidomimetics (Fauchere (1986) Adv. Drug Res. 15:29; Veber and Freidinger (1985) TINS p. 392; and Evans et al. (1987) J. Med. Chem. 30:1229, which are incorporated herein by reference) are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of: —CH2NH—, —CH2S-, —CH2-CH2-, —CH═CH— (cis and trans), —COCH2-, —CH(OH)CH2-, and —CH2SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982)(—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference. A particularly preferred non-peptide linkage is —CH2NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds to produce the therapeutic effect. Derivatization (e.g., labeling) of peptidomimetics should not substantially interfere with the desired biological or pharmacological activity of the peptidomimetic.

### Small Molecules

Also encompassed by the present invention are small molecules which can modulate (either enhance or inhibit) interactions. For example, small molecules such as those described or contemplated herein may bind to the cytokine, chemokine, and/or growth factor and prevent its interaction with its natural binding partners. Alternatively, the small molecules may target the natural binding partners of the cytokines, chemokines, and/or growth factors provided herein. The small molecules of the present invention can be obtained using any of the numerous approaches in combinatorial library methods known in the art, including: spatially addressable parallel solid phase or solution phase libraries; synthetic library methods requiring deconvolution; the ‘one-bead one-compound’ library method; and synthetic library methods using affinity chromatography selection. (Lam, K. S. (1997) Anticancer Drug Des. 12:145).

### Antibody

Unless otherwise specified here within, antibody or antibodies broadly encompass naturally-occurring forms of antibodies (e.g. IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody. An antibody refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. Framework or FR residues are those variable-domain residues other than the hypervariable residues as herein indicated. The variable regions of the heavy and light chains contain a binding domain that interacts with an antigen.

The definitional CDR boundaries and lengths are subject to different classification and numbering systems. CDRs may therefore be referred to by Kabat, Chothia, contact or any other boundary definitions. Despite differing boundaries, each of these systems has some degree of overlap in what constitutes the hypervariable regions within the variable sequences. CDR definitions according to these systems may therefore differ in length and boundary areas with respect to the adjacent framework region. See for example Kabat, Chothia, and/or MacCallum et al., (Kabat et al., in “Sequences of Proteins of Immunological Interest,” 5^{th} Edition, U.S. Department of Health and Human Services, 1992; Chothia et al. (1987) J. Mol. Biol. 196, 901; and MacCallum et al., J. Mol. Biol. (1996) 262, 732, each of which is incorporated by reference in its entirety).

Antibody as used herein also includes antigen-binding portion of an antibody. The antigen-binding portion refers to one or more fragments of an antibody that retain the ability to specifically bind to an antigen (e.g., a cytokine, chemokine, and/or growth factor).

It has been shown that the antigen-binding function of an antibody can be performed by fragments of a full-length antibody. Examples of binding fragments encompassed within the antigen-binding portion of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the antigen-binding portion of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov, S. M., et al. (1995) Human Antibodies and Hybridomas 6:93-101) and use of a cysteine residue, a marker peptide and a C-terminal polyhistidine tag to make bivalent and biotinylated scFv polypeptides (Kipriyanov, S. M., et al. (1994)Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g., humanized, chimeric, etc.). Antibodies may also be fully human. In one embodiment, antibodies of the present invention bind specifically or substantially specifically to a cytokine, chemokine, and/or growth factor provided herein. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

As used herein, the term “Fc region” is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. Suitable native-sequence Fc regions for use in the antibodies of the present invention include human IgG1, IgG2 (IgG2A, IgG2B), IgG3 and IgG4.

Humanized antibody is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. Humanized antibodies may include amino acid residues not encoded by human germline immunoglobulin sequences (e.g., mutations introduced by random or site-specific mutagenesis in vitro or by somatic mutation in vivo), for example in the CDRs. The humanized antibody, as used herein, also includes antibodies in which CDR sequences derived from the germline of another mammalian species, such as a mouse, have been grafted onto human framework sequences.

An isolated antibody refers to an antibody which is substantially free of other antibodies having different antigenic specificities. In addition, an isolated antibody is typically substantially free of other cellular material and/or chemicals.

### Commercially Available Inhibitors

Antibodies suitable for detecting and inhibiting cytokines, chemokines, and growth factors are well known in the art and include, for example, anti-IL-6 antibodies Cat #130-096-086, 130-096-093, 130-096-088, 130-099-646, 130-100-237, 130-108-048, 130-117-590, 130-117-589, 130-117-440, 130-117-592, 130-117-441, 130-117-442, 130-117-443, 130-117-591 (Miltenyi Biotec B.V. & Co. KG), antibody Cat #ITA6172 (G-Biosciences), antibodies Cat #MQ2-13A5, 10121-01, 10120-01 (SouthemBiotech), antibodies Cat #AM05676AF-N, AM33372PU-S, AM33372PU-T, AP26368PU-N, PP012B1, PP012B2, PP012P1, PP012P2, SM012AX, SM1695A, SM1695AX, TA319506, TA328488, TA328489, TA352780, AM31374AF-N, PP036B1, PP036B2, PP036P1, PP036P2, TA328572, TA328573, TA328574, AM05873PU-N, PP1028B1, PP1028B2, PP1028P1, PP1028P2, SP1250, AM05235FC-N, AM05235PU-N, AM05241BT-N, AM05241PU-N, AM05403PU-N, AM05404BT-N, AM05404PU-N, AM06012PU-N, AM06090SU-N, AM26304PU-N, AM26383PU-L, AM26384PU-L, AM26385PU-L, AM26386PU-L, AM26387PU-L, AM26388PU-L, AM26715PU-N, AM31262AF-N, AM31351AF-N, AM31351RP-N, AM50337PU-N, AM50337PU-S, AP01157BT-N, AP01157BT-S, AP01157PU-N, AP01157PU-S, AP52199PU-N, CF500066, CF500067, CF500071, SP2128A, TA319282, TA328214, TA328215, TA328216, TA328217, TA341222, TA341223, TA354416, TA500065, TA500066, TA500067, TA500071, TA500071S, TA590788, TA600023, TA700023 (OriGene), antibodies Cat #MAB206, AF-206-NA, AF-406-NA, NB600-1131, AF506, MAB2061, NBP-47810, NBP2-44953, AF686 (Novus Biologicals), and many more. Moreover, multiple siRNA, shRNA, and CRISPR constructs for reducing or eliminating IL-6 expression can be found in the commercial product lists of the above-referenced companies as well as many others, such as sgRNA CRISPR lentivector Cat #K1076401 to K1076408, K4709801 to K4709808, K7555701 to K7555708, sgRNA lentivirus Cat #K1076411 to K1076418, K4709811 to K4709818, K7555711 to K7555718, sgRNA CRISPR adenovirus Cat #K1076421, K2709821, K7555721, IL-6 CRISPR sgRNA AAV virus Cat #K107648101 to K107648111, K107648210 to K107648211, K107648301 to K107648311 (serotypes 1-11) (Abm), CRISPR knockouts Cat #KN202078, KN202078BN, KN202078LP, KN202078RB, KN308293, KN308293BN, KN308293LP, KN308293RB, KN402078, KN508293 (OriGene), miRNAs Cat #hsa-let-7a-5p (MIRT005420), hsa-mir-203a-3p (MIRT006061), hsa-mir-142-3p (MIRT006790), hsa-mir-26a-5p (MIRT007374), hsa-mir-335-5p (MIRT016949), hsa-mir-365a-3p (MIRT020073), hsa-mir-155-5p (MIRT021049), hsa-mir-124-3p (MIRT022183), hsa-mir-1-3p (MIRT023501), hsa-mir-98-5p (MIRT027449), bta-mir-15b (MIRT053807), bta-mir-16a (MIRT053833), bta-mir-155 (MIRT053955), bta-mir-223 (MIRT054007), hsa-mir-107 (MIRT054777), hsa-let-7c-5p (MIRT438050), hsa-mir-223-3p (MIRT438284), hsa-mir-149-5p (MIRT438344), mmu-let-7a-5p (MIRT438710), hsa-let-7f-5p (MIRT731223), hsa-mir-146b-5p (MIRT731232), hsa-mir-9-5p (MIRT731346), hsa-mir-146a-5p (MIRT732454), hsa-mir-125a-3p (MIRT733375), hsa-mir-106a-5p (MIRT734163), hsa-mir-136-5p (MIRT734880), hsa-mir-451a (MIRT735462) (miRTarBase). Additional constructs may be constructed using the sequences that are publicly available (e.g., NCBI).

Similarly, anti-TNF-α antibodies for detecting and inhibiting TNF-α are well known in the art and commercially available. For example, Anti TNF-α Antibody (130-095-749), Anti TNF-α Antibody (130-120-628), Anti TNF-α Antibody (130-120-489), Anti TNF-α Antibody (130-117-382), Anti TNF-α Antibody (130-120-490), Recombinant Anti TNF-α Antibody (130-119-151), Recombinant Anti TNF-α Antibody (130-118-974), Recombinant Anti TNF-α Antibody (130-120-063), Anti TNF-α Antibody (130-117-531), Anti TNF-α Antibody (130-120-491), Anti TNF-α Antibody (130-120-627), Recombinant Anti TNF-α Antibody (130-120-149), Anti TNF-α Antibody (130-120-629), Anti TNF-α Antibody (130-120-630), Anti TNF-α Antibody (130-120-492) (Miltenyi Biotec B. V. & Co. KG); anti-TNF-α antibodies TNFα (J1D9), TNFα (J1D9) L, TNFα (52B83), TNFα (AS1), TNFα (E7D2), TNFα (4E1), TNFα (C-4), TNFα (E-4) (Santa Cruz Biotechnology); Cat #A00002-2, A00002-3, PA1079, PB9010, PB9246, M00002, RP1000 (Boster Bio Antibodies); Cat #AF-410-NA, NBP1-19532, NBP2-75925, MAB610, NB600-587, AF-510-NA, AF-210-NA, NB600-1433, MAB4101, MAB210 (Novus Biologicals), and many more are available commercially. Moreover, multiple siRNA, shRNA, and CRISPR constructs for reducing or eliminating TNF-α expression can be found in the commercial product lists of the above-referenced companies as well as many others, such as sgRNA CRISPR lentivector Cat #K2424001 to K2424008, K5039001 to K5039008, K7626301 to K7626308, sgRNA CRISPR lentivirus Cat #K2414011 to K2414018, K5039011 to K5039018, K7626311 to K7626318, sgRNA CRISPR adenovirus Cat #K2414021, K5039021, K7626321, sgRNA CRISPR AAV Cat #K241408101 to K241408311 (Serotypes 1-11), CRISPR sgRNA AAV vectors Cat #K241408100, K241408200, K241408300 (Abm); miRNAs Cat #hsa-mir-19a-3p (MIRT006787), hsa-mir-203a-3p (MIRT006857), hsa-mir-452-5p (MIRT053456), bta-mir-15b (MIRT053814), bta-mir-16a (MIRT053838), bta-mir-21-5p (MIRT053891), bta-mir-146a (MIRT053927), bta-mir-146b (MIRT053943), bta-mir-155 (MIRT053960), bta-mir-223 (MIRT054009), hsa-mir-187-3p (MIRT054325), hsa-mir-130a-3p (MIRT054762), hsa-mir-143-3p (MIRT437418), hsa-mir-125b-5p (MIRT733472), hsa-mir-24-3p (MIRT733712), hsa-mir-34a-5p (MIRT733989), hsa-mir-17-5p (MIRT734730) (miRTarBase). Additional constructs may be constructed using the sequences that are publicly available (e.g., NCBI).

Anti-IFN-g antibodies for detecting and inhibiting IFN-g are well known in the art and commercially available. For example, Anti IFN-g Antibody (130-095-743) Recombinant Anti IFN-g Antibody (130-111-769) Recombinant Anti IFN-g Antibody (130-111-935) Recombinant Anti IFN-g Antibody (130-114-025) Recombinant Anti IFN-g Antibody (130-113-499) Anti IFN-g Antibody (130-114-019) Recombinant Anti IFN-g Antibody (130-113-498) Recombinant Anti IFN-g Antibody (130-114-024) Anti IFN-g Antibody (130-113-493) Recombinant Anti IFN-g Antibody (130-113-497) Anti IFN-g Antibody (130-114-017) Anti IFN-g Antibody (130-113-492) Anti IFN-g Antibody (130-114-018) Anti IFN-g Antibody (130-113-491) Recombinant Anti IFN-g Antibody (130-114-023) Recombinant Anti IFN-g Antibody (130-113-495) Anti IFN-g Antibody (130-113-494) Anti IFN-g Antibody (130-114-016) Recombinant Anti IFN-g Antibody (130-119-676) Recombinant Anti IFN-g Antibody (130-113-496) Recombinant Anti IFN-g Antibody (130-114-022) Anti IFN-g Antibody (130-113-490) Anti IFN-g Antibody (130-114-020) Recombinant Anti IFN-g Antibody (130-119-577) Recombinant Anti IFN-g Antibody (130-114-021) (Miltenyi Biotec B.V. & Co. KG); Cat #orb313869, orb10877, orb501002, orb389132, orb214082 (Biorbyt); Cat #IFN-γ (G-23), IFN-γ (3F1E3), IFN-γ (4S.B3), IFN-γ (H3-1), IFN-γ (A35), IFN-γ (B27), IFN-γ (F12), IFN-γ (G-30), IFN-γ (500-M90), IFN-γ (NYRhIFNγ), IFN-γ (LLO6Z), IFN-γ (E8H1), IFN-γ (D-3), IFN-γ (E-10), IFN-γ (A-9) (Santa Cruz Biotechnology), and many more are available commercially.

Moreover, multiple siRNA, shRNA, and CRISPR constructs for reducing or eliminating IFN-g expression can be found in the commercial product lists of the above-referenced companies as well as many others, such as sgRNA CRISPR lentivector set Cat #K1020801 to K1020808, K4437501 to K444437508, K6888601 to K688608, sgRNA CRISPR lentivirus set Cat #K1020811 to K1020818, K4437511 to K4437518, K6888611 to K6888618, sgRNA CRISPR adenovirus Cat #K1020821, K4437521, K6888621, sgRNA CRISPR AAV vector Cat #K102088100, K102088200, K102088300, K443758100, K443758200, K443758300, K688868100, K688868200, K688868300 (Abm); CRISPR knockout Cat #KN409993, KN508142 (OriGene); IFN-γ CRISPR/Cas9 KO Plasmid (h) IFN-γ CRISPR Activation Plasmid (h) IFN-γ CRISPR Activation Plasmid (h2) (Santa Cruz Biotechnology), miRNAs Cat #hsa-mir-16-5p (MIRT006912), hsa-mir-15a-5p (MIRT006913), hsa-mir-15b-5p (MIRT006914), hsa-mir-29b-3p (MIRT007034), hsa-mir-409-3p (MIRT007285), hsa-mir-26b-5p (MIRT029122), hsa-mir-125a-5p (MIRT437825), hsa-mir-27a-3p (MIRT437945), hsa-mir-181a-5p (MIRT437969), hsa-mir-24-3p (MIRT437970), ssc-mir-27b-3p (MIRT734368) (miRTarBase). Additional constructs may be constructed using the sequences that are publicly available (e.g., NCBI).

Anti-Rantes antibodies for detecting and inhibiting IFN-g are well known in the art and commercially available. For example, Recombinant Anti CCL5 (RANTES) Antibody (130-105-451), Recombinant Anti CCL5 (RANTES) Antibody (130-105-493), Recombinant Anti CCL5 (RANTES) Antibody (130-105-494), Recombinant Anti CCL5 (RANTES) Antibody (130-105-450) (Miltenyi Biotec); Anti-CCL5 (aa 24-91) polyclonal antibody (DPABH-06355), Anti-CCL5 polyclonal antibody (CPBT-65251RH), Anti-CCL5 monoclonal antibody clone 32529 (DCABH-201789), biotinylated anti-CCL5 polyclonal antibody (DPABY-440), Anti-CCL5 monoclonal antibody clone 32529 (DCABY-3941) (Creative Diagnostics); RANTES (CCL5) Antibody (515502), RANTES (CCL5) Antibody (515505), RANTES (CCL5) Antibody (515506), PerCP/Cyanine5.5 RANTES (CCL5) Antibody (515507), PerCP/Cyanine5.5 RANTES (CCL5) Antibody (515508), RANTES (CCL5) Antibody (515503), RANTES (CCL5) Antibody (515504), CCL5 (RANTES) Antibody (526402), CCL5 (RANTES) Antibody (519703) (BioLegend); Rantes antibody (AF-278-NA), Rantes antibody (MAB678), Rantes antibody (AF478), Rantes antibody (MAB478), Rantes antibody (MAB278), Rantes antibody (MAB4781), Rantes antibody (AF3819), Rantes antibody (AF1010), Rantes antibody (MAB2781), Rantes antibody (AB-278-NA), Rantes antibody (MAB60451), Rantes antibody (MAB6045), Rantes antibody (NB120-10394) (Novus Biologicals), and many more are available commercially.

Moreover, multiple siRNA, shRNA, and CRISPR constructs for reducing or eliminating Rantes expression can be found in the commercial product lists of the above-referenced companies as well as many others, such as Rantes human gene CRISPR knockout kit (KN403799), CCL5 mouse gene CRISPR knockout kit (KN502795) (OriGene); CRISPR AAV particles targeting human Rantes (HCP216626, HCP263848), CRISPR AAV particles targeting mouse Rantes (MCP230981), CRISPR AAV particles targeting rat Rantes (RCP251023), CRISPR lentiviral particles targeting human Rantes (HCP216626, HCP263848), CRISPR lentiviral particles targeting mouse Rantes (MCP230981), CRISPR lentiviral particles targeting rat Rantes (RCP251023) (GeneCopoeia); RANTES (CCL5) Human siRNA Oligo Duplex (SR304277), RANTES (CCL5) Human shRNA Plasmid Kit (TG319885), Ccl5 Rat shRNA Lentiviral Particle (TL711240V), Ccl5 Mouse siRNA Oligo Duplex (SR400775), Ccl5 Mouse shRNA Plasmid (TG519130), Ccl5 Rat siRNA Oligo Duplex (SR507234), RANTES (CCL5) Human shRNA Plasmid Kit (TL319885), RANTES (CCL5) Human shRNA Lentiviral Particle (TL319885V), RANTES (CCL5) Human shRNA Plasmid Kit (TR319885) (OriGene); miRNAs that target CCL5, such as hsa-mir-98-5p (MIRT005677) hsa-mir-125a-5p (MIRT005678) hsa-mir-214-3p (MIRT053786) hsa-mir-146a-5p (MIRT437504) hsa-mir-5089-5p (MIRT518342) hsa-mir-7151-3p (MIRT518343) hsa-mir-5095 (MIRT518344) hsa-mir-4775 (MIRT518345) hsa-mir-6508-3p (MIRT518346) hsa-mir-4726-5p (MIRT518347) hsa-mir-4640-5p (MIRT518348) hsa-mir-6853-5p (MIRT518349) hsa-mir-6506-5p (MIRT518350) hsa-mir-619-5p (MIRT518351) hsa-mir-4735-5p (MIRT518352) hsa-mir-6504-3p (MIRT518353) hsa-mir-4438 (MIRT518354) hsa-mir-590-3p (MIRT518355) hsa-mir-6807-5p (MIRT685341) hsa-mir-6720-5p (MIRT685342) hsa-mir-6512-3p (MIRT685343) hsa-mir-5589-5p (MIRT685344) hsa-mir-4731-5p (MIRT685345) hsa-mir-186-3p (MIRT685346) hsa-mir-6849-3p (MIRT685347) hsa-mir-520h (MIRT685348) hsa-mir-520g-3p (MIRT685349) hsa-mir-512-3p (MIRT685350) hsa-mir-520e (MIRT685351) hsa-mir-520d-3p (MIRT685352) hsa-mir-520c-3p (MIRT685353) hsa-mir-520b (MIRT685354) hsa-mir-520a-3p (MIRT685355) hsa-mir-373-3p (MIRT685356) hsa-mir-372-3p (MIRT685357) hsa-mir-302e (MIRT685358) hsa-mir-302d-3p (MIRT685359) hsa-mir-302c-3p (MIRT685360) hsa-mir-302b-3p (MIRT685361) hsa-mir-302a-3p (MIRT685362) hsa-mir-93-5p (MIRT685363) hsa-mir-526b-3p (MIRT685364) hsa-mir-519d-3p (MIRT685365) hsa-mir-20b-5p (MIRT685366) hsa-mir-20a-5p (MIRT685367) hsa-mir-17-5p (MIRT685368) hsa-mir-106b-5p (MIRT685369) hsa-mir-106a-5p (MIRT685370) hsa-let-7g-3p (MIRT732867) (miRTarBase). Additional constructs may be constructed using the sequences that are publicly available (e.g., NCBI).

Similarly, antibodies, RNAi products, CRISPR products, ELISA detection kits for detecting and inhibiting other cytokines, chemokines, and growth factors are available from the above-referenced companies as well as other commercial vendors.

### Gene Delivery

The instant inventions use gene delivery methods to introduce nucleic acid into cells (e.g., an exogenous nucleic acid molecule encoding shRNA or sgRNA in order to eliminate expression of a cytokine, chemokine, and/or growth factor). Any means for the introduction of a polynucleotide into mammals, human or non-human, or cells thereof may be adapted to the practice of this invention for the delivery of the various constructs of the present invention into the intended recipient. In some embodiments of the present invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), lentivirus, and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest may be in a plasmid or viral vector, or other vector as is known in the art. Such vectors are well known and any can be selected for a particular application. In certain embodiments of the present invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter. A promoter may be constitutive or inducible.

In other embodiments, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J. Biol. Chem. 264:16985-16987, 1989), lipid-DNA combinations (Felgner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In preferred embodiments, the growth factor gene delivery vehicle is a recombinant retroviral vector.

Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Other viral vector systems that can be used to deliver a polynucleotide of the present invention have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth,; Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al. (1990) J. Virol., 64:642-650).

In other embodiments, target DNA in the genome can be manipulated using well-known methods in the art. For example, the target DNA in the genome can be manipulated by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis.

Other Therapies for Neurodegenerative Disorders There is no treatment that can cure neurodegenerative diseases, but there are symptomatic treatments. These include dopaminergic treatments for Parkinson's disease and movement disorders, cholinesterase inhibitors for cognitive disorders, antipsychotic drugs for behavioral and psychological symptoms of dementia, analgesic drugs for pain, and even the use of deep brain stimulation to stop tremor and refractory movement disorders. Researchers have also aimed to produce medicines to slow the development of diseases, such as Riluzole for ALS, cerebellar ataxia and Huntington's disease, NSAIDs (nonsteroidal anti-inflammatory drugs) for Alzheimer's disease, and caffein A2A receptor antagonists and CERE-120 (adeno-associated virus serotype 2-neurturin) for the neuroprotection of Parkinson's disease.

In some embodiments, a condition such as neurodegenerative disorder is responsive to blockade of cytokines, chemokines, and/or growth factors alone. In certain such embodiments, the condition is significantly or synergistically more responsive when treated in combination with another therapy.

### Alzheimer's Disease (Ad)

Currently, there is no known cure for AD, and the drugs used within the scope of this disease are mainly to treat the cognitive manifestations or other symptoms and function better when administered at an early stage. One of the drugs that is marketed for the treatment of AD, galantamine was repurposed. In fact, this alkaloid, present in Galanthus sp., aroused interest when it was found that it could inhibit muscle acetylcholinesterase, being a good candidate for treating myopathies and peripheral neuropathies, and for the reversal of neuromuscular blockade after anaesthesia, due to the capability of galantamine to enhance nerve impulse transmission. As it has a tertiary ammonium base, galantamine can easily penetrate the blood-brain barrier and inhibit brain acetylcholinesterase.

Antimicrobials have also been studied for their potential suitability to treat AD and their symptoms. Both azithromycin and erythromycin, macrolide antibiotics, have shown inhibition of the amyloid precursor protein, resulting in the decrease of cerebral levels of amyloid-D. Tetracyclines have also been proven to reduce the formation of amyloid-0, as well as its resistance to trypsin digestion and an increase in the disassembly of preformed fibrils. They also decreased oxidative stress, suggesting a varied mechanism of action.

Doxycycline has shown potential in this respect, both alone and in combination with rifampicin. Rifampicin, most frequently prescribed for Mycobacterium infections, has shown effects in the reduction of amyloid-β fibrils, in a dose-dependent manner, probably due to the decreased production and increased clearance of amyloid-D. Dapsone is an antibiotic used to treat leprosy, and also received attention when a decreased incidence of dementia was noticed in leprosy patients that had been treated with dapsone. Conflicting data about whether or not dapsone was capable of decreasing senile plaques led to the hypothesis that this event could be a protective factor against amyloid deposition. This hypothesis was further corroborated by studies that showed similar instances of AD in leprosy and tuberculosis patients, in spite of the differences in the percentage of patients that have undergone drug treatments in the two groups. The antiviral drugs acyclovir, penciclovir and foscamet have been successful in reducing phosphorylated tau protein and amyloid-β in AD cell models, which can mean they are suitable for the treatment of AD. Clioquinol is an antifungal and antiparasitic drug that has been shown to cause a reduction in the amyloid-β plaques in the brain, with good tolerability in transgenic mice.

The antiepileptic drug valproic acid has been suggested as a neuroprotective agent for AD, as it has shown reduced formation of amyloid-β plaques and improvement in memory deficits in transgenic mice. The proposed mechanism of action was shown to be complex, but it might be through the enhancement of microglial phagocytosis of amyloid-D.

Valsartan is an angiotensin receptor blocker, and is used as an antihypertensive. The rationale behind the use of this class of drugs for AD comes from the fact that chronic adverse stress, one of the major environmental causes for the onset and progression of AD, is capable of causing elevations in brain angiotensin II, which act at AT1 and AT2 receptor subtypes. Furthermore, angiotensin II increases have been suggested to be linked with amyloidogenesis, and the use of angiotensin receptor blockers, blocking AT1, appears to be useful in delaying decline in cognitive processing. Apart from this mechanism of action, valsartan also inhibits inflammation, vasoconstriction and mitochondrial dysfunction, and promotes the release of acetylcholine. Reduced amyloid-β has been reported with in vitro and in vivo treatment of valsartan, and this evidence suggests a reduction of dementia. Additionally, this drug has good brain penetration, but further studies are required before this drug can be included in the therapy of AD. Calcium channel blockers are drugs used to treat hypertension and angina. The dihydropyridine calcium channel blockers, such as nilvadipine, can reduce the production, oligomerization and accumulation of amyloid-β in vitro, improve cell survival and reduce neurotoxicity, while having good blood-brain barrier penetration and increasing brain blood flow through its vasodilatory properties.

Trimetazidine is an anti-ischemic drug of the piperazine class. Its mechanism of action is diverse, ranging from increasing nitric oxide production, inhibiting cell apoptosis and being an antioxidant, which increases endothelial function. Apart from being able to pass through the blood-brain barrier, it can reduce the produce of free radicals, due to its antioxidant properties. It can also improve axonal regeneration and effective myelination in healthy and injured nerves.

Antidiabetics have also been repurposed for AD, since type 2 diabetes has been identified as a risk factor for AD. Studies have reported a desensitization of insulin signalling in the brains of AD patients. Insulin can also induce neuronal stem cell activation and cell growth and repair, and treatment with insulin has shown neuroprotection and a regulation on the levels of phosphorylated tau protein, as well as an improvement in memory and cognition. Given this, compounds that influence insulin release can also be useful for AD. Glucagon-like peptide 1 analogues, which promote insulin secretion, may also act in many pathways related to AD, such as the reduction of amyloid-0 and the impairment of neuronal function and cell death, as well as tau phosphorylation. Liraglutide meets these criteria, has established brain penetration and shows physiological effects in the brain, improving learning, and reducing amyloid-β formation and brain inflammation.

Other therapies include Ghrelin, hexarelin and its derivative EP80317, retinoid receptor activators, retinoic acid, zileuton (a drug that acts through the blockage of 5-lipooxygenase), sildenafil, tadalafil, and trazodone.

Parkinson's Disease (Pd) Nilotinib is a tyrosine kinase Abl inhibitor that is used for the treatment of chronic myeloid leukaemia. It was observed that Abl is activated in neurodegeneration through the increase in α-synuclein expression and, therefore, its accumulation. Since nilotinib inhibits Abl phosphorylation, it increases α-synuclein degradation Zonisamide is a sulphonamide antiepileptic drug, with a mixed mechanism of action, which makes it appropriate for use in different disorders. These mechanisms of action include the blockage of sodium and calcium channels, modulation of the GABAA receptor, inhibition of carbonic anhydrase and inhibition of glutamate release. Studies with rats have shown an increase in dopamine in the striatum when therapeutic doses were used. On the other hand, when higher doses were used, a decrease in intracellular dopamine was observed. Concerning PD, this drug has displayed good activity in both motor and non-motor symptoms, but the mechanism of action is still unclear. Zonisamide is also a monoamine oxidase-B inhibitor. This enzyme, mostly present in astrocytes, is responsible for the degradation of dopamine in neural and glial cells, which ultimately leads to the generation of free radicals, which can play a determinant role in the pathogenesis of PD. Its inhibition makes dopamine levels in the synaptic cleft stable and increases the effect of dopamine.

Methylphenidate is a central nervous system stimulant that acts through the blockage of the presynaptic dopamine transporter and the noradrenaline transporter, thus inhibiting dopamine and noradrenaline reuptake, in the striatum and the prefrontal cortex. It has been used to treat attention-deficit hyperactivity disorder. Multiple studies with this drug have shown that it is effective in reducing gait disorders of PD, as well as non-motor symptoms.

β2-adrenoreceptor agonists, have been studied for their anti-PD activity. Recent findings have linked the β2-adrenoreceptor with the regulation of the α-synuclein gene SNCA. More specifically, β2-adrenoreceptor activation was shown to display neuroprotection. From the drugs tested, three anti-asthmatics were the most promising, with salbutamol being the one capable of penetrating the blood-brain barrier and currently approved for treatment. The study undertaken showed that all three drugs were able to reduce the SNCA-mRNA and α-synuclein abundance.

### Huntington's Disease (Hd)

Tetrabenazine was first developed as part of research aiming to design simple compounds with reserpine-like antipsychotic activity, acting as a high-affinity, reversible inhibitor of monoamine uptake of presynaptic neurons, and as a weak blocker of the D2 dopamine postsynaptic neurons. Antipsychotic studies with this compound were equivocal, and this drug was then repurposed for diseases that manifest themselves by abnormal, involuntary hyperkinetic movements, such as HD. Furthermore, tetrabenazine is safer to use in HD than dopamine receptor blocker, since it has never been documented to cause dyskinetic symptoms. Given this, other drugs with dopamine antagonistic activity have been tested for the treatment of HD. This is the case of tiapride, a D2 receptor antagonist, used as an antipsychotic. However, in Europe, selegiline is a frequent choice for the treatment of Huntington's chorea. Clozapine is a neuroleptic drug used in the treatment of schizophrenia. It displays a high affinity for the dopamine D1 and D4 receptors, with low antagonistic activity for the D2 dopaminergic receptors. Due to its low incidence of extrapyramidal side effects, it was suggested to be a good symptomatic drug for chorea, although clinical trials showed conflicting results. Olanzapine, another antipsychotic drug, is also widely prescribed for the treatment of the motor and behavioural symptoms of HD. This drug has high affinity for serotoninergic receptor, but antagonizes dopamine D2 receptors. It is also safe and well tolerated, and can be recommended when irritability, sleep dysfunction and weight loss are present, as well as chorea. The antipsychotic risperidone, used in the treatment of schizophrenia and bipolar disorder, acts as a D2 receptor antagonist and a serotonin agonist, and therefore can be used for the treatment of HD chorea, as well. It showed beneficial effects on stabilizing motor decline and psychiatric symptoms.

Memantine is an adamantane derivative used for the treatment of AD. It is a non-competitive N-methyl-d-aspartate (NMDA) inhibitor. Excessive stimulation of NMDA receptor causes a great influx of calcium into the cell, which ultimately leads to cell death.

Therefore, memantine can prevent this calcium influx in neuronal cells, and prevent cerebral cell death. Memantine was studied for its efficacy in the treatment of HD, and it was noticed that it was able to decrease the vulnerability of neurons to glutamate-mediated excitotoxicity.

### Multiple Sclerosis (Ms)

Wide arrays of anticancer drugs have been repurposed for the treatment of MS and its symptoms. This is the case of the synthetic compounds mitoxantrone, an anthracenedione, established as a wide-spectrum antitumor agent used to treat breast and prostate cancer, acute leukaemia and lymphoma. Mitoxantrone has also been approved for the treatment of MS, particularly due to its immunosuppressant properties, associated with erratic responses of the central nervous system T- and B-cells to antigens, myelin damage mediated by macrophages, and axonal injuries. Mitoxantrone is capable of inhibiting the activation of T-cells, stopping the proliferation of T- and B-cells, lowering antibody production and deactivating macrophages. Mitoxantrone also displayed high tolerability. The alkylating agent cyclophosphamide is used to treat a variety of solid tumours, and is approved for the treatment of leukaemia, lymphomas, and breast carcinoma, among others. It is related to nitrogen mustards and binds to DNA, interfering with mitosis and cell replication, targeting mostly rapidly dividing cells. Its use in MS comes from cyclophosphamide being able to play an immunosuppressive and immunomodulatory role. Explicitly, it acts in T- and B-cells, supressing cell-mediated and humoral immunity. Cyclophosphamide can also permeate the blood-brain barrier, having a good bioavailability in the central nervous system, being able to exert its activity on neurons, thus stabilizing and preventing the progression of the disease.

Amiloride is a diuretic drug used to treat hypertension and swelling caused by heart failure or liver diseases. It has been studied for its neuroprotective properties in MS. Amiloride can block the neuronal proton-gated acid-sensing ion channel 1 (ASIC1), which is overexpressed in axons and oligodendrocytes in MS lesions, thus exerting its neuroprotective and myeloprotective effects.

The drug ibudilast was approved in some countries for the treatment of bronchial asthma and cerebrovascular disorders. It acts through the inhibition of phosphodiesterases, but can also inhibit leukotriene and nitric oxide synthesis mechanisms, which are connected to MS. In the brain, ibudilast can inhibit the release of the tumour necrosis factor from the microglia and the astrocytes, decreasing neuronal degeneration. Furthermore, it can protect astrocytes from apoptosis and inhibit oligodendrocyte apoptosis and demyelination, hence its usefulness in MS. Studies have shown its safety and tolerability, while reducing the rate of brain atrophy at a high dose.

### Amyotrophic Lateral Sclerosis (ALS)

Only two drugs, riluzole and edaravone, are currently available to delay the progression of the disease, although they cannot revert the symptoms once they have manifested. Riluzole prolongs ALS survival; it increases survival rates at 12 months by 10% and prolongs survival by 6 months. Similarly Edaravone is effective in treating ALS.

Masitinib is a tyrosine kinase inhibitor used to treat cancer in dogs. Its use in ALS resides in the fact that abnormal glial cells that proliferate in ALS might be sensitive to tyrosine kinase inhibitors. It was proven that mastinib inhibited glial cell activation in the appropriate rat model and increased survival.

Retigabine is an approved drug for epilepsy, and acts by binding to the voltage-gated potassium channels and increasing the M-current, thus leading to membrane hyperpolarization. Retigabine is able to prolong motor neuron survival and decrease excitability, which is advantageous in the treatment of ALS, since it is believed that, in this disease, neurons are hyper-excitable, firing more than normal and ultimately leading to cell death. This drug is still under clinical trial for the treatment of ALS.

Tamoxifen is an antioestrogen drug, approved for the chemotherapy and chemoprevention of breast cancer. The repurposing of this drug for the treatment of ALS arose serendipitously, after the observation of a neurological improvement in patients and disease stabilization in ALS patients with breast cancer treated with tamoxifen. Its neuroprotective properties appear to be related to inhibition of protein kinase C, which is overexpressed in the spinal cord of ALS patients. Moreover, tamoxifen was found to be able to modulate a proteinopathy present in ALS, through its capacity to be an autophagy modulator.

### Pharmaceutical Compositions

Agents that decrease the expression and/or activity of a cytokine, chemokine, and/or growth factor can be incorporated into pharmaceutical compositions suitable for administration to a subject. Such compositions typically comprise the antibody, peptide, fusion protein or small molecule and a pharmaceutically acceptable carrier. As used herein the pharmaceutically acceptable carrier is intended to include any and all solvents, dispersion media, coatings, antibacterial and antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary active compounds can also be incorporated into the compositions.

A pharmaceutical composition of the present invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition should be sterile and should be fluid to the extent that easy syringeability exists. It must be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Inhibition of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds including, e.g., viral particles are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, inhibitory agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the present invention are dictated by, and directly dependent on, the unique characteristics of the active compound, the particular therapeutic effect to be achieved, and the limitations inherent in the art of compounding such an active compound for the treatment of individuals.

The above described inhibitory agents may be administered in the form of expressible nucleic acids which encode said agents. Such nucleic acids and compositions in which they are contained, are also encompassed by the present invention. For instance, the nucleic acid molecules of the present invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

### Analyzing Biomarker Nucleic Acids and Polypeptides

Biomarker nucleic acids and/or biomarker polypeptides can be analyzed according to the methods described herein and other suitable techniques to identify such genetic or expression alterations useful for the present invention.

a. Methods for Detection of Copy Number

Methods of evaluating the copy number of a biomarker nucleic acid are well-known to those of skill in the art. The presence or absence of chromosomal gain or loss can be evaluated simply by a determination of copy number of the regions or markers identified herein.

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches. In addition, amplification-based assays can be used to measure copy number, e.g., quantitative amplication using PCR (such as real-time PCR). Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

b. Methods for Detection of Biomarker Nucleic Acid Expression

Biomarker expression may be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In preferred embodiments, the RNA population is enriched in marker sequences. Enrichment can be undertaken, e.g., by primer-specific cDNA synthesis, or multiple rounds of linear amplification based on cDNA synthesis and template-directed in vitro transcription.

The population of RNA, enriched or not in particular species or sequences, can further be amplified. As used herein, an amplification process is designed to increase the sensitivity of the detection. For example, where RNA is mRNA, an amplification process such as RT-PCR can be utilized to amplify the mRNA, such that a signal is detectable or detection is enhanced. Such an amplification process is beneficial particularly when the biological, or tissue sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used.

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts. Methods of preparing DNA arrays and their use are well-known in the art (see, e.g., U.S. Pat. Nos: 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. Patent Application 20030215858).

c. Methods for Detection of Biomarker Protein Expression

The activity or level of a biomarker protein can also be detected and/or quantified by detecting or quantifying the expressed polypeptide. The polypeptide can be detected and quantified by any of a number of means well-known to those of skill in the art. Aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response of a neurodegenerative disorder to at least one agent that decreases the level of cytokines, chemokines, and/or growth factors. Any method known in the art for detecting polypeptides can be used. Such methods include, without limitation, immunodiffusion, immunoelectrophoresis, a Western blot assay, an immunofluorescence assay, an enzyme immunoassay, an immunoprecipitation assay, a chemiluminescence assay, an immunohistochemical assay, a dot blot assay, or a slot blot assay. General techniques to be used in performing the various immunoassays noted above and other variations of the techniques, such as in situ proximity ligation assay (PLA), fluorescence polarization immunoassay (FPIA), fluorescence immunoassay (FIA), enzyme immunoassay (EIA), nephelometric inhibition immunoassay (NIA), enzyme linked immunosorbent assay (ELISA), radioimmunoassay (RIA), ELISA, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference) alone or in combination or alternatively with NMR, MALDI-TOF, LC-MS/MS, are known to those of ordinary skill in the art.

Such reagents can also be used to monitor protein levels in a cell or tissue, e.g., white blood cells or lymphocytes, as part of a clinical testing procedure, e.g., in order to monitor an optimal dosage of an inhibitory agent. Detection can be facilitated by coupling (e.g., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, 0-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Unless otherwise specified here within, antibody or antibodies broadly encompass naturally-occurring forms of antibodies (e.g. IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody. An antibody refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. Framework or FR residues are those variable-domain residues other than the hypervariable residues as herein indicated. The variable regions of the heavy and light chains contain a binding domain that interacts with an antigen.

Antibody as used herein also includes antigen-binding portion of an antibody. The antigen-binding portion refers to one or more fragments of an antibody that retain the ability to specifically bind to an antigen (e.g., a pro-inflammatory cytokine, chemokine, and/or growth factor). It has been shown that the antigen-binding function of an antibody can be performed by fragments of a full-length antibody. Examples of binding fragments encompassed within the antigen-binding portion of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the antigen-binding portion of an antibody.

In some embodiments, ELISA and RIA procedures may be conducted such that a desired biomarker protein standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

In some embodiments, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Other techniques may be used to detect biomarker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

Anti-biomarker protein antibodies may also be used for imaging purposes, for example, to detect the presence of biomarker protein in cells and tissues of a subject. Suitable labels include radioisotopes, iodine (¹²⁵I ¹²¹I), carbon (¹⁴C), sulphur (³⁵S), tritium (³H), indium (¹¹²In), and technetium (⁹⁹mTc), fluorescent labels, such as fluorescein and rhodamine, and biotin.

Antibodies are commercially available (listed above) or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies. For example, antibody fragments capable of binding to a biomarker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies. Antibodies produced from a library, e.g., phage display library, may also be used.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

### Sample

Biological samples can be collected from a variety of sources from a subject including a body fluid sample, cell sample, or a tissue sample comprising nucleic acids and/or proteins. Body fluids refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In some embodiments, the subject and/or control sample is selected from the group consisting of cells, cell lines, whole blood, serum, plasma, buccal scrape, saliva, cerebrospinal fluid, and bone marrow. In some embodiments, samples can contain live cells/tissue, fresh frozen cells, fresh tissue, biopsies, fixed cells/tissue, cells/tissue embedded in a medium, such as paraffin, histological slides, or any combination thereof. In preferred embodiments, blood or serum is used.

The samples can be collected from individuals repeatedly over a longitudinal period of time (e.g., once or more on the order of days, weeks, months, annually, biannually, etc.).

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids. In some embodiments, certain cell types are purified based on at least one marker present on the cell surface. In some embodiments, such purification is be preceded by centrifugation to concentrate and/or separate out other types of undesired cells or proteins. In some embodiments, the markers present on the cell surface are determined by flow cytometry. In some embodiments, one marker is determined. In preferred embodiments, at least two, three, four, five, six, or seven markers are determined.

A sample may comprise a fixed molecule. A molecule is “fixed” or “affixed” to a substrate if it is covalently or non-covalently associated with the substrate such the substrate can be rinsed with a fluid (e.g. standard saline citrate, pH 7.4) without a substantial fraction of the molecule dissociating from the substrate.

### Diagnostic Assays

One aspect of the present invention relates to diagnostic assays for determining the level of pro-inflammatory cytokines, chemokines, and/or growth factors in the context of a biological sample (e.g., blood, serum, cells, or tissue) to thereby determine whether an individual is afflicted with a disorder and/or to determine the state of such a disorder, such as a neurodegenerative disorder, indicated by such cytokine/chemokine/growth factor levels. For example, determining the level of cytokine/chemokine/growth factor would indicate whether a subject afflicted with a neurodegenerative disorder would likely respond to treatment with at least one agent that inhibits the cytokine/chemokine/growth factor.

The detected level of cytokine/chemokine/growth factor, and a statistical algorithm are useful for classifying whether a sample is associated with a disease or disorder mediated by an aberrant expression (e.g., upregulation or downregulation) of cytokine/chemokine/growth factor, and/or whether a subject afflicted with a neurodegenerative disorder would likely respond to treatment with at least one agent that inhibits the pro-inflammatory cytokine/chemokine/growth factor. In certain instances, the statistical algorithm is a single learning statistical classifier system. For example, a single learning statistical classifier system can be used to classify a sample as a cytokine/chemokine/growth factor sample based upon a prediction or probability value and the presence or level of cytokine/chemokine/growth factor. The use of a single learning statistical classifier system typically classifies the sample as a cytokine/chemokine/growth factor sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naïve learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the cytokine/chemokine/growth factor classification results to a clinician.

### Sequences

As used herein, coding region refers to regions of a nucleotide sequence comprising codons which are translated into amino acid residues, whereas noncoding region refers to regions of a nucleotide sequence that are not translated into amino acids (e.g., 5′ and 3′ untranslated regions).

Complement [to] or complementary refers to the broad concept of sequence complementarity between regions of two nucleic acid strands or between two regions of the same nucleic acid strand. It is known that an adenine residue of a first nucleic acid region is capable of forming specific hydrogen bonds (base pairing) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. In other embodiments, all nucleotide residues of the first portion are capable of base pairing with nucleotide residues in the second portion.

A nucleic acid is operably linked when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for nucleic acid and polypeptide molecules useful in the present invention are well-known in the art and readily available on publicly available databases, such as the National Center for Biotechnology Information (NCBI).

### EXAMPLES

*Samples: JC, RS, and WO are ALS patients; TC is a healthy twin of JC; rest of the samples are controls.

### Example 1: Materials and Methods

### Cell Lines, Reagents, and Antibodies

RPMI 1640 supplemented with 10% fetal bovine serum (FBS) (Gemini Bio-Products, CA) was used for the cultures of human NK cells. Human pancreatic cancer cell lines Panc-1, MIA PaCa-2 (MP2), BXPC3, HPAF, and Capan were generously provided by Dr. Guido Eibl (UCLA David Geffen School of Medicine) and PL12 was provided by Dr. Nicholas Cacalano (UCLA Jonsson Comprehensive Cancer Center). Panc-1, MP2 and BXPC3 were cultured with DMEM in supplement with 10% FBS and 1% Penicillin-Streptomycin (Gemini Bio-Products, CA). HPAF, Capan and PL12 were cultured in RMPI 1640 medium supplemented with 10% FBS and 1% penicillin-streptomycin. Recombinant human IL-2 was obtained from NIH-BRB. Human TNF-α and IFN-γ was obtained from Biolegend (San Diego, CA). Antibody to CD16 was purchased from Biolegend (San Diego, CA).

Fluorochrome-conjugated human and mouse antibodies for flow cytometry were obtained from Biolegend (San Diego, CA). Monoclonal antibodies to TNF-α and IFN-γ were prepared in our laboratory, and used at 1:100 dilutions to block rhTNF-α and rhIFN-γ functions. The human NK cells and monocytes purification kits were obtained from Stem Cell Technologies (Vancouver, Canada). Propidium iodide (PI) and N-Acetyl Cysteine (NAC) were purchased from Sigma Aldrich (St. Louis, MO). Cisplatin and paclitaxel were purchased from Ronald Reagan UCLA Medical Center Pharmacy (Los Angeles, CA).

### Purification of Human NK Cells And Monocytes

Informed consents and all the procedures were approved by UCLA Institutional Review Board (IRB). NK cells and monocytes were negatively selected from PBMCs using isolation kits from Stem Cell Technologies (Vancouver, BC, Canada). Greater than 96% purity was obtained both for purified NK cells and monocytes based on flow cytometric analysis.

### Analysis of Human Pancreatic Cancer Cells Growth In Immune-Deficient (NSG) and Humanized-BLT Mice

Humanized-BLT (hu-BLT; human bone marrow/liver/thymus) mice were generated as previously described, and animal research was performed under the written approval of the UCLA Animal Research Committee (ARC).

In vivo growth of pancreatic tumors was performed by orthotopic tumor implantation in the pancreas of NSG or hu-BLT mice. To establish orthotopic tumors, mice were anesthetized using isoflurane and tumors with 10p HC Matrigel (Corning, NY, USA) were injected in the pancreas using insulin syringe. Mice received 1.5×10⁶ super-charged NK cells via tail vein injection 7 to 10 days after the surgery. They were also fed AJ2 (5 billion/dose) orally. The first dose of AJ2 was given one or two weeks before tumor implantation, and feeding was continued throughout the experiment at an interval of every 48 hours. Mice were euthanized when signs of morbidity were evident. Pancreas, pancreatic tumors, bone marrow, spleen, and peripheral blood were harvested and single cell suspensions were prepared from each tissue as described previously and below.

Cell Dissociation and Cell Culture of Tissues from Hu-BLT And NSG Mice

Pancreatic tumors were harvested from NSG and hu-BLT mice and cut into 1 mm³ pieces and placed into a digestion buffer containing 1 mg/ml collagenase IV, 10 U/ml DNAse I, and 1% bovine serum albumin (BSA) in DMEM media for 20 minutes at 37° C. the samples were then filtered through a 40 mm cell strainer and centrifuged at 1500 rpm for 10 minutes at 4° C. To obtain single-cell suspensions from BM, femurs were flushed using media, and filtered through a 40 μm cell strainer. Spleens were removed and single cell suspensions were prepared and filtered through a 40 μm cell strainer and centrifuged at 1500 rpm for 5 minutes at 4° C. The pellets were re-suspended in ACK buffer to remove the red blood cells. Peripheral blood mononuclear cells (PBMCs) were isolated using ficoll-hypaque centrifugation.

Isolations of NK Cells, T Cells and Monocytes from Hu-BLT Mice

NK cells and T cells from hu-BLT splenocytes were obtained as described previously by using the human CD56+ and CD3+ selection kits respectively (Stem Cells Technologies, Canada). Monocytes from hu-BLT were selected from BM using human CD14 isolation kit (eBioscience, San Diego, CA).

### Generation of Osteoclasts and Expansion of Human and Hu-BLT NK Cells

Monocytes were purified form human peripheral blood or hu-BLT BM and cultured using alpha-MEM medium containing M-CSF (25 ng/mL) and RANKL (25 ng/mL) for 21 days (medium was refreshed every 3 days). NK cells were activated with rh-IL-2 (1000 U/ml) and anti-CD16mAb (3 μg/ml) for 18-20 hours before they were cultured with osteoclasts and sonicated-AJ2 to generate super-charged NK cells. The medium was refreshed every 3 days with RMPI containing rh-IL-2 (1000 U/ml).

### In Vitro MP2 And OSCSCs Cancer Stem Cell Differentiation

Differentiation of MP2 and OSCSCs (oral squamous carcinoma stem-cells) tumors was conducted as described previously. Briefly NK cells were treated with a combination of anti-CD16mAb (3 μg/mL) and IL-2 (1,000 U/mL) for 18 hours before the supernatants were removed and used for differentiation of the tumors. The amounts of IFN-γ produced by activated NK cells were assessed using ELISA kits purchased from Biolegend (CA, USA). To induce differentiation of tumors a total of 3,500 pg of IFN-γ containing supernatants were added for 4 days.

### Enzyme-Linked Immunosorbent Assays (Elisas) and Multiplex Cytokine Assay

Human ELISA kits for IFN-γ and IL-6 were purchased from Biolegend (San Diego, CA). The assay was conducted as recommended by the manufacturer. For certain experiments multiplex arrays were used to determine the levels of secreted cytokines and chemokines. Analysis was performed using MAGPIX (Millipore, MA) and data was analyzed using xPONENT 4.2.

### Surface Staining and Cell Death Assays

Staining was performed by staining the cells with antibodies as described previously, briefly, antibodies were added to 1×10⁴ cells in 50 μl of cold-PBS+1% BSA and cells were incubated on ice for 30 min. Thereafter cells were washed in cold PBS+1% BSA and flow cytometric analysis was performed using Beckman Coulter Epics XL cytometer (Brea, CA) and results were analyzed in FlowJo vX software (Ashland, OR).

### ⁵¹Cr Release Cytotoxicity Assay

The ⁵¹Cr release assay was performed as described previously. Patient-derived OSCSCs were used as a specific and sensitive NK targets to assess NK cell-mediated cytotoxicity. Briefly, different numbers of effector cells were incubated with ⁵¹Cr-labeled OSCSCs. After 4-hour incubation the supernatants were harvested from each sample and counted on a gamma counter. The percentage specific cytotoxicity was calculated using the following formula:

$$\% Cytotoxicity = \frac{Experimental cpm - Spontaneous cpm}{Total cpm - Spontaneous cpm}$$

Lytic unit 30/10⁶ is calculated by using the inverse of the number of effector cells needed to lyse 30% of tumor target cells×100.

### Statistical Analysis

An unpaired, two-tailed student t-test was performed for the statistical analysis. One-way ANOVA using Prism-7 software was used to compare different groups. (n) denotes the number of mice used for each condition in the experiment. The following symbols represent the levels of statistical significance within each analysis, ***(p-value <0.001), **(p-value 0.001-0.01), *(p-value 0.01-0.05).

### Example 2

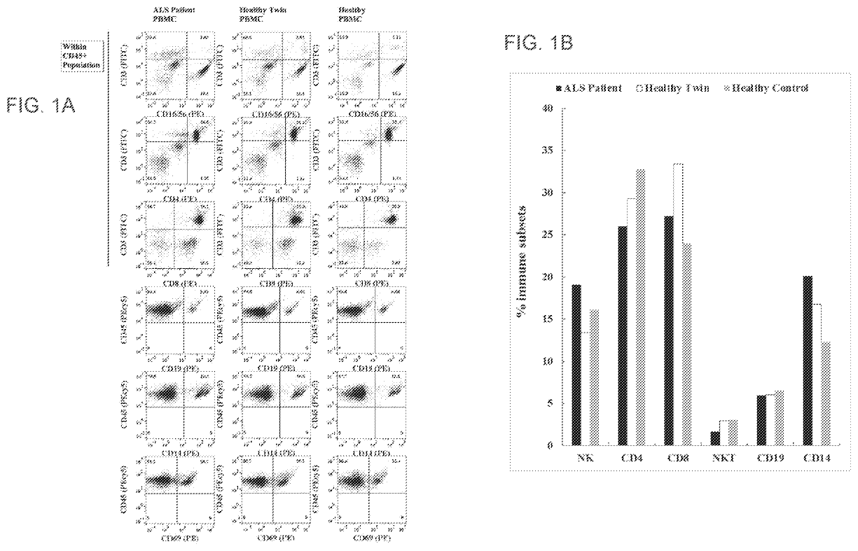

Percentage of immune subsets in PBMCs in lab blood drawn. PBMC were isolated from peripheral blood of donors using ficoll-hypaque gradient centrifugation. The percentages of each cell subset in PBMCs were determined by flow cytometric analysis after staining with specific fluorescent conjugated antibodies directed to the cell surface receptors. No significant differences in the percentages of immune subsets between the donors (FIG. 1A and FIG. 1B).

### Example 3

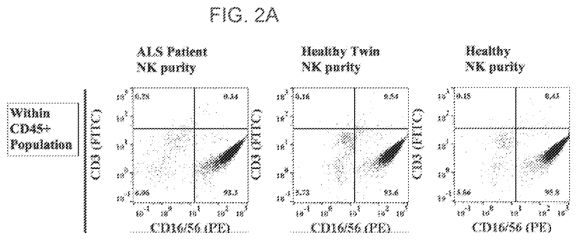

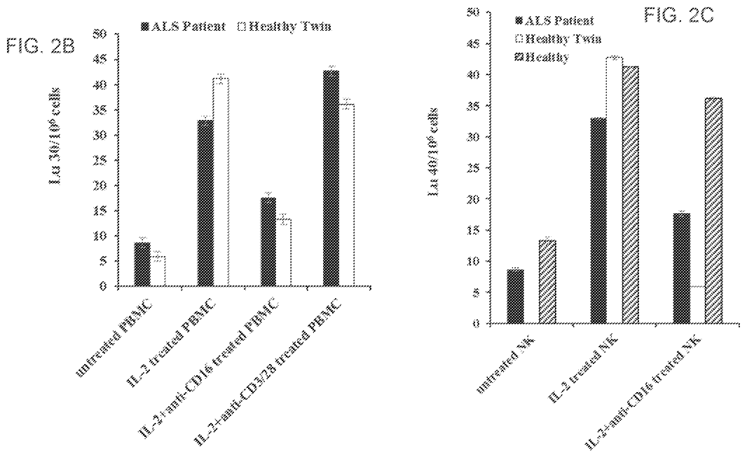

NK cells were purified from the peripheral blood of patients as described in the materials and methods section. The levels of purity were determined using NK specific antibodies (FIG. 2A).

PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100. No significant differences in primary NK cytotoxicity within PBMCs between the donors (FIG. 2B).

NK cells were isolated from PBMCs as described in the materials and methods section. Purified NK cells were left untreated, or treated with IL-2 (1000 U/mL) or treated with the combination of IL-2 and anti-CD16 mAb (3 μg/ml). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100. No significant differences in NK cytotoxicity by purified NK cells between the donors (FIG. 2C).

### Example 4

NK cells were activated with rh-IL-2 (1000 U/ml) and anti-CD16mAb (3 μg/ml) for 18-20 hours before they were cultured with osteoclasts and sonicated-AJ2 to generate super-charged NK cells. The medium was refreshed every 3 days with RMPI containing rh-IL-2 (1000 U/ml).

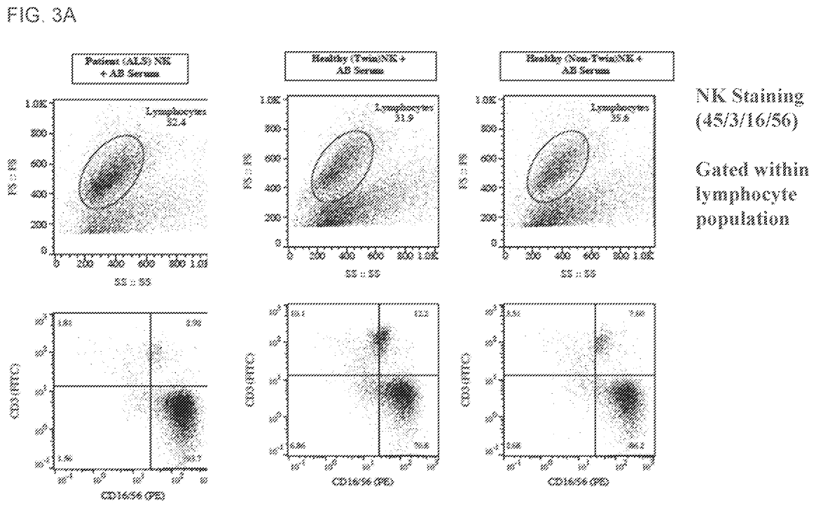

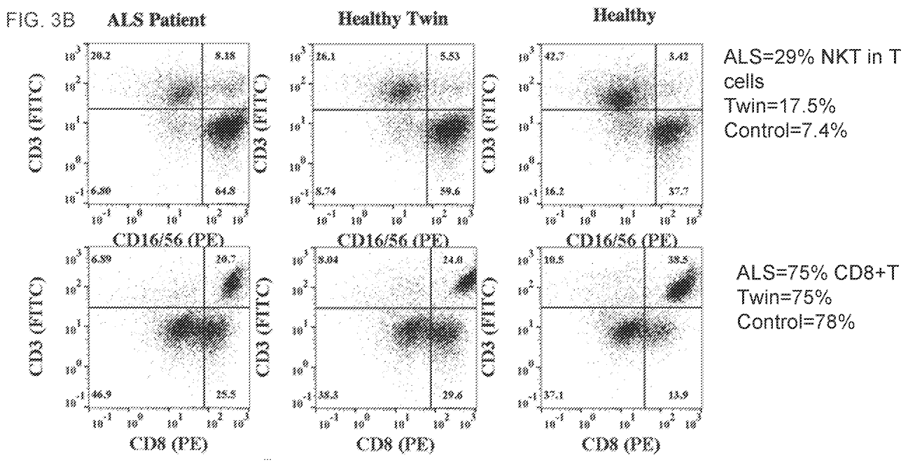

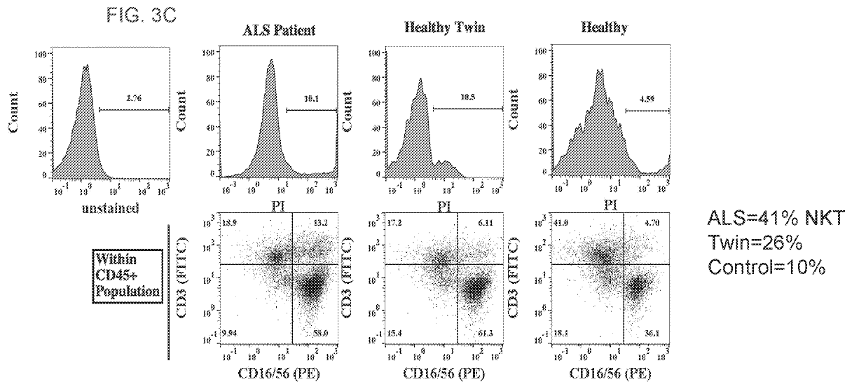

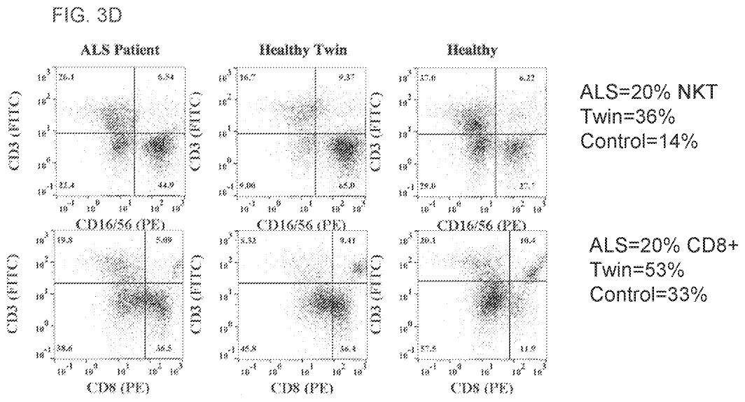

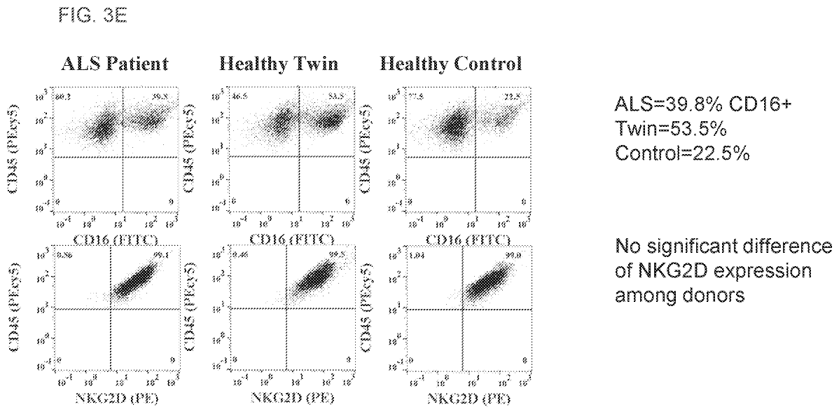

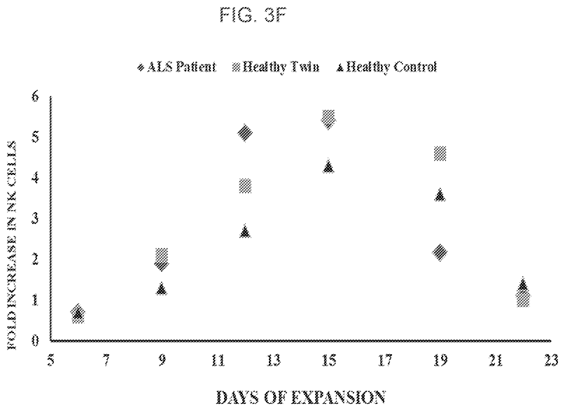

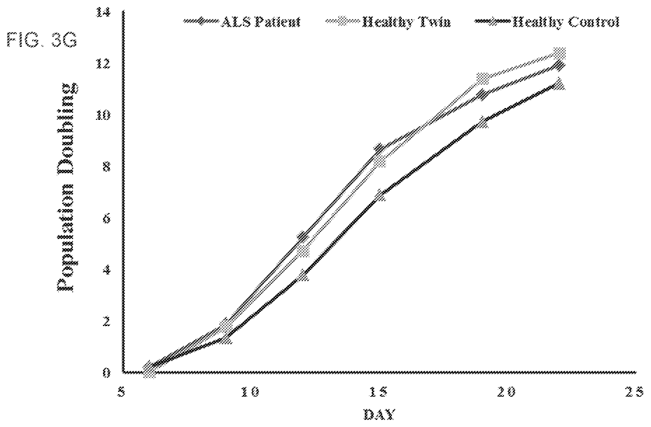

Day 6 NK Expansion, super-charging the NK cells (FIG. 3A). Day 9 NK Expansion, increased NKT subsets but no change in CD8+ T cells in ALS (FIG. 3B). Day 12 NK Expansion, NK cells expand NKT cell subset in all but particularly more in ALS patient (FIG. 3C). Similar or lower cell death in super-charged NK cells in ALS patient when compared to controls. Day 27 NK Expansion, increased contraction of NKT cells after expansion by the NK cells in ALS patient (FIG. 3D). Day 27 NK Expansion (FIG. 3E).

Percentages of CD8+ T-Cell expansion within super-charged NK cells (Table 1). No significant changes between the donors. Increased NKT cell expansion by super-charged NK cells in ALS (Table 2). The fold increase in expansion of super-charged NK cells by day (Table 3 and FIG. 3F). Population Doubling of super-charged NK cells is similar between the donors (FIG. 3G).

### Example 5

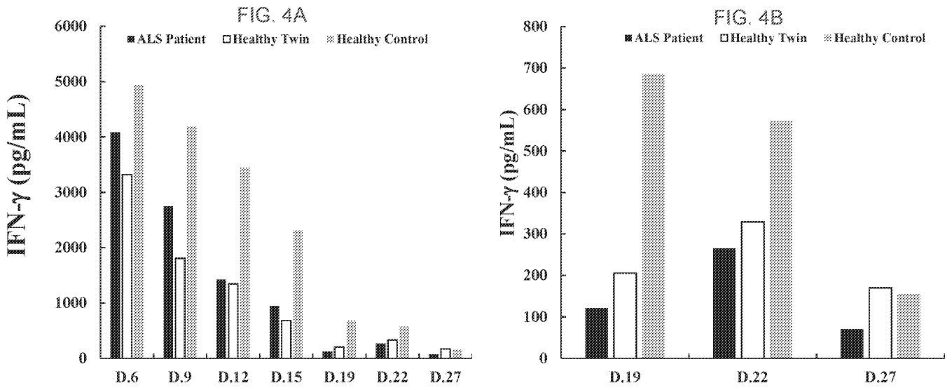

During supercharging of the NK cells no significant differences can be seen in secretion of IFN-g from NK cells at different days of expansion (FIG. 4A and FIG. 4B). Supernatants were harvested at the days indicated in the figures and the levels of IFN-g secretion were determined using specific ELISAs for INF-g.

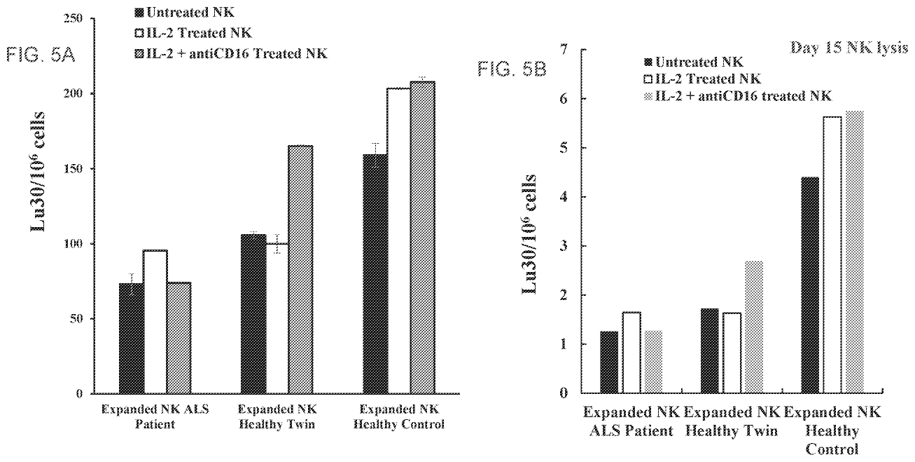

During supercharging of the NK cells no significant differences can be seen in lysis of OSCSCs by the NK cells at day 15 of NK expansion (FIG. 5A and FIG. 5B). Cytotoxicity of NK cells against OSCSCs were determined using 4-hour Cr release assay. The Figure in the right demonstrates the levels of NK cell cytotoxicity per percent of NK cells. The lytic units 30/10⁶ cells were determined using inverse number of NK cells required to lyse 30% of the target cells×100.

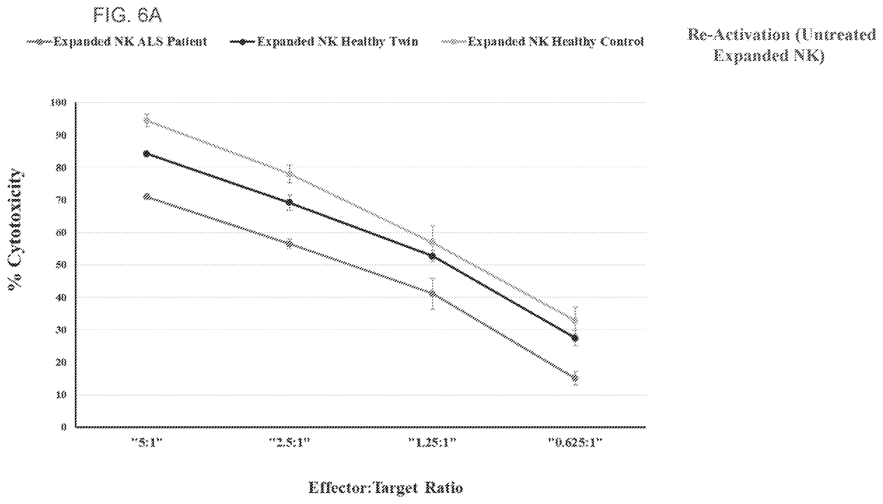

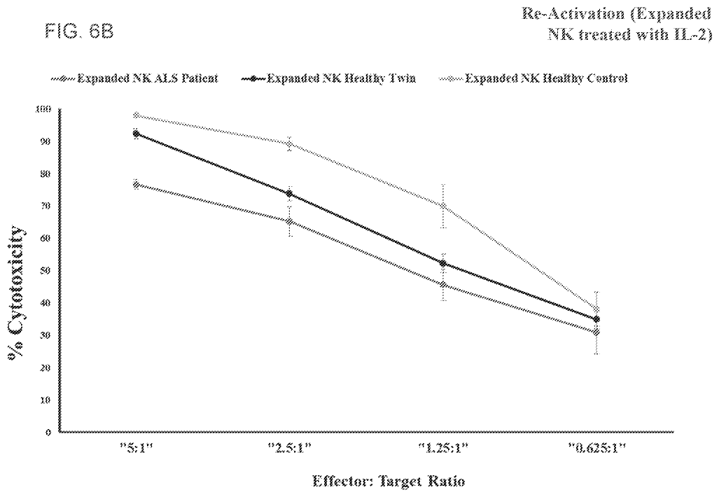

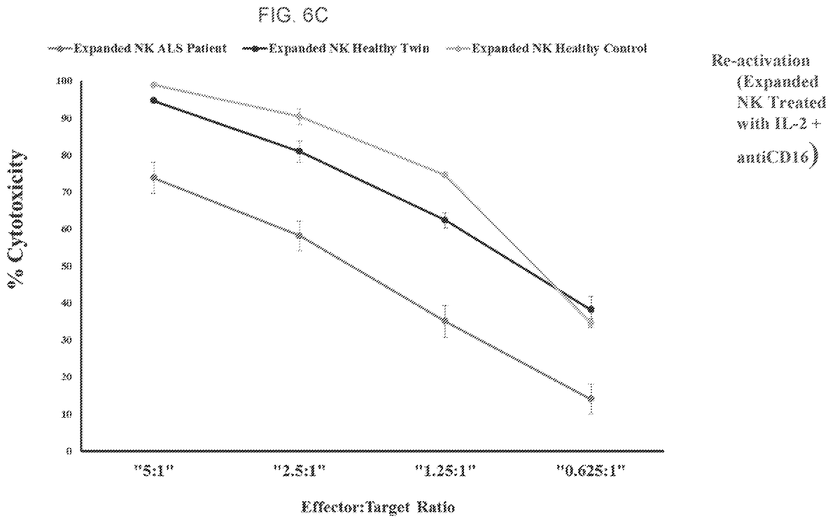

Decreased cytotoxicity of Untreated supercharged NK cells by ALS patient (FIG. 6A). Cytotoxicity of untreated supercharged NK cells against OSCSCs were determined using 4 hour Cr release assay. The lytic units 30/10⁶ cells were determined using inverse number of NK cells required to lyse 30% of the target cells×100.

Decreased cytotoxicity of IL-2 treated supercharged NK cells by ALS patient (FIG. 6B). Cytotoxicity of IL-2 treated supercharged NK cells against OSCSCs were determined using 4 hour Cr release assay. The lytic units 30/10⁶ cells were determined using inverse number of NK cells required to lyse 30% of the target cells×100.

Decreased cytotoxicity of IL-2+anti-CD16 treated supercharged NK cells by ALS patient (FIG. 6C). Cytotoxicity of IL-2 (1000 u/ml) and anti-CD16mAb (3 mg/ml) supercharged NK cells against OSCSCs were determined using 4 hour Cr release assay. The lytic units 30/10⁶ cells were determined using inverse number of NK cells required to lyse 30% of the target cells×100.

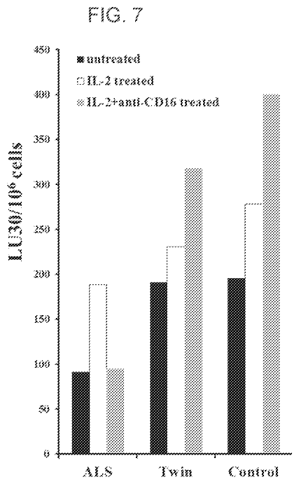

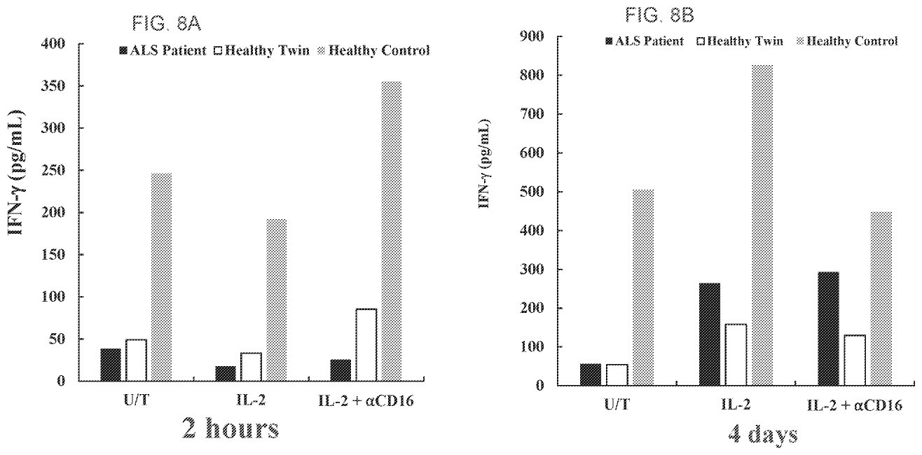

Decreased cytotoxicity of supercharged NK cells by ALS patient (FIG. 7). NK cell mediated cytotoxicity of OSCSCs. Cytotoxicity of supercharged NK cells against OSCSCs were determined using 4 hour Cr release assay. The lytic units 30/10⁶ cells were determined using inverse number of NK cells required to lyse 30% of the target cells×100. No or decreased secretion of IFN-g by supercharged NK cells by ALS patient (FIG. 8A and FIG. 8B). Supernatants were harvested at the times indicated in the figures and the levels of IFN-g secretion were determined using specific ELISAs for INF-g.

### Example 6

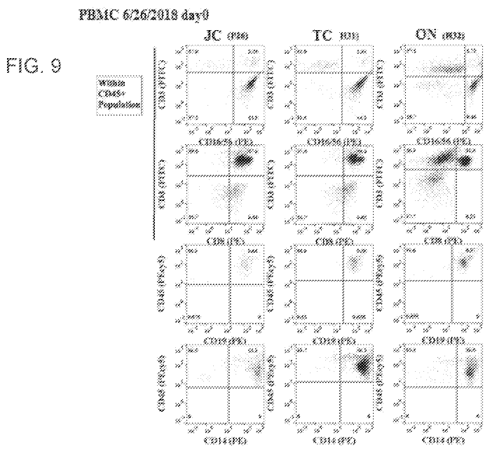

Flow cytometry of PBMCs isolated from a patient afflicted with ALS and healthy controls (FIG. 9 and Table 4). No therapy is shown. PBMC were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors.

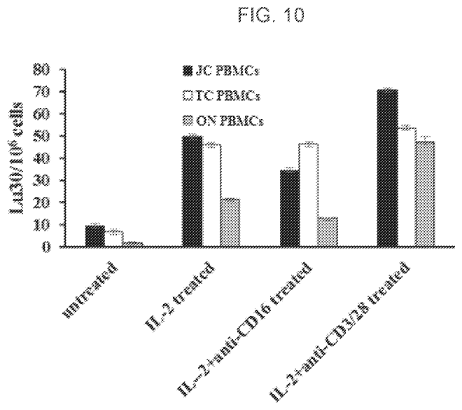

No differences in the NK cytotoxicity of PBMCs by ALS patient (FIG. 10). PBMC 51Cr release. PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100

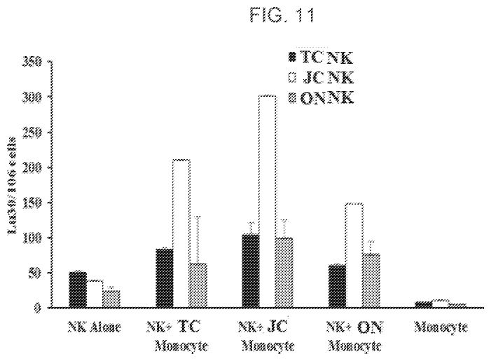

Increased cytotoxicity of NK cells cultured with autologous or allogeneic monocytes by ALS patient (FIG. 11). NK with different donor monocytes. Purified NK cells were treated with the combination of IL-2 and anti-CD16 mAb (3 μg/ml), and cultured with autologous and allogeneic monocytes at (1:1 NK:monocyte ratios). After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100.

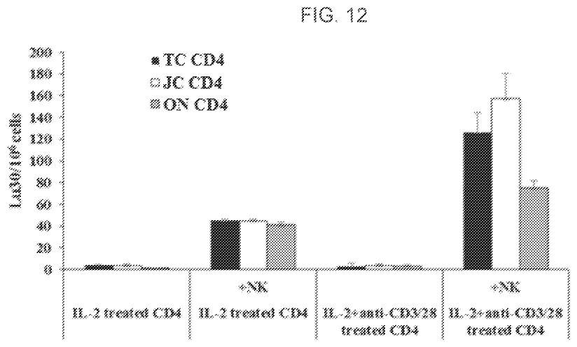

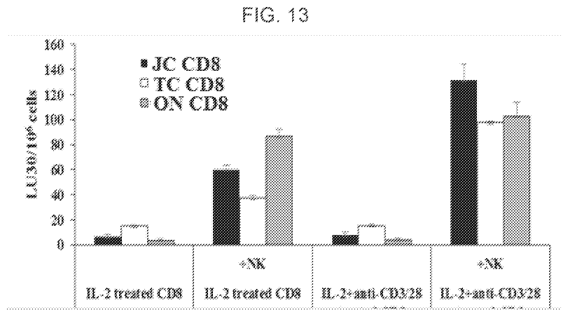

No differences in the cytotoxicity of NK cells cultured with CD4+ T cells by ALS patient (FIG. 12). Interaction of NK with CD4+ T cells. Purified NK cells were treated with IL-2 (1000 U/mL) and cultured with CD4+ T cells treated with the combination of IL-2+ anti-CD3/28 antibodies (25 μl/mL) at (1:1 NK:CD4) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100.

No differences in the cytotoxicity of NK cells cultured with CD8+ T cells by ALS patient (FIG. 13). Interaction of NK with CD8+ T cells. Purified NK cells were treated with IL-2 (1000 U/mL) and cultured with CD8+ T cells treated with the combination of IL-2+ anti-CD3/28 antibodies (25 μl/mL) at (1:1 NK:CD8) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100.

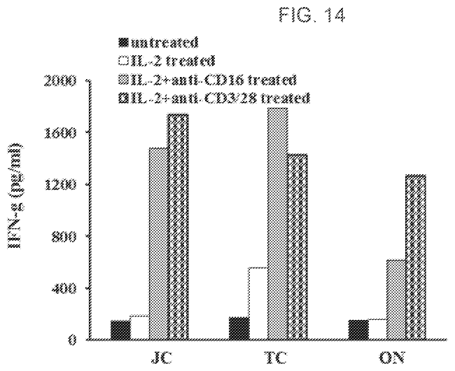

No significant differences in the IFN-g secretion with the exception of IL-2+anti-CD3/CD28 treated PBMCs of ALS patient (FIG. 14). PBMC-mediated IFN-g release. PBMC were left untreated, treated with IL-2 (1000 U/mL) or treated with the combination of IL-2 and anti-CD16 mAb (3 μg/ml), or IL-2 and anti-CD3/28 antibodies (25 μl/mL). After an overnight treatment at 37° C., the supernatants were removed and IFN-g secretion was measured using ELISA.

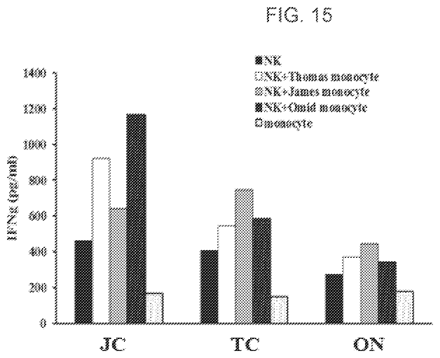

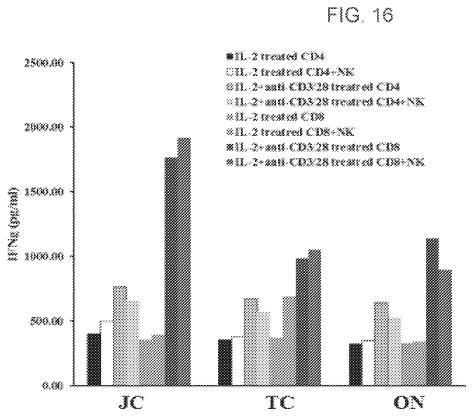

ALS patient's NK cells increase secretion of IFN-g with both autologous and allogeneic monocytes indicating potential priming of the NK cells (FIG. 15). IFN-g release of NK with different donor monocytes. Purified NK cells were treated with the combination of IL-2 and anti-CD16 mAb (3 μg/ml), and cultured with autologous and allogeneic monocytes at (1:1 NK:monocyte ratios). After an overnight treatment at 37° C., the supernatants were removed and IFN-g secretion was measured using ELISA.

Sorted CD8+ T cells from ALS patient secrete significant levels of IFN-g upon activation with IL-2+anti-CD3/CD28 in comparison to CD4+ T cells (FIG. 16). NK and/or CD4 and CD8 mediated IFN-g release. Purified NK cells were treated with IL-2 (1000 U/mL) and cultured with CD4+ and CD8+ T cells treated with the combination of IL-2+ anti-CD3/28 antibodies (25 μl/mL) at (1:1 NK:CD4 or CD8) as shown in the figure. After an overnight treatment at 37° C., the supernatants were removed and IFN-γ secretion was measured using ELISA.

### Example 7

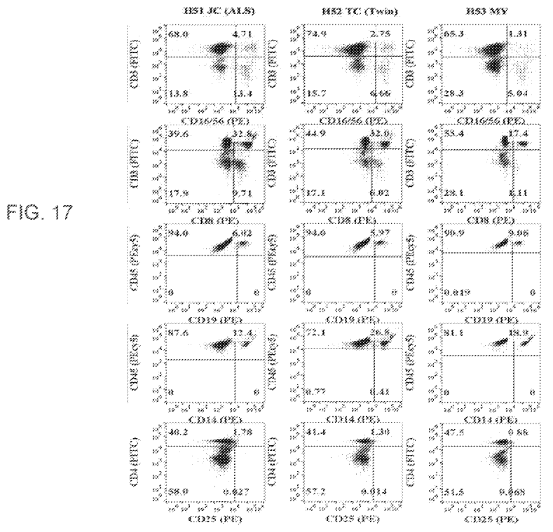

Flow cytometry of PBMCs isolated from a patient afflicted with ALS and healthy controls (FIG. 17 and Table 5). PBMC were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors.

Comparable or higher levels of NK cell cytotoxicity between ALS patient and healthy twin in the presence and absence of monocytes (Tables 6-13). Purified NK cells were treated with the combination of IL-2 and anti-CD16 mAb (3 μg/ml), and cultured with autologous monocytes at (1:1 NK:monocyte ratios). After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100.

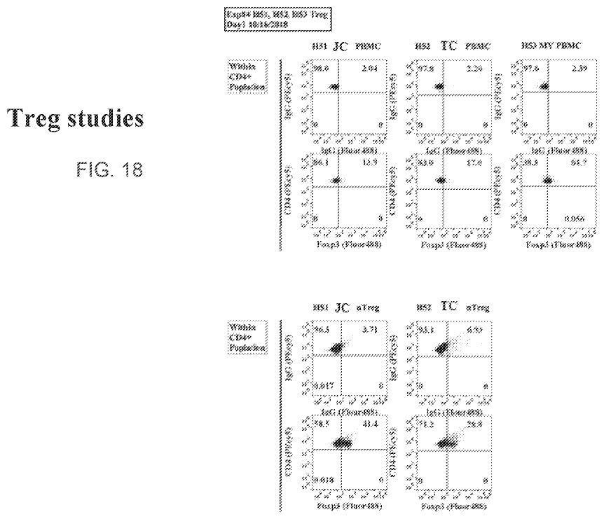

Comparable or higher percentages of Tregs within CD4+ T cells between ALS and healthy twin (FIG. 18). PBMC were isolated from peripheral blood of donors using ficoll-hypaque gradient centrifugation. The percentages of Treg subset within CD4+ T cells were determined by flow cytometric analysis after staining with specific fluorescent conjugated antibodies directed to the cell surface receptors.

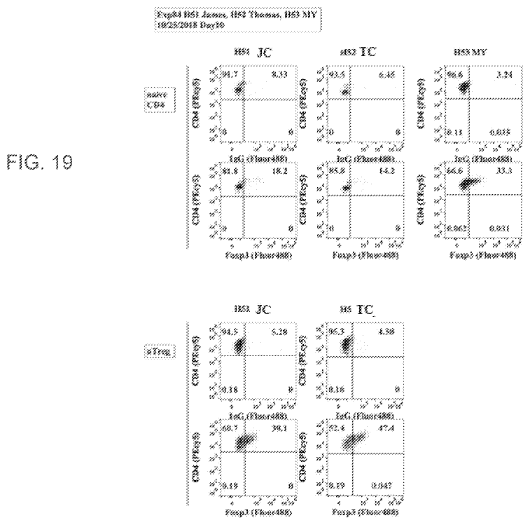

Comparable or higher percentages of Tregs within naïve CD4+ T cells differentiated to Tregs between ALS and healthy twin (FIG. 19). Naïve CD4+ T cells were isolated from peripheral blood of donors and differentiated to Treg cells using manufacturers recommendation. The percentages of Treg subset within CD4+ T cells were determined by flow cytometric analysis after staining with specific fluorescent conjugated antibodies directed to the cell surface receptors.

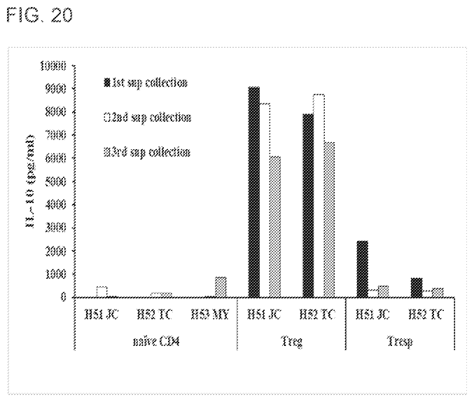

Comparable levels of IL-10 secretion from Tregs obtained from ALS and healthy twin (FIG. 20). IL-10 secretion by Tregs. Naïve CD4+ T cells were isolated from peripheral blood of donors and differentiated to Treg cells using manufacturers recommendation. After an overnight treatment at 37° C., the supernatants were removed and IL-10 secretion was measured using ELISA.

Comparable levels of IL-10 secretion from Tregs obtained from ALS and healthy twin (Table 14). ELISA for IL-10 secretion. Naïve CD4+ T cells were isolated from peripheral blood of donors and differentiated to Treg cells using manufacturers recommendation. After an overnight treatment at 37° C., the supernatants were removed and IL-10 secretion was measured using ELISA.

### Example 8

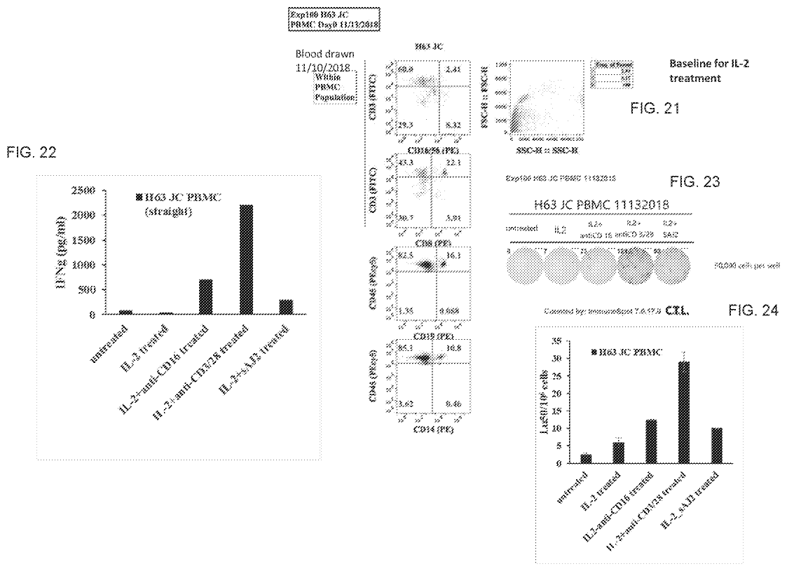

CD3/CD28 mediated increase in NK function (e.g., IFN-g secretion, NK cytotoxicity) by ALS patient PBMCs (Table 15 and FIG. 21-FIG. 24).

PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors (Table 15 and FIG. 21).

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-g secretion were measured using ELISA (FIG. 22). ELISA of IFN-g.

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay. ELISpot of IFN-g (FIG. 23).

PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (FIG. 24).

### Example 9

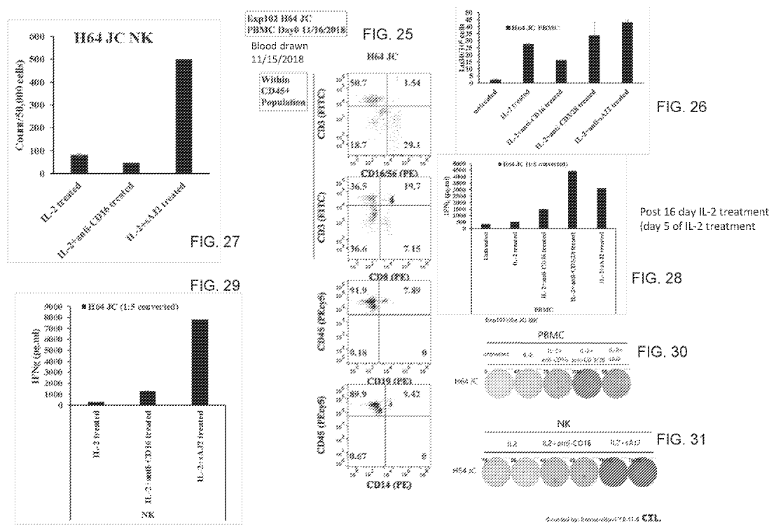

CD3/CD28 and AJ2 mediated increase in NK function from PBMCs and purified NK cells from an ALS patient (Table 16, FIG. 25-FIG. 30).

PBMC flow cytometry. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors (FIG. 25).

PBMC cytotoxicity. PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (FIG. 26).

NK cell cytotoxicity. NK cells were isolated from PBMCs as described in the materials and methods section. Purified NK cells were left untreated, or treated with IL-2 (1000 U/mL) or treated with the combination of IL-2 and anti-CD16 mAb (3 μg/ml) or IL-2+sAJ2 (Cells:sAJ2 1:20). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100 (FIG. 27).

ELISA of IFN-g secreted by PBMC from an ALS patient. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA (FIG. 28).

ELISA of IFN-g secreted by purified NK cells from an ALS patient. NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment at 37° C., the supernatants were harvested and the levels of IFN-γ secretion were measured using ELISA (FIG. 29).

ELISpot of IFN-g secreted by PBMC from an ALS patient. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay (FIG. 30).

ELISpot of IFN-g secreted by purified NK cells from an ALS patient. NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment, 50,000 cells per treatment group were used in ELISPOT assay to determine the number of spots expressing IFN-g (FIG. 31).

### Example 10

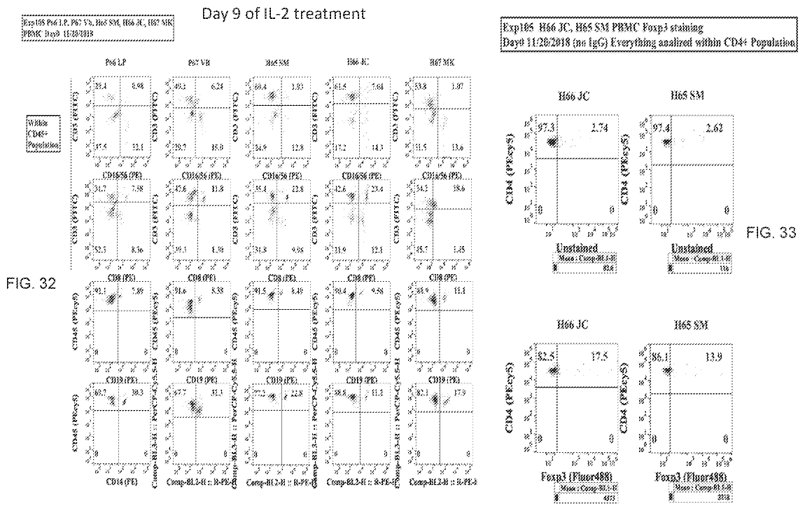

No difference in percentage of Treg cells between ALS and healthy control (Table 17 and FIG. 32-FIG. 33).

Flow cytometry of PBMCs from patients afflicted with ALS and healthy controls. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors (FIG. 32).

Tregs. PBMC were isolated from peripheral blood of donors using ficoll-hypaque gradient centrifugation. The percentages of Treg subset within CD4+ T cells were determined by flow cytometric analysis after staining with specific fluorescent conjugated antibodies directed to the cell surface receptors (FIG. 33).

### Example 11

Comparable cytotoxicity of NK cells from PBMCs and purified NK cells between ALS and healthy controls (Tables 18-19).

PBMC cytotoxicity. PBMC were left untreated, or treated with IL-2 (1000 U/mL) or treated with the combination of IL-2 and anti-CD16 mAb (3 μg/ml), or IL-2+anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (Table 18).

NK cytotoxicity. NK cells were isolated from PBMCs as described in the materials and methods section. Purified NK cells were left untreated, or treated with IL-2 (1000 U/mL) or treated with the combination of IL-2 and anti-CD16 mAb (3 μg/ml) or IL-2+sAJ2 (Cells:sAJ2 1:20). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100 (Table 19).

### Example 12

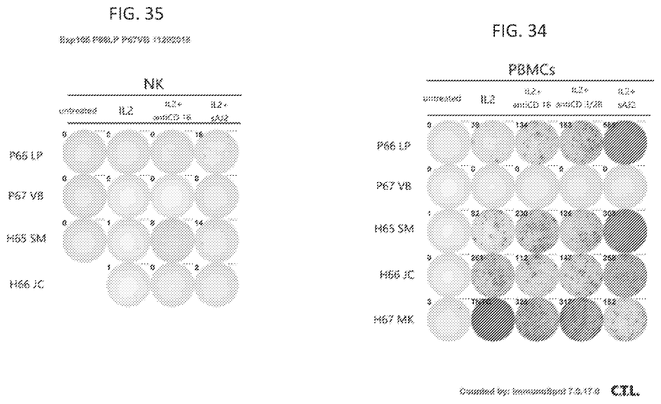

Comparable or higher secretion of IFN-g from the PBMCs and purified NK cells between ALS and healthy controls (Tables 20-21 and FIG. 34-FIG. 35).

PBMC ELISpot. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay (FIG. 34).

PBMC ELISA. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA (Table 20).

NK Elispot. NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment, 50,000 cells per treatment group were used in ELISPOT assay to determine the number of spots expressing IFN-γ (FIG. 35).

NK ELISA. NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment at 37° C., the supernatants were harvested and the levels of IFN-γ secretion were measured using ELISA (Table 21).

### Example 13

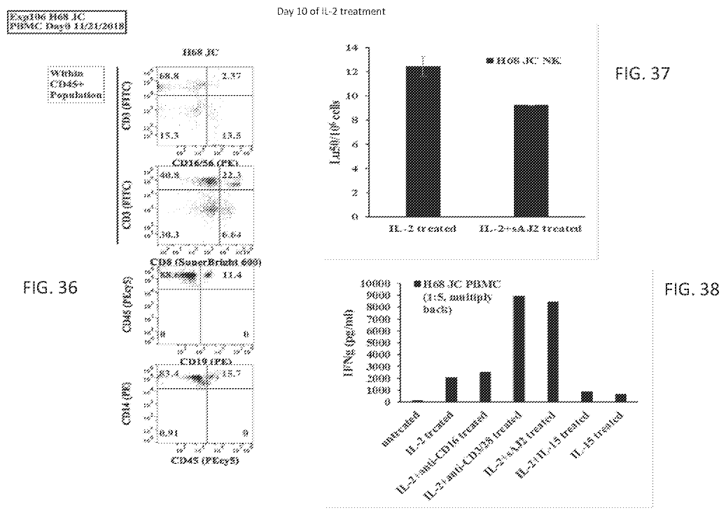

CD3/CD28 and AJ2 mediated increase in NK function in ALS patient (Table 22 and FIG. 36-FIG. 38).

PBMC Flow cytometry. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors.

NK cytotoxicity. NK cells were isolated from PBMCs as described in the materials and methods section. Purified NK cells were treated with IL-2 (1000 U/mL) or IL-2+sAJ2 (Cells:sAJ2 1:20). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100 (FIG. 37).

PBMC ELISA. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20) or IL-2+IL-15 (10 ng/ml) or IL-15 alone (10 ng/ml). After an overnight treatment at 37° C., the supernatants were harvested and the levels of IFN-γ secretion were measured using ELISA (FIG. 38).

### Example 14

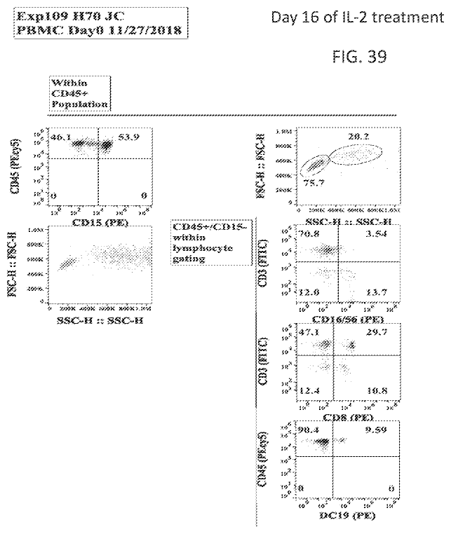

CD3/CD28 mediated increase in IFN-g secretion by CD8 in ALS patient (Tables 23-25 and FIG. 39-FIG. 42).

PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors (FIG. 39 and Table 23).

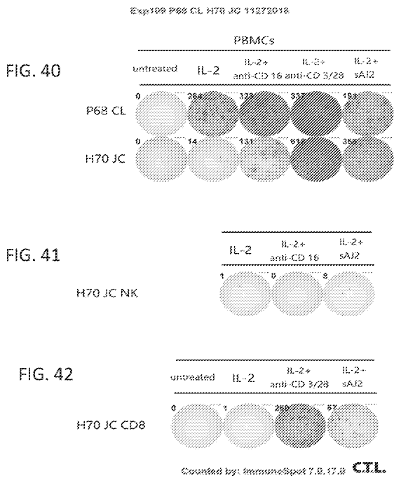

PBMC Elispot. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay (FIG. 40).

PBMC ELISA. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA (Table 24).

NK ELISpot. NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment, 50,000 cells per treatment group were used in ELISPOT assay to determine the number of spots expressing IFN-γ (FIG. 41).

NK cell ELISA. NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment at 37° C., the supernatants were harvested and the levels of IFN-γ secretion were measured using ELISA (Table 25).

CD8 Elispot. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISPOT assay to determine the number of spots expressing IFN-γ (FIG. 42).

### Example 15

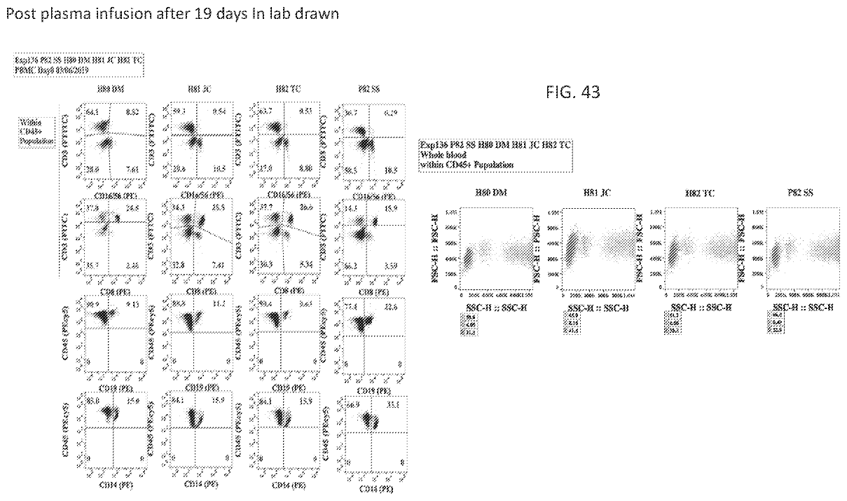

Comparable or increased NK cytotoxicity by ALS patient (Tables 26-28 and FIG. 43).

PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors (FIG. 43 and Table 26).

PBMC cytotoxicity. PBMC were left untreated, or treated with IL-2 (1000 U/mL) or treated with the combination of IL-2 and anti-CD16 mAb (3 μg/ml), or IL-2+anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (Table 27).

NK cell cytotoxicity. NK cells were isolated from PBMCs as described in the materials and methods section. Purified NK cells were left untreated, or treated with IL-2 (1000 U/mL) or treated with the combination of IL-2 and anti-CD16 mAb (3 μg/ml) or IL-2+sAJ2 (Cells:sAJ2 1:20). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100 (Table 28).

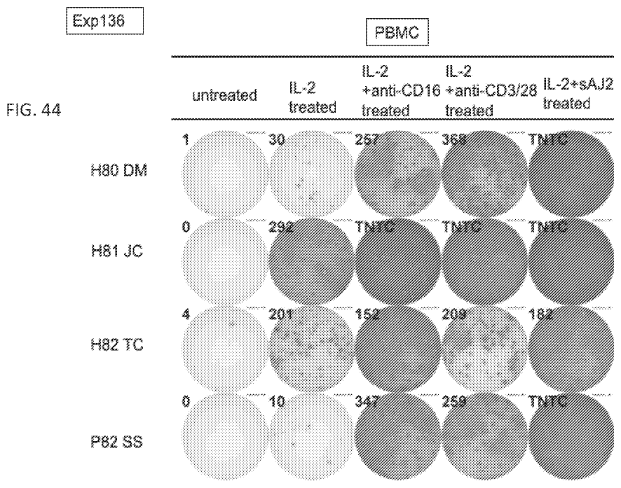

CD3/CD28 mediated increase in IFN-g spots and secretion by PBMCs of ALS patient (Table 29 and FIG. 44).

PBMC ELISpot. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay (FIG. 44).

PBMC ELISA. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA (Table 29).

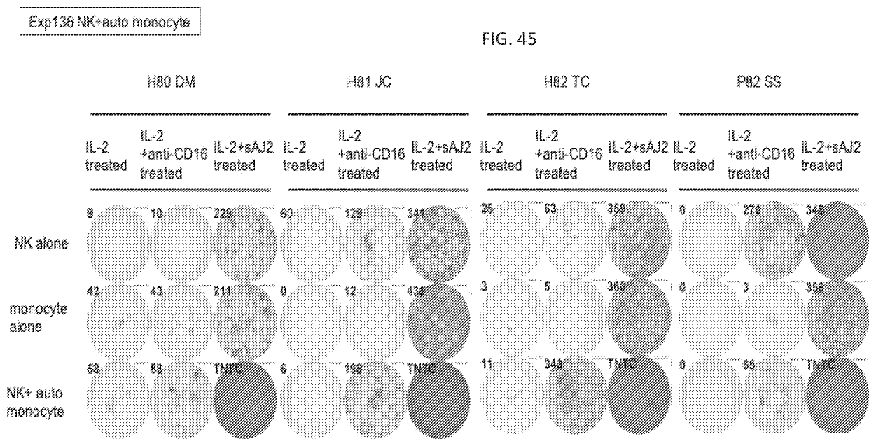

Comparable or increased IFN-g secretion by the NK cells with and without monocytes in ALS patient as compared to healthy twin (FIG. 45 and Table 30).

NK ELISpot. Purified NK cells were treated with IL-2 (1000 U/mL) or treated with the combination of IL-2 and anti-CD16 mAb (3 μg/ml) or IL-2+sAJ2 (Cells:sAJ2 1:20) and cultured with autologous monocytes at (1:1 NK:monocyte ratios). After overnight incubation, 50,000 cells per treatment group were seeded and the number of IFN-g spots were determined using ELISPOT assay (FIG. 45).

NK ELISA. Purified NK cells were treated with IL-2 (1000 U/mL) or treated with the combination of IL-2 and anti-CD16 mAb (3 μg/ml) or IL-2+sAJ2 (Cells:sAJ2 1:20) and cultured with autologous monocytes at (1:1 NK:monocyte ratios). After an overnight treatment at 37° C., the supernatants were harvested and the levels of IFN-γ secretion were measured using ELISA (Table 30).

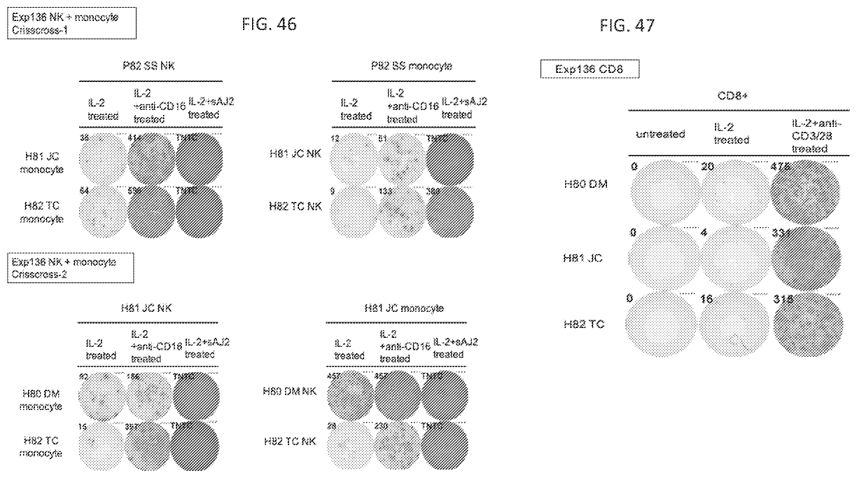

Comparable or increased IFN-g secretion by the autologous or allogeneic NK cells with and without autologous/allogeneic monocytes in ALS patient as compared to healthy twin, but increased IFN-g in CD8 T cells in ALS vs. healthy twin (FIG. 46-FIG. 47 and Tables 31-32).

NK ELISpot. Purified NK cells were treated with IL-2 (1000 U/mL) or treated with the combination of IL-2 and anti-CD16 mAb (3 μg/ml) or IL-2+sAJ2 (Cells:sAJ2 1:20) and cultured with allogeneic monocytes at (1:1 NK:monocyte ratios). After overnight incubation, 50,000 cells per treatment group were seeded and the number of IFN-g spots were determined using ELISPOT assay (FIG. 46).

NK ELISA. Purified NK cells were treated with IL-2 (1000 U/mL) or treated with the combination of IL-2 and anti-CD16 mAb (3 μg/ml) or IL-2+sAJ2 (Cells:sAJ2 1:20) and cultured with allogeneic monocytes at (1:1 NK:monocyte ratios). After an overnight treatment at 37° C., the supernatants were harvested and the levels of IFN-γ secretion were measured using ELISA (Table 31).

CD8 ELISpot. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISPOT assay to determine the number of spots expressing IFN-γ (FIG. 47).

CD8 ELISA. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA (Table 32).

### Example 16

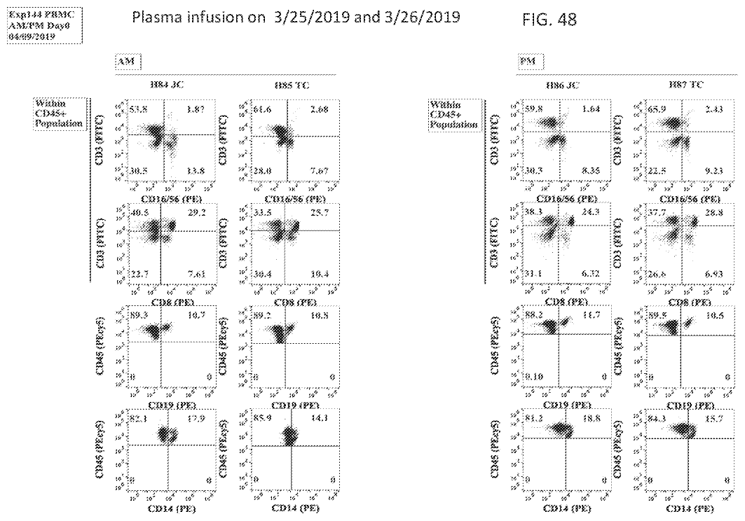

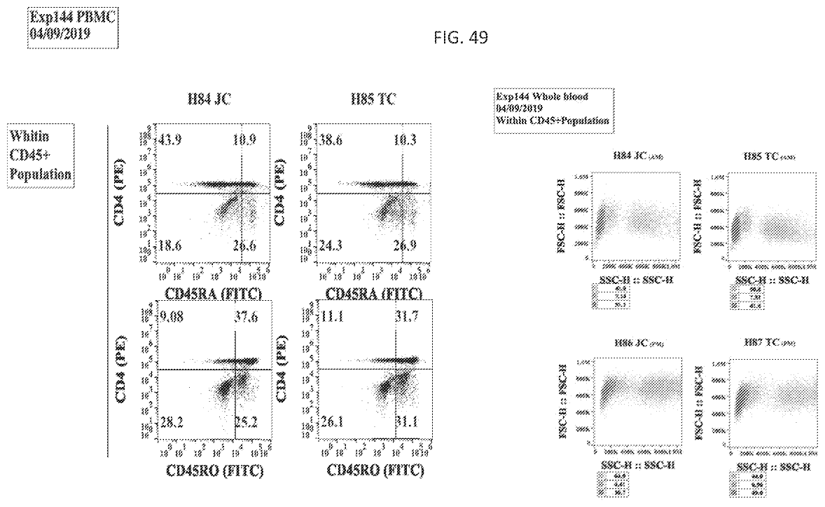

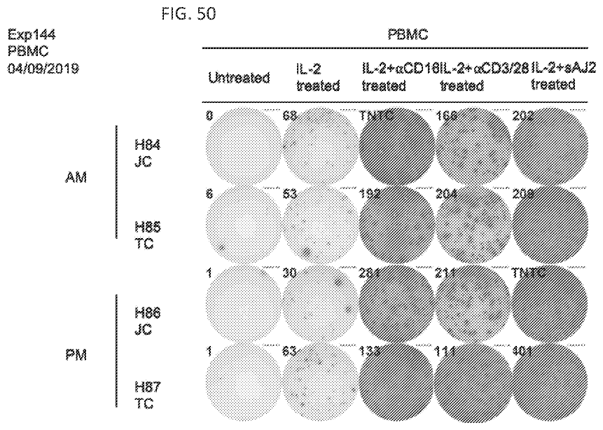

Comparable percentages of immune subsets in ALS patient as compared to healthy twin. PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors (FIG. 48 and Table 33).

Increased percentages of CD45RO in CD4+ T cells in ALS patient as compared to healthy twin (FIG. 49 and Tables 34-35). PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors (FIG. 49 and Tables 34-35).

Comparable cytotoxicity by PBMCs and increased or comparable cytotoxicity of sorted NK cells in the presence of autologous and allogeneic monocytes in ALS patient and healthy twin (Tables 36-37).

PBMC cytotoxicity. PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (Table 36).

NK cytotoxicity. NK cells were isolated from PBMCs as described in the materials and methods section. Purified NK cells were left untreated, or treated with IL-2 (1000 U/mL) or treated with the combination of IL-2 and anti-CD16 mAb (3 μg/ml) or IL-2+sAJ2 (Cells:sAJ2 1:20). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100 (Table 37).

Increased IFN-g spots and secretion by the PBMCs of ALS patient as compared to healthy twin (FIG. 50 and Table 38).

PBMC Elispot. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay (FIG. 50).

PBMC ELISA. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA (Table 38).

Increased IFN-g secretion by the CD8+ T cells in ALS patient as compared to healthy twin (FIG. 51 and Table 39).

CD8 Elispot. CD8+ T cells were isolated from PBMC following manufacturer's instruction and they were either left untreated, treated with IL-2 (1000 U/mL) or treated with the combination of IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay (FIG. 51).

PBMC ELISA. CD8+ T cells were isolated from PBMC following manufacturer's instruction and they were left untreated, treated with IL-2 (1000 U/mL) or treated with the combination of IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA (Table 39).

Comparable or increased IFN-g secretion by NK cells with and without autologous monocytes and naïve CD4+ T cells differentiated to Tregs in ALS patient as compared to healthy twin (Tables 40-41).

NK Elisa. Purified NK cells were treated with IL-2 (1000 U/mL) or treated with the combination of IL-2 and anti-CD16 mAb (3 μg/ml) or IL-2+sAJ2 (Cells:sAJ2 1:20) and cultured with autologous monocytes at (1:1 NK:monocyte ratios). After an overnight treatment at 37° C., the supernatants were harvested and the levels of IFN-γ secretion were measured using ELISA (Table 40).

Naïve CD4 Elisa. Naïve CD4+ T cells were isolated as suggested by the manufacturer and differentiated to T regulatory cells and supernatants were collected at the indicated days in the figure and the levels of IFN-γ secretion were measured using ELISA (Table 41).

### Example 17

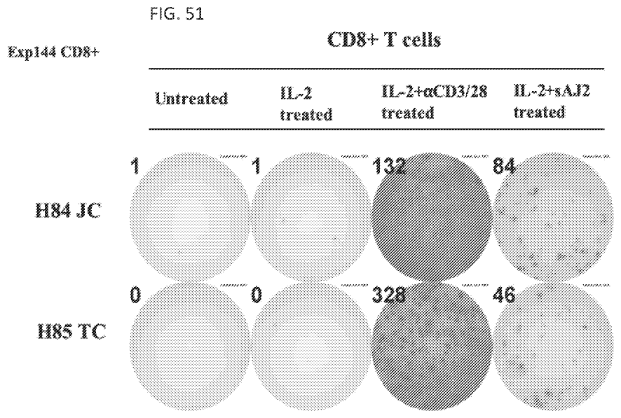

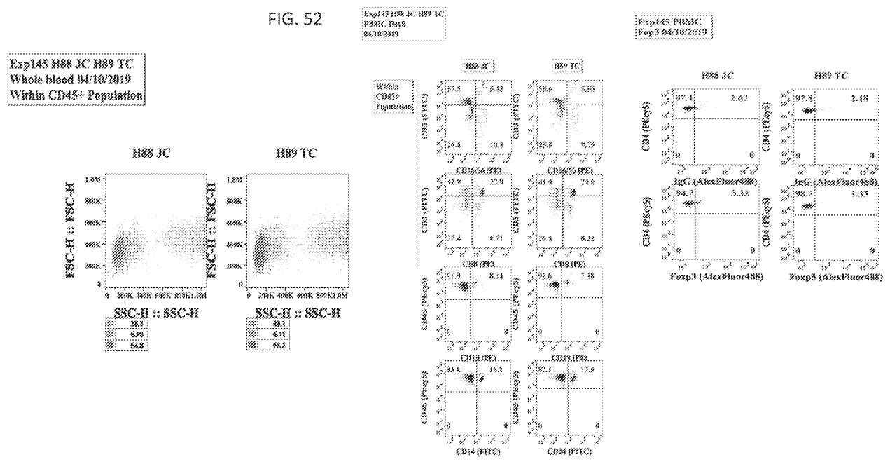

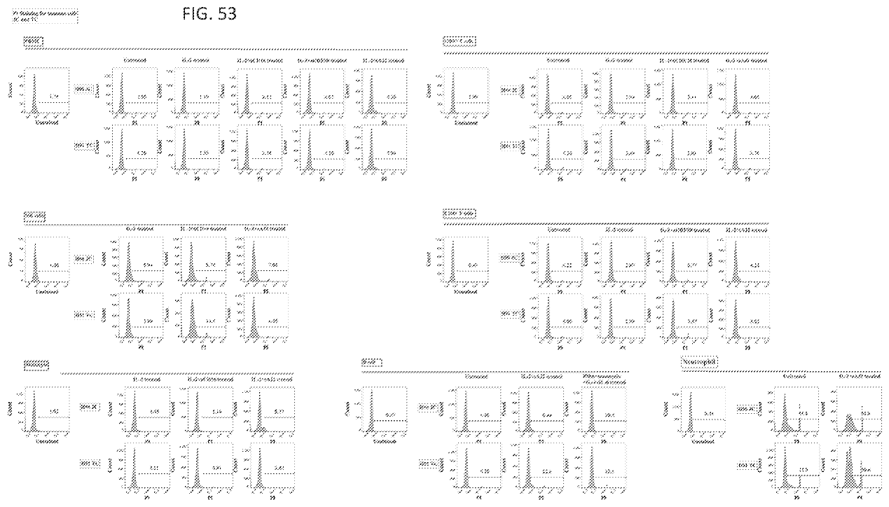

Similar proportions of immune subsets in ALS patient as compared to healthy twin (FIG. 52 and Table 42). PBMC Flow. PBMCs were isolated from peripheral blood of donors using ficoll-hypaque gradient centrifugation. The percentages of each cell subset including T reg in the PBMCs were determined by flow cytometric analysis after staining with specific fluorescent conjugated antibodies directed to the cell surface receptors (FIG. 52 and Table 42).

No significant differences in cell death in different immune subsets after activation in ALS patient as compared to healthy twin (FIG. 53). Propidium iodide staining for cell death. Each cell subset was isolated as per manufacturers suggestion and treated as described above and after an overnight incubation the cells were stained with PI and the fraction of cells stained were PI was determined by flow cytometric analysis (FIG. 53).

No significant differences in NK cell cytotoxicity within PBMCs in ALS patient as compared to healthy twin (Table 43). PBMC cytotoxicity. PBMC were left untreated, or treated with IL-2 (1000 U/mL) or treated with the combination of IL-2 and anti-CD16 mAb (3 μg/ml), or IL-2+anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (Table 43).

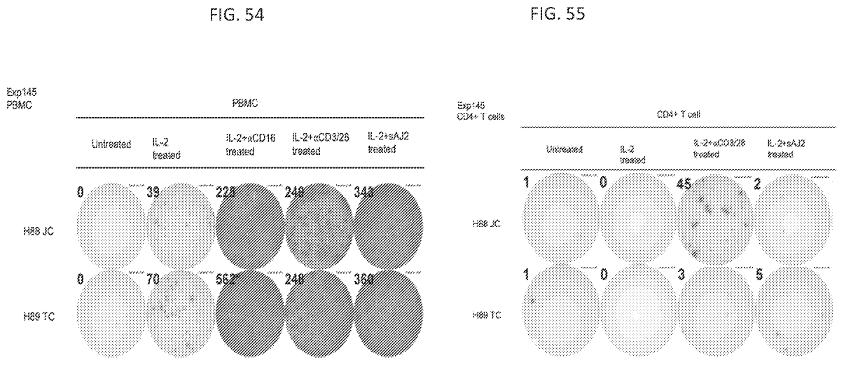

Increased IFN-g secretion by PBMCs and CD4+ T cells activated with anti-CD3/28 in ALS patient as compared to healthy twin (FIG. 54-FIG. 55 and Tables 44-45).

PBMCs and CD4+ Elispot. PBMCs and purified CD4+ T cells were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After an overnight incubation, 40,000 to 50,000 cells per treatment group were seeded and the number of IFN-g spots were determined using ELISPOT assay (FIG. 54-FIG. 55).

PBMC and CD4 T cell ELISA. PBMCs and purified CD4+ T cells were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA (Tables 44-45).

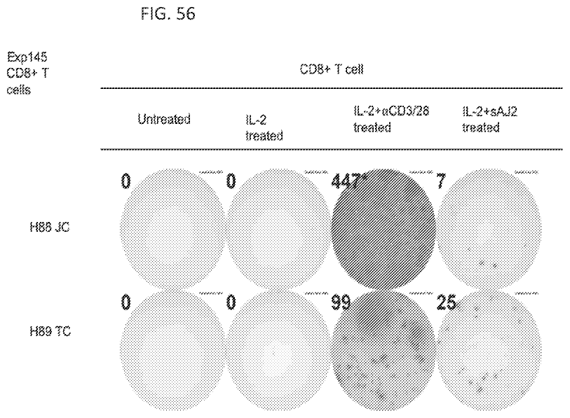

Increased IFN-g spots and secretion by CD8+ T cells activated with anti-CD3/28 in ALS patient as compared to healthy twin (FIG. 56 and Table 46).

CD8 Elispot. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISPOT assay to determine the number of spots expressing IFN-γ (FIG. 56).

CD8 Elisa. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA (Table 46).

### Example 18

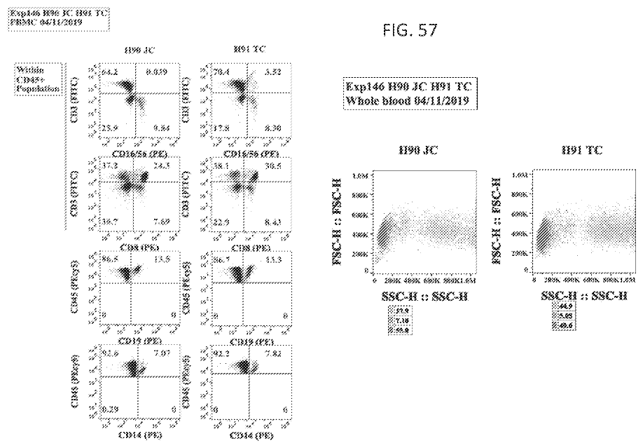

Similar proportions of immune subsets in ALS patient as compared to healthy twin (FIG. 57 and Table 47). PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors (FIG. 57 and Table 47).

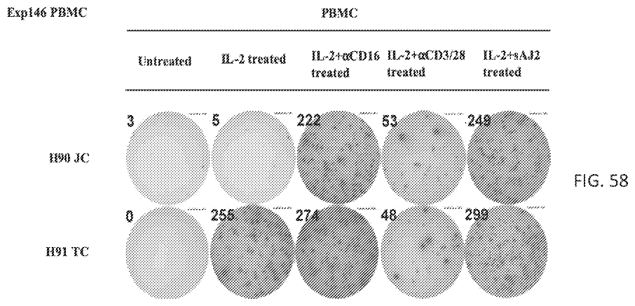

No significant differences in NK cell cytotoxicity and secretion of IFN-g within PBMCs in ALS patient as compared to healthy twin (FIG. 58 and Tables 48-49).

PBMC cytotoxicity. PBMC were left untreated, or treated with IL-2 (1000 U/mL) or treated with the combination of IL-2 and anti-CD16 mAb (3 μg/ml), or IL-2+anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (Table 48).

PBMC Elispot. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay (FIG. 58).

PBMC ELISA. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA (Table 49).

No significant increase in IFN-g secretion by Neutrophils in ALS patient as compared to healthy twin (Table 50). Neutrophil ELISA. Neutrophils were isolated from the PBMCs as suggested by the manufacturer and they were left untreated, or treated with IL-2+sAJ2 (Cells:sAJ2 1:20). After an overnight treatment at 37° C., the supernatants were harvested and the levels of IFN-γ secretion were measured using ELISA (Table 50).

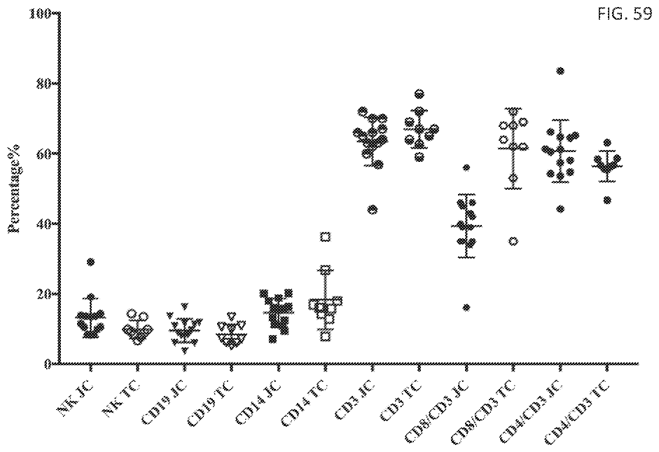

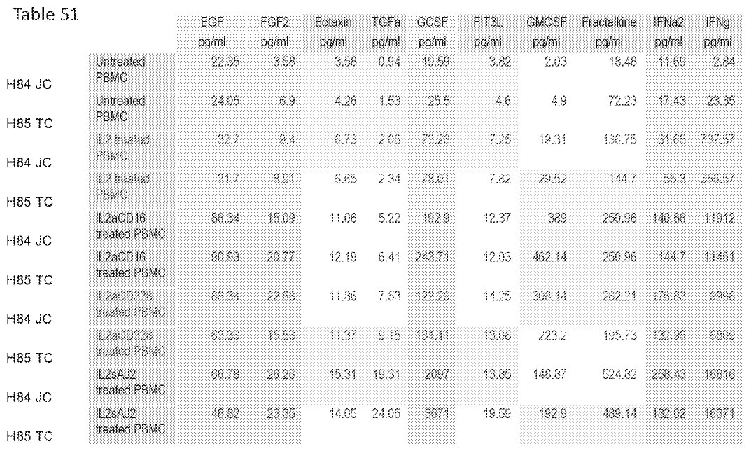

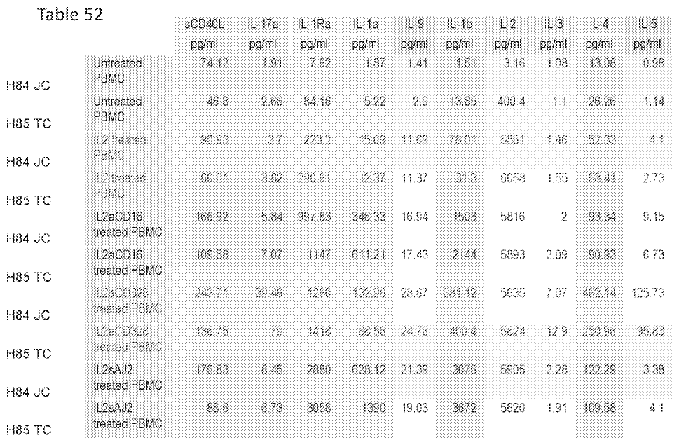

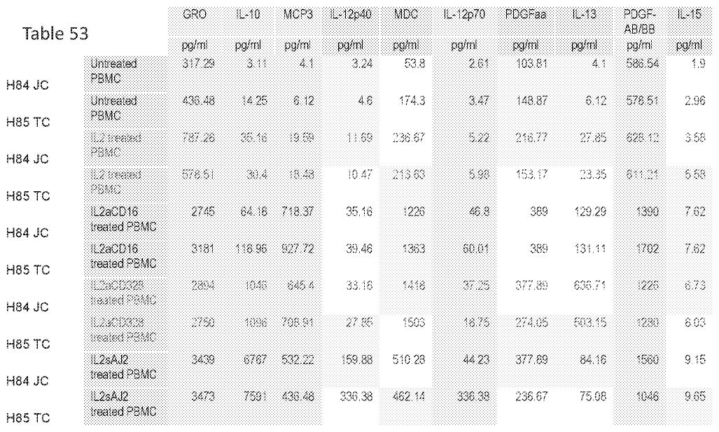

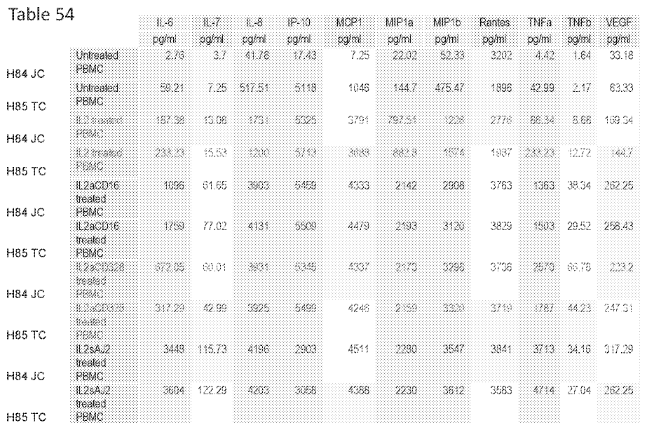

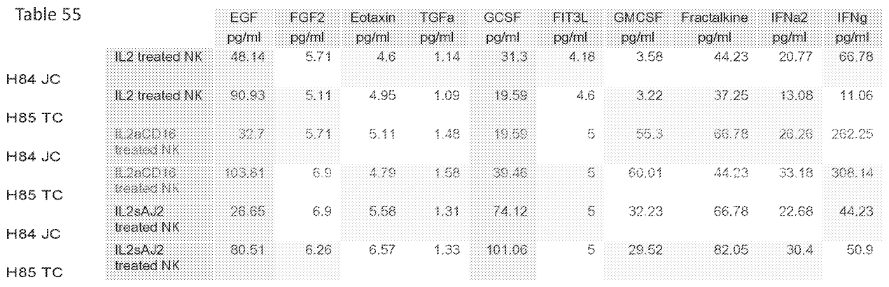

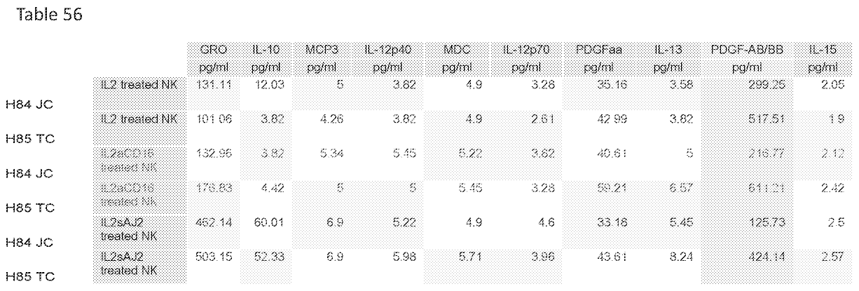

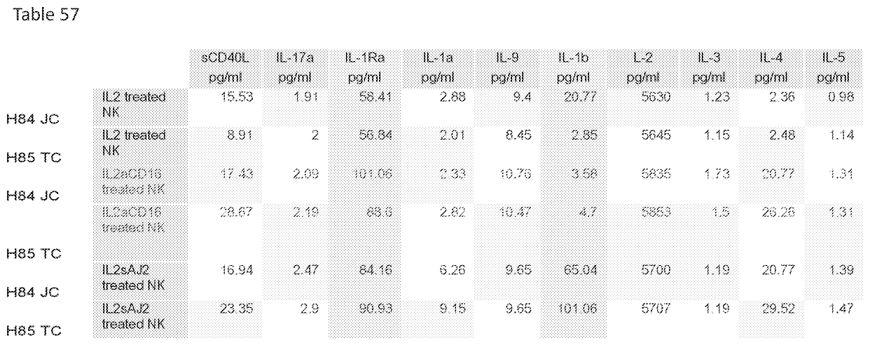

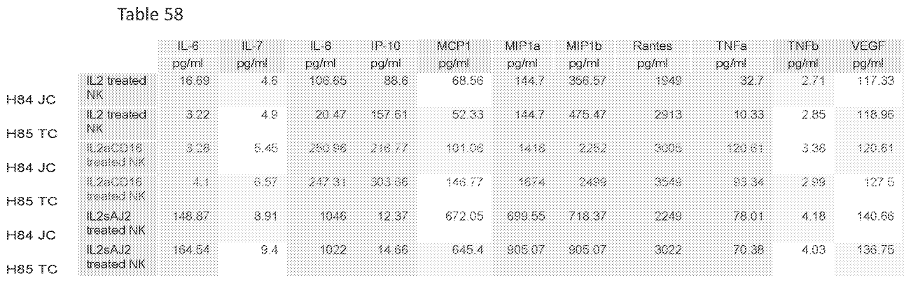

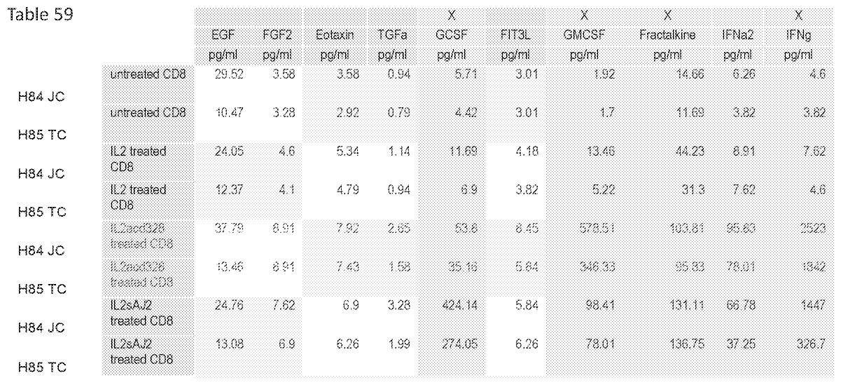

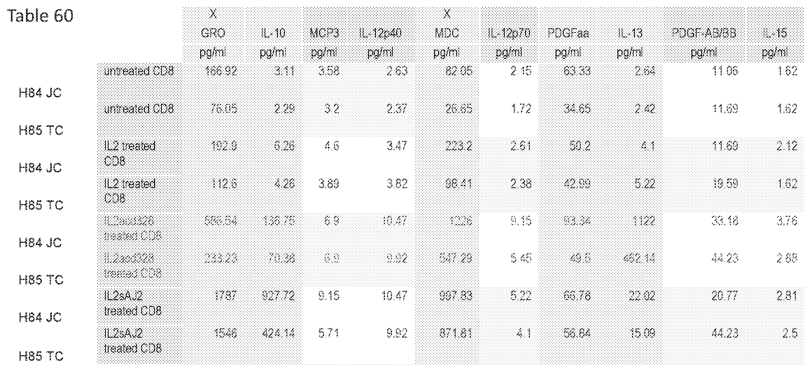

Similar proportions of immune subsets with the exception of CD8+ T cells in ALS patient as compared to healthy twin, Decrease in CD8+ T cell percentages in ALS patient (FIG. 59). PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors. The percentages of each subset in each donor were plotted (JC: n=14, TC: n=9) (FIG. 59).

### Example 19

Increased cytokine/chemokine/growth factor/ligands in PBMCs of patient activated with anti-CD3/28 and no difference between those activated with IL-2+anti-CD16 or IL-2+sAJ2 when compared to healthy twin (Tables 51-54). PBMC Luminex. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of cytokine/chemokine/growth factor/ligands secretion were measured using multiplex luminex assay (Millipore, MA) (Tables 51-54).

### Example 20

No overall differences in cytokine/chemokine/growth factor/ligands in sorted NK cells of patient activated with IL-2 or IL-2+antiCD16 or IL-2+sAJ2 when compared to healthy twin (Tables 55-58). NK Luminex. NK cells were left untreated, treated with IL-2 (1000 U/mL) or treated with the combination of IL-2 and anti-CD16 mAb (3 μg/ml), or IL-2+sAJ2 (Cells:sAJ2 1:20). After an overnight treatment at 37° C., the supernatants were harvested and the levels of cytokine/chemokine/growth factor/ligands secretion were measured using multiplex luminex assay (Millipore, MA) (Tables 55-58).

### Example 21

Increased overall cytokine/chemokine/growth factor/ligands in CD8+ T cells of patient untreated or activated with IL-2 or anti-CD3/28 or IL-2+sAJ2 when compared to healthy twin (Tables 59-62). CD8+ T cell Luminex. Sorted CD8+ T cells were left untreated, treated with IL-2 (1000 U/mL) or treated with the combination of IL-2+anti-CD3/28 antibodies (25 μl/mL) or IL-2+sAJ2 (Cells:sAJ2 1:20). After an overnight treatment at 37° C., the supernatants were harvested and the levels of cytokine/chemokine/growth factor/ligands secretion were measured using multiplex luminex assay (Millipore, MA) (Tables 59-62).

### Example 22

No differences or decreased cytokine/chemokine/growth factor/ligands in naïve CD4+ T cells differentiated to Tregs of patient activated with anti-CD3/28 when compared to healthy twin (Tables 63-66). T reg Luminex. Naïve CD4+ T cells were sorted out from PBMCs and differentiated to Tregs as per manufacturers suggestion. After several days of differentiation (nCD-1: overnight, nCD4-2: Day3) at 37° C., the supernatants were harvested and the levels of cytokine/chemokine/growth factor/ligands secretion were measured using multiplex luminex assay (Millipore, MA) (Tables 63-66).

### Example 23

Increased cytokine/chemokine/growth factor/ligands in Tregs sorted from the patient when compared to healthy twin (Tables 67-70). Treg Luminex Tregs were sorted out from the peripheral blood and treated with IL-2+anti-CD3/28 antibodies (25 μl/mL). After 72 hours treatment at 37° C., the supernatants were harvested and the levels of cytokine/chemokine/growth factor/ligands secretion were measured using multiplex luminex assay (Millipore, MA) (Tables 67-70).

### Example 24

Increased cytokine/chemokine/growth factor/ligands in B cells of patient activated with IL-2+sAJ2 or PMA/Ionomycin when compared to healthy twin (Tables 71-74). B cells Luminex. B cells were sorted out from the PBMCs as suggested by the manufacturer and left untreated, or treated with IL-2+sAJ2 (Cells:sAJ2 1:20) or PMA (10 ng/mL)+ Ionomycin (10 ng/mL). After an overnight treatment at 37° C., the supernatants were harvested and the levels of cytokine/chemokine/growth factor/ligands secretion were measured using multiplex luminex assay (Millipore, MA).

### Example 25

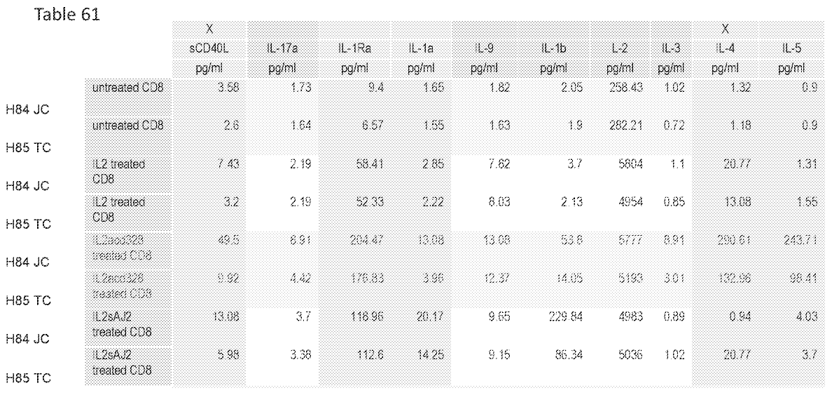

Comparable percentages of immune subsets with the exception of CD8+ T cells and Tregs in ALS patient as compared to healthy twin. Lower CD8+ and Tregs in ALS than control twin (Table 75 and FIG. 60). PBMC Flow. PBMCs were isolated from peripheral blood of donors using ficoll-hypaque gradient centrifugation. The percentages of each cell subset including T reg in the PBMCs were determined by flow cytometric analysis after staining with specific fluorescent conjugated antibodies directed to the cell surface receptors (Table 75 and FIG. 60).

Comparable cytotoxicity of NK cells from PBMCs and purified NK cells between ALS and healthy controls (Tables 76-77).

PBMC cytotoxicity. PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (Table 76).

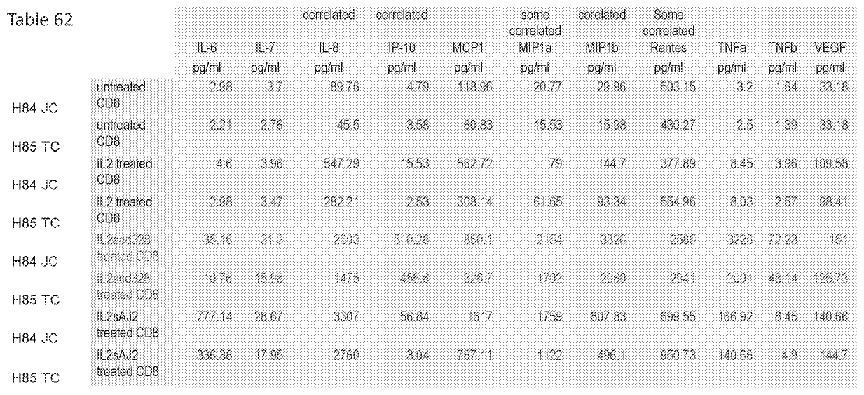

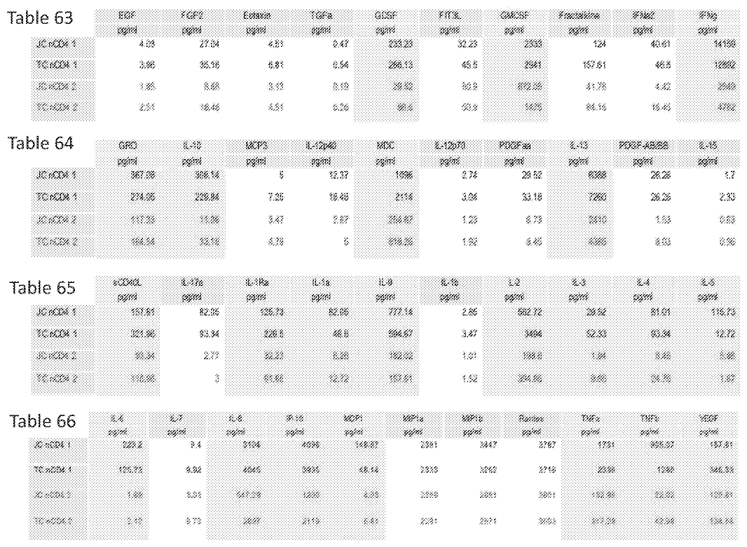

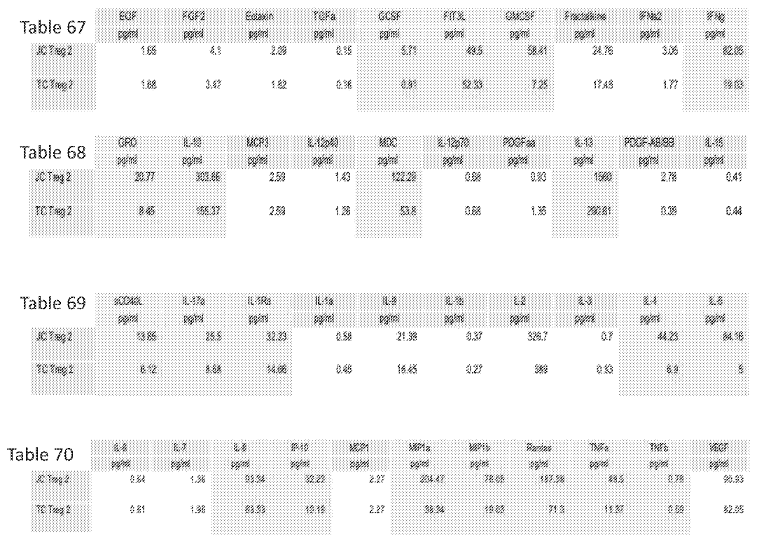

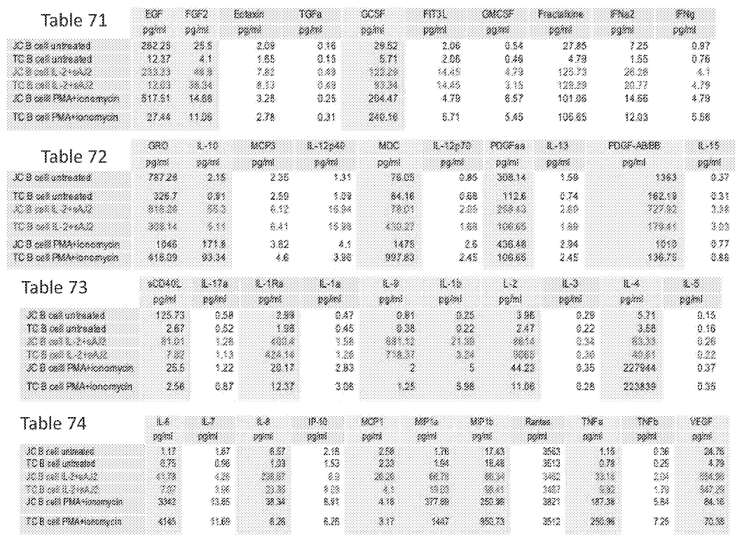

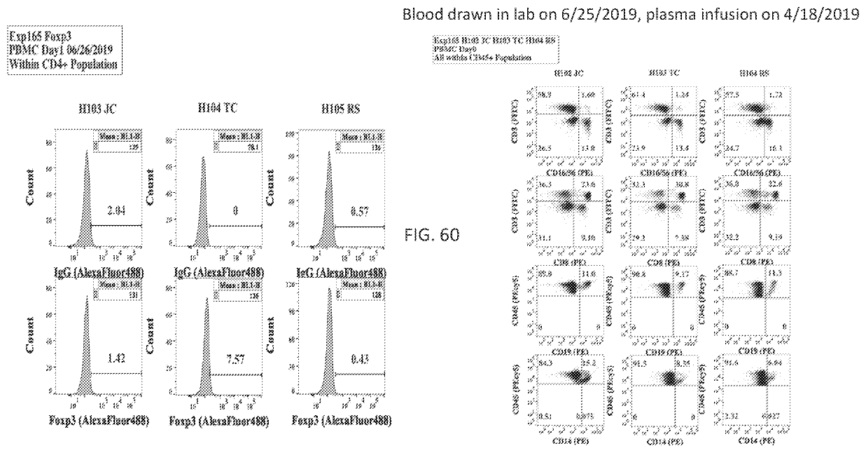

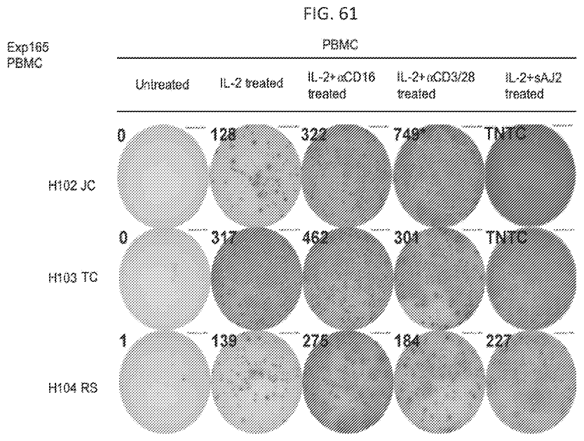

NK cytotoxicity. NK cells were isolated from PBMCs as described in the materials and methods section. Purified NK cells were left untreated, or treated with IL-2 (1000 U/mL) or treated with the combination of IL-2 and anti-CD16 mAb (3 μg/ml) or IL-2+sAJ2 (Cells:sAJ2 1:20). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100 (Table 77). Comparable or higher secretion and spots of IFN-g from the PBMCs between ALS and healthy twin. Differences between the two and RS another patient with ALS (FIG. 61 and Table 78).

PBMC Elispot. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay (FIG. 61).

PBMC ELISA. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA (Table 78).

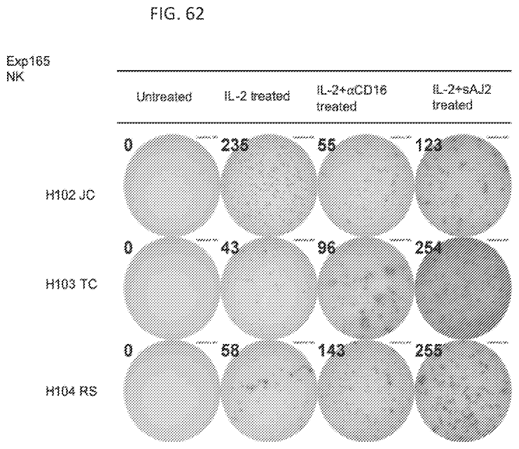

Comparable secretion and spots of IFN-g from the purified NK cells between ALS and healthy controls (FIG. 62 and Table 79).

NK Elispot. NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment, 50,000 cells per treatment group were used in ELISPOT assay to determine the number of spots expressing IFN-γ (FIG. 62).

NK Elisa. NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment at 37° C., the supernatants were harvested and the levels of IFN-γ secretion were measured using ELISA (Table 79).

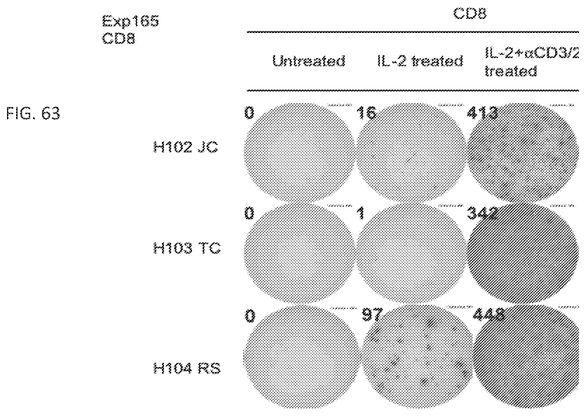

Increased IFN-g spots and secretion by CD8+ T cells activated with anti-CD3/28 in ALS patients as compared to healthy controls (FIG. 63 and Table 80).

CD8 Elispot. CD8+ T cells were isolated from PBMC following manufacturer's instruction and they were either left untreated, treated with IL-2 (1000 U/mL) or treated with the combination of IL-2+anti-CD3/28 antibodies (25 μl/mL). After overnight incubation, 40,000 cells per treatment group were seeded and the number of IFN-g spots were determined using ELISPOT assay (FIG. 63).

CD8 ELISA. CD8+ T cells were isolated from PBMC following manufacturer's instruction and they were left untreated, treated with IL-2 (1000 U/mL) or treated with the combination of IL-2+anti-CD3/28 antibodies (25 μl/mL). After an overnight treatment at 37° C., the supernatants were harvested and the levels of IFN-γ secretion were measured using ELISA (Table 80).

### Example 26

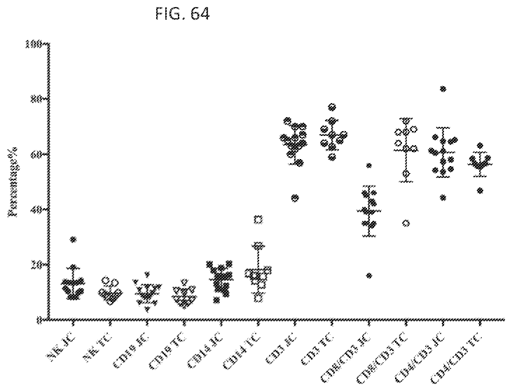

Similar proportions of immune subsets with the exception of CD8+ T cells in ALS patient as com. PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors. The percentages of each subset in each donor were plotted (JC:n=14, TC: n=9) pared to healthy twin, Decrease in CD8+ T cell percentages in ALS patient (FIG. 64).

### Example 27

Increased overall cytokine/chemokine/growth factor/ligands in CD8+ T cells of patient untreated or activated with IL-2 or anti-CD3/28 or IL-2+sAJ2 when compared to healthy twin (Tables 81-84). CD8+ T cell Luminex. Sorted CD8+ T cells were left untreated, treated with IL-2 (1000 U/mL) or treated with the combination of IL-2+anti-CD3/28 antibodies (25 μl/mL) or IL-2+sAJ2 (Cells:sAJ2 1:20). After an overnight treatment at 37° C., the supernatants were harvested and the levels of cytokine/chemokine/growth factor/ligands secretion were measured using multiplex luminex assay.

### Example 28

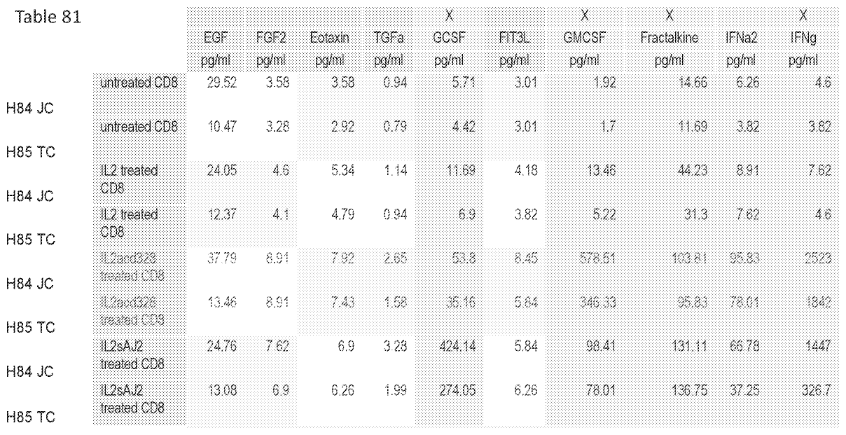

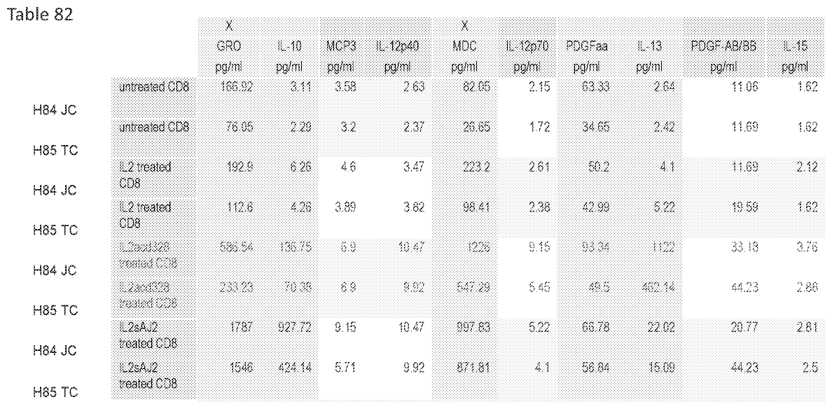

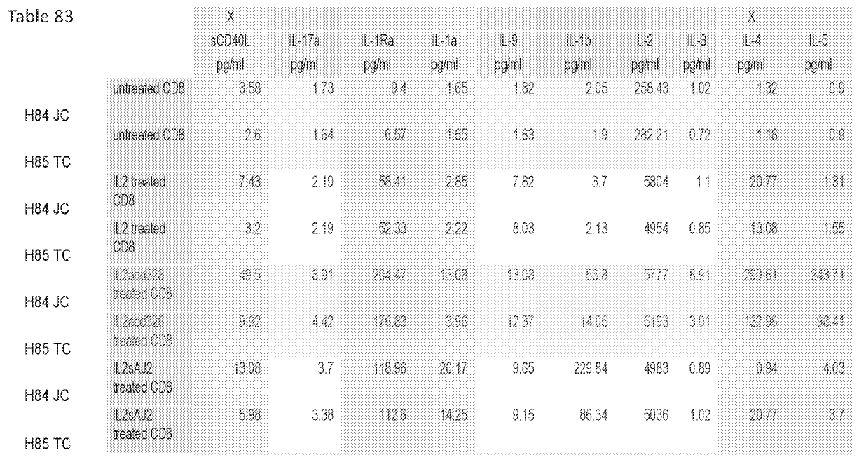

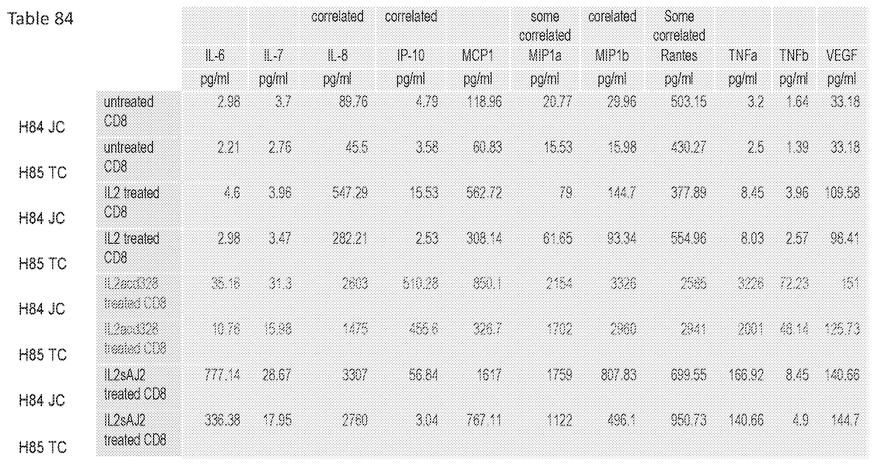

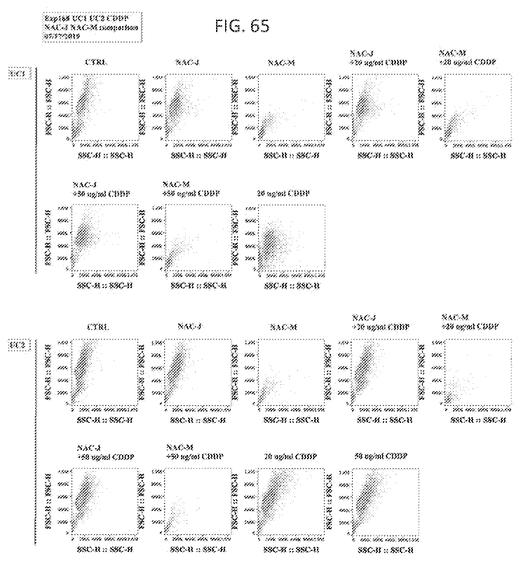

Testing and selection of the best NAC to be used in ALS treatment. NAC blocks CDDP mediated Cell death in Oral tumors (FIG. 65). Oral squamous carcinoma cells (OSCCs) and Oral squamous stem cell carcinoma (OSCSCs) tumors were cultured (0.1-0.2 million/ml) in 12 well plates overnight before they were treated with different concentrations of CDDP, a chemotherapeutic agents which is known to induce cell death as shown in the figure in the presence of NAC-M (20 mM, clinical grade obtained from dr. Maharaj for the treatment of the ALS patient) and NAC-J (20 mM, control NAC usually used at the Jewett laboratory) for 24 hours. Afterwards OSCCs and OSCSCs were detached, and the viability of cells was determined after PI staining followed by flow cytometric analysis. In this figure Forward and Side Scatters (Fs/ss) were assessed since decrease in the Fs/ss is seen in cells undergoing cell death. Initially, NAC-M preparations were not conducted correctly and therefore, we were able to ask the pharmacy to change the NAC preparations to prepare effective NAC-M for treatment of the patient. In addition, CDDP induces more of the cell death in OSCC than in OSCSCs.

### Example 29

Testing and selection of the best NAC to be used in ALS treatment. NAC blocks CDDP mediated Cell death in Oral tumors (FIG. 66). Oral squamous carcinoma cells (OSCCs) and Oral squamous stem cell carcinoma (OSCSCs) tumors were cultured (0.1-0.2 million/ml) in 12 well plates overnight before they were treated with different concentrations of CDDP, a chemotherapeutic agents which is known to induce cell death as shown in the figure in the presence of NAC-M (20 mM, clinical grade obtained from dr. Maharaj for the treatment of the ALS patient) and NAC-J (20 mM, control NAC usually used at the Jewett laboratory) for 24 hours. Afterwards OSCCs and OSCSCs were detached, and the viability of cells was determined after PI staining followed by flow cytometric analysis. In this figure the percentage of PI stained cells were assessed. Initially, NAC-M preparations were not conducted correctly and therefore, we were able to ask the pharmacy to change the NAC preparations to prepare effective NAC-M for treatment of the patient. In addition, CDDP induces more of the cell death in OSCC than in OSCSCs.

### Example 30

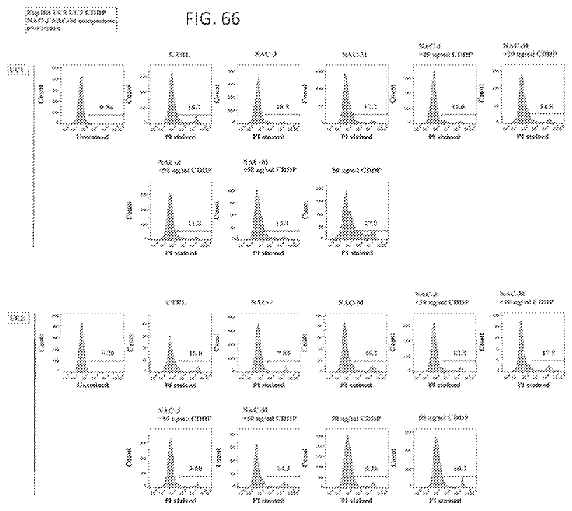

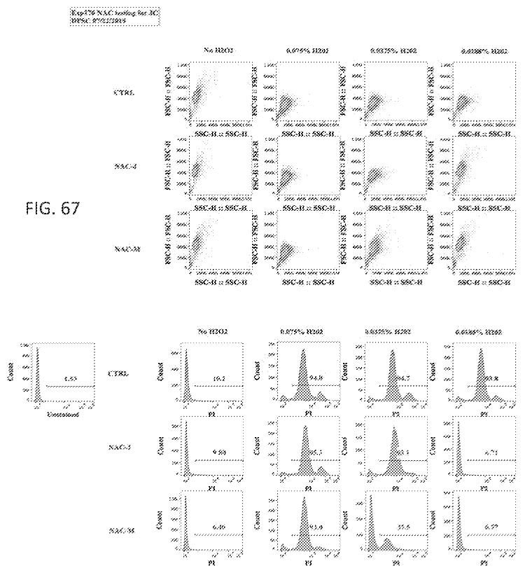

Testing and selection of the best NAC to be used in ALS treatment. NAC blocks H2O2 mediated Cell death in DPSCs (FIG. 67). Dental Pulp Stromal Cells (DPSCs) were cultured (0.1-0.2 million/ml) in 12 well plates overnight before they were treated with different concentrations of H2O2 used in bleaching teeth which is known to induce cell death as shown in the figure in the presence of NAC-M (20 mM, clinical grade obtained from dr. Maharaj for the treatment of the ALS patient) and NAC-J (20 mM, control NAC usually used at the Jewett laboratory) for 24 hours. Afterwards DPSCs were detached, and the viability of cells was determined after PI staining followed by flow cytometric analysis. In this figure the decrease in FS/ss and the percentage of PI stained cells were assessed. After optimizations of NAC-M preparations it was able to inhibit H2O2 mediated cell death effectively as assessed both by Fs/ss and PI positive DPSCs.

### Example 31

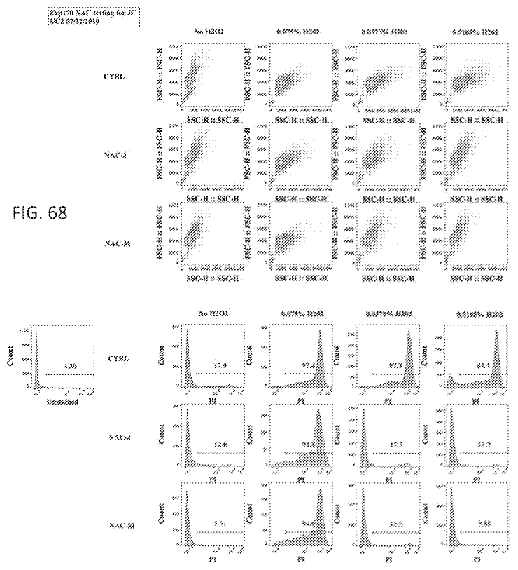

Testing and selection of the best NAC to be used in ALS treatment. NAC blocks H2O2 mediated Cell death in OSCSCs (FIG. 68). OSCSCs were cultured (0.1-0.2 million/ml) in 12 well plates overnight before they were treated with different concentrations of H2O2 used in bleaching teeth which is known to induce cell death as shown in the figure in the presence of NAC-M (20 mM, clinical grade obtained from dr. Maharaj for the treatment of the ALS patient) and NAC-J (20 mM, control NAC usually used at the Jewett laboratory) for 24 hours. Afterwards DPSCs were detached, and the viability of cells was determined after PI staining followed by flow cytometric analysis. In this figure the decrease in FS/ss and the percentage of PI stained cells were assessed. After optimizations of NAC-M preparations it was able to inhibit H2O2 mediated cell death effectively as assessed both by Fs/ss and PI positive DPSCs.

### Example 32

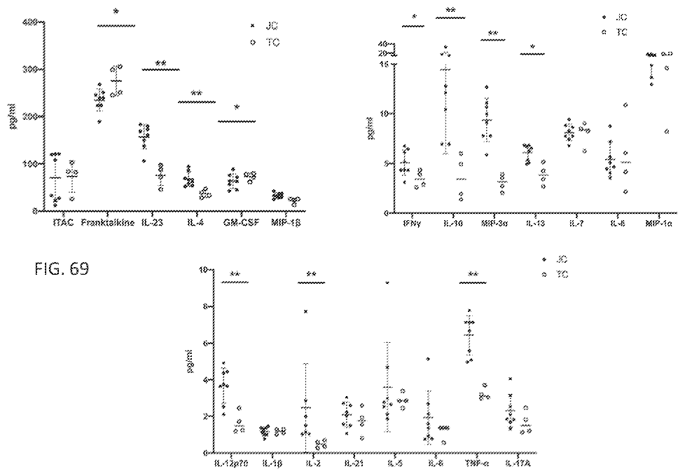

Increased cytokine/chemokine/growth factor/ligands in serum of ALS patient compared to healthy twin and control (Tables 85-86 and FIG. 69). Serum Luminex. Serum from patients were used to determine the levels of cytokine/chemokine/growth factor/ligands secretion using multiplex luminex assay. Scatter plot was prepared using the levels of cytokine/chemokine/growth factor/ligands in serum obtained at different dates from patients as compared to controls.

### Example 33

Increased cytokine/chemokine/growth factor/ligands in serum of ALS patients compared to healthy twin (Tables 87-89). Serum Luminex. Serum from patients were used to determine the levels of cytokine/chemokine/growth factor/ligands secretion using multiplex luminex assay.

### Example 34

Increased overall cytokine/chemokine/growth factor/ligands in CD8+ T cells of patients untreated or activated with IL-2 or anti-CD3/28 or IL-2+sAJ2 when compared to healthy twin (Tables 90-94). CD8+ T cell Luminex. Sorted CD8+ T cells were left untreated, treated with IL-2 (1000 U/mL) or treated with the combination of IL-2+anti-CD3/28 antibodies (25 μl/mL) or IL-2+sAJ2 (Cells:sAJ2 1:20). After an overnight treatment at 37° C., the supernatants were harvested and the levels of cytokine/chemokine/growth factor/ligands secretion were measured using multiplex luminex assay.

### Example 35

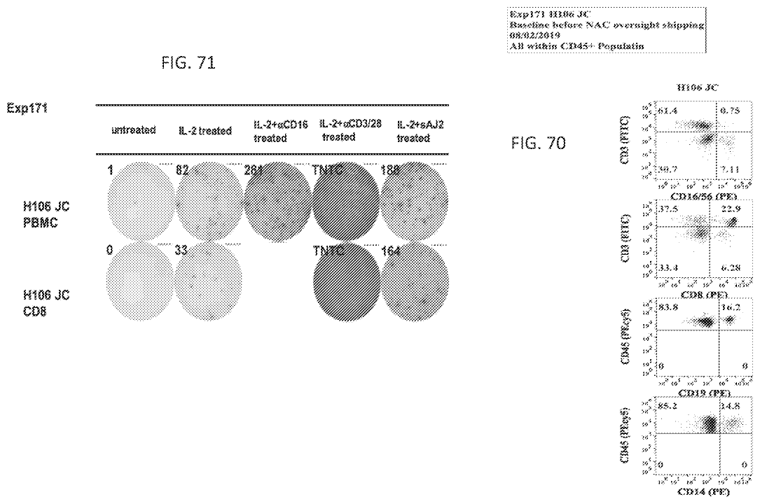

Higher secretion and spots of IFN-g and NK cell mediated cytotoxicity from the PBMCs and CD8+ T cells from ALS patient (FIG. 70-FIG. 71 and Tables 95-96).

PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors (Table 95 and FIG. 70).

PBMC chromium. PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (Table 96).

PBMC Elispot. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay (FIG. 71).

### Example 36

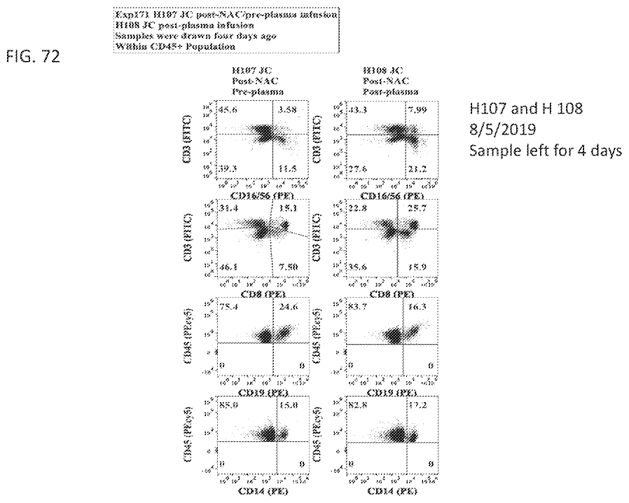

Percentages of different immune subsets in ALS patient (Table 97 and FIG. 72). PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors.

### Example 37

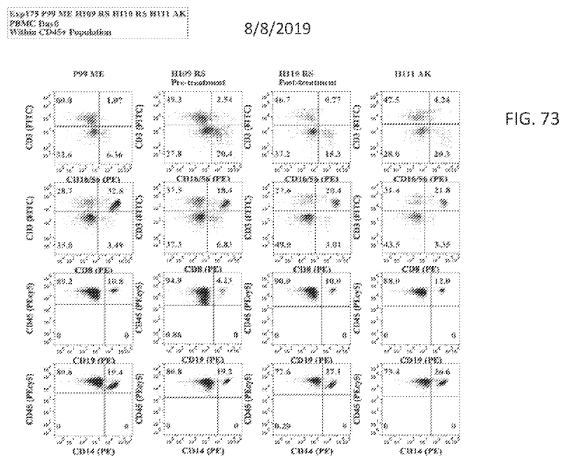

Percentages of different immune subsets in ALS patient pre and post NAC infusion compared to controls (FIG. 73 and Table 98). PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors.

NK cell mediated cytotoxicity from the PBMCs and NK cells from ALS patient before and after NAC infusion as compared to controls (Tables 99-100).

PBMC chromium. PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in the figure before low dose NAC infusion in patient (RS1) and after NAC infusion in patient (RS2). After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (Table 99).

NK cytotoxicity. NK cells were isolated from PBMCs as described in the materials and methods section. Purified NK cells were left untreated, or treated with IL-2 (1000 U/mL) or treated with the combination of IL-2 and anti-CD16 mAb (3 μg/ml) or IL-2+sAJ2 (Cells:sAJ2 1:20) before low dose NAC infusion in patient (RS1) and after NAC infusion in patient (RS2). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100 (Table 100).

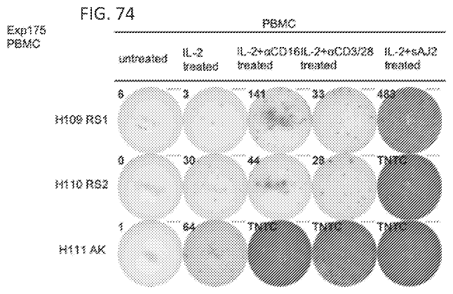

IFN-g spots from the PBMCs of ALS patient pre and post NAC infusion (FIG. 74). PBMC Elispot. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20) as shown in the figure before low dose NAC infusion in patient (RS1) and after NAC infusion in patient (RS2). After overnight incubation, 40,000 cells per treatment group were seeded and the number of IFN-g spots were determined using ELISPOT assay.

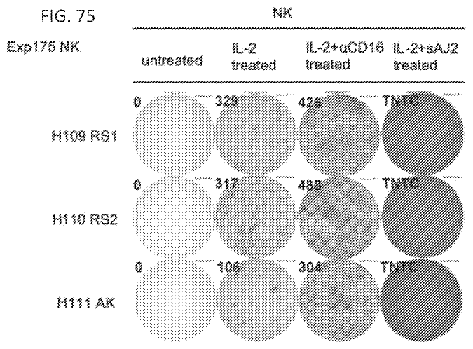

Similar spots of IFN-g in NK cell s from ALS patient pre and post NAC infusion (FIG. 75). NK Elispot. NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20) as shown in the figure before low dose NAC infusion in patient (RS1) and after NAC infusion in patient (RS2). After an overnight treatment, 50,000 cells per treatment group were used in ELISPOT assay to determine the number of spots expressing IFN-γ.

### Example 38

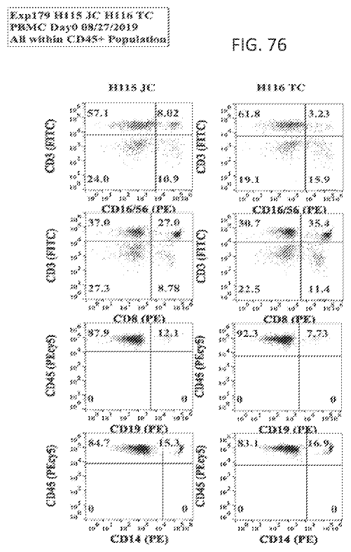

No differences in the percentages of immune subsets between ALS and control twin (FIG. 76 and Table 101). PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors.

No significant differences in the cytotoxicity of NK cells between ALS and control twin (Table 102). PBMC cytotoxicity. PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100.

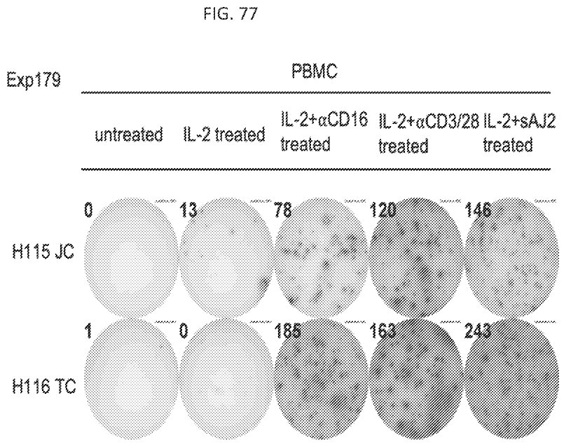

IFN-g spots are higher in IL-2 activated PBMCs but not under all other activation in PBMCs (FIG. 77). PBMC Elispot. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay.

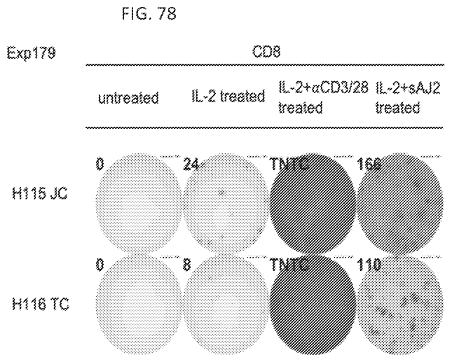

IFN-g spots and secretion are higher in CD8+ T cells from ALS patient in comparison to twin control (FIG. 78 and Tables 103-104).

CD8 Elispot. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISPOT assay to determine the number of spots expressing IFN-γ (FIG. 78).

PBMC ELISA. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA (Table 103).

CD8 Elisa. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA (Table 104).

### Example 39

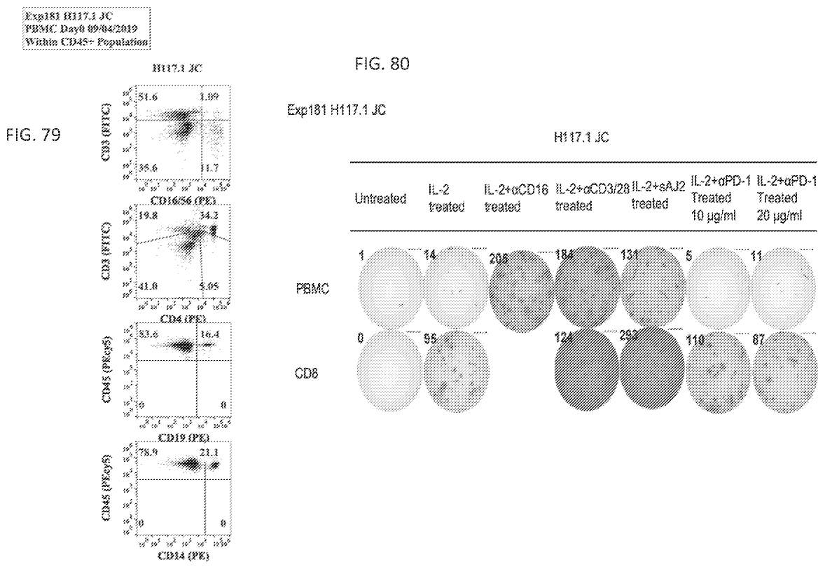

PBMC Flow (FIG. 79 and Table 105). PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors (FIG. 79 and Table 105).

IFN-g spots are higher in activated CD8s when compared to PBMCs and anti-PD-1 antibody increases CD8+ T cell mediated IFN-g spots and release (FIG. 80 and Tables 10⁶-108).

PBMC cytotoxicity. PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) and IL-2+anti-PD-1 antibody (10 and 20 mg/ml) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (Table 10⁶).

PBMC Elispot. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20) and IL-2+anti-PD-1 antibody (10 and 20 mg/ml). After overnight incubation, 40,000 cells per treatment group were seeded and the number of IFN-g spots were determined using ELISPOT assay (FIG. 80, upper panel).

PBMC ELISA. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20) and IL-2+anti-PD-1 antibody (10 and 20 mg/ml). After an overnight treatment at 37° C., the supernatants were harvested and the levels of IFN-γ secretion were measured using ELISA (Table 107).

CD8 Elispot. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20) and IL-2+anti-PD-1 antibody (10 and 20 mg/ml). After an overnight treatment, 50,000 cells per treatment group were used in ELISPOT assay to determine the number of spots expressing IFN-γ (FIG. 80, lower panel).

CD8 Elisa. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20) and IL-2+anti-PD-1 antibody (10 and 20 mg/ml). After an overnight treatment at 37° C., the supernatants were harvested and the levels of IFN-γ secretion were measured using ELISA (Table 108).

### Example 40

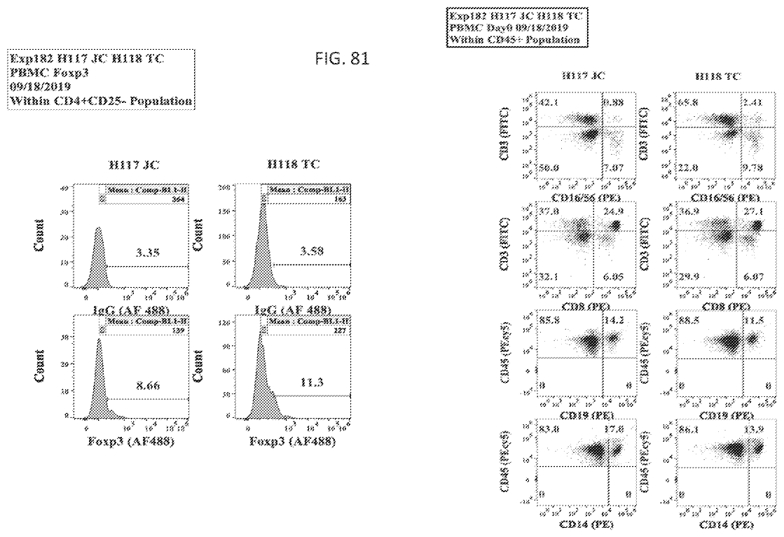

No differences in the percentages of key immune subsets between ALS and control twin, however, lower percentages of Tregs in ALS (Table 109 and FIG. 81). PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors, including the percentages of T regs.

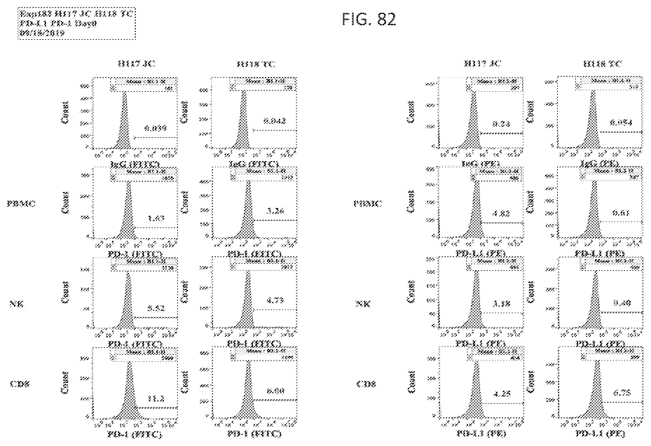

No differences in the percentages of PD-1 expression on PBMCs and NK cells with the exception of CD8+ T cells between ALS and control twin (FIG. 82). PBMC Flow. PBMCs were isolated from peripheral blood of donors using ficoll-hypaque gradient centrifugation. The percentages of PD-1 expression in PBMCs, NK and CD8+ T cells were determined by flow cytometric analysis after staining with specific fluorescent conjugated antibody directed to the PD-1 receptors.

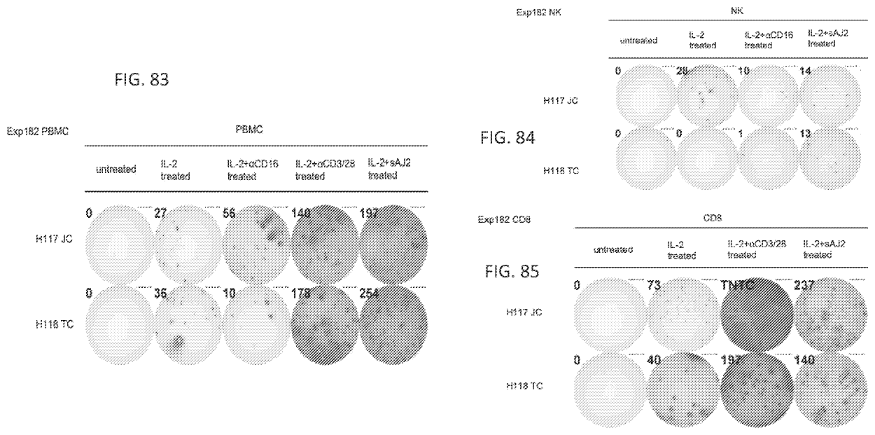

No significant differences in the NK cytotoxicity and IFN-g spots in PBMCs between ALS and control twin. Decreased NK cell mediated cytotoxicity in ALS for purified NK cells (FIG. 83 and Tables 110-111).

PBMC cytotoxicity. PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (Table 110).

NK cytotoxicity. NK cells were isolated from PBMCs as described in the materials and methods section. Purified NK cells were treated with IL-2 (1000 U/mL) or IL-2+sAJ2 (Cells:sAJ2 1:20). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100 (Table 111).

PBMC Elispot. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay (FIG. 83).

Increased IFN-g spots in CD8+ T cells in ALS when compared to control twin (FIG. 84-FIG. 85).

NK Elispot. NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment, 50,000 cells per treatment group were used in ELISPOT assay to determine the number of spots expressing IFN-γ (FIG. 84).

CD8 Elispot. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISPOT assay to determine the number of spots expressing IFN-γ (FIG. 85).

Increased IFN-g release in CD8+ T cells in ALS when compared to control twin (Tables 112-114).

PBMC ELISA. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA (Table 112).

NK Elisa. NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment at 37° C., the supernatants were harvested and the levels of IFN-γ secretion were measured using ELISA (Table 113).

CD8 Elisa. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA (Table 114).

### Example 41

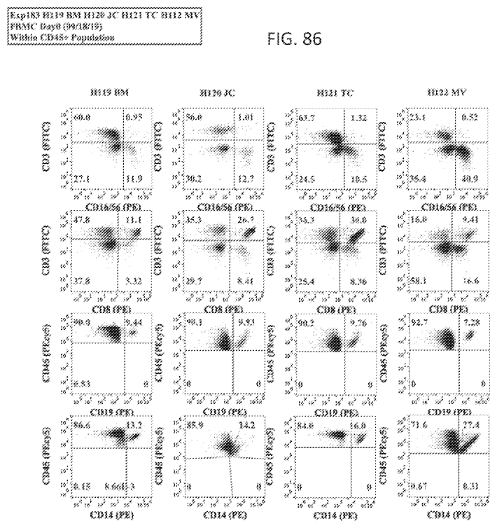

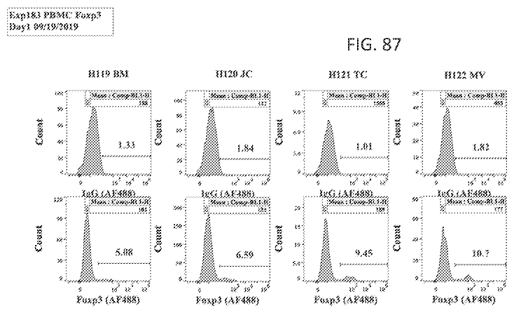

No differences in the percentages of key immune subsets between ALS and control twin (FIG. 86 and Table 115). PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors. Decreased percentages of Treg subsets between ALS and control twin (FIG. 87).

PBMC Flow. PBMCs were isolated from peripheral blood of donors using ficoll-hypaque gradient centrifugation. The percentages of Tregs in the PBMCs were determined by flow cytometric analysis after staining with specific fluorescent conjugated antibody directed to the Foxp3 intracellular staining.

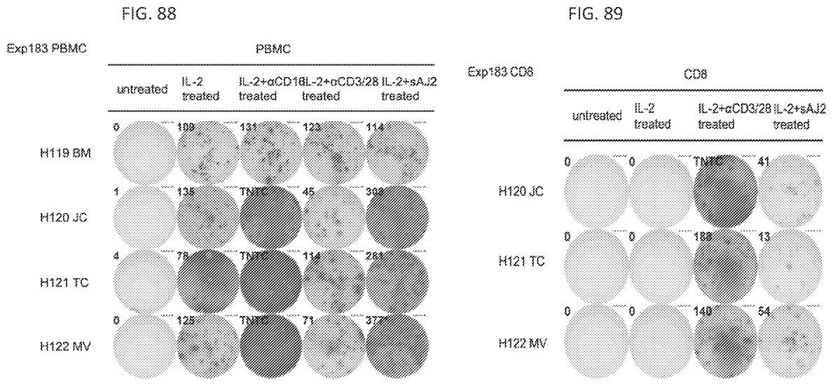

No differences in the IFN-g spots in PBMCs between ALS and control twin, and lower NK cytotoxicity in ALS (FIG. 88 and Table 116).

PBMC cytotoxicity. PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (Table 116).

PBMC Elispot. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay (FIG. 88).

No differences in the secretion of IFN-g by PBMCs but significant increases in INF-g spots and secretion in CD8+ T cells in ALS when compared to control twin (FIG. 89 and Tables 117-118).

CD8 Elispot. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISPOT assay to determine the number of spots expressing IFN-γ (FIG. 89).

PBMC ELISA. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA (Table 117).

CD8 Elisa. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA (Table 118).

### Example 42

Decreased cytokines/chemokines and growth factors with untreated Treg sorted and differentiated with Treg differentiation media by ALS patient when compared to control twin. Tregs treated with anti-CD3/antiCD28 mabs increase certain cytokines and decrease others (red colored) in ALS patient indicating potential priming effect in vivo (Tables 118-120). Treg Luminex. Tregs were sorted out from the peripheral blood and treated with IL-2+anti-CD3/28 antibodies (25 μl/mL). After an overnight treatment at 37° C., the supernatants were harvested and the levels of cytokine/chemokine/growth factor/ligands secretion were measured using multiplex luminex assay.

### Example 43

Increased cytokines/chemokines and growth factors by nCD4 sorted from ALS patient (day 1 sup) when compared to control twin (red color), the rest of cytokines and chemokines are within the range of control twin (TC) and an unrelated control (Tables 121-123). Naïve CD4+ Luminex. Naïve CD4+ T cells were sorted out from PBMCs and differentiated to Tregs as per manufacturers suggestion. After a day of differentiation at 37° C., the supernatants were harvested and the levels of cytokine/chemokine/growth factor/ligands secretion were measured using multiplex luminex assay.

### Example 44

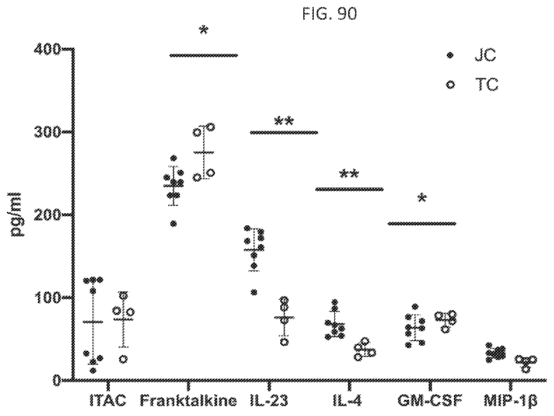

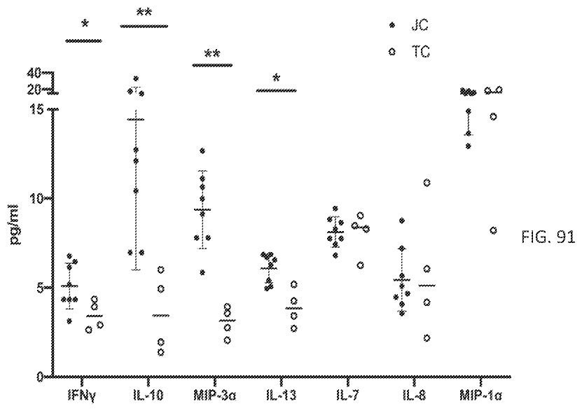

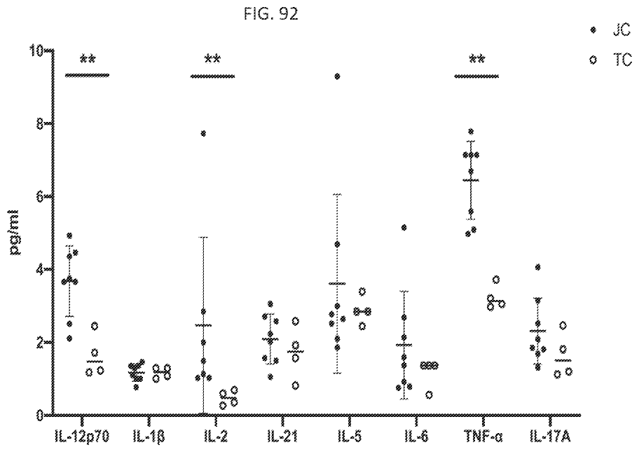

Significantly increased cytokines and chemokines in serum from ALS patient as compared to healthy twin before NAC treatment (FIG. 90-FIG. 92 and Table 124-126). 21 plex before NAC. Serum Luminex. Serum from patients were used to determine the levels of cytokine/chemokine/growth factor/ligands secretion using multiplex luminex assay. Scatter plot was prepared using the levels of cytokine/chemokine/growth factor/ligands in serum obtained at different dates from patients as compared to controls.

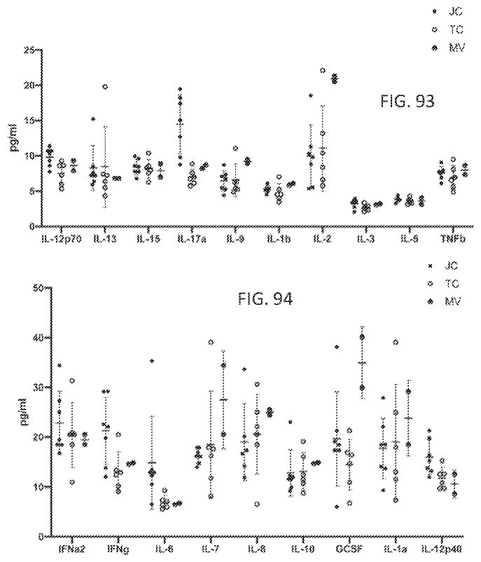

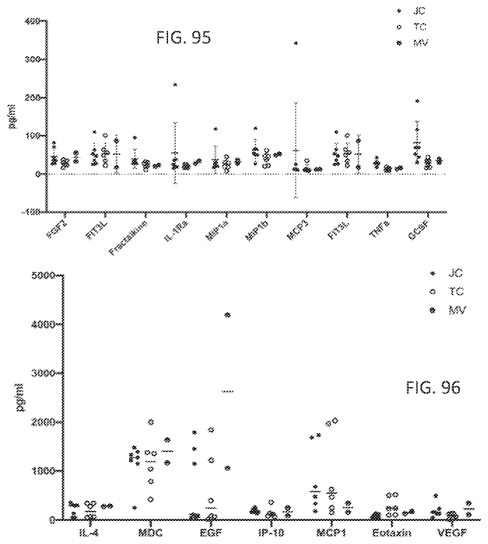

Significantly increased cytokines and chemokines in serum from ALS patient as compared to healthy twin after NAC treatment (FIGS. 93-97 and Tables 127-136). Serum Luminex. Serum from patients were used to determine the levels of cytokine/chemokine/growth factor/ligands secretion using multiplex luminex assay. Scatter plot was prepared using the levels of cytokine/chemokine/growth factor/ligands in serum obtained at different dates from patients as compared to controls.

Fold increase in respective cytokines before and after NAC treatment in serum from ALS patient as compared to healthy twin. After NAC treatment only IFN-g and TNF-α remained higher in ALS patient when compared to healthy twin (Table 137). Serum Luminex. Serum from patients were used to determine the levels of cytokine/chemokine/growth factor/ligands secretion using multiplex luminex assay. Scatter plot was prepared using the levels of cytokine/chemokine/growth factor/ligands in serum obtained at different dates from patients as compared to controls.

Decrease in respective cytokines after NAC treatment in serum from ALS patient Red colored cytokines have not changed whereas the blue colored cytokines have decreased (Table 138). Serum Luminex. Serum from patients were used to determine the levels of cytokine/chemokine/growth factor/ligands secretion using multiplex luminex assay. Scatter plot was prepared using the levels of cytokine/chemokine/growth factor/ligands in serum obtained at different dates from patients as compared to controls.

### Example 45

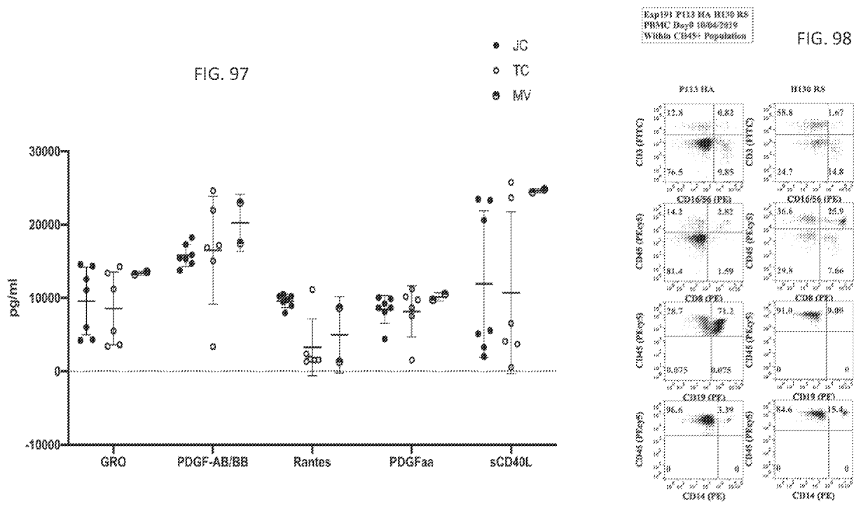

PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors (Table 129 and FIG. 98).

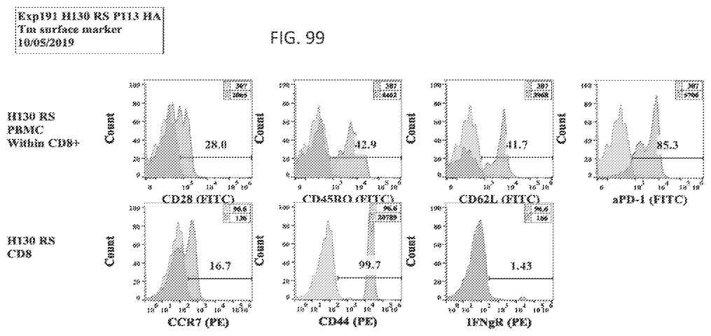

Detection of central memory CD8+ T cells and lack or decreased of IFN-g R expression on the CD8+ T cells (FIG. 99). PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors. Detection of central memory CD8+ T cells and lack or decreased of IFN-g R expression on the CD8+ T cells.

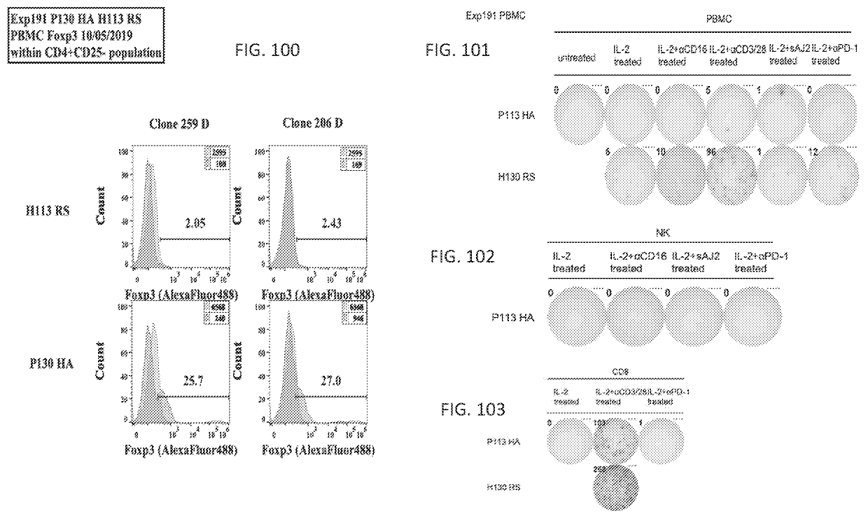

Decreased levels of Tregs in PBMCs from ALS patient (FIG. 100). PBMC Flow.

PBMCs were isolated from peripheral blood of donors using ficoll-hypaque gradient centrifugation. The percentages of Tregs in the PBMCs were determined by flow cytometric analysis after staining with specific fluorescent conjugated antibody directed to the FOXp3.

PBMC cytotoxicity. PBMC were left untreated, or treated with IL-2 (1000 U/mL) or treated with the combination of IL-2 and anti-CD16 mAb (3 μg/ml), or IL-2+anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100.

PBMC Elispot. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay (FIG. 101).

PBMC ELISA. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA (Table 140).

NK cytotoxicity. NK cells were isolated from PBMCs as described in the materials and methods section. Purified NK cells were left untreated, or treated with IL-2 (1000 U/mL) or treated with the combination of IL-2 and anti-CD16 mAb (3 μg/ml) or IL-2+sAJ2 (Cells:sAJ2 1:20). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100 (Table 141).

NK Elispot. NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment, 50,000 cells per treatment group were used in ELISPOT assay to determine the number of spots expressing IFN-γ (FIG. 102).

NK Elisa. NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment at 37° C., the supernatants were harvested and the levels of IFN-γ secretion were measured using ELISA (Table 142).

CD8 Elispot. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISPOT assay to determine the number of spots expressing IFN-γ (FIG. 103).

CD8 Elisa. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA (Table 143).

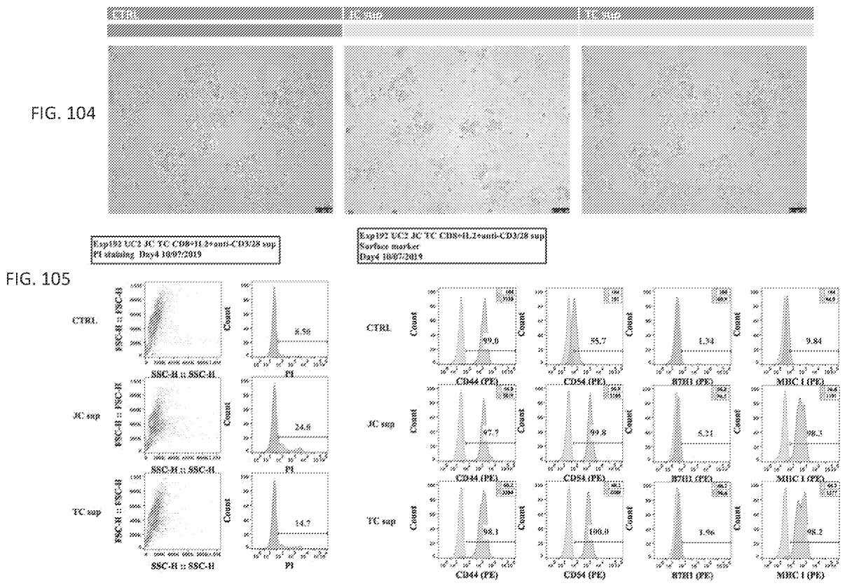

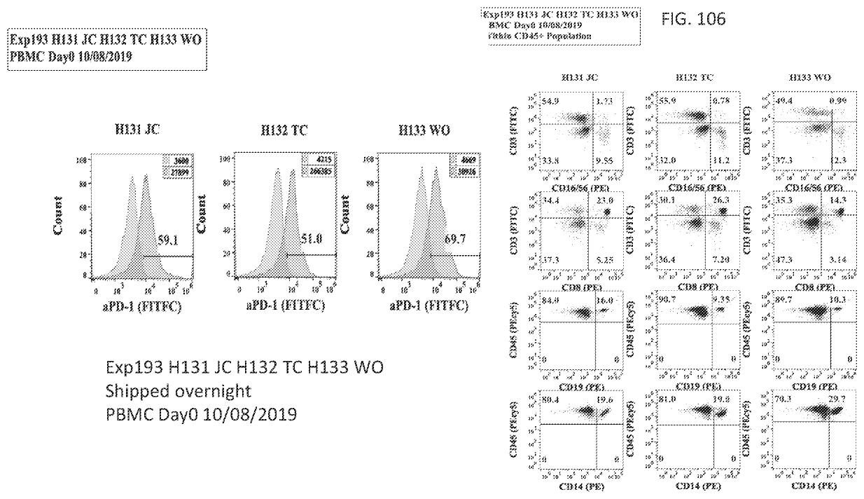

Increased cell death of OSCSCs by supernatants from CD8+ T cells from ALS patient as compared to healthy twin and increased CD54 expression on OSCSCs (FIG. 104 and FIG. 105). Supernatants containing equal amounts of IFN-γ from the healthy twin and ALS patients' CD8+ T cells treated with IL-2 (1000 U/ml) and anti-CD3/CD28mAb (25 microliter/ml) for 18 hours were added to OSCSCs for 4 days, to induce differentiation. After differentiation with the CD8+ T cells OSCSCs were detached and the levels of cell death was determined using Propidium iodide staining and the levels of differentiation were determine using staining with anti-CD44, anti-CD54, anti-B7H1 and anti-MHC class I.

### Example 46

Increased PD-1 expression on ALS (FIG. 10⁶ and Table 144). PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors.

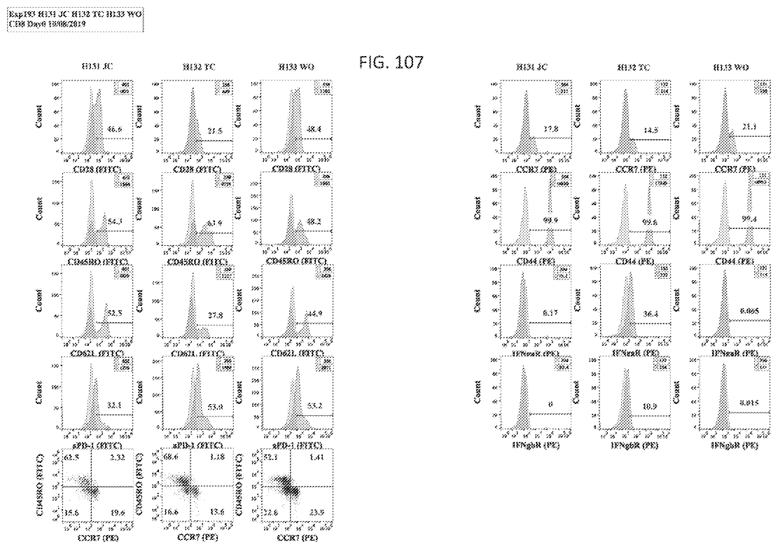

Detection of central memory CD8+ T cells and lack or decreased of IFN-g R expression on the CD8+ T cells (FIG. 107). PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors. Detection of central memory CD8+ T cells and lack or decreased IFN-g R expression on the CD8+ T cells on ALS patient when compared to healthy twin.

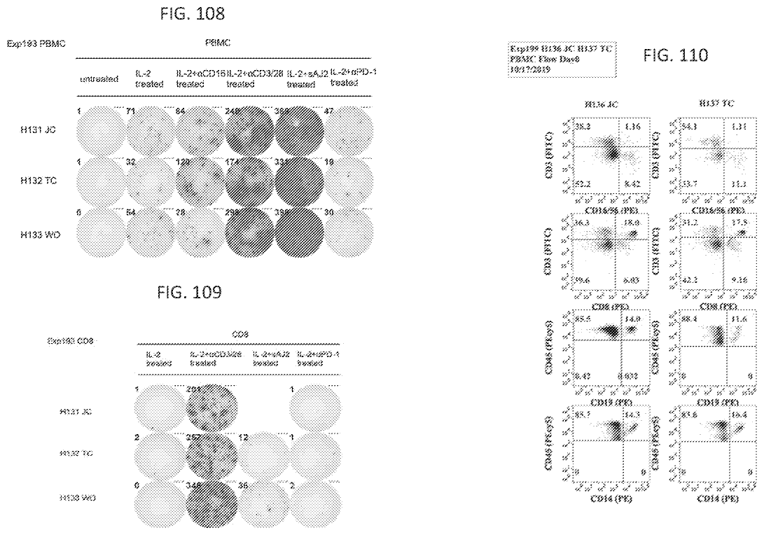

Increased IFN-g spots and secretion in ALS patients JC and WO when compared to TC (FIG. 108 and Tables 145-146).

PBMC cytotoxicity. PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (Table 145).

PBMC Elispot. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay (FIG. 108).

PBMC ELISA. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA (Table 146).

Increased IFN-g secretion by CD8+ T cells from ALS (JC and WO) when compared to TC (FIG. 109 and Table 147).

CD8 Elispot. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISPOT assay to determine the number of spots expressing IFN-γ (FIG. 109).

CD8 Elisa. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA (Table 147).

### Example 47

PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors (Table 148 and FIG. 110).

### Example 48

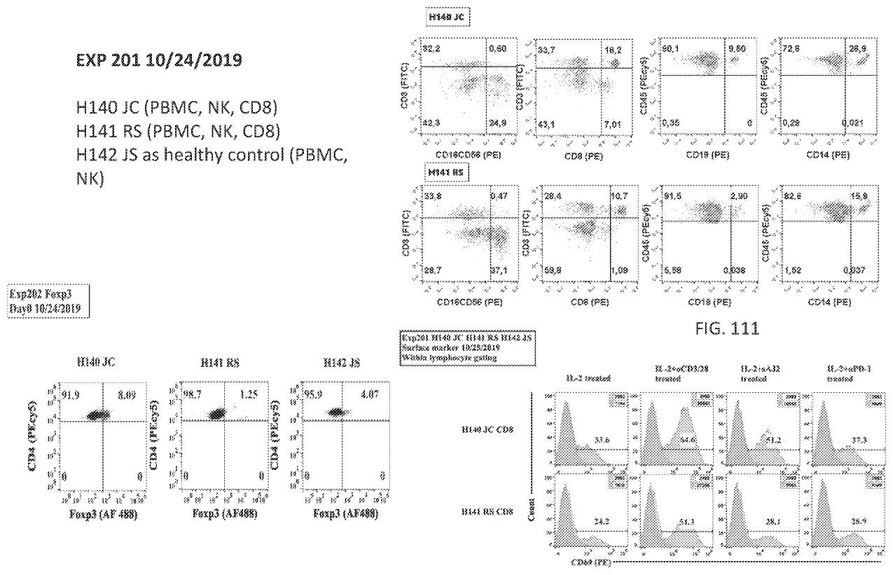

Decreased CD69 expression in RS when compared to JC ALS patient. RS received mobilization of Bone marrow derived stem cells by GCSF. Had very high levels of peripheral blood white blood cells (FIG. 111 and Table 149). PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors. The percentages of Treg and CD69 activation antigen expression were also determined (FIG. 111 and Table 149).

Decreased cytotoxicity of PBMCs and NK cells in RS when compared to JC ALS patient or JS healthy control. RS received mobilization of Bone marrow derived stem cells by GCSF. Had very high levels of peripheral blood white blood cells (Table 150).

PBMC cytotoxicity. PBMC were left untreated, or treated with IL-2 (1000 U/mL) or treated with the combination of IL-2 and anti-CD16 mAb (3 μg/ml), or IL-2+anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (Table 150).

NK cytotoxicity. NK cells were isolated from PBMCs as described in the materials and methods section. Purified NK cells were left untreated, or treated with IL-2 (1000 U/mL) or treated with the combination of IL-2 and anti-CD16 mAb (3 μg/ml) or IL-2+sAJ2 (Cells:sAJ2 1:20). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100 (Table 150).

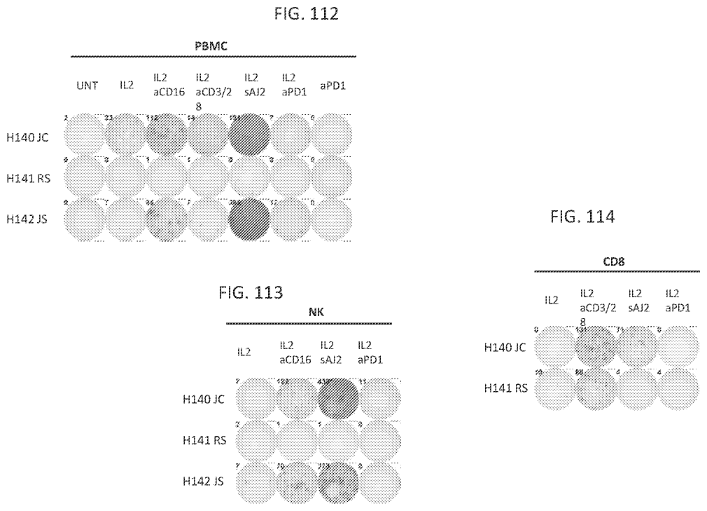

Decreased IFN-g spots in RS when compared to JC ALS patient and JS healthy control. RS received mobilization of Bone marrow derived stem cells by GCSF. RS Had very high levels of peripheral blood white blood cells. Even though no spots could be observed in PBMCs and NK cells of RS we still saw increased spots from CD8+ T cells. JC (ALS) was in general higher than JS healthy control (FIG. 112-FIG. 114).

PBMC Elispot. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay (FIG. 112).

NK Elispot. NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment, 50,000 cells per treatment group were used in ELISPOT assay to determine the number of spots expressing IFN-γ (FIG. 113).

CD8 Elispot. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISPOT assay to determine the number of spots expressing IFN-γ (FIG. 114).

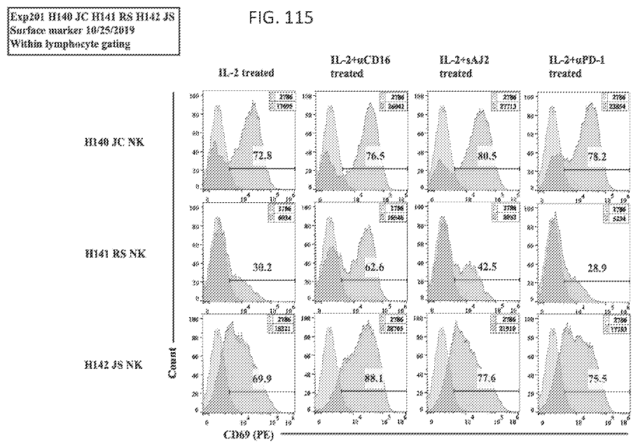

JC (ALS) had higher expression of CD69 when compared to RS (ALS) or JS healthy control (FIG. 115). PBMC Flow. PBMCs were isolated from peripheral blood of donors using ficoll-hypaque gradient centrifugation. The percentages of CD69 activation antigen expression in the NK cells were determined by flow cytometric analysis after staining with specific fluorescent conjugated antibody directed to the cell surface receptors.

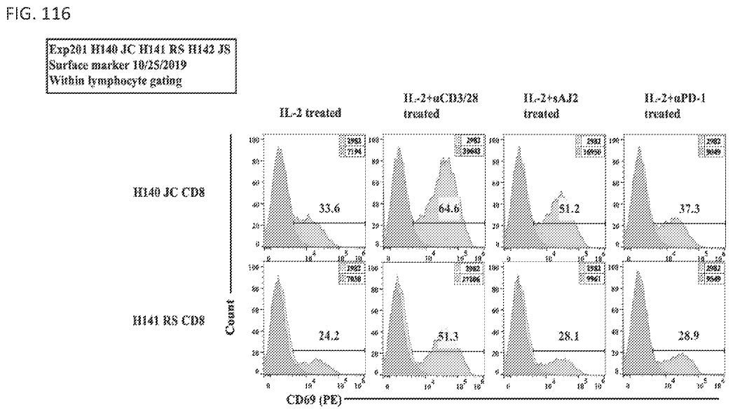

PBMC Flow. PBMCs were isolated from peripheral blood of donors using ficoll-hypaque gradient centrifugation. The percentages of CD69 activation antigen expression on the CD8+ T cells were determined by flow cytometric analysis after staining with specific fluorescent conjugated antibody directed to the cell surface receptors (FIG. 116).

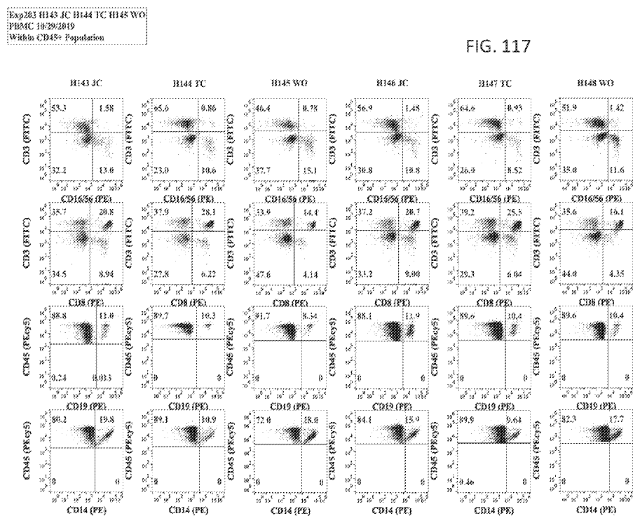

Lower percentages of CD8+ T cells in JC and WO ALS patients when compared to TC (Table 151 and FIG. 117). PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors.

### Example 49

Higher PD-1 expression in JC and WO ALS patients compared to TC healthy control. Higher T regs in PBMCs in JC and WO when compared to TC. No differences in naïve CD4 differentiation (FIG. 118). PBMC Flow. PBMCs were isolated from peripheral blood of donors using ficoll-hypaque gradient centrifugation. The percentages of PD-1, T reg in PBMCs were determined by flow cytometric analysis after staining with specific fluorescent conjugated antibodies directed to the cell surface receptors. Naïve CD4 were sorted out and differentiated with the differentiation media provided by the manufacturer and the level of Tregs were determined after differentiation.

No major differences in cytotoxicity of PBMCs and NK cells in JC and WO ALS patients when compared to TC (Tables 152-153).

PBMC cytotoxicity. PBMC were left untreated, or treated with IL-2 (1000 U/mL) or treated with the combination of IL-2 and anti-CD16 mAb (3 μg/ml), or IL-2+anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (Table 152).

NK cytotoxicity. NK cells were isolated from PBMCs as described in the materials and methods section. Purified NK cells were left untreated, or treated with IL-2 (1000 U/mL) or treated with the combination of IL-2 and anti-CD16 mAb (3 μg/ml) or IL-2+sAJ2 (Cells:sAJ2 1:20). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100 (Table 153).

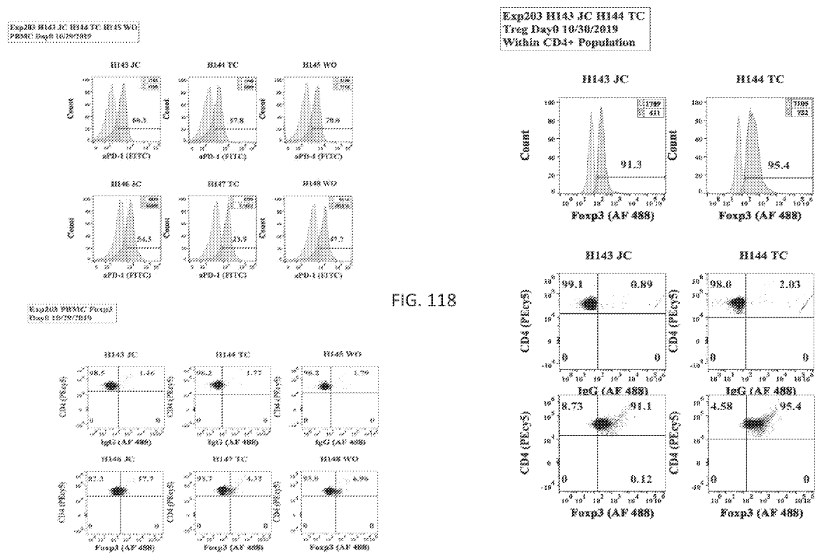

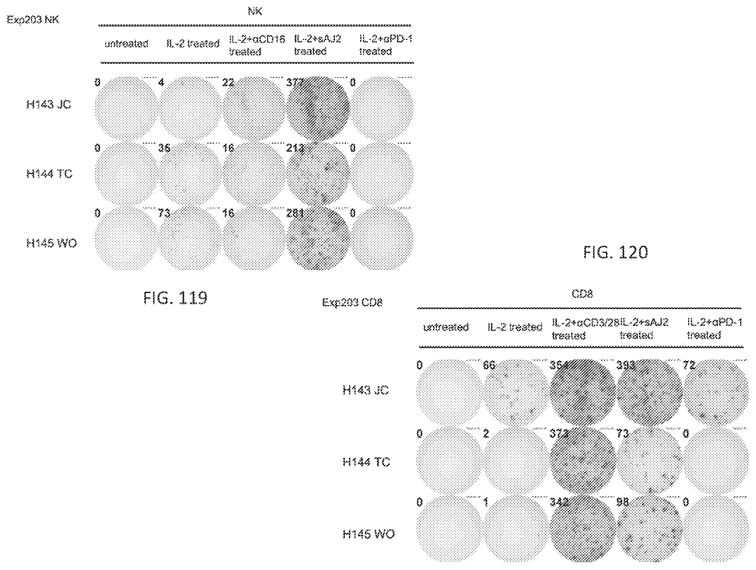

Increased IFN-g spots in NK and CD8+ T cells from JC and WO when compared to TC (FIG. 119-120).

NK Elispot. NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment, 50,000 cells per treatment group were used in ELISPOT assay to determine the number of spots expressing IFN-γ (FIG. 119).

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISPOT assay to determine the number of spots expressing IFN-γ (FIG. 120).

Increased IFN-g spots in NK and CD8+ T cells from JC and WO when compared to TC (Tables 154-156).

PBMC ELISA. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA (Table 154).

CD8 Elisa. CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA (Table 155).

NK Elisa. NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment at 37° C., the supernatants were harvested and the levels of IFN-γ secretion were measured using ELISA (Table 156).

Increased pro-inflammatory cytokines in NK and CD8+ T cells from WO when compared to TC. JC is improving but still has higher levels of IL-6 when compared to TC. Both JC and WO ALS patients have higher IL-6 secretion (Table 157).

NK Luminex. NK cells were treated with IL-2 (1000 U/mL) or treated with the combination of IL-2 and anti-CD16 mAb (3 μg/ml). After an overnight treatment at 37° C., the supernatants were harvested and the levels of cytokine/chemokine/growth factor/ligands secretion were measured using multiplex luminex assay (Table 157).

CD8+ T cell Luminex. Sorted CD8+ T cells were treated with the combination of IL-2+anti-CD3/28 antibodies (25 μl/mL). After an overnight treatment at 37° C., the supernatants were harvested and the levels of cytokine/chemokine/growth factor/ligands secretion were measured using multiplex luminex assay (Table 157).

### Example 50

CD8+ T cells significantly have higher levels of cytokine induction when compared to NK per cell basis (Table 158).

NK Luminex. NK cells were treated with IL-2 (1000 U/mL) or treated with the combination of IL-2 and anti-CD16 mAb (3 μg/ml). After an overnight treatment at 37° C., the supernatants were harvested and the levels of cytokine/chemokine/growth factor/ligands secretion were measured using multiplex luminex assay (Table 158).

CD8+ T cell Luminex. Sorted CD8+ T cells were treated with the combination of IL-2+anti-CD3/28 antibodies (25 μl/mL). After an overnight treatment at 37° C., the supernatants were harvested and the levels of cytokine/chemokine/growth factor/ligands secretion were measured using multiplex luminex assay (Table 158).

### Example 51

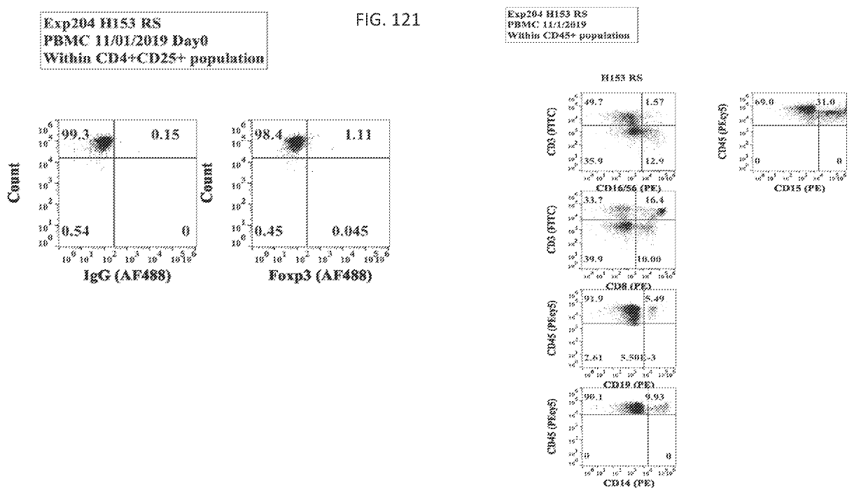

PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors (FIG. 121 and Table 159).

PBMC cytotoxicity. PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100 (Table 160).

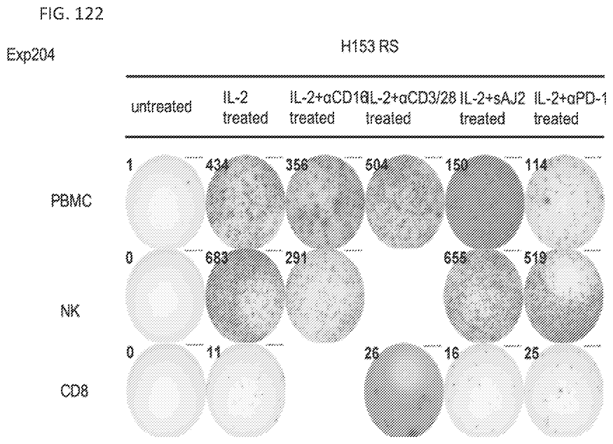

PBMC Elispot. PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-g spots were determined using ELISPOT assay (FIG. 122).

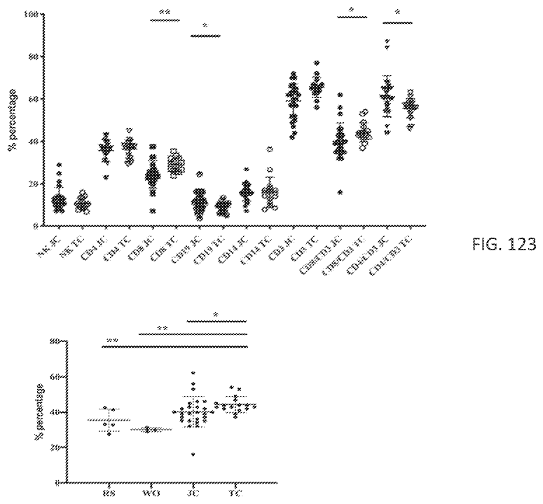

Percentages of CD8+ T cells are lower in ALS patients RS, WO and JC when compared to TC healthy control (FIG. 123). PBMC Flow. PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors.

### Example 52

### PBMC Flow Cytometry

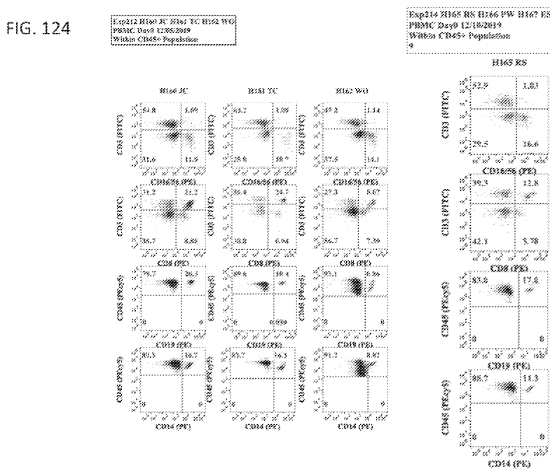

PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors. Results show decreased or no change in the percentages of CD8+ subsets in ALS patients when compared to control (FIG. 124; Tables 161 and 162).

### Example 53

### PBMC Cytotoxicity

PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in Tables 163 and 164. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100. Results show no significant differences or higher NK cytotoxicity between ALS patients and control.

### Example 54

### PBMC ELISpot

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-γ spots were determined using ELISpot assay.

### PBMC ELISA

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA.

### Conclusion

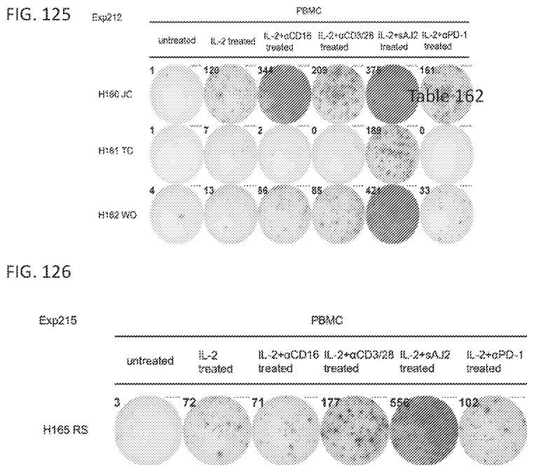

Results show IFN-γ spots and secretion are increased in PBMCs of ALS patients with all treatments tested in comparison to twin control (FIGS. 125 and 126; Tables 165 and 166).

### Example 55A

### CD8 ELISpot

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISpot assay to determine the number of spots expressing IFN-γ.

### CD8 ELISA

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA.

### Conclusion

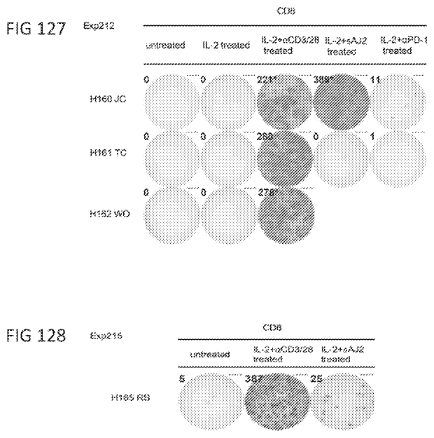

Results show similar or increased IFN-γ spots and increased secretion of IFN-γ in CD8+ T cells from all ALS patients in comparison to twin control (FIGS. 127 and 128; Tables 167 and 168).

### Example 55B

### PBMC Flow Cytometry

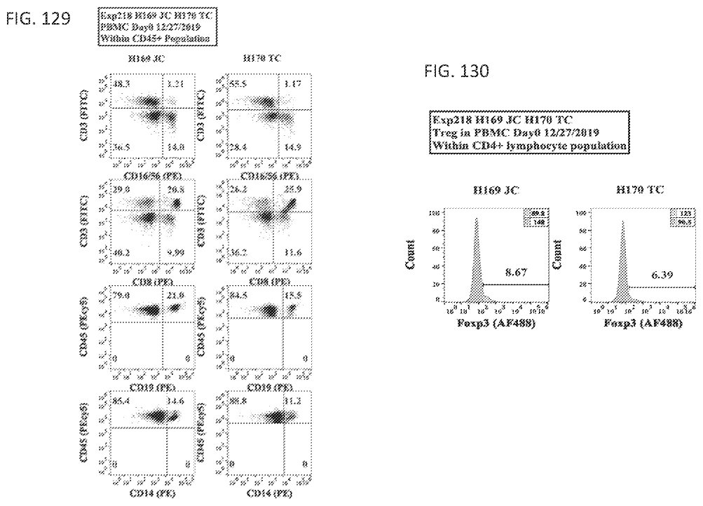

PBMCs were isolated from peripheral blood of donors using ficoll-hypaque gradient centrifugation. The percentages of Tregs in the PBMCs were determined by flow cytometric analysis after staining with specific fluorescent conjugated antibody directed to the FOXp3. Results show decrease in the percentages of CD8+ subsets in ALS patient when compared to control and increase in the percentages of Treg subset in ALS patient when compared to control (FIGS. 129 and 130; Table 169).

### Example 56

### PBMC ELISpot

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-γ spots were determined using ELISpot assay.

### PBMC ELISA

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA.

### Conclusion

Results show no significant differences in the cytotoxicity of NK cells in PBMCs and IFN-γ spots in ALS and control twin. However, significant secretion of IFN-γ in ALS patient was observe when compared to control (FIG. 131; Tables 170 and 171).

### Example 57

### CD8 ELISpot

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISpot assay to determine the number of spots expressing IFN-γ.

### CD8 ELISA

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA.

### Conclusion

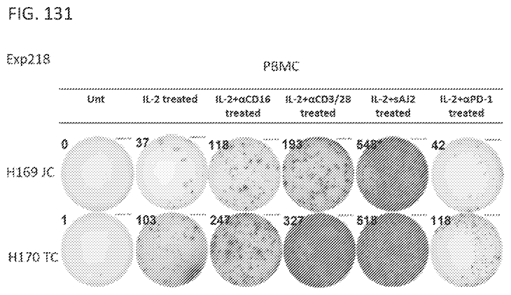

Results show increased IFN-γ spots and release in CD8+ T cells in ALS when compared to control twin (FIG. 132 and Table 172).

### Example 58

### PBMC Flow Cytometry

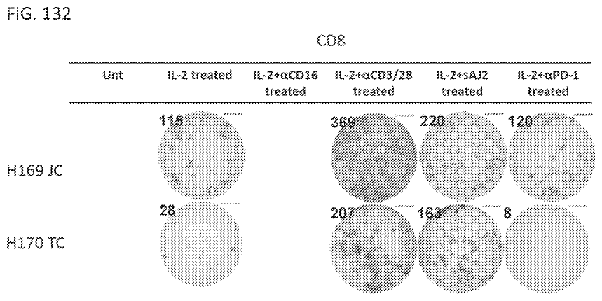

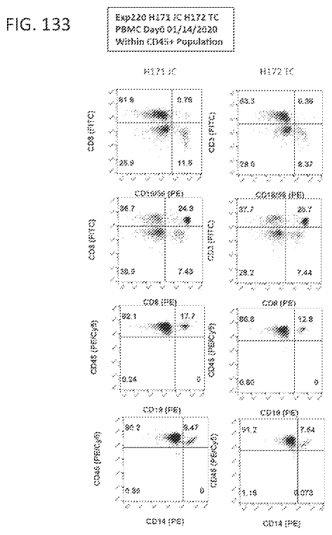

PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors. Results show decreased in the percentages of CD8+ subsets in ALS patients when compared to control (FIG. 133 and Table 173).

### Example 59

### PBMC Cytotoxicity

PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100.

### NK Cytotoxicity

NK cells were isolated from PBMCs as described in the materials and methods section. Purified NK cells were treated with IL-2 (1000 U/mL) or IL-2+sAJ2 (Cells:sAJ2 1:20). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100.

### Conclusion

Results show no significant differences in the NK cytotoxicity in PBMCs and purified NK cells between ALS and control twin (Tables 174 and 175).

### Example 60

### PBMC ELISpot

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-γ spots were determined using ELISpot assay.

### NK ELISpot

NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment, 50,000 cells per treatment group were used in ELISpot assay to determine the number of spots expressing IFN-γ.

### CD8 ELISpot

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISpot assay to determine the number of spots expressing IFN-γ.

### Conclusion

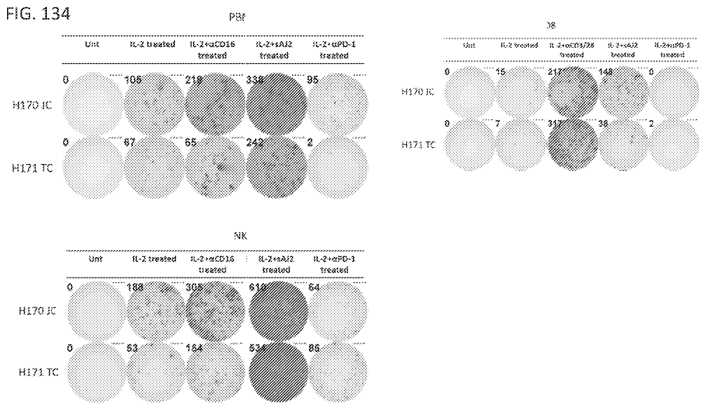

Results show increased IFN-γ spots in PBMCs, NK and CD8+ T cells in the majority of treatments in ALS when compared to control twin (FIG. 134).

### Example 61

### PBMC ELISA

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA.

NK ELISA NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment at 37° C., the supernatants were harvested and the levels of IFN-γ secretion were measured using ELISA.

### CD8 ELISA

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA.

### Conclusion

Results show increased IFN-γ release in PBMCs, NK and CD8+ T cells in the majority of treatments in ALS when compared to control twin (Tables 176, 177, and 178).

### Example 62

### PBMC Flow Cytometry

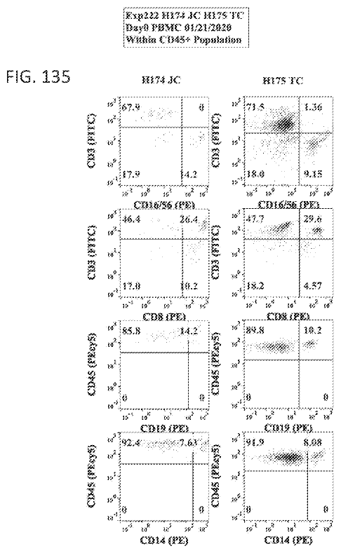

PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors. Results show decrease in the percentages of CD8+subsets in ALS patients when compared to control (Table 179 and FIG. 135).

### Example 63

### PBMC Cytotoxicity

PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100.

### NK Cytotoxicity

NK cells were isolated from PBMCs as described in the materials and methods section. Purified NK cells were treated with IL-2 (1000 U/mL) or IL-2+sAJ2 (Cells:sAJ2 1:20). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100.

### Conclusion

Results show increased or no change in the cytotoxicity of NK cells in PBMCs and NK cells in ALS when compared to control twin (Tables 180 and 181).

### Example 64

### PBMC ELISpot

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-γ spots were determined using ELISpot assay.

### NK ELISpot

NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment, 50,000 cells per treatment group were used in ELISpot assay to determine the number of spots expressing IFN-γ.

### CD8 ELISpot

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISpot assay to determine the number of spots expressing IFN-γ.

### Conclusion

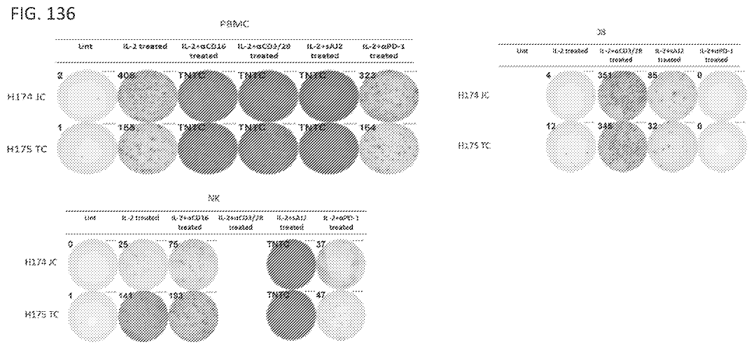

Results show increased IFN-γ spots in PBMCs, and CD8+ T cells in the majority of treatments but not in NK cells in ALS when compared to control twin (FIG. 136).

### Example 65

### PBMC ELISA

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA.

### NK ELISA

NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment at 37° C., the supernatants were harvested and the levels of IFN-γ secretion were measured using ELISA.

### CD8 ELISA

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA.

### Conclusion

Results show increased IFN-γ release in PBMCs, and CD8+ T cells in the majority of treatments but decreased secretion in NK cells in ALS when compared to control twin (Tables 182, 183, and 184).

### Example 66

### PBMC Flow Cytometry

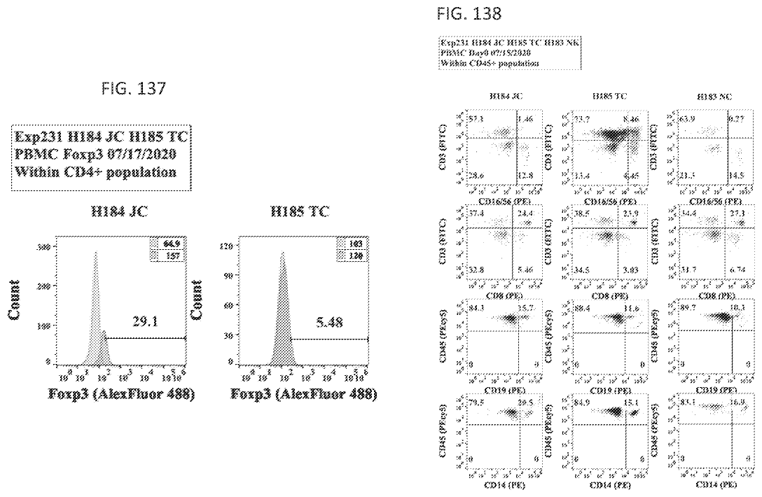

PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors. Results show similar percentages of CD8+ subsets in ALS patients when compared to control (FIGS. 137 and 138; Table 190).

### Example 67

### PBMC Cytotoxicity

PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100.

### PBMC Elispot

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-γ spots were determined using ELISPOT assay.

### PBMC ELISA

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA.

### Conclusion

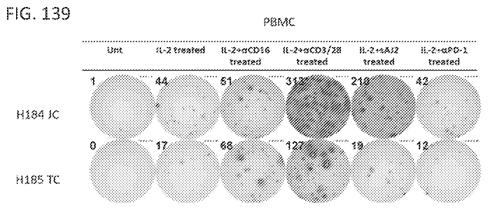

Results show increased NK cytotoxicity, IFN-γ spots, and secretion in PBMCs of ALS patients in the majority of treatments as compared to control (Tables 191 and 192; FIG. 139).

### Example 68

### CD8 ELISpot

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISpot assay to determine the number of spots expressing IFN-γ.

### CD8 ELISA

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA.

### Conclusion

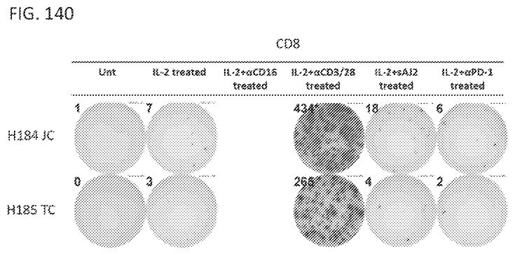

Results show increased IFN-γ spots and higher secretion of IFN-γ in CD8+ T cells from ALS patient within all treatments in comparison to twin control. Similar or increased IFN-γ spots and higher secretion of IFN-γ in CD8+ T cells from all ALS patients in comparison to twin control was observed (FIG. 140 and Table 193).

### Example 69

### PBMC Flow Cytometry

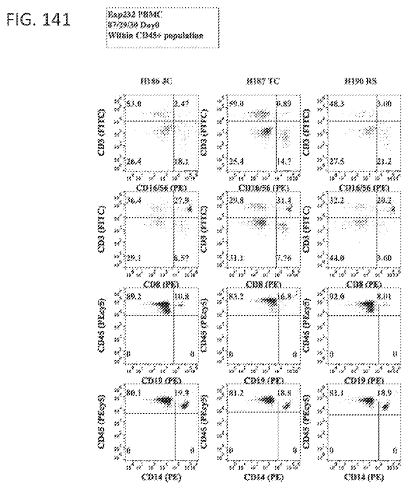

PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors. Results show decreased percentages of CD8+ subsets in ALS patients when compared to control (Table 194 and FIG. 141).

### Example 70

### NK Cytotoxicity

NK cells were isolated from PBMCs as described in the materials and methods section. Purified NK cells were treated with IL-2 (1000 U/mL) or IL-2+sAJ2 (Cells:sAJ2 1:20). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100. Results show higher NK cell cytotoxicity in all treatments between ALS patients and control (Table 195).

### Example 71

### PBMC ELISpot

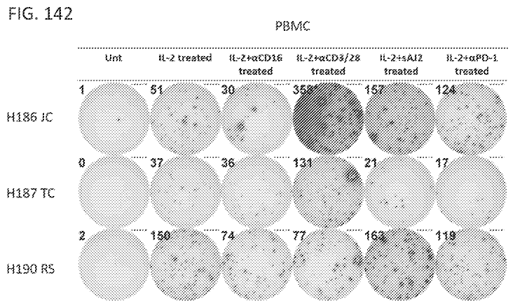

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-γ spots were determined using ELISpot assay. Results show IFN-γ spots are higher in PBMCs of ALS patients with most treatments tested in comparison to twin control (FIG. 142).

### Example 72

### PBMC ELISA

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA. IFN-γ secretion are higher in PBMCs of ALS patients in most treatments tested in comparison to twin control (Table 196 and 197).

### Example 73

### CD8 ELISpot

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISpot assay to determine the number of spots expressing IFN-γ.

### CD8 ELISA

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA.

### Conclusion

Results show increased IFN-γ spots with the exception of one treatment, and higher secretion of IFN-γ in all treatments from CD8+ T cells from all ALS patients in comparison to twin control (Tables 198 and 199; FIG. 143).

### Example 74

JC and RS's blood were received and processed. TC's blood was drawn and saved.

CD8 isolation and NK isolation were performed.

PBMC 1 million/group
1. Untreated
2. IL-2 treated
3. IL-2+αCD16 treated
4. IL-2+αCD3/28 treated
5. IL-2+sAJ2 treated
6. IL-2+αsPD-1 treated

CD8 0.5 million/group
1. Untreated
2. IL-2 treated
3. IL-2+αCD3/28 treated
4. IL-2+sAJ2 treated
5. IL-2+αPD-1 treated

NK 0.5 million/group
1. Untreated
2. IL-2 treated
3. IL-2+αCD16 treated
4. IL-2+sAJ2 treated
5. IL-2+αPD-1 treated

Cell count, sup collection, chromium, ELISpot, ELISA, and Treg assays were performed on H194 JC, H195 RS, H196 TC, H197 YC, and H198 MK.

### Example 75

### PBMC Flow Cytometry

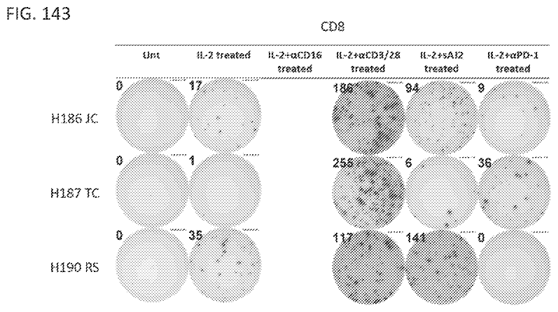

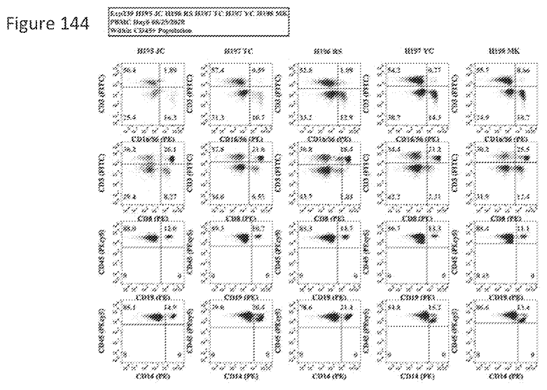

PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors. Results show decreased or no change in the percentages of CD8+ subsets in ALS patients when compared to control (Table 200 and FIG. 144).

### Example 76

### T Reg Flow Cytometry

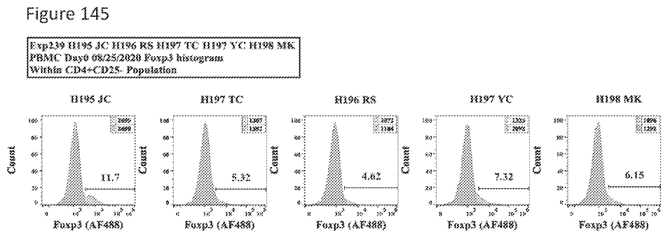

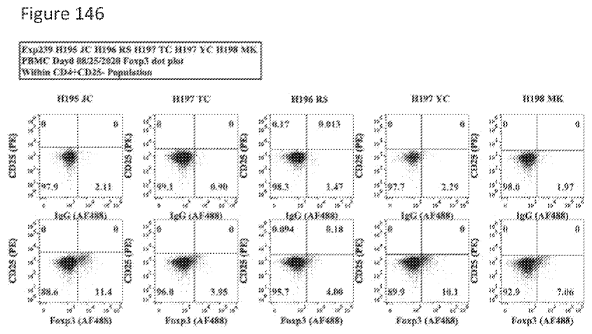

PBMCs were isolated from peripheral blood of donors using ficoll-hypaque gradient centrifugation. The percentages of Tregs in the PBMCs were determined by flow cytometric analysis after staining with specific fluorescent conjugated antibody directed to the FOXp3. Results show variable levels of Tregs in PBMCs from ALS patients when compared to Twin control or other healthy controls (FIGS. 145 and 146).

### Example 77

### PBMC Cytotoxicity

PBMC were left untreated, or treated with IL-2 (1000 U/mL) or treated with the combination of IL-2 and anti-CD16 mAb (3 μg/ml), or IL-2+anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100. Results show no significant differences or higher NK cytotoxicity between ALS patients and control (Table 201).

### Example 78

### PBMC ELISpot

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-γ spots were determined using ELISpot assay.

### PBMC ELISA

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA.

### Conclusion

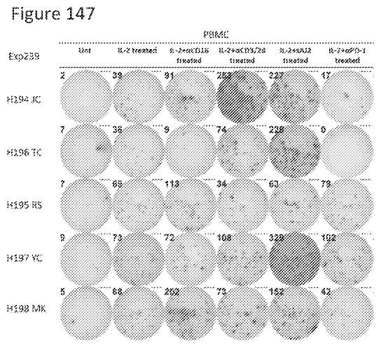

Results show IFN-γ spots and secretion are higher in PBMCs of ALS patients with all treatments tested in comparison to twin control. Variable IFN-γ spots and secretion in PBMCs of ALS patients with most treatments in comparison to twin control and other controls was observed (Tables 202 and 203; FIG. 147).

### Example 79

NK cytotoxicity

NK cells were isolated from PBMCs as described in the materials and methods section. Purified NK cells were treated with IL-2 (1000 U/mL) or IL-2+sAJ2 (Cells:sAJ2 1:20). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100.

### NK ELISpot

NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment, 50,000 cells per treatment group were used in ELISpot assay to determine the number of spots expressing IFN-γ.

### NK ELISA

NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment at 37° C., the supernatants were harvested and the levels of IFN-γ secretion were measured using ELISA.

### Conclusion

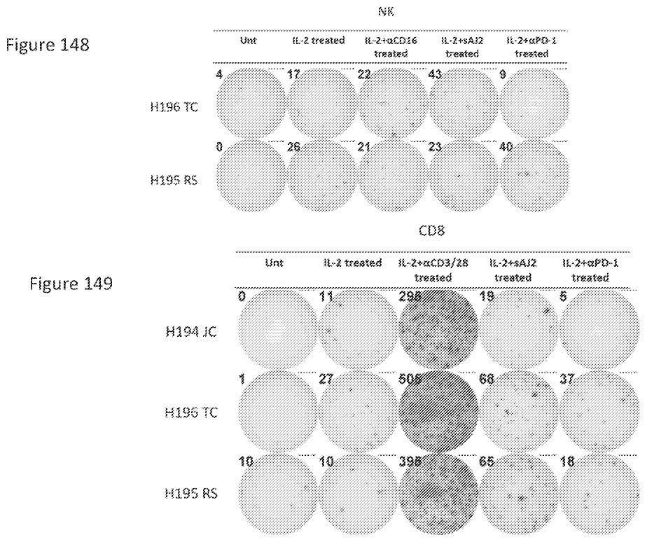

Results show no significant differences in NK cytotoxicity, NK IFN-γ spots and IFN-γ secretion by the NK cells between ALS patients and control (Tables 204 and 205; FIG. 148).

### Example 80

### CD8 ELISpot

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISpot assay to determine the number of spots expressing IFN-γ.

### CD8 ELISA

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA.

### Conclusion

Similar IFN-γ spots and higher secretion of IFN-γ in CD8+ T cells from all ALS patients in IL-2+anti-CD3/CD28 or IL-2+sAJ2 treated samples in comparison to twin control (Tables 206 and 207; FIG. 149).

### Example 81

Table 208 shows decreased numbers of cells after differentiation of nCD4 to Tregs in JC but not in RS and control twin. RS had the highest increase in the numbers of cells obtained after differentiation.

### Example 82

T regs Flow Cytometry

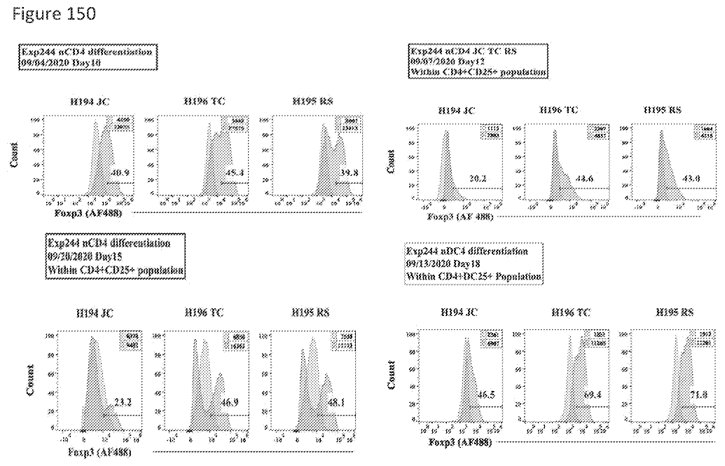

PBMCs were isolated from peripheral blood of donors using ficoll-hypaque gradient centrifugation. The percentages of Tregs in the PBMCs were determined by flow cytometric analysis after staining with specific fluorescent conjugated antibody directed to the FOXp3. Results show similar or decreased levels of Tregs in differentiated nCD4 in JC when compared to RS ALS patient or control twin (FIG. 150).

### Example 83

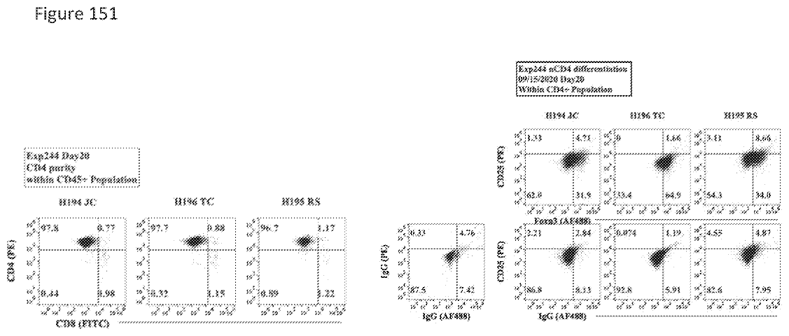

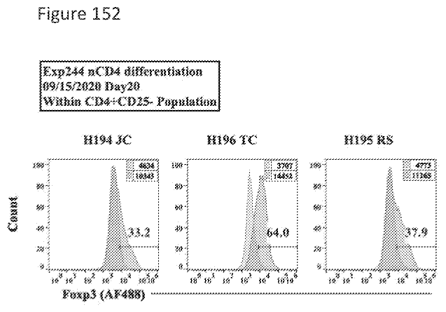

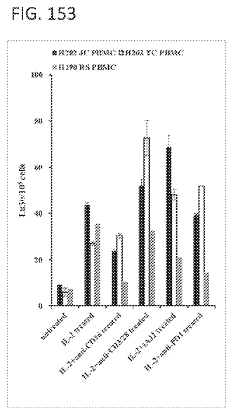

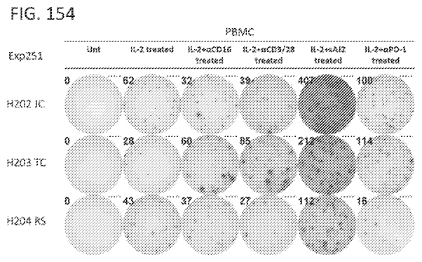

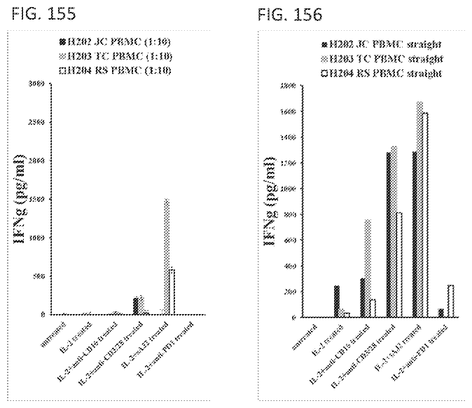

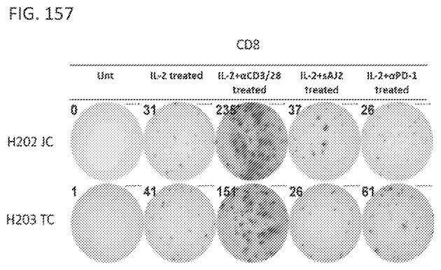

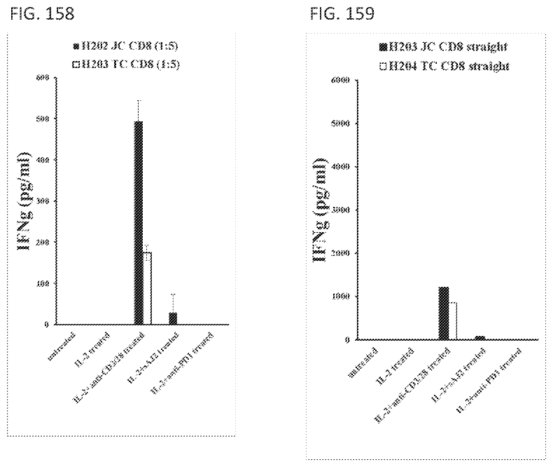

FIGS. 151 and 152 show decreased levels of Tregs in differentiated nCD4 in JC and RS when compared to control twin at day 20.

### Example 84

Differentiated nCD4 IL-10 ELISA cells were resuspended into 0.5 million per ml with IL-2, anit-CD3/28 and differentiation supplement every time when splitting the cultures, and at days indicated in the figuref the supernatants were harvested and the levels of IL-10 secretion were measured using ELISA. Results show similar levels of IL-10 secretion from differentiated Tregs from nCD4 in JC when compared to control twin; RS ALS patient had much lower secretion when compared to JC and TC. JC has lower levels of expansion for Treg but good function, RS has very fast expansion of Treg but lower function (Tables 209, 210, and 211).

### Example 85

Differentiated nCD4 IL-6 ELISA cells were resuspended into 0.5 million per ml with IL-2, anit-CD3/28 and differentiation supplement every time when splitting the cultures, and at days indicated in the figure the supernatants were harvested and the levels of IL-6 secretion were measured using ELISA. Results show higher levels of IL-6 secretion from differentiated Tregs from nCD4 in JC and RS when compared to control twin (Tables 212, 213, and 214).

### Example 86

Differentiated nCD4 IFN-γ ELISA cells were resuspended into 0.5 million per ml with IL-2, anit-CD3/28 and differentiation supplement every time when splitting the cultures, and at days indicated in the figure the supernatants were harvested and the levels of IFN-γ secretion were measured using ELISA. Results show higher levels of IFN-γ secretion from differentiated Tregs from nCD4 in JC and RS when compared to control twin (Tables 215, 216, and 217).

### Example 87

CD8 isolation was performed.

### PBMC 1 Million/Group

1. Untreated
2. IL-2 treated
3. IL-2+αCD16 treated
4. IL-2+αCD3/28 treated
5. IL-2+sAJ2 treated
6. IL-2+αPD-1 treated

### CD8 0.5 Million/Group

1. Untreated
2. IL-2 treated
3. IL-2+αCD3/28 treated
4. IL-2+sAJ2 treated
5. IL-2+αPD-1 treated

Cell count, sup collection, chromium, ELISpot, ELISA, Treg assays were performed on H202 JC, H203 TC, and H204 RS.

### Example 88

CD8 isolation was performed.

### PBMC 1 Million/Group

1. Untreated
2. IL-2 treated
3. IL-2+αCD16 treated
4. IL-2+αCD3/28 treated
5. IL-2+sAJ2 treated
6. IL-2+αPD-1 treated

### CD8 0.6 Million/Group

1. Untreated with PTX
2. IL-2 treated with PTX
3. IL-2+αCD3/28 treated with PTX
4. Untreated without PTX
5. IL-2 treated without PTX
6. IL-2+αCD3/28 treated without PTX

JC and TS were treated with PTX.

Cell count, sup collection, chromium, ELISpot, ELISA, Treg assays were performed.

### Example 89

Patient started PTX treatment.

### PBMC Flow Cytometry

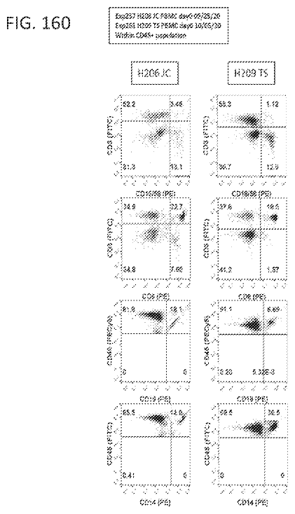

PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors. Results show no significant change in the percentages of CD8+ subsets in ALS patient when compared to control (Table 223 and FIG. 160).

### Example 90

### PBMC ELISA

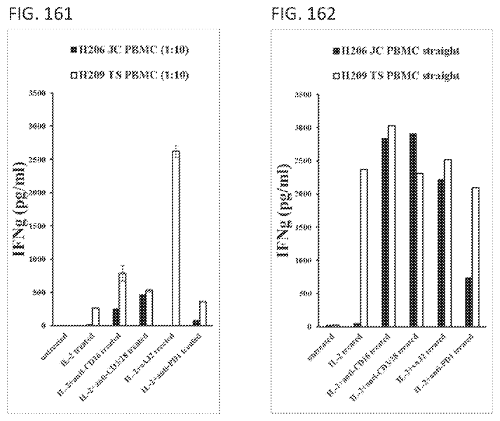

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA. Results show similar or decreased secretion of IFN-γ in PBMCs of ALS patient with different treatments tested in comparison to twin control (FIGS. 161 and 162; Tables 224 and 225).

### Example 91

### CD8 ELISA

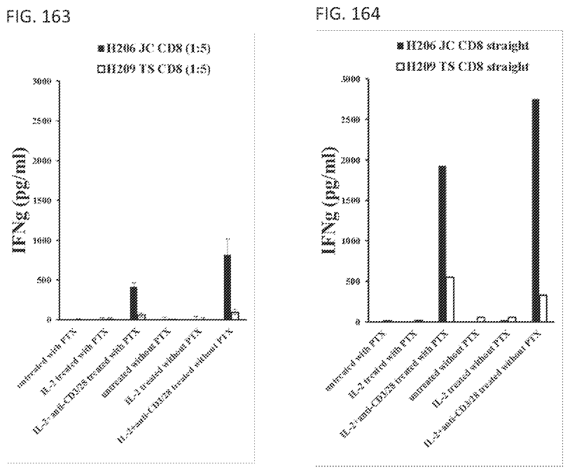

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA. Results show increased secretion of IFN-γ in CD8+ T cells from ALS patient in all treatments in comparison to twin control (Tables 226 and 227; FIGS. 163 and 164).

### Example 92

JC's blood is received and processed. TC's blood was drawn, purified

CD8 isoloation and NK isolation were performed.

PBMC 1 million/group
1. Untreated
2. IL-2 treated
3. IL-2+αCD16 treated
4. IL-2+αCD3/28 treated
5. IL-2+sAJ2 treated
6. IL-2+αPD-1 treated

CD8 0.6 million/group
1. Untreated
2. IL-2 treated
3. IL-2+αCD3/28 treated
4. IL-2+sAJ2 treated
5. IL-2+αPD-1 treated

NK 1 million/group
1. Untreated
2. IL-2 treated
3. IL-2+αCD16 treated
4. IL-2+sAJ2 treated
5. IL-2+αPD-1 treated

Cell count, sup collection, chromium, ELISpot, ELISA, and Treg assays were performed.

### Example 93

### PBMC Flow Cytometry

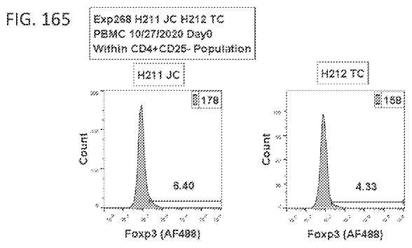

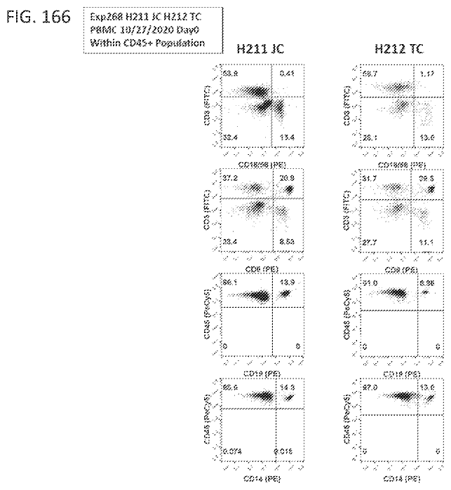

PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors. Results show decrease in the percentage of CD8+ subsets and increased Treg subsets in ALS patient when compared to control (Table 228; FIGS. 165 and 166).

### Example 94

PBMC cytotoxicity

PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100.

NK cytotoxicity

NK cells were isolated from PBMCs as described in the materials and methods section. Purified NK cells were treated with IL-2 (1000 U/mL) or IL-2+sAJ2 (Cells:sAJ2 1:20). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100.

### Conclusion

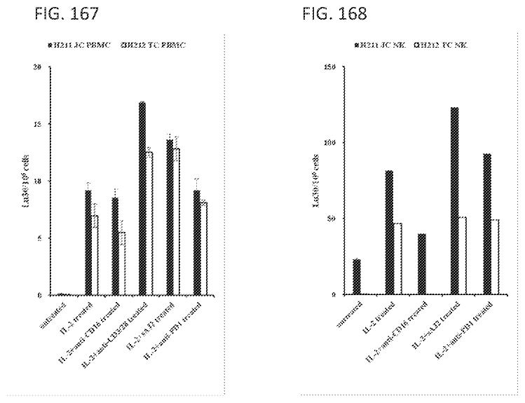

Results show higher NK cytotoxicity in ALS patient in all treatments in PBMCs and in sorted NK cells when compared to control (Tables 229 and 230; FIGS. 167 and 168). No significant differences or higher NK cytotoxicity between ALS patients and control was observed.

### Example 95

### PBMC ELISpot

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-γ spots were determined using ELISpot assay.

### PBMC ELISA

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA.

### Conclusion

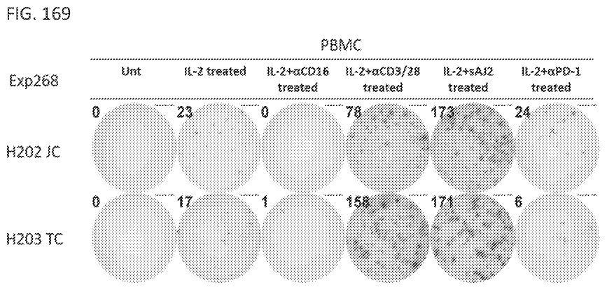

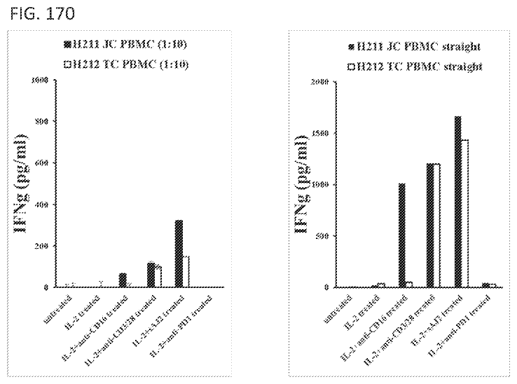

Results show increased or no difference in IFN-γ spots but higher secretion of IFN-γ in PBMCs of ALS patient with the majority of treatments tested in comparison to twin control (FIGS. 169 and 170; Tables 231 and 232).

### Example 96

### NK ELISpot

NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment, 50,000 cells per treatment group were used in ELISpot assay to determine the number of spots expressing IFN-γ.

### NK ELISA

NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment at 37° C., the supernatants were harvested and the levels of IFN-γ secretion were measured using ELISA.

### Conclusion

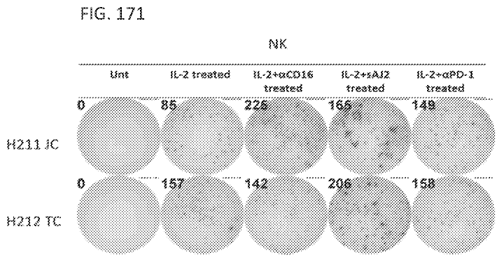

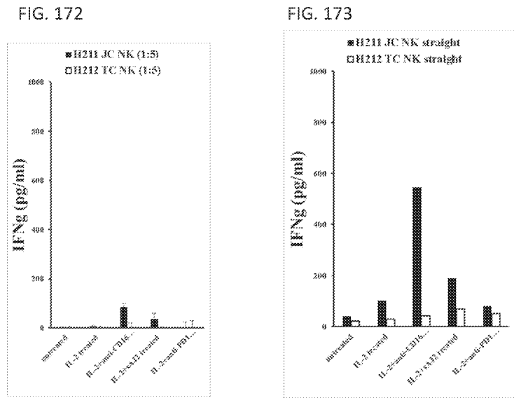

Results show no significant differences in IFN-γ spots but higher secretion of IFN-γ by NK cells in ALS patient with the majority of treatments tested in comparison to twin control (FIGS. 171, 172, and 173; Tables 233 and 234).

### Example 97

### CD8 ELISpot

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISpot assay to determine the number of spots expressing IFN-γ.

### CD8 ELISA

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA.

### Conclusion

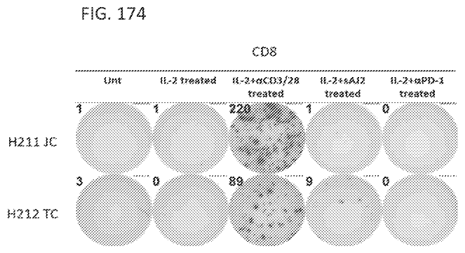

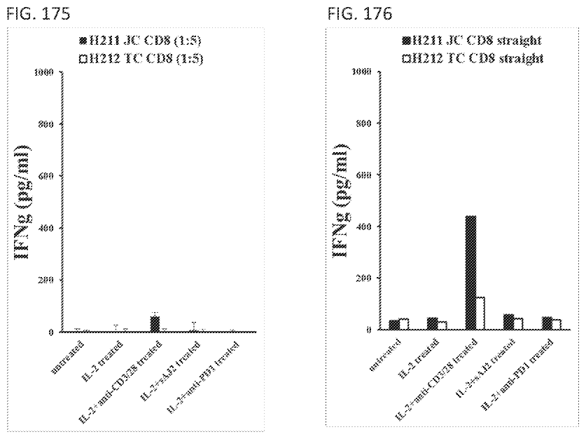

Results show increased IFN-γ spots and higher secretion of IFN-γ in CD8+ T cells in all treatments from ALS patient in comparison to twin control (FIGS. 174, 175, and 176; Tables 235 and 236).

### Example 98

Patient was treated PTX and Amylax.

### PBMC Flow

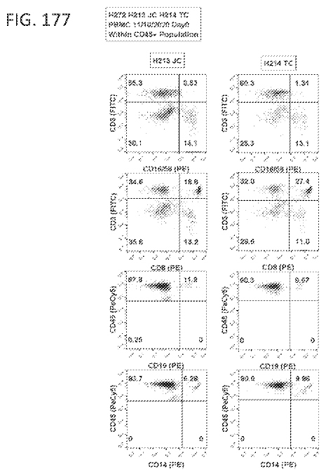

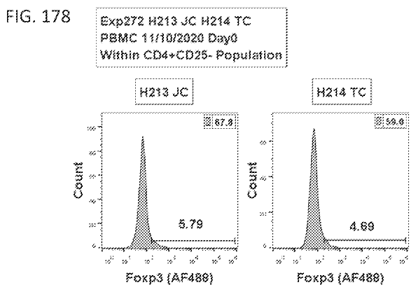

PBMCs were isolated from peripheral blood of donors using ficoll-hypaque gradient centrifugation. The percentages of each cell subset in the PBMCs were determined by flow cytometric analysis after staining with specific fluorescent conjugated antibodies directed to the cell surface receptors. Results show decrease in the percentage of CD8+ subsets and increased Treg subsets in ALS patient when compared to control (FIGS. 177 and 178; Table 237).

### Example 99

PBMC cytotoxicity

PBMC were left untreated, or treated with IL-2 (1000 U/mL) or treated with the combination of IL-2 and anti-CD16 mAb (3 μg/ml), or IL-2+ anti-CD3/28 antibodies (25 μl/mL) and IL-2+sAJ2 (1:20 PBMC:sAJ2 ratio) as shown in the figure. After an overnight incubation, the NK cell mediated cytotoxicity was determined by the standard 4-hour chromium release assay using oral squamous carcinoma stem cells (OSCSCs) as targets. The lytic units 30/10⁶ cells were determined using inverse number of NK cells required to lyse 30% of the target cells×100.

NK cytotoxicity

NK cells were isolated from PBMCs as described in the materials and methods section. Purified NK cells were treated with IL-2 (1000 U/mL) or IL-2+sAJ2 (Cells:sAJ2 1:20). After an overnight incubation, the NK cell mediated cytotoxicity was measured using a standard 4-hour chromium release assay against oral squamous carcinoma stem cells (OSCSCs). The lytic units 30/10⁶ cells were determined using inverse number of NK cells required to lyse 30% of the target cells×100.

### Conclusion

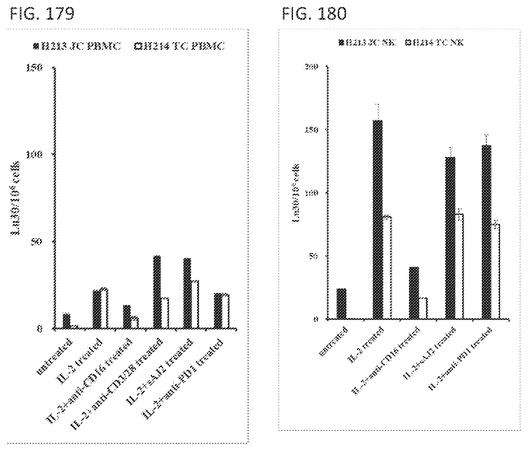

Results show higher NK cytotoxicity in PBMCs and NK cells in all treatments in ALS patient when compared to control twin (FIGS. 179 and 180; Table 238 and 239).

### Example 100

### PBMC ELISpot

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (PBMCs:sAJ2 1:20). After overnight incubation, 40,000 cells per treatment group were seeded and the number of IFN-γ spots were determined using ELISpot assay.

### PBMC ELISA

PBMCs were left untreated, treated with IL-2 (1000 U/mL) or treated with the combination of IL-2 and anti-CD16 mAb (3 μg/ml), IL-2+anti-CD3/28 antibodies (25 μl/mL) or IL-2+sAJ2 (Cells:sAJ2 1:20). After an overnight treatment at 37° C., the supernatants were harvested and the levels of IFN-γ secretion were measured using ELISA.

### Conclusion

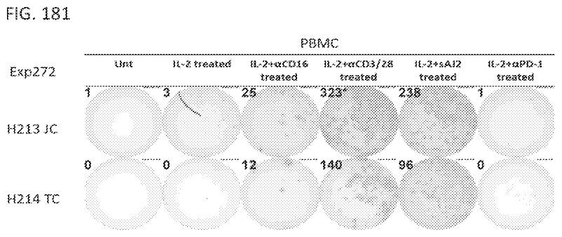

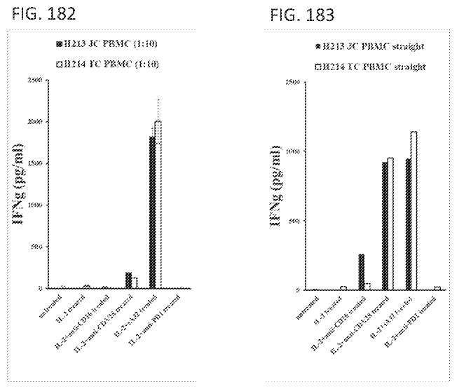

Results show increased IFN-γ spots in all treatments and increased secretion of IFN-γ in some treatments in PBMCs from ALS patient in comparison to twin control (FIGS. 181, 182, and 183; and Tables 240 and 241).

### Example 101

### NK ELISpot

NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment, 50,000 cells per treatment group were used in ELISpot assay to determine the number of spots expressing IFN-γ.

### NK ELISA

NK cells were isolated from PBMC following manufacturer's instruction. Purified NK cells were left untreated, treated with IL-2 (1000 U/mL) or treated with the combination of IL-2 and anti-CD16 mAb (3 μg/ml) or IL-2+sAJ2 (Cells:sAJ2 1:20). After an overnight treatment at 37° C., the supernatants were harvested and the levels of IFN-γ secretion were measured using ELISA.

### Conclusion

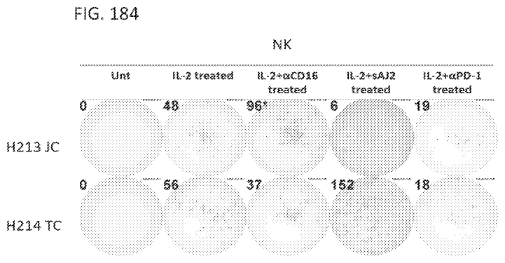

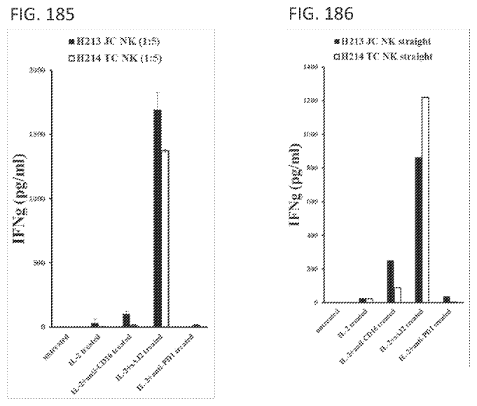

Results show variable results in IFN-γ spots but higher secretion of IFN-γ by the majority of treatments in NK cells in ALS patient in comparison to twin control (FIGS. 184, 185, and 186; Tables 242 and 243).

### Example 102

### CD8 ELISpot

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment, 50,000 cells per treatment group were used in ELISpot assay to determine the number of spots expressing IFN-γ.

### CD8 ELISA

CD8+ T cells were isolated from PBMC following manufacturer's instruction. Purified CD8+ T cells were left untreated, treated with IL-2 (1000 U/mL) or treated with the combination of IL-2 and anti-CD3/28 mAb (25 μl/ml) or IL-2+sAJ2 (Cells:sAJ2 1:20). After an overnight treatment at 37° C., the supernatants were harvested and the levels of IFN-γ secretion were measured using ELISA.

### Conclusion

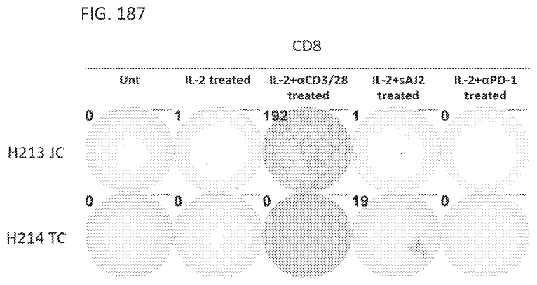

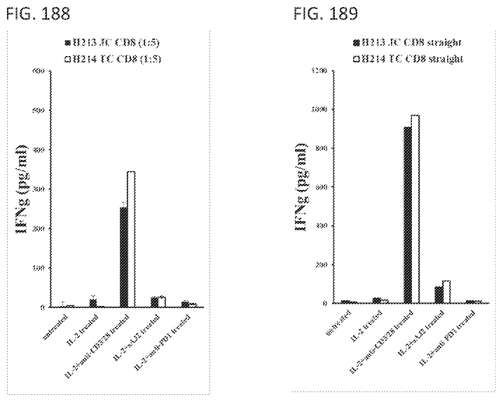

Results show increased IFN-γ spots and higher secretion of IFN-γ in some treatments in CD8+ T cells from ALS patient in comparison to twin control (FIGS. 187, 188, and 189; Tables 244 and 245).

TABLE 1
Percentages of CD8+ T-Cell expansion
within super-charged NK cells

| | ALS Patient | Healthy Twin | Healthy Control |
|---|---|---|---|
| PBMCs | 46 | 52 | 36 |
| Day 9 | 75 | 75 | 79 |
| Expanded NK Day 27 | 20 | 53 | 34 |

TABLE 2
Increased NKT cell expansion by super-charged NK cells in ALS

| | | ALS | Healthy | Healthy |
|---|---|---|---|---|
| | | Patient | Twin | Control |
| PBMCs | | 2.9 | 4.6 | 4.6 |
| | Day 9 | 28 | 17 | 7 |
| | Day 12 | 41 | 26 | 10 |
| Expanded NK | Day 27 | 20 | 36 | 14 |

TABLE 3
The fold increase in expansion of super-charged NK cells by day

| Day | ALS | Healthy twin | Healthy control |
|---|---|---|---|
| 6 | 0.69 | 0.59 | 0.7 |
| 9 | 1.9 | 2.1 | 1.3 |
| 12 | 5.1 | 3.8 | 2.7 |
| 15 | 5.4 | 5.5 | 4.3 |
| 19 | 2.16 | 4.6 | 3.6 |
| 22 | 1.1 | 1 | 1.4 |

TABLE 4
Jun. 26, 2018 PBMC (Day 0) - No therapy

| | JC (ALS) | TC(healthy twin) | ON(healthy control) |
|---|---|---|---|
| NK | 13.5% | 14.3% | 9.49% |
| CD4 | 29.8% | 37.9% | 30.3% |
| CD8 | 37.6% | 29.4% | 31.8% |
| NKT | 2.29% | 1.31% | 3.72% |
| CD19 | 3.64% | 5.20% | 8.37% |
| CD14 | 13.1% | 36.3% | 16.9% |

TABLE 5
Oct. 16, 2018 (exp83) In lab drawn Jul.
16, 2018 Neulasta shot, no IL-2

| | H51 JC | H52 TC | H53 MY |
|---|---|---|---|
| NK | 13.4% | 6.66% | 5.04% |
| CD4 | 39.6% | 44.9% | 53.4% |
| CD8 | 32.8% | 32.0% | 17.4% |
| CD19 | 6.02% | 5.97% | 9.06% |
| CD14 | 12.4% | 26.8% | 18.9% |

TABLE 6

| | H53 NK alone | | |
|---|---|---|---|
| | IL-2 treated | IL-2 + anti-CD16 treated | IL-2 + sAJ2 treated |
| 5 | 43.7 | 19.9 | 53.6 |
| 2.5 | 26.5 | 8.1 | 30.6 |

TABLE 7

| | H53 NK alone | | |
|---|---|---|---|
| | IL-2 treated | IL-2 + anti-CD16 treated | IL-2 + sAJ2 treated |
| 5 | 43.7 | 19.9 | 53.6 |
| 2.5 | 26.5 | 8.1 | 30.6 |

TABLE 8

| | H53 NK alone | | |
|---|---|---|---|
| | IL-2 treated | IL-2 + anti-CD16 treated | IL-2 + sAJ2 treated |
| 5 | 43.7 | 19.9 | 53.6 |
| 2.5 | 26.5 | 8.1 | 30.6 |

TABLE 9

| | H51 NK + H51 monocyte | | |
|---|---|---|---|
| | IL-2 treated | IL-2 + anti-CD16 treated | IL-2 + sAJ2 treated |
| 5 | 87.0 | 57.9 | 83.5 |
| 2.5 | 57.9 | 39.2 | 61.2 |

TABLE 10

| | H52 TC NK alone | | |
|---|---|---|---|
| | IL-2 treated | IL-2 + anti-CD16 treated | IL-2 + sAJ2 treated |
| 5 | 77.7 | 30.5 | 84.5 |
| 2.5 | 51.4 | 14.2 | 68.3 |

TABLE 11

| | H52 TC NK alone | | |
|---|---|---|---|
| | IL-2 treated | IL-2 + anti-CD16 treated | IL-2 + sAJ2 treated |
| 5 | 77.7 | 30.5 | 84.5 |
| 2.5 | 51.4 | 14.2 | 68.3 |

TABLE 12

| | H53 NK + H51 JC monocyte | | |
|---|---|---|---|
| | IL-2 treated | IL-2 + anti-CD16 treated | IL-2 + sAJ2 treated |
| 5 | 68.1 | 56.8 | 57.6 |
| 2.5 | 48.1 | 34.1 | 34.1 |

TABLE 13

| | H53 NK + H52 TC monocyte | | |
|---|---|---|---|
| | IL-2 treated | IL-2 + anti-CD16 treated | IL-2 + sAJ2 treated |
| 5 | 62.7 | 55.1 | 57.0 |
| 2.5 | 44.2 | 37.8 | 42.6 |

TABLE 14

| | naïve CD4 | | | Treg | | Tresp | |
|---|---|---|---|---|---|---|---|
| | H51 | H52 | H53 | H51 | H52 | H51 | H52 |
| | JC | TC | MY | JC | TC | JC | TC |
| 1st sup | | | | 9048.8 | 7915.7 | 2435.7 | 821.2 |
| collection | | | | | | | |
| 2nd sup | 452.1 | 187.8 | 11.6 | 8356.2 | 8752.6 | 300.2 | 277.4 |
| collection | | | | | | | |
| 3rd sup | 54.2 | 183.3 | 863.7 | 6062.7 | 6676.4 | 467.3 | 367.1 |
| collection | | | | | | | |

TABLE 15

| | H63 JC |
|---|---|
| NK | 8.32% |
| CD4 | 43.3% |
| CD8 | 22.1% |
| CD19 | 16.1% |
| CD14 | 10.8% |
| total | 100.62% |

TABLE 16

| | H64 JC |
|---|---|
| NK | 29.1% |
| CD4 | 36.5% |
| CD8 | 7.15% |
| CD19 | 7.89% |
| CD14 | 9.42% |
| total | 102.61% |

TABLE 17

| | P66 LP | P67 VB | H65 SM | H66 JC | H67 MK |
|---|---|---|---|---|---|
| NK | 32.1% | 15.0% | 12.8% | 14.3% | 13.6% |
| CD4 | 31.7% | 47.6% | 35.4% | 42.6% | 34.2% |
| CD8 | 7.58% | 11.8% | 22.8% | 23.4% | 18.6% |
| CD19 | 7.89% | 8.38% | 8.49% | 9.58% | 11.1% |
| CD14 | 30.3% | 32.3% | 22.8% | 11.2% | 17.9% |
| Total | 109.57% | 115.08% | 102.29% | 101.08% | 95.4% |

TABLE 18

| | | | IL--2 + | IL-2 + | IL-2 + |
|---|---|---|---|---|---|
| Cr release | | IL-2 | anti-CD16 | anti-CD3/28 | sAJ2 |
| assay | untreated | treated | treated | treated | treated |
| P66 LP | 2.46 | 61.31 | 33.65 | 65.96 | 67.94 |
| PBMC | | | | | |
| H65 SM | 4.17 | 36.23 | 14.75 | 36.94 | 35.21 |
| PBMC | | | | | |
| H66 JC | 3.38 | 35.36 | 19.41 | | 32.46 |
| PBMC | | | | | |
| H67 MK | 7.69 | 44.41 | | 14.05 | 39.56 |
| PBMC | | | | | |

TABLE 19

| | | IL-2 | IL--2 + anti- | IL-2 + sAJ2 |
|---|---|---|---|---|
| | untreated | treated | CD16 treated | treated |
| P66 LP NK | 26.94 | 218.17 | 68.33 | 203.83 |
| H66 JC NK | | 117.87 | 67.09 | 164.14 |

TABLE 20

| | | | IL-2 + | IL-2 + | IL-2 + |
|---|---|---|---|---|---|
| | | IL-2 | anti-CD16 | anti-CD3/28 | sAJ2 |
| 1:5 dilution | untreated | treated | treated | treated | treated |
| P66 LP | 44.9 | 135.2 | 252.8 | 669.4 | 1649.9 |
| PBMC | | | | | |
| P65 SM | 29.7 | 35.5 | 763.3 | 940.2 | 2093.2 |
| PBMC | | | | | |
| H66 JC | 75.2 | 734.4 | 413.1 | 688.2 | 1652.8 |
| PBMC | | | | | |
| H67 MK | 10.3 | 120.7 | 1369.8 | 1140.9 | 1923.6 |
| PBMC | | | | | |

TABLE 21
(1:5)

| | P65 | H65 | H66 |
|---|---|---|---|
| | LP | SM | JC |
| | NK | NK | NK |
| untreated | | | |
| IL-2 treated | 56.2 | 24.6 | 137.4 |
| IL-2 + anti-CD16 treated | 193.8 | 687.8 | 265.4 |
| IL-2 + sAJ2 treated | 79.6 | 122.3 | 167.7 |

TABLE 22

| | H68 JC |
|---|---|
| NK | 13.5% |
| CD4 | 40.8% |
| CD8 | 22.3% |
| CD19 | 11.4% |
| CD14 | 15.7% |
| total | 103.7% |

TABLE 23

| | P68 CL | H70 JC (adjusted) |
|---|---|---|
| NK | 7.27% | 11.4% |
| CD4 | 32.6% | 39.25% |
| CD8 | 48.3% | 24.75% |
| CD19 | 3.59% | 7.99% |
| CD14 | 9.91% | 20.2% |
| total | 101.67% | 103.59% |

TABLE 24

| | | | IL-2 + | IL-2 + | IL-2 + |
|---|---|---|---|---|---|
| | | IL-2 | anti-CD16 | anti-CD3/28 | sAJ2 |
| | untreated | treated | treated | treated | treated |
| P68 CL | 73.56 | 1447.46 | 1565.62 | 1999.77 | 2036.87 |
| PBMC | | | | | |
| H70 JC | 76.31 | 132.64 | 778.37 | 1973.67 | 1652.17 |
| PBMC | | | | | |

TABLE 25

| | | IL-2 + | |
|---|---|---|---|
| | IL-2 | anti-CD 16 | IL-2 + sAJ2 |
| | treated | treated | treated |
| H70 JC NK | 1478.8 | 425.2 | 1120.0 |
| (1:5) | | | |

| | | | IL-2 + | |
|---|---|---|---|---|
| | | IL-2 | anti-CD3/28 | IL-2 + sAJ2 |
| | untreated | treated | treated | treated |
| H70 JC CD8 | 154.4 | | 4003.6 | 196.8 |
| (1:5) | | | | |

TABLE 26

| | H80 DM | H81 JC | H82 TC | P82 SS |
|---|---|---|---|---|
| NK | 7.61% | 10.5% | 8.80% | 10.5% |
| CD4 | 37.9% | 34.3% | 37.7% | 14.3% |
| CD8 | 24.8% | 25.5% | 26.6% | 15.9% |
| CD19 | 9.13% | 11.2% | 9.63% | 22.6% |
| CD14 | 15.0% | 15.9% | 15.9% | 33.1% |
| total | 94.44% | 97.4% | 98.63% | 96.4% |

TABLE 27

| | | | IL--2 + | IL-2 + | IL-2 + |
|---|---|---|---|---|---|
| | | IL-2 | anti-CD16 | anti-CD3/28 | sAJ2 |
| PBMC | untreated | treated | treated | treated | treated |
| H80 DM | 2.25 | 7.04 | 5.07 | 5.79 | 6.26 |
| H81 JC | 4.42 | 15.09 | 15.13 | 16.82 | 14.94 |
| H82 TC | 4.21 | 9.04 | 6.82 | 9.39 | 7.55 |
| P82 SS | 3.94 | 12.53 | 9.12 | 11.18 | 9.74 |

TABLE 28

| | IL-2 | IL--2 + anti- | IL-2 + sAJ2 |
|---|---|---|---|
| NK | treated | CD16 treated | treated |
| H80 DM | 25.28 | 14.20 | 30.01 |
| H81 JC | 44.80 | 29.40 | 68.79 |
| H82 TC | 36.37 | 18.59 | 63.68 |
| P82 SS | 37.57 | 31.58 | 44.15 |
| KZ sNK (day 17) | 89.02 | | |
| RO sNK (day 17) | 45.50 | | |

TABLE 29

| | | | IL-2 + | IL-2 + | IL-2 + |
|---|---|---|---|---|---|
| | | IL-2 | anti-CD16 | anti-CD3/28 | sAJ2 |
| 1:5 dilution | untreated | treated | treated | treated | treated |
| H80 DM | 90.73 | 479.84 | 1017.35 | 1880.28 | 3348.85 |
| PBMC | | | | | |
| H81 JC | 181.80 | 2831.09 | 3522.71 | 2453.44 | 3683.20 |
| PBMc | | | | | |
| H82 TC | 89.46 | 1454.23 | 1949.69 | 984.87 | 2937.45 |
| PBMC | | | | | |
| P82 SS | 35.32 | 33.41 | 2077.70 | 2109.54 | 4079.96 |
| PBMC | | | | | |

TABLE 30

| | 1:5 dilution | | | | | |
|---|---|---|---|---|---|---|
| | H80 DM | | | H81 JC | | |
| | | IL-2 + | IL-2 + | | IL-2 + | IL-2 + |
| | IL-2 | antiCD16 | sAJ2 | IL-2 | antiCD16 | sAJ2 |
| | treated | treated | treated | treated | treated | treated |
| NK alone | 228.72 | 507.90 | 779.67 | 400.84 | 484.84 | 1229.33 |
| NK + auto | 430.49 | 227.90 | 4059.05 | 166.13 | 338.25 | 4581.18 |
| monocyte | | | | | | |
| | H82 TC | | | P82 SS | | |
| | | IL-2 + | IL-2 + | | IL-2 + | IL-2 + |
| | IL-2 | antiCD16 | sAJ2 | IL-2 | antiCD16 | sAJ2 |
| | treated | treated | treated | treated | treated | treated |
| NK alone | 337.43 | 436.25 | 848.85 | 526.84 | 884.26 | 3759.28 |
| NK + auto | 224.60 | 653.67 | 4441.18 | 180.13 | 331.66 | 4052.46 |
| monocyte | | | | | | |

TABLE 31

| | P82 SS NK | | | | P83 SS monocyte | | |
|---|---|---|---|---|---|---|---|
| | | IL-2 + | IL-2 + | | | IL-2 + | IL-2 + |
| | Il-2 | antiCD16 | sAJ2 | | il-2 | antiCD16 | sAJ2 |
| | treated | treated | treated | | treated | treated | treated |
| H81 JC | 123.73 | 696.86 | 4786.01 | H81 JC | 238.57 | | 4910.24 |
| monocyte | | | | NK (1:5) | | | |
| (1:5) | | | | | | | |
| H82 TC | 396.21 | 1620.76 | 4674.31 | H82 TC | 107.03 | 260.49 | 4064.65 |
| monocyte | | | | NK (1:5) | | | |
| (1:5) | | | | | | | |
| | H83 JC NK (1:5) | | | | H83 JC monocyte (1:5) | | |
| | | IL-2 + | IL-2 + | | | IL-2 + | IL-2 + |
| | il-2 | antiCD16 | sAJ2 | | il-2 | antiCD16 | sAJ2 |
| | treated | treated | treated | | treated | treated | treated |
| H80 DM | 163.40 | 235.44 | 4909.20 | H80 DM | 155.05 | 1050.76 | 4967.66 |
| monocyte | | | | NK (1:5) | | | |
| H82 TC | 80.93 | 430.66 | 4391.40 | H82 TC | 79.89 | 213.51 | 4378.87 |
| monocyte | | | | NK (1:5) | | | |

TABLE 32

| | | IL-2 | IL-2 + anti- |
|---|---|---|---|
| | untreated | treated | CD3/28 treated |
| H80 DM CD8 (1:5) | 71.60 | 283.94 | 1254.95 |
| H81 JC CD8 (1:5) | 325.79 | 476.90 | 1880.34 |
| H82 TC CD8 (1:5) | 235.89 | 84.00 | 1247.20 |

TABLE 33

| | |
|---|---|
| AM | PM |

| | H84 JC | H85 TC | H86 JC | H87 TC |
|---|---|---|---|---|
| NK | 13.8% | 7.67% | 8.35% | 9.23% |
| CD4 | 40.5% | 33.5% | 38.3% | 37.7% |
| CD8 | 29.2% | 25.7% | 24.3% | 28.8% |
| CD19 | 10.7% | 10.8% | 11.7% | 10.5% |
| CD14 | 17.9% | 14.1% | 18.8% | 15.7% |
| total | 112.1% | 91.67% | 101.45% | 101.93% |

TABLE 34

| | H84 JC | H85 TC |
|---|---|---|
| CD45RA+/CD4+ | 19.89% | 21.06% |

TABLE 35

| | H84 JC | H85 TC |
|---|---|---|
| CD45RO+/CD4+ | 80.55% | 74.07% |

TABLE 36

| | | | IL-2 + | IL-2 + | IL-2 + |
|---|---|---|---|---|---|
| PBMC | | IL-2 | anti-CD16 | anti-CD3/28 | sAJ2 |
| (Cr51) | untreated | treated | treated | treated | treated |
| H84 JC | 8.87 | 21.96 | 13.47 | 26.25 | 34.50 |
| AM | | | | | |
| H85 TC | 6.60 | 25.98 | 19.30 | 34.31 | 24.62 |
| AM | | | | | |
| H86 JC | 2.90 | 13.68 | 9.93 | 22.85 | 24.32 |
| PM | | | | | |
| H87 TC | 6.79 | 17.09 | 13.12 | 9.75 | 23.12 |
| PM | | | | | |

TABLE 37

| | IL-2 | IL-2 + anti- | IL-2 + sAJ2 |
|---|---|---|---|
| | treated | CD16 treated | treated |
| H84 JC NK alone | 104.22 | 20.22 | 105.50 |
| H84 JC NK + auto monocyte | 169.51 | 50.03 | 115.23 |
| H84 JC NK + allo monocyte | 195.55 | 64.84 | 164.18 |
| H85 TC NK alone | 72.62 | 26.84 | 144.04 |
| H85 TC NK + auto monocyte | 168.11 | 87.29 | 148.86 |
| H85 TC NK + allo monocyte | 127.14 | 135.73 | 223.53 |

TABLE 38

| | | | IL-2 + | IL-2 + | IL-2 + |
|---|---|---|---|---|---|
| 1:8 | | IL-2 | anti-CD16 | anti-CD3/28 | sAJ2 |
| dilution | untreated | treated | treated | treated | treated |
| H84 JC | 9.28 | 215.18 | 1572.90 | 2201.84 | 3191.43 |
| PBMC | | | | | |
| H85 TC | 276.33 | 268.84 | 1438.13 | 1031.31 | 2219.31 |
| PBMC | | | | | |
| H86 JC | 644.46 | 817.92 | 1946.02 | 1102.44 | 2285.45 |
| PBMC | | | | | |
| H87 TC | 353.70 | 232.66 | 1471.82 | 770.50 | 2392.77 |
| PBMC | | | | | |

TABLE 39

| | 1:8 dilution | | | | | | | |
|---|---|---|---|---|---|---|---|---|
| | H84 JC CD8 | | | | H85 TC CD8 | | | |
| | | | IL-2 + | IL-2 + | | | IL-2 + | IL-2 + |
| | | IL-2 | anti-CD3/28 | sAJ2 | | IL-2 | anti-CD3/28 | sAJ2 |
| | untreated | treated | treated | treated | untreated | treated | treated | treated |
| Day1 | 23.09 | 55.59 | 615.75 | 437.95 | 237.22 | 193.24 | 546.93 | 64.20 |
| Day4 | 121.55 | 222.88 | 1918.64 | 564.13 | 566.05 | 437.00 | 1705.48 | 303.17 |

TABLE 40

| | 1:8 dilution | | | | | |
|---|---|---|---|---|---|---|
| | H84 JC | | | H85 TC | | |
| | | IL-2 + | IL-2 + | | IL-2 + | IL-2 + |
| | IL-2 | antiCD16 | sAJ2 | IL-2 | antiCD16 | sAJ2 |
| | treated | treated | treated | treated | treated | treated |
| NK alone | 248.16 | 448.22 | 529.98 | 342.10 | 335.14 | 441.26 |
| NK + auto | 862.26 | 759.62 | 3374.31 | 322.96 | 926.62 | 3868.37 |
| monocyte | | | | | | |
| monocyte | 571.73 | 545.64 | 533.46 | 616.97 | 373.41 | 1396.33 |
| alone | | | | | | |

TABLE 41

| 1:8 dilution | H86 JC nCD4 | H87 TC nCD4 |
|---|---|---|
| Day 4 | 638.69 | 454.21 |
| Day 10 | 561.27 | 500.09 |
| Day 12 | 440.82 | 662.59 |

TABLE 42

| | H88 JC | H89 TC |
|---|---|---|
| NK | 10.4% | 9.79% |
| CD4 | 42.9% | 41.0% |
| CD8 | 22.9% | 24.0% |
| CD19 | 8.14% | 7.38% |
| CD14 | 16.2% | 17.9% |
| total | 100.54% | 100.7% |

TABLE 43

| | | | IL-2 + | IL-2 + | IL-2 + |
|---|---|---|---|---|---|
| | | IL-2 | anti-CD16 | anti-CD3/28 | sAJ2 |
| | untreated | treated | treated | treated | treated |
| H88 | 0.59 | 17.85 | 17.79 | 24.87 | 19.91 |
| JC | | | | | |
| H89 | 5.22 | 23.85 | 16.87 | 34.31 | 18.84 |
| TC | | | | | |

TABLE 44

| | | | IL-2 + | IL-2 + | IL-2 + |
|---|---|---|---|---|---|
| 1:8 | | IL-2 | anti-CD16 | anti-CD3/28 | sAJ2 |
| dilution | untreated | treated | treated | treated | treated |
| H88 JC | 498.46 | 479.74 | 1106.19 | 2210.58 | 1922.31 |
| PBMC | | | | | |
| H89 TC | 251.37 | 678.16 | 1717.66 | 1468.08 | 2768.39 |
| PBMC | | | | | |

TABLE 45

| | 1:8 dilution | | | | | | | |
|---|---|---|---|---|---|---|---|---|
| | H88 JC CD4 | | | | H89 TC CD4 | | | |
| | | | IL-2 + | IL-2 + | | | IL-2 + | IL-2 + |
| | | IL-2 | anti-CD3/28 | sAJ2 | | IL-2 | anti-CD3/28 | sAJ2 |
| | untreated | treated | treated | treated | untreated | treated | treated | treated |
| Day1 | 228.94 | 371.58 | 896.63 | 605.27 | 518.27 | 470.72 | 704.42 | 611.34 |
| Day4 | 281.55 | 326.06 | 1058.49 | 534.46 | 641.69 | 547.61 | 1093.90 | 377.65 |
| Day10 | 289.64 | 437.34 | 602.24 | 622.47 | 646.75 | 612.35 | 463.64 | 345.28 |
| Day12 | 406.99 | 505.12 | 609.32 | 555.70 | 558.74 | 622.47 | 537.49 | 467.69 |

TABLE 46

| | 1:8 dilution | | | | | | | |
|---|---|---|---|---|---|---|---|---|
| | H88 JC CD8 | | | | H89 TC CD8 | | | |
| | | | IL-2 + | IL-2 + | | | IL-2 + | IL-2 + |
| | | IL-2 | anti-CD3/28 | sAJ2 | | IL-2 | anti-CD3/28 | sAJ2 |
| | untreated | treated | treated | treated | untreated | treated | treated | treated |
| Day1 | 237.70 | 312.43 | 1398.18 | 538.02 | 631.44 | 497.89 | 991.98 | 489.58 |
| Day4 | 284.75 | 386.48 | 1845.90 | 728.32 | 664.66 | 618.99 | 952.53 | 396.86 |
| Day10 | 347.03 | 452.22 | 624.52 | 746.32 | 711.72 | 769.15 | 683.34 | 494.43 |
| Day12 | 376.79 | 567.09 | 862.57 | 885.41 | 758.77 | 663.28 | 645.28 | 478.51 |

TABLE 47

| | H90 JC | H91 TC |
|---|---|---|
| NK | 9.84% | 8.30% |
| CD4 | 37.2% | 38.1% |
| CD8 | 24.3% | 30.5% |
| CD19 | 13.5% | 13.3% |
| CD14 | 7.07% | 7.82% |
| total | 91.91% | 98.02% |

TABLE 48
LU30 Averages

| | | | IL--2 + | IL-2 + | IL-2 + |
|---|---|---|---|---|---|
| | | IL-2 | anti-CD16 | anti-CD3/28 | sAJ2 |
| PBMC | untreated | treated | treated | treated | treated |
| H90 | 2.31 | 7.28 | 7.09 | 12.85 | 16.68 |
| JC | | | | | |
| H91 | 2.39 | 22.55 | 12.13 | 13.80 | 20.54 |
| TC | | | | | |

TABLE 49

| | | | IL-2 + | IL-2 + | IL-2 + |
|---|---|---|---|---|---|
| 1:8 | | IL-2 | anti-CD16 | anti-CD3/28 | sAJ2 |
| dilution | untreated | treated | treated | treated | treated |
| H90 JC | 565.84 | 347.46 | 1023.82 | 925.24 | 1344.53 |
| PBMC | | | | | |
| H91 TC | 649.45 | 1052.53 | 1514.25 | 919.00 | 1186.05 |
| PBMC | | | | | |

TABLE 50

| 1:2 dilution | untreated | IL-2 + sAJ2 treated |
|---|---|---|
| H90 JC neutrophils | 26.6 | 72.3 |
| H91 TC neutrophils | 26.6 | 77.7 |

TABLE 51

| | | EGF | FGF2 | Eotaxin | TGFa | GCSF | FIT3L | GMCSF | Fractalkine | IFNa2 | IFNg |
|---|---|---|---|---|---|---|---|---|---|---|---|
| | | pg/ml | pg/ml | pg/ml | pg/ml | pg/ml | pg/ml | pg/ml | pg/ml | pg/ml | pg/ml |
| H84 | Untreated | 22.35 | 3.58 | 3.58 | 0.94 | 19.59 | 3.82 | 2.03 | 18.48 | 11.69 | 2.84 |
| JC | PBMC | | | | | | | | | | |
| H85 | Untreated | 24.05 | 6.9 | 4.26 | 1.53 | 25.5 | 4.6 | 4.9 | 72.23 | 17.43 | 23.35 |
| TC | PBMC | | | | | | | | | | |
| H84 | IL2 treated | 32.7 | 9.4 | 6.73 | 2.06 | 72.23 | 7.25 | 19.31 | 136.75 | 61.65 | 737.57 |
| JC | PBMC | | | | | | | | | | |
| H85 | IL2 treated | 21.7 | 8.91 | 6.65 | 2.34 | 78.01 | 7.82 | 29.52 | 144.7 | 55.3 | 356.57 |
| TC | PBMC | | | | | | | | | | |
| H84 | IL2aCD16 | 86.34 | 15.09 | 11.06 | 5.22 | 192.9 | 12.37 | 389 | 250.96 | 140.66 | 11912 |
| JC | treated | | | | | | | | | | |
| | PBMC | | | | | | | | | | |
| H85 | IL2aCD16 | 90.93 | 20.77 | 12.19 | 6.41 | 243.71 | 12.03 | 462.14 | 250.96 | 144.7 | 11461 |
| TC | treated | | | | | | | | | | |
| | PBMC | | | | | | | | | | |
| H84 | IL2aCD328 | 86.34 | 22.68 | 11.86 | 7.53 | 122.29 | 14.25 | 308.14 | 282.21 | 176.83 | 9998 |
| JC | treated | | | | | | | | | | |
| | PBMC | | | | | | | | | | |
| H85 | IL2aCD328 | 63.33 | 15.53 | 11.37 | 9.15 | 131.11 | 13.08 | 223.2 | 195.73 | 132.96 | 6809 |
| TC | treated | | | | | | | | | | |
| | PBMC | | | | | | | | | | |
| H84 | IL2sAJ2 | 66.78 | 26.26 | 15.31 | 19.31 | 2097 | 13.85 | 148.87 | 524.82 | 258.43 | 16816 |
| JC | treated | | | | | | | | | | |
| | PBMC | | | | | | | | | | |
| H85 | IL2sAJ2 | 48.82 | 23.35 | 14.05 | 24.05 | 3671 | 19.59 | 192.9 | 489.14 | 182.02 | 16371 |
| TC | treated | | | | | | | | | | |
| | PBMC | | | | | | | | | | |

TABLE 52

| | | Scd40L | IL-17a | IL-1Ra | IL-fa | IL-9 | IL-1b | L-2 | IL-3 | IL-4 | IL-5 |
|---|---|---|---|---|---|---|---|---|---|---|---|
| | | pg/ml | pg/ml | pg/ml | pg/ml | pg/ml | pg/ml | pg/ml | pg/ml | pg/ml | pg/ml |
| H84 | Untreated | 74.12 | 1.91 | 7.62 | 1.87 | 1.41 | 1.51 | 3.16 | 1.08 | 13.08 | 0.98 |
| JC | PBMC | | | | | | | | | | |
| H85 | Untreated | 46.8 | 2.66 | 84.16 | 5.22 | 2.9 | 13.85 | 400.4 | 1.1 | 26.26 | 1.14 |
| TC | PBMC | | | | | | | | | | |
| H84 | IL2 treated | 90.93 | 3.7 | 223.2 | 15.09 | 11.69 | 78.01 | 5861 | 1.46 | 52.33 | 4.1 |
| JC | PBMC | | | | | | | | | | |
| H85 | IL2 treated | 60.01 | 3.82 | 290.61 | 12.37 | 11.37 | 31.3 | 6058 | 1.55 | 58.41 | 2.73 |
| TC | PBMC | | | | | | | | | | |
| H84 | IL2aCD16 | 166.92 | 5.84 | 997.83 | 346.33 | 16.94 | 1503 | 5816 | 2 | 93.34 | 9.15 |
| JC | treated | | | | | | | | | | |
| | PBMC | | | | | | | | | | |
| H85 | IL2aCD16 | 109.58 | 7.07 | 1147 | 611.21 | 17.43 | 2144 | 5893 | 2.09 | 90.93 | 6.73 |
| TC | treated | | | | | | | | | | |
| | PBMC | | | | | | | | | | |
| H84 | IL2aCD328 | 243.71 | 39.46 | 1280 | 132.96 | 28.67 | 681.12 | 5635 | 7.07 | 462.14 | 125.73 |
| JC | treated | | | | | | | | | | |
| | PBMC | | | | | | | | | | |
| H85 | IL2aCD328 | 136.75 | 79 | 1418 | 68.56 | 24.76 | 400.4 | 5824 | 12.9 | 250.96 | 95.83 |
| TC | treated | | | | | | | | | | |
| | PBMC | | | | | | | | | | |
| H84 | IL2sAJ2 | 176.83 | 8.45 | 2880 | 628.12 | 21.39 | 3076 | 5905 | 2.28 | 122.29 | 3.38 |
| JC | treated | | | | | | | | | | |
| | PBMC | | | | | | | | | | |
| H85 | IL2sAJ2 | 88.6 | 6.73 | 3058 | 1390 | 19.03 | 3672 | 5620 | 1.91 | 109.58 | 4.1 |
| TC | treated | | | | | | | | | | |
| | PBMC | | | | | | | | | | |

TABLE 53

| | | | | | | | | | | PDGF- | |
|---|---|---|---|---|---|---|---|---|---|---|---|
| | | GRO | IL-10 | MCP3 | IL-12p40 | MDC | IL-12p70 | PDGFaa | IL-13 | AB/BB | IL-15 |
| | | pg/ml | pg/ml | pg/ml | pg/ml | pg/ml | pg/ml | pg/ml | pg/ml | pg/ml | pg/ml |
| H84 | Untreated | 317.29 | 3.11 | 4.1 | 3.24 | 53.8 | 2.61 | 103.81 | 4.1 | 586.54 | 1.9 |
| JC | PBMC | | | | | | | | | | |
| H85 | Untreated | 436.48 | 14.25 | 6.12 | 4.6 | 174.3 | 3.47 | 148.87 | 6.12 | 578.51 | 2.96 |
| TC | PBMC | | | | | | | | | | |
| H84 | IL2 treated | 787.28 | 35.16 | 19.59 | 11.69 | 236.67 | 5.22 | 216.77 | 27.85 | 628.12 | 3.58 |
| JC | PBMC | | | | | | | | | | |
| H85 | IL2 treated | 578.51 | 30.4 | 18.48 | 10.47 | 213.63 | 5.98 | 153.17 | 23.35 | 611.21 | 5.58 |
| TC | PBMC | | | | | | | | | | |
| H84 | IL2aCD16 | 2745 | 64.18 | 718.37 | 35.16 | 1226 | 46.8 | 389 | 129.29 | 1390 | 7.62 |
| JC | treated | | | | | | | | | | |
| | PBMC | | | | | | | | | | |
| H85 | IL2aCD16 | 3181 | 118.96 | 927.72 | 39.46 | 1363 | 60.01 | 389 | 131.11 | 1702 | 7.62 |
| TC | treated | | | | | | | | | | |
| | PBMC | | | | | | | | | | |
| H84 | IL2aCD328 | 2894 | 1046 | 645.4 | 33.18 | 1418 | 37.25 | 377.89 | 636.71 | 1226 | 6.73 |
| JC | treated | | | | | | | | | | |
| | PBMC | | | | | | | | | | |
| H85 | IL2aCD328 | 2750 | 1096 | 708.91 | 27.85 | 1503 | 18.75 | 274.05 | 503.15 | 1280 | 8.03 |
| TC | treated | | | | | | | | | | |
| | PBMC | | | | | | | | | | |
| H84 | IL2sAJ2 | 3439 | 6767 | 532.22 | 159.88 | 510.28 | 44.23 | 377.89 | 84.16 | 1560 | 9.15 |
| JC | treated | | | | | | | | | | |
| | PBMC | | | | | | | | | | |
| H85 | IL2sAJ2 | 3473 | 7591 | 436.48 | 336.38 | 462.14 | 336.38 | 236.67 | 75.08 | 1045 | 9.65 |
| TC | treated | | | | | | | | | | |
| | PBMC | | | | | | | | | | |

TABLE 54

| | | IL-6 | IL-7 | IL-8 | IP-10 | MCP1 | MCP1a | MIP1b | Rantes | TNFa | TNFb | VEGF |
|---|---|---|---|---|---|---|---|---|---|---|---|---|
| | | pg/ml | pg/ml | pg/ml | pg/ml | pg/ml | pg/ml | pg/ml | pg/ml | pg/ml | pg/ml | pg/ml |
| H84 | Untreated | 2.76 | 3.7 | 41.78 | 17.43 | 7.25 | 22.02 | 52.33 | 3202 | 4.42 | 1.64 | 33.18 |
| JC | PBMC | | | | | | | | | | | |
| H85 | Untreated | 59.21 | 7.25 | 517.51 | 5118 | 1046 | 144.7 | 475.47 | 1896 | 42.99 | 2.17 | 63.33 |
| TC | PBMC | | | | | | | | | | | |
| H84 | IL2 treated | 187.38 | 13.08 | 1731 | 5325 | 3791 | 797.51 | 1226 | 2776 | 86.34 | 8.68 | 169.34 |
| JC | PBMC | | | | | | | | | | | |
| H85 | IL2 treated | 233.23 | 15.53 | 1200 | 5713 | 3688 | 882.8 | 1574 | 1987 | 233.23 | 12.72 | 144.7 |
| TC | PBMC | | | | | | | | | | | |
| H84 | IL2aCD16 | 1096 | 61.65 | 3903 | 5459 | 4333 | 2142 | 2908 | 3763 | 1363 | 38.34 | 262.25 |
| JC | treated | | | | | | | | | | | |
| | PBMC | | | | | | | | | | | |
| H85 | IL2aCD16 | 1759 | 77.02 | 4131 | 5509 | 4479 | 2193 | 3120 | 3829 | 1503 | 29.52 | 258.43 |
| TC | treated | | | | | | | | | | | |
| | PBMC | | | | | | | | | | | |
| H84 | IL2aCD328 | 672.05 | 60.01 | 3931 | 5345 | 4337 | 2173 | 3298 | 3736 | 2570 | 66.78 | 223.2 |
| JC | treated | | | | | | | | | | | |
| | PBMC | | | | | | | | | | | |
| H85 | IL2aCD328 | 317.29 | 42.99 | 3925 | 5499 | 4246 | 2159 | 3320 | 3719 | 1787 | 44.23 | 247.31 |
| TC | treated | | | | | | | | | | | |
| | PBMC | | | | | | | | | | | |
| H84 | IL2sAJ2 | 3448 | 115.73 | 4196 | 2903 | 4511 | 2280 | 3547 | 3841 | 3713 | 34.16 | 317.29 |
| JC | treated | | | | | | | | | | | |
| | PBMC | | | | | | | | | | | |
| H85 | IL2sAJ2 | 3604 | 122.29 | 4203 | 3058 | 4388 | 2230 | 3612 | 3583 | 4714 | 27.04 | 262.25 |
| TC | treated | | | | | | | | | | | |
| | PBMC | | | | | | | | | | | |

TABLE 55

| | | EGF | FGF2 | Eotaxin | TGFa | GCSF | FIT3L | GMCSF | Fractalkine | IFNa2 | IFNg |
|---|---|---|---|---|---|---|---|---|---|---|---|
| | | pg/ml | pg/ml | pg/ml | pg/ml | pg/ml | pg/ml | pg/ml | pg/ml | pg/ml | pg/ml |
| H84 | IL2 | 48.14 | 5.71 | 4.6 | 1.14 | 31.3 | 4.18 | 3.58 | 44.23 | 20.77 | 66.78 |
| JC | treated | | | | | | | | | | |
| | NK | | | | | | | | | | |
| H85 | IL2 | 90.93 | 5.11 | 4.95 | 1.09 | 19.59 | 4.6 | 3.22 | 37.25 | 13.08 | 11.06 |
| TC | treated | | | | | | | | | | |
| | NK | | | | | | | | | | |
| H84 | IL2aCD16 | 32.7 | 5.71 | 5.11 | 1.48 | 19.59 | 5 | 55.3 | 6678 | 26.26 | 262.25 |
| JC | treated | | | | | | | | | | |
| | NK | | | | | | | | | | |
| H85 | IL2aCD16 | 103.81 | 6.9 | 4.79 | 1.58 | 39.46 | 5 | 60.01 | 44.23 | 33.18 | 308.14 |
| TC | treated | | | | | | | | | | |
| | NK | | | | | | | | | | |
| H84 | IL2sAJ2 | 26.65 | 6.9 | 5.58 | 1.31 | 74.12 | 5 | 32.23 | 66.78 | 22.68 | 44.23 |
| JC | treated | | | | | | | | | | |
| | NK | | | | | | | | | | |
| H85 | IL2sAJ2 | 80.51 | 6.26 | 6.57 | 1.33 | 101.06 | 5 | 29.52 | 82.05 | 30.4 | 50.9 |
| TC | treated | | | | | | | | | | |
| | NK | | | | | | | | | | |

TABLE 56

| | | | | | IL- | | IL- | | | PDGF- | |
|---|---|---|---|---|---|---|---|---|---|---|---|
| | | GRO | IL-10 | MCP3 | 12p40 | MDC | 12p70 | PDGFaa | IL-13 | AB/BB | IL-15 |
| | | pg/ml | pg/ml | pg/ml | pg/ml | pg/ml | pg/ml | pg/ml | pg/ml | pg/ml | pg/ml |
| H84 | IL2 | 131.11 | 12.03 | 5 | 3.82 | 4.9 | 3.28 | 35.16 | 3.58 | 299.25 | 2.05 |
| JC | treated | | | | | | | | | | |
| | NK | | | | | | | | | | |
| H85 | IL2 | 101.06 | 3.82 | 4.26 | 3.82 | 4.9 | 2.61 | 42.99 | 3.82 | 517.51 | 1.9 |
| TC | treated | | | | | | | | | | |
| | NK | | | | | | | | | | |
| H84 | IL2aCD16 | 132.96 | 3.82 | 5.34 | 5.45 | 5.22 | 3.82 | 40.61 | 5 | 216.77 | 2.12 |
| JC | treated | | | | | | | | | | |
| | NK | | | | | | | | | | |
| H85 | IL2aCD16 | 176.83 | 4.42 | 5 | 5 | 5.45 | 3.28 | 59.21 | 6.57 | 611.21 | 2.42 |
| TC | treated | | | | | | | | | | |
| | NK | | | | | | | | | | |
| H84 | IL2sAJ2 | 462.14 | 60.01 | 6.9 | 5.22 | 4.9 | 4.6 | 33.18 | 5.45 | 125.73 | 2.5 |
| JC | treated | | | | | | | | | | |
| | NK | | | | | | | | | | |
| H85 | IL2sAJ2 | 503.15 | 52.33 | 6.9 | 5.98 | 5.71 | 3.96 | 43.61 | 8.24 | 424.14 | 2.57 |
| TC | treated | | | | | | | | | | |
| | NK | | | | | | | | | | |

TABLE 57

| | | sCD40 | IL-17a | IL-1Ra | IL-1a | IL-9 | IL-1b | L-2 | IL-3 | IL-4 | IL-5 |
|---|---|---|---|---|---|---|---|---|---|---|---|
| | | pg/ml | pg/ml | pg/ml | pg/ml | pg/ml | pg/ml | pg/ml | pg/ml | pg/ml | pg/ml |
| H84 | IL2 | 15.53 | 1.91 | 58.41 | 2.88 | s9.4 | 20.77 | 5630 | 1.23 | 2.36 | 0.98 |
| JC | treated | | | | | | | | | | |
| | NK | | | | | | | | | | |
| H85 | IL2 | 8.91 | 2 | 56.84 | 2.01 | 8.45 | 2.85 | 5645 | 1.15 | 2.48 | 1.14 |
| TC | treated | | | | | | | | | | |
| | NK | | | | | | | | | | |
| H84 | IL2aCD16 | 17.43 | 2.09 | 101.06 | 2.33 | 10.76 | 3.58 | 5835 | 1.73 | 20.77 | 1.31 |
| JC | treated | | | | | | | | | | |
| | NK | | | | | | | | | | |
| H85 | IL2aCD16 | 28.67 | 2.19 | 88.6 | 2.82 | 10.47 | 4.7 | 5853 | 1.5 | 26.26 | 1.31 |
| TC | treated | | | | | | | | | | |
| | NK | | | | | | | | | | |
| H84 | IL2sAJ2 | 16.94 | 2.47 | 84.16 | 6.26 | 9.65 | 65.04 | 5700 | 1.19 | 20.77 | 1.39 |
| JC | treated | | | | | | | | | | |
| | NK | | | | | | | | | | |
| H85 | IL2sAJ2 | 23.35 | 2.9 | 90.93 | 9.15 | 9.65 | 101.06 | 5707 | 1.19 | 29.52 | 1.47 |
| TC | treated | | | | | | | | | | |
| | NK | | | | | | | | | | |

TABLE 58

| | | IL-6 | IL-7 | IL-8 | IP-10 | MCP1 | MIP1a | MIP1b | Rantes | TNFa | TNFb | VEGF |
|---|---|---|---|---|---|---|---|---|---|---|---|---|
| | | pg/ml | pg/ml | pg/ml | pg/ml | pg/ml | pg/ml | pg/ml | pg/ml | pg/ml | pg/ml | pg/ml |
| H84 | IL2 | 16.69 | 46 | 106.65 | 88.6 | 68.56 | 144.7 | 356.57 | 1949 | 32.7 | 2.71 | 117.33 |
| JC | treated | | | | | | | | | | | |
| | NK | | | | | | | | | | | |
| H85 | IL2 | 3.22 | 4.9 | 20.47 | 157.61 | 52.33 | 144.7 | 475.47 | 2913 | 10.33 | 2.85 | 118.96 |
| TC | treated | | | | | | | | | | | |
| | NK | | | | | | | | | | | |
| H84 | IL2aCD16 | 3.28 | 5.45 | 250.96 | 216.77 | 101.06 | 1418 | 2252 | 3005 | 120.61 | 3.38 | 120.61 |
| JC | treated | | | | | | | | | | | |
| | NK | | | | | | | | | | | |
| H85 | IL2aCD16 | 4.1 | 6.57 | 247.31 | 303.66 | 146.77 | 1674 | 2499 | 3549 | 93.34 | 2.99 | 127.5 |
| TC | treated | | | | | | | | | | | |
| | NK | | | | | | | | | | | |
| H84 | IL2sAJ2 | 148.87 | 8.91 | 1046 | 12.37 | 672.05 | 699.55 | 71837 | 2249 | 78.01 | 4.18 | 140.66 |
| JC | treated | | | | | | | | | | | |
| | NK | | | | | | | | | | | |
| H85 | IL2sAJ2 | 164.54 | 9.4 | 1022 | 14.66 | 645.4 | 905.07 | 905.07 | 3022 | 70.38 | 4.03 | 136.75 |
| TC | treated | | | | | | | | | | | |
| | NK | | | | | | | | | | | |

TABLE 59

| | | | | | | X | | X | X | | X |
|---|---|---|---|---|---|---|---|---|---|---|---|
| | | EGF | FGF2 | Eotaxi [symbol] | TGFa | GCSF | FIT3L | GMCSF | Fractalkin [symbol] | IFNa2 | IFNg |
| | | pg/ml | pg/ml | pg/ml | pg/ml | pg/ml | pg/ml | pg/ml | pg/ml | pg/ml | pg/ml |
| H84 [symbol] | untreate [symbol] | 29.52 | 3.58 | 3.58 | 0.94 | 5.71 | 3.01 | 1.92 | 14.66 | 6.26 | 4.6 |
| H85 [symbol] | untreate [symbol] | 10.47 | 3.28 | 2.92 | 0.79 | 4.42 | 3.01 | 1.7 | 11.69 | 3.82 | 3.82 |
| H84 | IL2 | 24.05 | 4.6 | 5.34 | 1.14 | 11.69 | 4.18 | 13.46 | 44.23 | 8.91 | 7.62 |
| JC | treated | | | | | | | | | | |
| | CD8 | | | | | | | | | | |
| H85 | IL2 | 12.37 | 4.1 | 4.79 | 0.94 | 6.9 | 3.82 | 5.22 | 31.3 | 7.62 | 4.6 |
| TC | treated | | | | | | | | | | |
| | CD8 | | | | | | | | | | |
| H84 | IL2acd32 | 37.79 | 8.91 | 7.92 | 2.65 | 53.8 | 8.45 | 578.51 | 103.81 | 95.83 | 2523 |
| JC | 8 treated | | | | | | | | | | |
| | CD8 | | | | | | | | | | |
| H85 | IL2acd32 | 13.46 | 8.91 | 7.43 | 1.58 | 35.16 | 5.84 | 346.33 | 95.83 | 78.01 | 1842 |
| TC | 8 treated | | | | | | | | | | |
| | CD8 | | | | | | | | | | |
| H84 | IL2sAJ2 | 24.76 | 7.62 | 6.9 | 3.28 | 424.14 | 5.84 | 98.41 | 131.11 | 66.78 | 1447 |
| JC | treated | | | | | | | | | | |
| | CD8 | | | | | | | | | | |
| H85 | IL2sAJ2 | 13.08 | 6.9 | 6.26 | 1.99 | 274.05 | 6.26 | 78.01 | 136.75 | 37.25 | 326.7 |
| TC | treated | | | | | | | | | | |
| | CD8 | | | | | | | | | | |

[symbol]

indicates data missing or illegible when filed

TABLE 60

| | | X | | | IL- | X | IL- | | | PDGF- | |
|---|---|---|---|---|---|---|---|---|---|---|---|
| | | GRO | IL-10 | MCP3 | 12p40 | MDC | 12p70 | PDGFaa | IL-13 | AB/BB | IL-15 |
| | | pg/ml | pg/ml | pg/ml | pg/ml | pg/ml | pg/ml | pg/ml | pg/ml | pg/ml | pg/ml |
| H84 | untreated | 166.92 | 3.11 | 3.58 | 2.63 | 82.05 | 2.15 | 63.33 | 2.64 | 11.06 | 1.62 |
| JC | CD8 | | | | | | | | | | |
| H85 | untreated | 76.05 | 2.29 | 3.2 | 2.37 | 26.65 | 1.72 | 34.65 | 2.42 | 11.69 | 1.62 |
| TC | CD8 | | | | | | | | | | |
| H84 | IL2 | 192.9 | 6.26 | 4.6 | 3.47 | 223.2 | 2.61 | 50.2 | 4.1 | 11.69 | 2.12 |
| JC | treated | | | | | | | | | | |
| | CD8 | | | | | | | | | | |
| H85 | IL2 | 112.6 | 4.26 | 3.89 | 3.82 | 98.41 | 2.38 | 42.99 | 5.22 | 19.59 | 1.62 |
| TC | treated | | | | | | | | | | |
| | CD8 | | | | | | | | | | |
| H84 | IL2acd328 | 586.54 | 136.75 | 6.9 | 10.47 | 1226 | 9.15 | 93.34 | 1122 | 33.18 | 3.76 |
| JC | treated | | | | | | | | | | |
| | CD8 | | | | | | | | | | |
| H85 | IL2acd328 | 233.23 | 70.38 | 6.9 | 9.92 | 547.29 | 5.45 | 49.5 | 462.14 | 44.23 | 2.88 |
| TC | treated | | | | | | | | | | |
| | CD8 | | | | | | | | | | |
| H84 | IL2sAJ2 | 1787 | 927.72 | 9.15 | 10.47 | 997.83 | 5.22 | 66.78 | 22.02 | 20.77 | 2.81 |
| JC | treated | | | | | | | | | | |
| | CD8 | | | | | | | | | | |
| H85 | IL2sAJ2 | 1546 | 424.14 | 5.71 | 9.92 | 871.81 | 4.1 | 56.84 | 15.09 | 44.23 | 2.5 |
| TC | treated | | | | | | | | | | |
| | CD8 | | | | | | | | | | |

TABLE 61

| | | X | | | | | | | | X | |
|---|---|---|---|---|---|---|---|---|---|---|---|
| | | sCD40L | IL-17a | IL-1Ra | IL-1a | IL-9 | IL-1b | L-2 | IL-3 | IL-4 | IL-5 |
| | | pg/ml | pg/ml | pg/ml | pg/ml | pg/ml | pg/ml | pg/ml | pg/ml | pg/ml | pg/ml |
| H84 | untreated | 3.58 | 1.73 | 9.4 | 1.65 | 1.82 | 2.05 | 258.43 | 1.02 | 1.32 | 0.9 |
| JC | CD8 | | | | | | | | | | |
| H85 | untreated | 2.6 | 1.64 | 6.57 | 1.55 | 1.63 | 1.9 | 282.21 | 0.72 | 1.18 | 0.9 |
| TC | CD8 | | | | | | | | | | |
| H84 | IL2 | 7.43 | 2.19 | 58.41 | 2.85 | 7.82 | 3.7 | 5804 | 1.1 | 20.77 | 1.31 |
| JC | treated | | | | | | | | | | |
| | CD8 | | | | | | | | | | |
| H85 | IL2 | 3.2 | 2.19 | 52.33 | 2.22 | 8.03 | 2.13 | 4954 | 0.85 | 13.08 | 1.55 |
| TC | treated | | | | | | | | | | |
| | CD8 | | | | | | | | | | |
| H84 | IL2acd328 | 49.5 | 8.91 | 204.47 | 13.08 | 13.08 | 53.8 | 5777 | 8.91 | 290.61 | 243.71 |
| JC | treated | | | | | | | | | | |
| | CD8 | | | | | | | | | | |
| H85 | IL2acd328 | 9.92 | 4.42 | 176.83 | 3.96 | 12.37 | 14.05 | 5193 | 3.01 | 132.96 | 98.41 |
| TC | treated | | | | | | | | | | |
| | CD8 | | | | | | | | | | |
| H84 | IL2sAJ2 | 13.08 | 3.7 | 118.96 | 20.17 | 9.65 | 229.84 | 4983 | 0.89 | 0.94 | 4.03 |
| JC | treated | | | | | | | | | | |
| | CD8 | | | | | | | | | | |
| H85 | IL2sAJ2 | 5.98 | 3.38 | 112.6 | 14.25 | 9.15 | 86.34 | 5036 | 1.02 | 20.77 | 3.7 |
| TC | treated | | | | | | | | | | |
| | CD8 | | | | | | | | | | |

TABLE 62

| | | | | | | | some | | some | | | |
|---|---|---|---|---|---|---|---|---|---|---|---|---|
| | | | | correlated | correlated | | correlated | correlated | correlated | | | |
| | | L-6 | L-7 | L-8 | P-10 | MCP1 | MIP1a | MIP1b | Rantes | TNFa | TNFb | VEGF |
| | | pg/ml | pg/ml | pg/ml | pg/ml | pg/ml | pg/ml | pg/ml | pg/ml | pg/ml | pg/ml | pg/ml |
| H84 | untreated | 2.98 | 3.7 | 89.76 | 4.79 | 118.96 | 20.77 | 29.96 | 503.15 | 3.2 | 1.64 | 33.18 |
| JC | CD8 | | | | | | | | | | | |
| H85 | untreated | 2.21 | 2.76 | 45.5 | 3.58 | 60.83 | 15.53 | 15.98 | 430.27 | 2.5 | 1.39 | 33.18 |
| TC | CD8 | | | | | | | | | | | |
| H84 | IL2 | 4.6 | 3.96 | 547.29 | 15.53 | 562.72 | 79 | 144.7 | 377.89 | 8.45 | 3.96 | 109.58 |
| JC | treated | | | | | | | | | | | |
| | CD8 | | | | | | | | | | | |
| H85 | IL2 | 2.98 | 3.47 | 282.21 | 2.53 | 308.14 | 61.65 | 93.34 | 554.96 | 8.03 | 2.57 | 98.41 |
| TC | treated | | | | | | | | | | | |
| | CD8 | | | | | | | | | | | |
| H84 | IL2acd328 | 35.10 | 31.m | 2603 | 510.200 | 850.1 | 2154 | 3326 | 2585 | 3220 | 72.23 | 151 |
| JC | treated | | | | | | | | | | | |
| | CD8 | | | | | | | | | | | |
| H85 | IL2acd328 | 10.76 | 15.98 | 1475 | 455.6 | 326.7 | 1702 | 2960 | 2941 | 2001 | 48.14 | 125.73 |
| TC | treated | | | | | | | | | | | |
| | CD8 | | | | | | | | | | | |
| H84 | IL2sAJ2 | 777.14 | 28.67 | 3307 | 56.84 | 1617 | 1759 | 807.83 | 699.55 | 166.92 | 8.45 | 140.66 |
| JC | treated | | | | | | | | | | | |
| | CD8 | | | | | | | | | | | |
| H85 | IL2sAJ2 | 336.38 | 17.95 | 2760 | 3.04 | 767.11 | 1122 | 496.1 | 950.7m | 140.66 | 4.9 | 144.7 |
| TC | treated | | | | | | | | | | | |
| | CD8 | | | | | | | | | | | |

TABLE 63

| | EGF | FGF2 | Eotaxin | TGFa | GCSF | FIT3L | GMCSF | Fractalkine | 1FNa2 | 1FNg |
|---|---|---|---|---|---|---|---|---|---|---|
| | pg/ml | pg/ml | pg/ml | pg/ml | pg/ml | pg/ml | pg/ml | pg/ml | pg/ml | pg/ml |
| JCnCD4 1 | 4.03 | 27.04 | 4.51 | 0.47 | 233.23 | 32.23 | 2333 | 124 | 40.61 | 14159 |
| TC nCD4 1 | 3.96 | 35.16 | 6.81 | 0.54 | 266.13 | 45.5 | 2941 | 157.61 | 46.8 | 12892 |
| JC nCD4 2 | 1.95 | 8.68 | 3.13 | 0.19 | 29.52 | 50.9 | 672.05 | 41.78 | 4.42 | 2549 |
| TC nCD4 2 | 2.51 | 18.48 | 4.51 | 0.26 | 88.6 | 50.9 | 1475 | 84.16 | 16.45 | 4782 |

TABLE 64

| | GRO | IL-10 | MCP3 | IL-12p40 | MDC | IL-12p70 | PDGFaa | IL-13 | PDGF-AB/BB | IL-15 |
|---|---|---|---|---|---|---|---|---|---|---|
| | pg/ml | pg/ml | pg/ml | pg/ml | pg/ml | pg/ml | pg/ml | pg/ml | pg/ml | pg/ml |
| JC nCD4 1 | 367.08 | 308.14 | 5 | 12.37 | 1096 | 2.74 | 29.52 | 6388 | 26.26 | 1.7 |
| TC nCD4 1 | 274.05 | 229.84 | 7.25 | 18.48 | 2114 | 3.04 | 33.18 | 7260 | 26.26 | 2.33 |
| JC nCD4 2 | 117.33 | 11.06 | 3.47 | 2.67 | 254.67 | 1.23 | 6.73 | 2410 | 1.53 | 0.63 |
| TC nCD4 2 | 164.54 | 33.18 | 4.79 | 5 | 818.26 | 1.92 | 8.45 | 4385 | 8.03 | 0.96 |

TABLE 65

| | sCD40L | IL-17a | IL-1Ra | IL-1a | IL-9 | IL-1b | L-2 | IL-3 | IL-4 | IL-5 |
|---|---|---|---|---|---|---|---|---|---|---|
| | pg/ml | pg/ml | pg/ml | pg/ml | pg/ml | pg/ml | pg/ml | pg/ml | pg/ml | pg/ml |
| JC nCD4 1 | 157.61 | 82.05 | 125.73 | 82.05 | 777.14 | 2.85 | 562.72 | 29.52 | 81.01 | 115.73 |
| TC nCD4 1 | 321.96 | 93.34 | 226.5 | 46.8 | 594.67 | 3.47 | 3494 | 52.33 | 93.34 | 12.72 |
| JC nCD4 2 | 93.34 | 2.77 | 32.23 | 6.26 | 182.02 | 1.01 | 198.6 | 1.94 | 8.45 | 5.98 |
| TC nCD4 2 | 118.96 | 3 | 61.65 | 12.72 | 157.61 | 1.52 | 394.66 | 9.65 | 24.76 | 1.67 |

TABLE 66

| | IL-6 | IL-7 | IL-8 | IP-10 | MCP1 | MP1a | MP1b | Rantes | TNFa | TNFb | VEGF |
|---|---|---|---|---|---|---|---|---|---|---|---|
| | pg/ml | pg/ml | pg/ml | pg/ml | pg/ml | pg/ml | pg/ml | pg/ml | pg/ml | pg/ml | pg/ml |
| JC nCD4 1 | 223.2 | 9.4 | 3104 | 4096 | 148.87 | 2391 | 3447 | 3787 | 1731 | 905.07 | 157.61 |
| TC nCD4 1 | 125.73 | 9.92 | 4045 | 3935 | 48.14 | 2333 | 3262 | 3716 | 2336 | 1280 | 346.33 |
| JC nCD4 2 | 1.69 | 3.03 | 547.29 | 1200 | 4.03 | 2289 | 2681 | 3801 | 132.96 | 22.02 | 120.81 |
| TC nCD4 2 | 2.12 | 6.73 | 2897 | 2119 | 6.41 | 2291 | 2871 | 3603 | 317.29 | 42.99 | 134.84 |

TABLE 67

| | EGF | FGF2 | Eotaxin | TGFa | GCSF | FIT3L | GMCSF | Fractalkine | IFNa2 | IFNg |
|---|---|---|---|---|---|---|---|---|---|---|
| | pg/ml | pg/ml | pg/ml | pg/ml | pg/ml | pg/ml | pg/ml | pg/ml | pg/ml | pg/ml |
| JC Treg 2 | 1.65 | 4.1 | 2.09 | 0.15 | 5.71 | 49.5 | 58.41 | 24.76 | 3.05 | 82.05 |
| TC Treg 2 | 1.68 | 3.47 | 1.82 | 0.16 | 0.91 | 52.33 | 7.25 | 17.43 | 1.77 | 19.03 |

TABLE 68

| | | | | IL- | | IL- | | | PDGF- | |
|---|---|---|---|---|---|---|---|---|---|---|
| | GRO | IL-10 | MCP3 | 12p40 | MDC | 12p70 | PDGFaa | IL-13 | AB/BB | IL-15 |
| | pg/ml | pg/ml | pg/ml | pg/ml | pg/ml | pg/ml | pg/ml | pg/ml | pg/ml | pg/ml |
| JC Treg 2 | 20.77 | 303.66 | 2.59 | 1.43 | 122.29 | 0.68 | 0.93 | 1560 | 2.78 | 0.41 |
| TC Treg 2 | 8.45 | 155.37 | 2.59 | 1.26 | 53.8 | 0.68 | 1.35 | 290.61 | 0.39 | 0.44 |

TABLE 69

| | sCD40L | IL-17a | IL-1Ra | IL-1a | IL-9 | IL-1b | L-2 | IL-3 | IL-4 | IL-5 |
|---|---|---|---|---|---|---|---|---|---|---|
| | pg/ml | pg/ml | pg/ml | pg/ml | pg/ml | pg/ml | pg/ml | pg/ml | pg/ml | pg/ml |
| JC Treg | 13.85 | 25.5 | 32.23 | 0.58 | 21.39 | 0.37 | 326.7 | 0.7 | 44.23 | 84.16 |
| TC Treg | 6.12 | 8.68 | 14.66 | 0.45 | 16.45 | 0.27 | 389 | 0.33 | 6.9 | 5 |

TABLE 70

| | IL-6 | IL-7 | IL-8 | IP-10 | MCP1 | MP1a | MP1b | Rantes | TNFa | TNFb | VEGF |
|---|---|---|---|---|---|---|---|---|---|---|---|
| | pg/ml | pg/ml | pg/ml | pg/ml | pg/ml | pg/ml | pg/ml | pg/ml | pg/ml | pg/ml | pg/ml |
| JC Treg 2 | 0.64 | 1.36 | 93.34 | 32.23 | 2.27 | 204.47 | 76.05 | 187.3 | 49.5 | 0.78 | 90.93 |
| TC Treg 2 | 0.61 | 1.96 | 63.33 | 10.19 | 2.27 | 38.34 | 19.03 | 71.3 | 11.37 | 0.59 | 82.05 |

TABLE 71

| | EGF | FGF2 | Eotaxin | TGFa | GCSF | FIT3L | GMCSF | Fractalkine | IFNa2 | IFNg |
|---|---|---|---|---|---|---|---|---|---|---|
| | pg/ml | pg/ml | pg/ml | pg/ml | pg/ml | pg/ml | pg/ml | pg/ml | pg/ml | pg/ml |
| JC B cell | 262.25 | 25.5 | 2.09 | 0.16 | 29.52 | 2.06 | 0.54 | 27.85 | 7.25 | 0.97 |
| untreated | | | | | | | | | | |
| TC B cell | 12.37 | 4.1 | 1.65 | 0.15 | 5.71 | 2.06 | 0.46 | 4.79 | 1.55 | 0.76 |
| untreated | | | | | | | | | | |
| JC B cell IL- | 233.23 | 46.8 | 7.82 | 0.49 | 122.29 | 14.45 | 4.79 | 125.73 | 26.26 | 4.1 |
| 2 + sA/2 | | | | | | | | | | |
| TC B cell IL- | 12.03 | 38.34 | 8.13 | 0.49 | 93.34 | 14.45 | 3.15 | 129.29 | 20.77 | 4.79 |
| 2 + sA/2 | | | | | | | | | | |
| JC B cell | 517.51 | 14.66 | 3.28 | 0.25 | 204.47 | 4.79 | 6.57 | 101.06 | 14.66 | 4.79 |
| PMA + ionomycin | | | | | | | | | | |
| TC B cell | 27.44 | 11.06 | 2.78 | 0.31 | 240.16 | 5.71 | 5.45 | 106.65 | 12.03 | 5.58 |
| PMA + ionomycin | | | | | | | | | | |

TABLE 72

| | | | | IL- | | IL- | | | PDGF- | |
|---|---|---|---|---|---|---|---|---|---|---|
| | GRO | IL-10 | MCP3 | 12p40 | MDC | 12p70 | PDGFaa | IL-13 | AB/BB | IL-15 |
| | pg/ml | pg/ml | pg/ml | pg/ml | pg/ml | pg/ml | pg/ml | pg/ml | pg/ml | pg/ml |
| JC B cell | 787.28 | 2.15 | 2.35 | 1.31 | 76.05 | 0.85 | 308.14 | 1.59 | 1363 | 0.37 |
| untreated | | | | | | | | | | |
| TC B cell | 326.7 | 0.91 | 2.59 | 1.09 | 84.16 | 0.68 | 112.6 | 0.74 | 162.19 | 0.31 |
| untreated | | | | | | | | | | |
| JC B cell IL- | 818.26 | 55.3 | 6.12 | 16.94 | 78.01 | 2.05 | 258.43 | 2.69 | 727.92 | 3.38 |
| 2 + sA/2 | | | | | | | | | | |
| TC B cell IL- | 308.14 | 5.11 | 6.41 | 15.98 | 430.27 | 1.68 | 106.65 | 1.89 | 179.41 | 3.03 |
| 2 + sA/2 | | | | | | | | | | |
| JC B cell | 1046 | 171.8 | 3.82 | 4.1 | 1475 | 2.6 | 436.48 | 2.94 | 1010 | 0.77 |
| PMA + ionomycin | | | | | | | | | | |
| TC B cell | 418.09 | 93.34 | 4.6 | 3.96 | 997.83 | 2.45 | 106.65 | 2.45 | 136.75 | 0.86 |
| PMA + ionomycin | | | | | | | | | | |

TABLE 73

| | sCD40L | IL-17a | IL-1Ra | IL-1a | IL-9 | IL-1b | L-2 | IL-3 | IL-4 | IL-5 |
|---|---|---|---|---|---|---|---|---|---|---|
| | pg/ml | pg/ml | pg/ml | pg/ml | pg/ml | pg/ml | pg/ml | pg/ml | pg/ml | pg/ml |
| JC B cell | 125.73 | 0.58 | 2.99 | 0.47 | 0.81 | 0.25 | 3.96 | 0.29 | 5.71 | 0.15 |
| untreated | | | | | | | | | | |
| TC B cell | 2.67 | 0.52 | 1.98 | 0.45 | 0.38 | 0.22 | 2.47 | 0.22 | 3.58 | 0.16 |
| untreated | | | | | | | | | | |
| JC B cell IL- | 81.01 | 1.28 | 400.4 | 1.58 | 681.12 | 21.39 | 8614 | 0.34 | 63.33 | 0.26 |
| 2 + sA/2 | | | | | | | | | | |
| TC B cell IL- | 7.82 | 1.13 | 424.14 | 1.26 | 718.37 | 3.24 | 9065 | 0.36 | 40.61 | 0.22 |
| 2 + sA/2 | | | | | | | | | | |
| JC B cell | 25.2 | 1.22 | 20.17 | 2.83 | 2 | 5 | 44.23 | 0.35 | 227944 | 0.37 |
| PMA + ionomycin | | | | | | | | | | |
| TC B cell | 2.56 | 0.87 | 12.37 | 3.06 | 1.25 | 5.98 | 11.06 | 0.28 | 223839 | 0.35 |
| PMA + ionomycin | | | | | | | | | | |

TABLE 74

| | IL-6 | IL-7 | IL-8 | IP-10 | MCP1 | MP1a | MP1b | Rantes | TNFa | TNFb | VEGF |
|---|---|---|---|---|---|---|---|---|---|---|---|
| | pg/ml | pg/ml | pg/ml | pg/ml | pg/ml | pg/ml | pg/ml | pg/ml | pg/ml | pg/ml | pg/ml |
| JC B cell | 1.17 | 1.87 | 6.57 | 2.18 | 2.58 | 1.76 | 17.43 | 3563 | 1.15 | 0.36 | 24.76 |
| untreated | | | | | | | | | | | |
| TC B cell | 0.75 | 0.98 | 1.03 | 1.53 | 2.33 | 1.94 | 18.48 | 3513 | 0.78 | 0.25 | 4.79 |
| untreated | | | | | | | | | | | |
| JC B cell IL- | 41.78 | 4.26 | 236.67 | 6.9 | 26.26 | 66.78 | 86.34 | 3462 | 33.18 | 2.04 | 554.96 |
| 2 + sA/2 | | | | | | | | | | | |
| TC B cell IL- | 7.07 | 3.96 | 23.35 | 8.03 | 4.1 | 19.03 | 98.41 | 3467 | 9.92 | 1.79 | 547.29 |
| 2 + sA/2 | | | | | | | | | | | |
| JC B cell | 3342 | 13.85 | 38.34 | 8.91 | 4.18 | 377.89 | 250.96 | 3821 | 187.38 | 5.84 | 84.16 |
| PMA + ionomycin | | | | | | | | | | | |
| TC B cell | 4145 | 11.69 | 6.26 | 6.26 | 3.17 | 1447 | 950.73 | 3512 | 250.96 | 7.25 | 70.38 |
| PMA + ionomycin | | | | | | | | | | | |

TABLE 75

| | H102 JC | H103 TC | H104 RS |
|---|---|---|---|
| NK | 13.0% | 13.4% | 16.1% |
| CD4 | 36.3% | 32.3% | 36.0% |
| CD8 | 23.6% | 30.8% | 22.6% |
| CD19 | 11.0% | 9.17% | 11.3% |
| CD14 | 15.2% | 8.55% | 6.04% |
| total | 99.1% | 94.22% | 92.4% |
| CD4/Foxp3 | 1.42% | 7.57% | 0.43% |

TABLE 76

| 51Cr | | | IL-2 | | IL2-anti- | | IL-2 + anti- | | IL-2 + sAJ2 | |
|---|---|---|---|---|---|---|---|---|---|---|
| release | untreated | | treated | | CD16 treated | | CD3/28 treated | | treated | |
| PBMC | Mean | SD | Mean | SD | Mean | SD | Mean | SD | Mean | SD |
| H102 JC | 7.67 | 0.28 | 44.25 | 5.73 | 28.07 | 1.70 | 49.67 | 18.17 | 53.27 | 13.32 |
| H103 TC | 7.23 | 0.64 | 49.87 | 9.31 | 18.68 | 4.82 | 66.24 | 4.90 | 21.84 | 2.12 |
| H104 RS | 13.74 | 1.05 | 66.24 | 6.40 | 42.22 | 7.35 | 73.12 | 10.27 | 45.08 | 6.78 |

TABLE 77

| 51Cr release | untreated | | IL-2 treated | | IL2-anti-CD16 treated | | IL-2 + sAJ2 treated | |
|---|---|---|---|---|---|---|---|---|
| NK | Mean | SD | Mean | SD | Mean | SD | Mean | SD |
| H102 JC | 15.55 | 0.62 | 49.62 | 3.74 | 33.60 | 0.90 | 626.90 | 96.33 |
| H103 TC | 29.08 | 1.06 | 157.59 | 18.80 | 48.28 | 0.62 | 357.75 | 85.72 |
| H104 RS | 22.34 | 0.00 | 144.55 | 10.72 | 50.29 | 0.03 | 211.94 | 10.78 |

TABLE 78

| | | | | | | | IL-2 + | | | |
|---|---|---|---|---|---|---|---|---|---|---|
| | | | | | IL-2 + antiCD 16 | | antiCD3/28 | | IL-2 + sAJ2 | |
| PBMC | untreated | | IL-2 treated | | treated | | treated | | treated | |
| (1:8) | Mean | SD | Mean | SD | Mean | SD | Mean | SD | Mean | SD |
| H102 JC | 67.40 | 0.00 | 1975.27 | 87.26 | 2094.26 | 188.68 | | | 2289.78 | 64.03 |
| H103 TC | 128.30 | 0.00 | 1825.43 | 64.59 | 2145.95 | 91.79 | 1260.12 | 33.43 | 2205.24 | 3.40 |
| H104 RS | 74.61 | 0.00 | 328.22 | 96.89 | 1115.49 | 65.73 | 733.27 | 41.93 | 2039.78 | 20.96 |

TABLE 79

| | | | | | IL-2 + | | IL-2 + | |
|---|---|---|---|---|---|---|---|---|
| | untreated | | IL-2 treated | | anti-CD16 treated | | sAJ2 treated | |
| NK1:5 | Mean | SD | Mean | SD | Mean | SD | Mean | SD |
| H102 JC | 79.32 | 38.70 | 121.79 | 46.71 | 253.91 | 74.74 | 710.66 | 60.06 |
| H103 TC | 120.37 | 0.00 | 73.19 | 10.01 | 231.73 | 10.01 | 1864.82 | 44.04 |
| H104 RS | 52.43 | 43.37 | 97.72 | 23.36 | 302.51 | 27.36 | 781.91 | 51.38 |

TABLE 80

| | |
|---|---|
| untreated | IL-2 treated |

| CD8 1:5 | Mean | SD | Mean | SD |
|---|---|---|---|---|
| H102 JC | 129.34 | 96.09 | 232.20 | 97.43 |
| H103 TC | 126.51 | 38.70 | 145.85 | 62.06 |
| H104 RS | 162.37 | 30.70 | 278.44 | 36.03 |

TABLE 81

| | | | | | | X | | X | X | | X |
|---|---|---|---|---|---|---|---|---|---|---|---|
| | | EGF | FGF2 | Eotaxin | TGFa | GCSF | FIT3L | GMCSF | Fractalkine | IFNa2 | IFNg |
| | | pg/ml | pg/ml | pg/ml | pg/ml | pg/ml | pg/ml | pg/ml | pg/ml | pg/ml | pg/ml |
| H84 JC | untreated CD8 | 29.52 | 3.58 | 3.58 | 0.94 | 5.71 | 3.01 | 1.92 | 14.66 | 6.26 | 4.6 |
| H85 TC | untreated CD8 | 10.47 | 3.28 | 2.92 | 079 | 4.42 | 3.01 | 1.7 | 11.69 | 3.82 | 3.82 |
| H84 JC | IL2 treated CD8 | 24.05 | 4.6 | 5.34 | 1.14 | 11.69 | 4.18 | 13.46 | 44.23 | 8.91 | 7.62 |
| H85 TC | IL2 treated CD8 | 12.37 | 4.1 | 4.79 | 0.94 | 6.9 | 3.82 | 5.22 | 31.3 | 7.62 | 4.6 |
| H84 JC | IL2acd328 treated CD8 | 37.79 | 8.91 | 7.92 | 2.65 | 53.8 | 8.45 | 578.51 | 103.81 | 95.83 | 2523 |
| H85 TC | IL2acd328 treated CD8 | 13.46 | 8.91 | 7.43 | 1.58 | 35.16 | 5.84 | 346.33 | 95.83 | 78.01 | 1842 |
| H84 JC | IL2sAJ2 treated CD8 | 24.76 | 7.62 | 6.9 | 3.28 | 424.14 | 5.84 | 98.41 | 131.11 | 66.78 | 1447 |
| H85 TC | IL2sAJ2 treated CD8 | 13.08 | 6.9 | 6.26 | 1.99 | 274.05 | 6.26 | 78.01 | 136.75 | 37.25 | 326.7 |

TABLE 82

| | | X | | | IL- | X | IL- | | | PDGF- | |
|---|---|---|---|---|---|---|---|---|---|---|---|
| | | GRO | IL-10 | MCP3 | 12p40 | MDC | 12p70 | PDGFaa | IL-13 | AB/B | IL-15 |
| | | pg/ml | pg/ml | pg/ml | pg/ml | pg/ml | pg/ml | pg/ml | pg/ml | pg/ml | pg/ml |
| H84 JC | untreated CD8 | 166.92 | 3.11 | 3.58 | 2.63 | 82.05 | 2.15 | 63.33 | 2.64 | 11.06 | 1.62 |
| H85 TC | untreated CD8 | 76.05 | 2.29 | 3.2 | 2.37 | 26.65 | 1.72 | 34.65 | 2.42 | 11.69 | 1.62 |
| H84 JC | IL2 treated CD8 | 192.9 | 6.26 | 4.6 | 3.47 | 223.2 | 2.61 | 50.2 | 4.1 | 11.69 | 2.12 |
| H85 TC | IL2 treated CD8 | 112.6 | 4.26 | 3.89 | 3.82 | 98.41 | 2.38 | 42.99 | 5.22 | 19.59 | 1.62 |
| H84 JC | IL2acd328 treated CD8 | 586.54 | 136.75 | 6.9 | 10.47 | 1226 | 9.15 | 93.34 | 1122 | 33.18 | 3.76 |
| H85 TC | IL2acd328 treated CD8 | 233.23 | 70.38 | 6.9 | 9.92 | 547.29 | 5.45 | 49.5 | 462.14 | 44.23 | 2.88 |
| H84 JC | IL2sAJ2 treated CD8 | 1787 | 927.72 | 9.15 | 10.47 | 997.83 | 5.22 | 66.78 | 22.02 | 20.77 | 2.81 |
| H85 TC | IL2sAJ2 treated CD8 | 1546 | 424.14 | 5.71 | 9.92 | 871.81 | 4.1 | 56.84 | 15.09 | 44.23 | 2.5 |

TABLE 83

| | | X | | | | | | | | X | |
|---|---|---|---|---|---|---|---|---|---|---|---|
| | | sCD40L | IL-17a | IL-1Ra | IL-1a | IL-9 | IL-1b | L-2 | IL-3 | IL-4 | IL-5 |
| | | pg/ml | pg/ml | pg/ml | pg/ml | pg/ml | pg/ml | pg/ml | pg/ml | pg/ml | pg/ml |
| H84 JC | untreated CD8 | 3.58 | 1.73 | 9.4 | 1.65 | 1.82 | 2.05 | 258.43 | 1.02 | 1.32 | 0.9 |
| H85 TC | untreated CD8 | 2.6 | 1.64 | 6.57 | 1.55 | 1.63 | 1.9 | 282.21 | 0.72 | 1.18 | 0.9 |
| H84 JC | IL2 treated CD8 | 7.43 | 2.19 | 58.41 | 2.85 | 7.82 | 3.7 | 5804 | 1.1 | 20.77 | 1.31 |
| H85 TC | IL2 treated CD8 | 3.2 | 2.19 | 52.33 | 2.22 | 8.03 | 2.13 | 4954 | 0.85 | 13.08 | 1.55 |
| H84 JC | IL2acd328 treated CD8 | 49.5 | 8.91 | 204.47 | 13.08 | 13.08 | 53.8 | 5777 | 8.91 | 290.61 | 243.71 |
| H85 TC | IL2acd328 treated CD8 | 9.92 | 4.42 | 176.83 | 3.96 | 12.37 | 14.05 | 5193 | 3.01 | 132.96 | 98.41 |
| H84 JC | IL2sAJ2 treated CD8 | 13.08 | 3.7 | 118.96 | 20.17 | 9/65 | 229.84 | 4983 | 0.89 | 0.94 | 4.03 |
| H85 TC | IL2sAJ2 treated CD8 | 5.98 | 3.38 | 112.6 | 14.25 | 9.15 | 86.34 | 5036 | 1.02 | 20.77 | 3.7 |

TABLE 84

| | | | | | | | some | | some | | | |
|---|---|---|---|---|---|---|---|---|---|---|---|---|
| | | | | correlated | correlated | | correlated | correlated | correlated | | | |
| | | IL-6 | IL-7 | IL-8 | IP-10 | MCP1 | MIP1a | MIP1b | Rantes | TNFa | TNFb | VEGF |
| | | pg/ml | pg/ml | pg/ml | pg/ml | pg/ml | pg/ml | pg/ml | pg/ml | pg/ml | pg/ml | pg/ml |
| H84 JC | untreated CD8 | 2.98 | 3.7 | 89.76 | 4.79 | 118.96 | 20.77 | 29.96 | 503.15 | 3.2 | 1.64 | 33.18 |
| H85 TC | untreated CD8 | 2.21 | 2.76 | 45.5 | 3.58 | 60.83 | 15.53 | 15.98 | 430.27 | 2.5 | 1.39 | 33.18 |
| H84 JC | IL2 treated CD8 | 4.6 | 3.96 | 547.29 | 15.53 | 562.72 | 79 | 144.7 | 377.89 | 8.45 | 3.96 | 109.58 |
| H85 TC | IL2 treated CD8 | 2.98 | 3.47 | 282.21 | 2.53 | 308.14 | 61.65 | 93.34 | 554.96 | 8.03 | 2.57 | 98.41 |
| H84 JC | IL2acd328 treated CD8 | 35.16 | 31.3 | 2603 | 510.200 | 850.1 | 2154 | 3326 | 2585 | 3226 | 72.23 | 151 |
| H85 TC | IL2acd328 treated CD8 | 10.76 | 15.98 | 1475 | 455.6 | 326.7 | 1702 | 2960 | 2941 | 2001 | 48.14 | 125.73 |
| H84 JC | IL2sAJ2 treated CD8 | 777.14 | 28.67 | 3307 | 56.84 | 1617 | 1759 | 807.83 | 699.55 | 166.92 | 8.45 | 140.66 |
| H85 TC | IL2sAJ2 treated CD8 | 336.38 | 17.95 | 2760 | 3.04 | 767.11 | 1122 | 496.1 | 950.73 | 140.66 | 4.9 | 144.7 |

TABLE 85

| Analyte | ITAC | GM-CSF | Franktalkine | IFNg | IL-10 | MIP-3a | IL-12p70 | IL-13 | IL-17A | IL-1b | IL-2 | IL-21 |
|---|---|---|---|---|---|---|---|---|---|---|---|---|
| Sample | pg/ml | pg/ml | pg/ml | pg/ml | pg/ml | pg/ml | pg/ml | pg/ml | pg/ml | pg/ml | pg/ml | pg/ml |
| H51 JC sera Oct. 16, 2018 | 107.92 | 56.45 | 245.11 | 6.46 | 12.13 | 7.8 | 4.36 | 6.57 | 3.15 | 1.29 | 1.03 | 2.58 |
| H52 TC sera Oct. 16, 2018 | 102.24 | 78.12 | 299.38 | 4.35 | 6 | 2.05 | 2.44 | 5.18 | 2.46 | 1.29 | 0.6 | 2.58 |
| H53 MY sera Oct. 16, 2018 | 66.29 | 39.06 | 175.85 | 7.11 | 6.63 | 5.06 | 1.64 | 9.09 | 7.75 | 1.26 | 0.69 | 3.66 |
| H63 JC sera Nov. 13, 2018 | 11.98 | 71.64 | 239.55 | 4.35 | 15.13 | 7.8 | 3.75 | 6.86 | 2.08 | 1.1 | 1.03 | 2.23 |
| H64 JC sera Nov. 16, 2018 | 26.69 | 62.91 | 223.43 | 6.78 | 12.75 | 5.86 | 3.66 | 6.86 | 4.06 | 1.01 | 7.73 | 1.5 |
| H68 JC sera Nov. 21, 2018 | 22.44 | 42.6 | 223.43 | 4.35 | 33.15 | 9.14 | 4.46 | 16.29 | 1.85 | 1.01 | 1.49 | 2.71 |
| H70 JC sera Nov. 27, 2018 | 32.44 | 76.45 | 268.3 | 6.15 | 17.87 | 11.12 | 4.92 | 6.71 | 2.52 | 1.46 | 31.25 | 3.05 |
| H84 JC sera Apr. 9, 2019 | 120.27 | 88.97 | 239.55 | 5.18 | 6.98 | 10.65 | 2.51 | 5.07 | 1.69 | 1.36 | 2 | 1.57 |
| H85 TC sera Apr. 9, 2019 | 25.56 | 71.64 | 250.77 | 2.91 | 1.94 | 2.76 | 1.23 | 4.26 | 1.12 | 1.08 | 0.35 | 1.57 |
| H88 JC sera Apr. 10, 2019 | 121.58 | 45.46 | 250.77 | 4.35 | 10.44 | 12.67 | 3.66 | 4.96 | 1.81 | 1.36 | 2.84 | 2.02 |
| H89 TC sera Apr. 10, 2019 | 82.32 | 61.56 | 245.11 | 2.63 | 1.38 | 3.93 | 1.18 | 2.71 | 1.2 | 1.01 | 0.27 | 0.82 |
| H90 JC sera Apr. 11, 2019 | 121.58 | 64.29 | 189.14 | 3.13 | 6.98 | 9.98 | 2.1 | 5.41 | 1.31 | 0.77 | 1.13 | 1.05 |
| H91 TC sera Apr. 11, 2019 | 84.13 | 79.83 | 305.94 | 3.93 | 4.93 | 3.56 | 1.72 | 3.42 | 1.81 | 1.29 | 0.69 | 1.92 |

TABLE 86

| | IL-4 | IL-23 | IL-5 | IL-6 | IL-7 | IL-8 | MIP-1a | MIP-1b | TNFa |
|---|---|---|---|---|---|---|---|---|---|
| Analyte Sample | pg/ml | pg/ml | pg/ml | pg/ml | pg/ml | pg/ml | pg/ml | pg/ml | pg/ml |
| H51 JC sera Oct. 16, 2018 | 69.68 | 161.11 | 2.1 | 1.59 | 7.76 | 8.76 | 15.06 | 31.6 | 7.14 |
| H52 TC sera Oct. 16, 2018 | 47.18 | 96.82 | 3.39 | 1.37 | 8.47 | 10.88 | 19.32 | 25.17 | 3.72 |
| H53 MY sera Oct. 16, 2018 | 63.9 | 248.46 | 4.36 | 1.24 | 12.51 | 9.98 | 15.39 | 25.72 | 5.21 |
| H63 JC sera Nov. 13, 2018 | 53.73 | 168.24 | 2.51 | 1.37 | 7.76 | 5.1 | 12.94 | 28.05 | 5.59 |
| H64 JC sera Nov. 16, 2018 | 62.53 | 171.93 | 9.29 | 5.15 | 6.82 | 3.55 | 13.66 | 24.63 | 6.69 |
| H68 JC sera Nov. 21, 2018 | 86.53 | 183.47 | 2.99 | 2.68 | 8.84 | 7.21 | 18.11 | 29.3 | 7.14 |
| H70 JC sera Nov. 27, 2018 | 94.37 | 179.54 | 4.69 | 2.13 | 8.29 | 4.67 | 15.06 | 42.34 | 4.97 |
| H84 JC sera Apr. 9, 2019 | 66.73 | 138.44 | 2.64 | 0.92 | 8.65 | 4.09 | 16.07 | 37.99 | 7.14 |
| H85 TC sera Apr. 9, 2019 | 33.36 | 72.56 | 2.84 | 1.37 | 6.25 | 2.18 | 8.21 | 13.41 | 2.97 |
| H88 JC sera Apr. 10, 2019 | 58.59 | 150.97 | 2.77 | 0.79 | 9.44 | 5.68 | 18.7 | 37.18 | 7.78 |
| H89 TC sera Apr. 10, 2019 | 28.05 | 46.24 | 2.44 | 0.56 | 8.29 | 4.18 | 14.58 | 23.08 | 3.05 |
| H90 JC sera Apr. 11, 2019 | 52.58 | 106.21 | 1.86 | 0.75 | 7.44 | 4.47 | 14.9 | 33 | 5.09 |
| H91 TC sera Apr. 11, 2019 | 39.68 | 88.08 | 2.84 | 1.37 | 9.04 | 6.06 | 18.11 | 25.45 | 3.2 |

TABLE 87

| | | | | | | | | | | | | | | IL- | |
|---|---|---|---|---|---|---|---|---|---|---|---|---|---|---|---|
| Analyte | EGF | FGF2 | Eotaxin | TGFa | GCSF | FIT3L | GMCSF | Fractalkine | IFNa2 | IFNg | GRO | IL-10 | MCP3 | 12p40 | MDC |
| Sample | pg/ml | pg/ml | pg/ml | pg/ml | pg/ml | pg/ml | pg/ml | pg/ml | pg/ml | pg/ml | pg/ml | pg/ml | pg/ml | pg/ml | pg/ml |
| H103 JC | 118 | 27 | 31 | 2 | 31 | 53 | 6 | 33 | 19 | 12 | 11077 | 9 | 25 | 15 | 255 |
| SERA | | | | | | | | | | | | | | | |
| H104 TC | 397 | 29 | 104 | 2 | 16 | 56 | 7 | 33 | 21 | 9 | 11187 | 9 | 35 | 15 | 421 |
| SERA | | | | | | | | | | | | | | | |
| H105 RS | 115 | 37 | 86 | 5 | 308 | 32 | 12 | 92 | 31 | 14 | 1945 | 60 | 34 | 65 | 390 |
| SERA | | | | | | | | | | | | | | | |

TABLE 88

| | | | | PDGF- | | | IL- | IL- | | | | |
|---|---|---|---|---|---|---|---|---|---|---|---|---|
| Analyte | IL-12p70 | PDGFaa | IL-13 | AB/BB | IL-15 | sCD40L | 17a | 1Ra | IL-1a | IL-9 | IL-1b | L-2 |
| Sample | pg/ml | pg/ml | pg/ml | pg/ml | pg/ml | pg/ml | pg/ml | pg/ml | pg/ml | pg/ml | pg/ml | pg/ml |
| H103 JC | 8 | 4414 | 15 | 14701 | 10 | 2002 | 9 | 16 | 9 | 4 | 5 | 9 |
| H104 TC | 6 | 7516 | 20 | 16865 | 10 | 4091 | 6 | 26 | 7 | 5 | 5 | 13 |
| H105 RS | 13 | 7801 | 60 | 31056 | 37 | 5608 | 11 | 29 | 14 | 17 | 8 | 10 |

TABLE 89

| Analyte | IL-3 | IL-4 | IL-5 | IL-6 | IL-7 | IL-8 | IP-10 | MCP1 | MIP1a | MIP1b | Rantes | TNFa | TNFb | VEGF |
|---|---|---|---|---|---|---|---|---|---|---|---|---|---|---|
| Sample | pg/ml | pg/ml | pg/ml | pg/ml | pg/ml | pg/ml | pg/ml | pg/ml | pg/ml | pg/ml | pg/ml | pg/ml | pg/ml | pg/ml |
| H103 JC | 2 | 352 | 4 | 13 | 16 | 17 | 262 | 184 | 18 | 26 | 8934 | 17 | 7 | 54 |
| H104 TC | 2 | 343 | 4 | 9 | 18 | 21 | 71 | 154 | 20 | 22 | 2377 | 10 | 8 | 45 |
| H105 RS | 8 | 181 | 5 | 25 | 31 | 44 | 176 | 189 | 23 | 20 | 9480 | 15 | 7 | 65 |

TABLE 90

| Analyte | EGF | FGF2 | Eotaxin | TGFa | GCSF | FIT3L | GMCSF | Fractalkine | IFNa2 | IFNg | GRO | IL-10 | MCP3 |
|---|---|---|---|---|---|---|---|---|---|---|---|---|---|
| Sample | pg/ml | pg/ml | pg/ml | pg/ml | pg/ml | pg/ml | pg/ml | pg/ml | pg/ml | pg/ml | pg/ml | pg/ml | pg/ml |
| H103 JC CD8 UNT | 81 | 16 | 10 | 2 | 31 | 15 | 6 | 21 | 16 | 7 | 284 | 13 | 13 |
| H103 JC CD8 IL2 | 78 | 52 | 31 | 6 | 292 | 50 | 26 | 228 | 66 | 31 | 3406 | 51 | 112 |
| H103 JC CD8 IL2aCD328 | 91 | 58 | 35 | 11 | 361 | 57 | 135 | 284 | 102 | 665 | 1682 | 171 | 237 |
| H103 JC CD8 IL2sAJ2 | 241 | 77 | 39 | 21 | 12851 | 58 | 206 | 512 | 168 | 12869 | 13543 | 8654 | 128 |
| H104 TC CD8 UNT | 65 | 14 | 10 | 2 | 15 | 15 | 6 | 19 | 14 | 6 | 150 | 10 | 13 |
| H104 TC CD8 IL2 | 63 | 48 | 27 | 4 | 181 | 43 | 21 | 157 | 43 | 22 | 288 | 21 | 26 |
| H104 TC CD8 IL2aCD328 | 69 | 57 | 34 | 8 | 343 | 55 | 248 | 228 | 87 | | 2246 | 104 | 83 |
| H104 TC CD8 IL2sAJ2 | 70 | 73 | 35 | 15 | 3936 | 53 | 99 | 352 | 410 | 4871 | 10763 | 3786 | 88 |
| H105 RS CD8 UNT | 69 | 16 | 9 | 2 | 16 | 15 | 6 | 16 | 18 | 8 | 189 | 17 | 13 |
| H105 RS CD8 IL2 | 97 | 52 | 31 | 5 | 248 | 51 | 61 | 228 | 65 | 334 | 611 | 23 | 28 |
| H105 RS CD8 IL2aCD328 | 131 | 57 | 33 | 7 | 277 | 57 | 209 | 248 | 94 | 3056 | 1331 | 87 | 34 |
| H105 RS CD8 IL2sAJ2 | 105 | 67 | 36 | 9 | 2446 | 58 | 118 | 361 | 203 | 15853 | 6265 | 2002 | 37 |

TABLE 91

| | IL- | | IL- | | IL- | PDGF- | IL- | | IL- | IL- | IL- |
|---|---|---|---|---|---|---|---|---|---|---|---|
| Analyte | 12p40 | MDC | 12p70 | PDGFaa | 13 | AB/BB | 15 | sCD40L | 17a | 1Ra | 1a |
| Sample | pg/ml | pg/ml | pg/ml | pg/ml | pg/ml | pg/ml | pg/ml | pg/ml | pg/ml | pg/ml | pg/ml |
| H103 JC CD8 UNT | 10 | 221 | 6 | 95 | 10 | 42 | 5 | 15 | 6 | 33 | 7 |
| H103 JC CD8 IL2 | 47 | 910 | 12 | 86 | 23 | 54 | 21 | 37 | 11 | 1022 | 16 |
| H103 JC CD8 IL2aCD328 | 57 | 2061 | 16 | 104 | 548 | 69 | 28 | 52 | 17 | 1541 | 28 |
| H103 JC CD8 IL2sAJ2 | 73 | 120 | 26 | 317 | 50 | 835 | 29 | 80 | 16 | 3227 | 684 |
| H104 TC CD8 UNT | 10 | 77 | 5 | 54 | 10 | 25 | 5 | 9 | 6 | 14 | 5 |
| H104 TC CD8 IL2 | 47 | 325 | 10 | 58 | 20 | 52 | 21 | 26 | 10 | 723 | 13 |
| H104 TC CD8 IL2aCD328 | 55 | 965 | 20 | 63 | 540 | 31 | 24 | 34 | 16 | 1255 | 25 |
| H104 TC CD8 IL2sAJ2 | 65 | 380 | 18 | 65 | 30 | 64 | 23 | 31 | 12 | 1610 | 197 |
| H105 RS CD8 UNT | 11 | 54 | 5 | 87 | 14 | 107 | 5 | 52 | 6 | 15 | 6 |
| H105 RS CD8 IL2 | 50 | 197 | 15 | 152 | 26 | 197 | 21 | 92 | 11 | 965 | 16 |
| H105 RS CD8 IL2aCD328 | 55 | 300 | 22 | 200 | 115 | 248 | 23 | 150 | 18 | 993 | 31 |
| H105 RS CD8 IL2sAJ2 | 95 | 65 | 50 | 143 | 52 | 385 | 29 | 101 | 15 | 1149 | 176 |

TABLE 92

| Analyte | IL-9 | IL-1b | L-2 | IL-3 | IL-4 | IL-5 | IL-6 | IL-7 | IL-8 | IP-10 | MCP1 |
|---|---|---|---|---|---|---|---|---|---|---|---|
| Sample | pg/ml | pg/ml | pg/ml | pg/ml | pg/ml | pg/ml | pg/ml | pg/ml | pg/ml | pg/ml | pg/ml |
| H103 JC CD8 UNT | 5 | 4 | 11 | 2 | 23 | 2 | 12 | 13 | 343 | 15 | 273 |
| H103 JC CD8 IL2 | 1149 | 41 | | 3 | 209 | 6 | 82 | 32 | 6674 | 81 | 9873 |
| H103 JC CD8 IL2aCD328 | 1149 | 46 | | 6 | 334 | 118 | 66 | 43 | 12367 | 1350 | 11255 |
| H103 JC CD8 IL2sAJ2 | 1149 | 9480 | | 4 | 656 | 13 | 10959 | 80 | 12584 | 81 | 6674 |
| H104 TC CD8 UNT | 4 | 3 | 10 | 2 | 27 | 2 | 8 | 12 | 209 | 14 | 231 |
| H104 TC CD8 IL2 | 1084 | 18 | | 3 | 113 | 4 | 15 | 29 | 1084 | 163 | 1835 |
| H104 TC CD8 IL2aCD328 | 1084 | 68 | | 5 | 317 | 166 | 45 | 52 | 7516 | 300 | 3786 |
| H104 TC CD8 IL2sAJ2 | 1053 | 993 | | 3 | 334 | 9 | 2736 | 54 | 11168 | 109 | 7801 |
| H105 RS CD8 UNT | 4 | 5 | 15 | 2 | 23 | 4 | 10 | 14 | 152 | 36 | 107 |
| H105 RS CD8 IL2 | 1149 | 62 | | 3 | 124 | 6 | 61 | 30 | 3406 | 192 | 2893 |
| H105 RS CD8 IL2aCD328 | 1053 | 131 | | 5 | 234 | 43 | 128 | 41 | 7516 | 385 | 3786 |
| H105 RS CD8 IL2sAJ2 | 1053 | 3592 | | 4 | 380 | 9 | 3688 | 65 | 10239 | 192 | 2182 |

TABLE 93

| | MIP1a | MIP1b | Rantes | TNFa | TNFb | VEGF |
|---|---|---|---|---|---|---|
| Analyte Sample | pg/ml | pg/ml | pg/ml | pg/ml | pg/ml | pg/ml |
| H103 JC CD8 UNT | 88 | 176 | 1889 | 10 | 4 | 21 |
| H103 JC CD8 IL2 | 212 | 455 | 2377 | 37 | 15 | 1370 |
| H103 JC CD8 IL2aCD328 | 9480 | 9744 | 8371 | 811 | 30 | 1496 |
| H103 JC CD8 IL2sAJ2 | 11565 | 7516 | 12262 | 2246 | 28 | 1219 |
| H104 TC CD8 UNT | 58 | 74 | 1149 | 9 | 3 | 18 |
| H104 TC CD8 IL2 | 135 | 234 | 1889 | 29 | 12 | 1022 |
| H104 TC CD8 IL2aCD328 | 9480 | 12643 | 8794 | | 56 | 1116 |
| H104 TC CD8 IL2sAJ2 | 10677 | 5356 | 3592 | 426 | 22 | 1183 |
| H105 RS CD8 UNT | 56 | 117 | 4754 | 14 | 4 | 19 |
| H105 RS CD8 IL2 | 317 | 744 | 7801 | 80 | 17 | 1166 |
| H105 RS CD8 IL2aCD328 | 6674 | 9210 | 10843 | 1889 | 39 | 1219 |
| H105 RS CD8 IL2sAJ2 | 6674 | 2893 | 9210 | 1219 | 24 | 1311 |

TABLE 94

| | PDGFaa | PDGF-AB/BB | sCD40L |
|---|---|---|---|
| Analyte Sample | pg/ml | pg/ml | pg/ml |
| H103 JC CD8 UNT | 95 | 42 | 15 |
| H103 JC CD8 IL2 | 86 | 54 | 37 |
| H103 JC CD8 IL2aCD328 | 104 | 69 | 52 |
| H103 JC CD8 IL2sAJ2 | 317 | 835 | 80 |
| H104 TC CD8 UNT | 54 | 25 | 9 |
| H104 TC CD8 IL2 | 58 | 52 | 26 |
| H104 TC CD8 IL2aCD328 | 63 | 31 | 34 |
| H104 TC CD8 IL2sAJ2 | 65 | 64 | 31 |
| H105 RS CD8 UNT | 87 | 107 | 52 |
| H105 RS CD8 IL2 | 152 | 197 | 92 |
| H105 RS CD8 IL2aCD328 | 200 | 248 | 150 |
| H105 RS CD8 IL2sAJ2 | 143 | 385 | 101 |

TABLE 95
Plasma infusion Jun. 27, 2019

| | H106 JC |
|---|---|
| NK | 7.11% |
| CD4 | 22.9% |
| CD8 | 37.5% |
| CD19 | 16.2% |
| CD14 | 14.8% |
| total | 98.51% |

TABLE 96

| | | | IL-2 | | IL2-anti-CD16 | | IL-2 + anti- | | IL-2 + sAJ2 | |
|---|---|---|---|---|---|---|---|---|---|---|
| 51Cr | untreated | | treated | | treated | | CD3/28 treated | | treated | |
| PBMC | Mean | SD | Mean | SD | Mean | SD | Mean | SD | Mean | SD |
| H106 JC | 2.94 | 0.29 | 16.36 | 0.56 | 14.64 | 0.15 | 25.48 | 0.88 | 17.31 | 0.97 |

TABLE 97

| | H107 JC | H108 JC |
|---|---|---|
| | Post-NAC | Post-NAC |
| | Pre-young plasma | Post-young plasma |
| NK | 11.5% | 21.2% |
| CD4 | 31.4% | 22.8% |
| CD8 | 15.1% | 25.7% |
| CD19 | 24.6% | 16.3% |
| CD14 | 15.0% | 17.2% |
| total | 97.6% | 103.2% |
| NKT | 3.58% | 7.99% |

TABLE 98

| | | H109 RS | H110 RS | |
|---|---|---|---|---|
| | P99 ME | Pre-NAC | Post-NAC | H111 AK |
| NK | 6.36% | 20.4% | 15.3% | 20.3% |
| CD4 | 28.7% | 37.5% | 27.6% | 31.4% |
| CD8 | 32.8% | 18.4% | 20.4% | 21.8% |
| CD19 | 10.8% | 4.23% | 10.0% | 12.0% |
| CD14 | 19.4% | 19.2% | 27.1% | 26.6% |
| Total | 98.06% | 99.73% | 100.4% | 112.1% |

TABLE 99

| | | | IL-2 | | IL2-anti-CD16 | | IL-2 + anti- | | IL-2 + sAJ2 | |
|---|---|---|---|---|---|---|---|---|---|---|
| | untreated | | treated | | treated | | CD3/28 treated | | treated | |
| PBMC | Mean | SD | Mean | SD | Mean | SD | Mean | SD | Mean | SD |
| P99 ME | 1.70 | 0.62 | 17.22 | 0.15 | 11.34 | 0.74 | 26.27 | 1.75 | 20.01 | 2.15 |
| H109 RS1 | 2.57 | 0.37 | 15.02 | 0.80 | 8.54 | 0.14 | 45.79 | 8.53 | 26.01 | 3.48 |
| H110 RS2 | 4.47 | 0.67 | 27.60 | 2.16 | 7.06 | 0.09 | 31.82 | 1.91 | 54.76 | 2.49 |
| H111 AK | 2.35 | 0.21 | 26.01 | 0.96 | 24.76 | 1.17 | 23.44 | 7.48 | 43.13 | 0.51 |

TABLE 100

| | untreated | | IL-2 treated | | IL2-anti-CD16 treated | | IL-2 + sAJ2 treated | |
|---|---|---|---|---|---|---|---|---|
| NK | Mean | SD | Mean | SD | Mean | SD | Mean | SD |
| P99 ME | 7.21 | 0.77 | 72.37 | 0.84 | 47.50 | 0.00 | 474.87 | 0.00 |
| H109 RS1 | 27.95 | 0.69 | 331.64 | 100.95 | 97.97 | 4.94 | 327.48 | 0.03 |
| H110 RS2 | 33.20 | 3.39 | 373.11 | 85.06 | 91.64 | 1.68 | 589.50 | 72.26 |
| H111 AK | | | 131.16 | 5.70 | 62.48 | 8.03 | 475.45 | 0.00 |

TABLE 101
Aug. 27, 2019 Day 0
PBMC, samples were shipped overnight

| | H115 JC | H116 TC |
|---|---|---|
| NK | 10.9% | 15.9% |
| CD4 | 37.0% | 30.7% |
| CD8 | 27.0% | 35.4% |
| CD19 | 12.1% | 7.73% |
| CD14 | 15.3% | 16.9% |
| total | 102.3% | 106.63% |

TABLE 102

| | | | IL-2 | | IL2-anti-CD16 | | IL-2 + anti- | | IL-2 + sAJ2 | |
|---|---|---|---|---|---|---|---|---|---|---|
| | untreated | | treated | | treated | | CD3/28 treated | | treated | |
| | Mean | SD | Mean | SD | Mean | SD | Mean | SD | Mean | SD |
| H105 JC | 4.56 | 0.22 | 15.37 | 0.60 | 16.13 | 0.47 | 24.04 | 2.12 | 21.53 | 3.09 |
| H106 TC | 6.61 | 0.50 | 12.76 | 1.79 | 18.84 | 2.33 | 28.32 | 3.19 | 25.76 | 0.25 |

TABLE 103

| | | | IL-2 + | IL-2 + | |
|---|---|---|---|---|---|
| | | IL-2 | anti-CD16 | anti-CD3/28 | IL-2 + sAJ2 |
| | untreated | treated | treated | treated | treated |
| H115 JC | 36.93 | 124.62 | 455.52 | 773.66 | 825.67 |
| PBMC | | | | | |
| (1:10) | | | | | |
| H116 TC | 178.16 | 200.59 | 670.16 | 489.68 | 1161.15 |
| PBMC | | | | | |
| (1:10) | | | | | |

TABLE 104

| | | IL-2 | IL-2 + anti- | IL-2 + sAJ2 |
|---|---|---|---|---|
| | untreated | treated | CD3/28 treated | treated |
| H115 JC CD8 | 241.5 | 204.4 | 1792.0 | 1529.8 |
| (1:10) | | | | |
| H116 TC CD8 | 299.9 | 414.4 | 1808.0 | 374.2 |
| (1:10) | | | | |

TABLE 105

| | H117.1 JC |
|---|---|
| NK | 11.7% |
| CD4 | 34.2% |
| CD8 | 19.8% |
| CD19 | 16.4% |
| CD14 | 21.1% |
| total | 103.2% |

TABLE 106

H117.1 JC PBMC

| | Mean | SD |
|---|---|---|
| untreated | 2.44 | 0.71 |
| IL-2 treated | 3.93 | 0.13 |
| IL-2 + αCD16 treated | 8.17 | 1.20 |
| IL-2 + αCD3/28 treated | 12.22 | 0.66 |
| IL-2 + sAJ2 treated | 9.37 | 1.29 |
| IL-2 + αPD-1 (10 μg/ml) treated | 6.17 | 1.08 |
| IL-2 + αPD-1 (20 μg/ml) treated | 7.10 | 0.10 |

TABLE 107

| | | | | | | | | | | | IL- | | IL- | |
|---|---|---|---|---|---|---|---|---|---|---|---|---|---|---|
| | | | | | | | | | | | 2 + αPD-1 | | 2 + αPD-1 | |
| | | | | | IL- | | IL- | | IL- | | (10 | | (20 | |
| | | | IL-2 | | 2 + αCD16 | | 2 + αCD3/28 | | 2 + sAJ2 | | μg/ml) | | μg/ml) | |
| 1:10 | untreated | | treated | | treated | | treated | | treated | | treated | | treated | |
| PBMC | Mean | SD | Mean | SD | Mean | SD | Mean | SD | Mean | SD | Mean | SD | Mean | SD |
| H117.1 JC | 55.42 | 7.46 | 88.6 | 9.59 | 273.14 | 10.65 | 691.25 | 15.98 | 909.72 | 18.11 | 64.46 | 30.90 | 48.64 | 0.00 |

TABLE 108

| | | | | | | | | | IL-2 + αPD- | | IL-2 + αPD- | |
|---|---|---|---|---|---|---|---|---|---|---|---|---|
| | | | | | IL- | | | | 1 (10 | | 1 (20 | |
| | | | IL-2 | | 2 + αCD3/28 | | IL-2 + sAJ2 | | μg/ml) | | μg/ml) | |
| | untreated | | treated | | treated | | treated | | treated | | treated | |
| | Mean | SD | Mean | SD | Mean | SD | Mean | SD | Mean | SD | Mean | SD |
| H117.1 JC | 44.88 | 20.24 | 229.45 | 25.57 | 1398.65 | 123.59 | 1245.72 | 52.20 | 361.28 | 11.72 | 368.06 | 19.18 |
| (1:10) | | | | | | | | | | | | |
| H117.1 JC | 75.01 | 28.77 | 331.90 | 21.31 | 1825.04 | 0.00 | 1985.51 | 13.85 | 556.40 | 2.13 | 530.79 | 31.96 |
| (1:5) | | | | | | | | | | | | |

TABLE 109
Exp182 H117 JC H118 TC
Sep. 18, 2019 (shipped)

| | H117 JC | H118 TC |
|---|---|---|
| NK | 7.07% | 9.78% |
| CD4 | 37.0% | 36.9% |
| CD8 | 24.9% | 27.1% |
| CD19 | 14.2% | 11.5% |
| CD14 | 17.0% | 13.9% |
| total | 100.17% | 99.18% |

TABLE 110

| | | | IL-2 | | IL2-anti-CD16 | | IL-2 + anti- | | IL-2 + sAJ2 | |
|---|---|---|---|---|---|---|---|---|---|---|
| | untreated | | treated | | treated | | CD3/28 treated | | treated | |
| PBMC | Mean | SD | Mean | SD | Mean | SD | Mean | SD | Mean | SD |
| H117 JC | 1.41 | 0.28 | 9.22 | 0.26 | 5.07 | 0.20 | 12.09 | 0.30 | 8.74 | 0.30 |
| H118 TC | 1.44 | 0.03 | 8.97 | 0.57 | 2.68 | 0.16 | 14.48 | 0.19 | 11.12 | 0.19 |

TABLE 111

| | | | | | | | IL-2 + | |
|---|---|---|---|---|---|---|---|---|
| | | | IL-2 | | IL2-anti-CD16 | | sAJ2 | |
| | untreated | | treated | | treated | | treated | |
| NK | Mean | SD | Mean | SD | Mean | SD | Mean | SD |
| H117 JC | 13.52 | 1.88 | 36.93 | 1.32 | 11.35 | 1.26 | 53.52 | 1.59 |
| H118 TC | 13.21 | 1.14 | 55.72 | 1.85 | 11.73 | 0.43 | 84.11 | 9.25 |

TABLE 112

| | | | | | | | IL- | | | |
|---|---|---|---|---|---|---|---|---|---|---|
| | | | | | IL-2 + αCD16 | | 2 + αCD3/28 | | IL-2 + sAJ2 | |
| 1:10 | untreated | | IL-2 treated | | treated | | treated | | treated | |
| dilution | Mean | SD | Mean | SD | Mean | SD | Mean | SD | Mean | SD |
| H117 JC PBMC | 169.66 | 51.04 | 236.97 | 127.88 | 475.81 | 47.02 | 933.21 | 52.76 | 1593.37 | 84.87 |
| H118 TC PBMC | 195.61 | 15.48 | 168.44 | 9.18 | 240.62 | 17.20 | 1014.31 | 56.20 | 1392.24 | 58.49 |

TABLE 113

| | | | IL-2 + αCD16 | IL-2 + sAJ2 |
|---|---|---|---|---|
| straight | untreated | IL-2 treated | treated | treated |
| H117 JC NK | 305.5 | 496.896 | 361.459 | 666.395 |
| H118 TC NK | 367.136 | 406.875 | 535.824 | 728.031 |

TABLE 114

| | | | | | IL-2 + αCD3/28 | | IL-2 + sAJ2 | |
|---|---|---|---|---|---|---|---|---|
| | untreated | | IL-2 treated | | treated | | treated | |
| 1:5 dilution | Mean | SD | Mean | SD | Mean | SD | Mean | SD |
| H117 JC | 308.34 | 96.92 | 310.37 | 48.17 | 1595.40 | 33.83 | 504.20 | 4.59 |
| CD8 | | | | | | | | |
| H118 TC | 214.26 | 55.63 | 239.40 | 16.63 | 1116.50 | 13.76 | 911.32 | 43.58 |
| CD8 | | | | | | | | |

TABLE 115

| | H119 BM | H120 JC | H121 TC | H122 MV |
|---|---|---|---|---|
| NK | 11.9% | 12.7% | 10.5% | 40.9% |
| CD4 | 47.8% | 35.3% | 36.3% | 16.0% |
| CD8 | 11.1% | 26.7% | 30.0% | 9.41% |
| CD19 | 9.44% | 9.93% | 9.76% | 7.28% |
| CD14 | 13.2% | 14.2% | 16.0% | 27.4% |
| Total | 93.44% | 98.83% | 102.86% | 100.99% |

TABLE 116

| | | | IL-2 | | IL-2 + αCD16 | | IL-2 + αCD3/28 | | IL-2 + sAJ2 | |
|---|---|---|---|---|---|---|---|---|---|---|
| | untreated | | treated | | treated | | treated | | treated | |
| PBMC | Mean | SD | Mean | SD | Mean | SD | Mean | SD | Mean | SD |
| H119 | 4.05 | 0.61 | 10.88 | 0.14 | 11.33 | 0.73 | 13.12 | 0.73 | 14.77 | 0.45 |
| BM | | | | | | | | | | |
| H120 | 4.39 | 0.01 | 20 | 0.0 | 18.10 | 2.06 | 29.71 | 4.63 | 29.39 | 1.36 |
| JC | | | | | | | | | | |
| H121 | 3.62 | 0.01 | 28.10 | 7.26 | 21.23 | 3.32 | 54.71 | 18.95 | 45.58 | 14.38 |
| TC | | | | | | | | | | |
| H122 | 11.81 | 1.33 | 175.4 | .027 | 70.33 | 7.50 | 116.9 | 4.6 | 86.81 | 17.87 |
| MV | | | | | | | | | | |

TABLE 117

| | | | | | IL-2 + αCD16 | | IL-2 + αCD3/28 | | IL-2 + sAJ2 | |
|---|---|---|---|---|---|---|---|---|---|---|
| PBMC | untreated | | IL-2 treated | | treated | | treated | | treated | |
| (1:10) | Mean | SD | Mean | SD | Mean | SD | Mean | SD | Mean | SD |
| H119 BM | 78.94 | 22.72 | 438.45 | 12.69 | 332.16 | 21.38 | 617.97 | 43.43 | 967.56 | 31.40 |
| H120 JC | 97.84 | 28.06 | 1111.18 | 66.14 | 1416.36 | 38.08 | 1177.32 | 112.91 | 2219.47 | 112.91 |
| H121 TC | 109.65 | 31.40 | 863.16 | 74.83 | 1315.74 | 85.52 | 1053.07 | 72.16 | 1879.80 | 88.19 |
| H122 MV | 119.57 | 65.47 | 820.64 | 36.08 | 1655.88 | 8.02 | 494.67 | 8.02 | 2302.15 | 58.79 |

TABLE 118A

| | | | | | IL-2 + αCD3/28 | | IL-2 + sAJ2 | |
|---|---|---|---|---|---|---|---|---|
| | untreated | | IL-2 treated | | treated | | treated | |
| 1:5 | Mean | SD | Mean | SD | Mean | SD | Mean | SD |
| H120 JC | 95.42 | 5.49 | 140.63 | 4.50 | 844.86 | 102.39 | 624.13 | 192.79 |
| CD8 | | | | | | | | |
| H121 TC | 80.94 | 0.00 | 40.68 | 0.00 | 691.58 | 90.40 | 137.80 | 6.49 |
| CD8 | | | | | | | | |
| H122 MV | 168.53 | 45.95 | 152.29 | 2.00 | 588.81 | 16.98 | 534.42 | 8.99 |
| CD8 | | | | | | | | |

TABLE 118B

| Analyte | EGF | FGF2 | Eotaxin | TGFa | GCSF | FIT3L | GMCSF | Fractalkine | IFNa2 | IFNg | GRO | IL-10 | MCP3 | IL-12p40 |
|---|---|---|---|---|---|---|---|---|---|---|---|---|---|---|
| Sample | pg/ml | pg/ml | pg/ml | pg/ml | pg/ml | pg/ml | pg/ml | pg/ml | pg/ml | pg/ml | pg/ml | pg/ml | pg/ml | pg/ml |
| Exp183 | 22.34 | 26.68 | 21.91 | 2.5 | 308.26 | 7.7 | 342.82 | 73.31 | 65.42 | 715.2 | 4525 | 22.34 | 26.68 | 21.91 |
| JC Treg | | | | | | | | | | | | | | |
| unt Day 1 | | | | | | | | | | | | | | |
| Exp183 | 43.39 | 87.17 | 32.85 | 5.26 | 646.43 | 16.88 | 2973 | 325.18 | 261.67 | 4525 | 3786 | 43.39 | 87.17 | 32.85 |
| JC Treg | | | | | | | | | | | | | | |
| diff Day 1 | | | | | | | | | | | | | | |
| Exp183 | 40.13 | 165.89 | 48.92 | 6.39 | 2551 | 36.25 | 1862 | 361.17 | 333.91 | 1453 | 9210 | 40.13 | 165.89 | 48.92 |
| JC Treg | | | | | | | | | | | | | | |
| diff il2 | | | | | | | | | | | | | | |
| aj2 | | | | | | | | | | | | | | |
| Exp183 | 27.27 | 44.55 | 28.53 | 3.32 | 1835 | 8.7 | 936.9 | 170.78 | 113.39 | 32.07 | 11651 | 27.27 | 44.55 | 28.53 |
| TC Treg | | | | | | | | | | | | | | |
| unt | | | | | | | | | | | | | | |
| Exp183 | 28.86 | 77.65 | 32.46 | 5.89 | 1183 | 15.62 | 1391 | 180.96 | 258.07 | 1541 | 9072 | 28.86 | 77.65 | 32.46 |
| TC Treg | | | | | | | | | | | | | | |
| diff | | | | | | | | | | | | | | |

TABLE 119

| | | IL- | | IL- | PDGF- | IL- | | IL- | IL- | | | |
|---|---|---|---|---|---|---|---|---|---|---|---|---|
| | MDC | 12p70 | PDGFaa | 13 | AB/BB | 15 | sCD40L | 17a | 1Ra | IL-1a | IL-9 | IL-1b |
| | pg/ml | pg/ml | pg/ml | pg/ml | pg/ml | pg/ml | pg/ml | pg/ml | pg/ml | pg/ml | pg/ml | pg/ml |
| Exp183 | 170.78 | 14.42 | 2.56 | 10.27 | 2.77 | 5.28 | 29.88 | 10.08 | 7405.0 | 492.03 | 10.44 | 7188.9 |
| JC Treg | | | | | | | | | | | | |
| unt Day 1 | | | | | | | | | | | | |
| Exp183 | 897.04 | 308.26 | 8.21 | 6131 | 28.53 | 10.62 | 3406 | 188.97 | 1116 | 1889 | 646.43 | 519.05 |
| JC Treg | | | | | | | | | | | | |
| diff Day 1 | | | | | | | | | | | | |
| Exp183 | 1149 | 49.58 | 8.14 | 4754 | 31.31 | 18.8 | 1586 | 135.21 | 2182 | 3271 | 2377 | 2121 |
| JC Treg | | | | | | | | | | | | |
| diff il2 | | | | | | | | | | | | |
| aj2 | | | | | | | | | | | | |
| Exp183 | 505.32 | 20.14 | 5.56 | 17.37 | 116.77 | 7 | 34.49 | 12.67 | 4788.0 | 1889 | 17.37 | 443 |
| TC Treg | | | | | | | | | | | | |
| unt | | | | | | | | | | | | |
| Exp183 | 2002 | 69.24 | 6.39 | 2551 | 175.8 | 10.09 | 1219 | 145.48 | 1518 | 3271 | 2147.6 | 646.43 |
| TC Treg | | | | | | | | | | | | |
| diff | | | | | | | | | | | | |

TABLE 120

| L-2 | IL-3 | IL-4 | IL-5 | IL-6 | IL-7 | IL-8 | IP-10 | | MIP1a | MIP1b | Rantes | TNFa | TNFb | VEGF |
|---|---|---|---|---|---|---|---|---|---|---|---|---|---|---|
| pg/ml | pg/ml | pg/ml | pg/ml | pg/ml | pg/ml | pg/ml | pg/ml | pg/ml | pg/ml | pg/ml | pg/ml | pg/ml | pg/ml | pg/ml |
| 16.88 | 4.11 | 46.98 | 3.14 | 1889 | 24.48 | 9480 | 56.87 | 143.37 | 9480 | 5232 | 22.12 | 2973 | 9.46 | 103.85 |
| 4639 | 208.92 | 7944 | 2377 | 1889 | 52.36 | 10690 | 4639 | 3688 | 10389 | 5110 | 1053 | 13085 | 993 | 788.04 |
| 18071 | 99.39 | 3688 | 2246 | 3498 | 59.29 | 11210 | 1311 | 3592 | 9873 | 4525 | 884.16 | 7801 | 563.01 | 2061 |
| 48.26 | 5.5 | 165.89 | 4.45 | 4305 | 45.74 | 10073 | 308.26 | 1292 | 10860 | 7092 | 479.17 | 3886 | 13.58 | 186.26 |
| 325.18 | 62.71 | 1658 | 1037 | 2516 | 46.98 | 9210 | 4525 | 8229 | 11179 | 6400 | 2246 | 6537 | 431.72 | 351.91 |

TABLE 121

| Analyte | EGF | FGF2 | Eotaxin | TGFa | GCSF | FIT3L | GMCSF | Fractalkine | IFNa2 | IFNg | GRO | IL-10 |
|---|---|---|---|---|---|---|---|---|---|---|---|---|
| Sample | pg/ml | pg/ml | pg/ml | pg/ml | pg/ml | pg/ml | pg/ml | pg/ml | pg/ml | pg/ml | pg/ml | pg/ml |
| Exp183 | 48.92 | 116.77 | 32.85 | 6.52 | 1917 | 18.49 | 1183 | 300.06 | 276.51 | 2377 | 9744 | 936.9 |
| JC nCD4 | | | | | | | | | | | | |
| Exp183 | 48.26 | 116.77 | 37.17 | 7.8 | 2311 | 21.29 | 1183 | 370.61 | 390.03 | 1707 | 10417 | 1022 |
| TC nCD4 | | | | | | | | | | | | |
| Exp183 | 42.82 | 95.13 | 28.86 | 8.57 | 1757 | 23.01 | 1634 | 333.91 | 269.01 | 431.72 | 10813 | 1149 |
| MV nCD4 | | | | | | | | | | | | |

TABLE 122

| | | | | | | PDGF- | | | | | | | | |
|---|---|---|---|---|---|---|---|---|---|---|---|---|---|---|
| MCP3 | IL-12p40 | MDC | IL-12p70 | PDGFaa | IL-13 | AB/BB | IL-15 | sCD40L | IL-17a | IL-1Ra | IL-1a | IL-9 | IL-1b | L-2 |
| pg/ml | pg/ml | pg/ml | pg/ml | pg/ml | pg/ml | pg/ml | pg/ml | pg/ml | pg/ml | pg/ml | pg/ml | pg/ml | pg/ml | pg/ml |
| 43.96 | 97.95 | 3936 | 113.39 | 50.95 | 1183 | 884.16 | 12.93 | 964.52 | 56.87 | 1917 | 4144 | 139.23 | 325.18 | 1586 |
| 62.71 | 87.17 | 4414 | 71.25 | 69.24 | 1116 | 1682 | 16 | 1732 | 63.6 | 2377 | 1255 | 161.13 | 254.51 | 1783 |
| 88.45 | 53.81 | 5356 | 69.24 | 43.39 | 1411 | 1331 | 11.16 | 744.33 | 276.51 | 2278 | 1068 | 156.5 | 143.37 | 884.16 |

TABLE 123

| IL-3 | IL-4 | IL-5 | IL-6 | IL-7 | IL-8 | IP-10 | MCP1 | MIP1a | MIP1b | Rantes | TNFa | TNFb | VEGF |
|---|---|---|---|---|---|---|---|---|---|---|---|---|---|
| pg/ml | pg/ml | pg/ml | pg/ml | pg/ml | pg/ml | pg/ml | pg/ml | pg/ml | pg/ml | pg/ml | pg/ml | pg/ml | pg/ml |
| 26.68 | 897.04 | 342.82 | 5110 | 54.56 | 8371 | 8934 | 5867 | 11162 | 9480 | 9480 | 9210 | 284.19 | 578.64 |
| 34.49 | 611.45 | 261.67 | 4639 | 81.08 | 9873 | 8229 | 7801 | 12248 | 10162 | 9480 | 8934 | 448.79 | 637.48 |
| 103.85 | 858.99 | 454.67 | 4305 | 64.5 | 9210 | 3014 | 8654 | 11757 | 8934 | 6674 | 6674 | 152 | 533.23 |

TABLE 124

| | ITAC | | Franktalkine | | IL-23 | | IL-4 | | GM-CSF | | MIP-1β | |
|---|---|---|---|---|---|---|---|---|---|---|---|---|
| | JC | TC | JC | TC | JC | TC | JC | TC | JC | TC | JC | TC |
| Mean | 70.61 | 73.56 | 234.9 | 275.3 | 157.5 | 75.93 | 68.09 | 37.07 | 63.6 | 72.79 | 33.01 | 21.78 |
| SD | 50.99 | 33.24 | 23.52 | 31.79 | 25.45 | 22.19 | 15.13 | 8.249 | 15.56 | 8.275 | 5.853 | 5.678 |

TABLE 125

| | IFNγ | | IL-10 | | MIP-3α | | IL-13 | | IL-7 | | IL-8 | | MIP-1α | |
|---|---|---|---|---|---|---|---|---|---|---|---|---|---|---|
| | JC | TC | JC | TC | JC | TC | JC | TC | JC | TC | JC | TC | JC | TC |
| Mean | 5.094 | 3.455 | 14.43 | 3.563 | 9.378 | 3.075 | 6.091 | 3.893 | 8.125 | 8.013 | 5.441 | 5.825 | 15.56 | 15.06 |
| SD | 1.273 | 0.8174 | 8.433 | 2.251 | 2.18 | 0.8399 | 0.8124 | 1.067 | 0.8436 | 1.218 | 1.743 | 3.724 | 2.001 | 4.987 |

TABLE 126

| | IL-12p70 | | IL-1β | | IL-2 | | IL-21 | | IL-5 | | IL-6 | | TNF-α | | IL-17A | |
|---|---|---|---|---|---|---|---|---|---|---|---|---|---|---|---|---|
| | JC | TC | JC | TC | JC | TC | JC | TC | JC | TC | JC | TC | JC | TC | JC | TC |
| Mean | 3.678 | 1.643 | 1.17 | 1.168 | 6.063 | 0.4775 | 2.089 | 1.723 | 3.606 | 2.878 | 1.923 | 1.168 | 6.443 | 3.235 | 2.309 | 1.648 |
| SD | 0.9621 | 0.5848 | 0.2351 | 0.1443 | 10.42 | 0.1996 | 0.6836 | 0.733 | 2.45 | 0.3902 | 1.469 | 0.405 | 1.071 | 0.3371 | 0.9033 | 0.6232 |

TABLE 127
IL-12p70

| JC | TC | MV |
|---|---|---|
| 9.801 | 7.485 | 8.575 |
| 1.318 | 1.586 | 0.9687 |

TABLE 128
IL-17a

| JC | TC | MV |
|---|---|---|
| 14.45 | 6.978 | 8.425 |
| 4.195 | 1.104 | 0.3323 |

TABLE 129
IFNa2

| JC | TC | MV |
|---|---|---|
| 22.87 | 20.44 | 19.5 |
| 6.389 | 6.515 | 1.428 |

TABLE 130
IFNg

| JC | TC | MV |
|---|---|---|
| 21.25 | 13.08 | 14.67 |
| 6.702 | 3.998 | 0.1697 |

TABLE 131
IL-6

| JC | TC | MV |
|---|---|---|
| 14.86 | 6.95 | 6.64 |
| 9.353 | 1.336 | 0.198 |

TABLE 132
IL-12p40

| JC | TC | MV |
|---|---|---|
| 15.96 | 11.78 | 10.61 |
| 3.463 | 2.142 | 2.793 |

TABLE 133
Fractalkine

| JC | TC | MV |
|---|---|---|
| 39.89 | 24.21 | 22.24 |
| 24.68 | 8.025 | 2.447 |

TABLE 134
TNFa

| JC | TC | MV |
|---|---|---|
| 29.18 | 13.19 | 14.81 |
| 7.641 | 3.316 | 1.98 |

TABLE 135
GCSF

| JC | TC | MV |
|---|---|---|
| 82.17 | 28.17 | 35.01 |
| 55.41 | 11.4 | 7.248 |

TABLE 136
Rantes

| JC | TC | MV |
|---|---|---|
| 9578 | 3268 | 4973 |
| 3503 | 4775 | 5206 |

TABLE 137

| | 21 plex before NAC | 41 plex after NAC |
|---|---|---|
| IL-4 | 1.8 | 1.1 |
| IL-10 | 4 | 0.95 |
| IL-12 p70 | 2.24 | 1.3 |
| IL-2 | 12.6 | 0.9 |
| TNF-a | 2 | 2.2 |
| IFN-g | 1.47 | 1.6 |
| IL-13 | 1.56 | 0.99 |

TABLE 138A

| Analyte | | EGF | FGF2 | Eotaxin | TGFa | GCSF | FIT3L | GMCSF | Fractalkine | IFNa2 |
|---|---|---|---|---|---|---|---|---|---|---|
| Sample | Date | pg/ml | pg/ml | pg/ml | pg/ml | pg/ml | pg/ml | pg/ml | pg/ml | pg/ml |
| H109 RS | Aug. 8, 2019 | 49.58 | 65.42 | 145.48 | 41.72 | 5232 | 20.51 | 35.36 | 69.24 | 49.58 |
| pre NAC | | | | | | | | | | |
| H110 RS | Aug. 8, 2019 | 21.91 | 44.55 | 116.77 | 22.12 | 6131 | 15.11 | 26.68 | 61.83 | 31.31 |
| post NAC | | | | | | | | | | |

TABLE 138B

| | | | | | | | | | PDGF- | | | | |
|---|---|---|---|---|---|---|---|---|---|---|---|---|---|
| IFNg | GRO | IL-10 | MCP3 | IL-12p40 | CMD | IL-12p70 | PDGFaa | IL-13 | AB/BB | IL-15 | sCD40L | IL-17a | IL-1Ra |
| pg/ml | pg/ml | pg/ml | pg/ml | pg/ml | pg/ml | pg/ml | pg/ml | pg/ml | pg/ml | pg/ml | pg/ml | pg/ml | pg/ml |
| 25.54 | 2246 | 103.85 | 18.49 | 84.68 | 1732 | 16.21 | 9480 | 40.13 | 21269 | 25 | 12629 | 16.42 | 351.91 |
| 20.14 | 2246 | 97.95 | 16.88 | 82.26 | 1391 | 13.65 | 8654 | 33.66 | 23823 | 23.97 | 3545 | 12.34 | 247.51 |

TABLE 138C

| IL-1a | IL-9 | IL-1b | L-2 | IL-3 | IL-4 | IL-5 | IL-6 |
|---|---|---|---|---|---|---|---|
| pg/ml | pg/ml | pg/ml | pg/ml | pg/ml | pg/ml | pg/ml | pg/ml |
| 53.81 | 19.79 | 8.23 | 31.31 | 9.34 | 106.93 | 55.31 | 28.53 |
| 43.39 | 16.88 | 6.64 | 14.54 | 7.88 | 53.81 | 38.12 | 23.49 |

TABLE 138D

| IL-7 | IL-8 | IP-10 | MCP1 | MIP1a | MIP1b | Rantes | TNFa | TNFb | VEGF |
|---|---|---|---|---|---|---|---|---|---|
| pg/ml | pg/ml | pg/ml | pg/ml | pg/ml | pg/ml | pg/ml | pg/ml | pg/ml | pg/ml |
| 25 | 34.07 | 834.6 | 1149 | 18.49 | 40.13 | 9210 | 34.49 | 8.8 | 533.23 |
| 21.7 | 16.64 | 646.43 | 846.7 | 17.37 | 34.07 | 9480 | 30.58 | 6.89 | 247.51 |

TABLE 139

| | P113 HA | H130 RS |
|---|---|---|
| NK | 9.85% | 14.8% |
| CD4 | 14.2% | 36.6% |
| CD8 | 2.82% | 25.9% |
| CD19 | 71.2% | 9.00% |
| CD14 | 3.39% | 15.4% |

TABLE 140

| | | | | | IL-2 + | | IL-2 + | | IL-2 + | | IL-2 + | |
|---|---|---|---|---|---|---|---|---|---|---|---|---|
| | | | IL-2 | | αCD16 | | αCD3/28 | | sAJ2 | | αPD-1 | |
| | untreated | | treated | | treated | | treated | | treated | | treated | |
| PBMC | Mean | SD | Mean | SD | Mean | SD | Mean | SD | Mean | SD | Mean | SD |
| P113 HA | 4.55 | 0.89 | 17.31 | 0.41 | 1.67 | 0.09 | 29.04 | 2.01 | 15.52 | 0.13 | 20.80 | 0.28 |
| H130 RS | | | 42.75 | 2.15 | 28.81 | 1.50 | 126.61 | | 33.56 | 2.19 | 39.52 | 7.36 |

TABLE 141

| | | | IL-2 + | | IL-2 + | | IL-2 + | |
|---|---|---|---|---|---|---|---|---|
| | IL-2 | | αCD16 | | sAJ2 | | αPD-1 | |
| | treated | | treated | | treated | | treated | |
| | Mean | SD | Mean | SD | Mean | SD | Mean | SD |
| P113 | 218.54 | 0.01 | 21.56 | 1.02 | 243.34 | 18.10 | 99.04 | 6.06 |
| HA NK | | | | | | | | |

TABLE 142

| | | | IL-2 + | | IL-2 + | | IL-2 + | |
|---|---|---|---|---|---|---|---|---|
| | IL-2 | | αCD16 | | sAJ2 | | αPD-1 | |
| | treated | | treated | | treated | | treated | |
| Straight | Mean | SD | Mean | SD | Mean | SD | Mean | SD |
| P113 | 0.00 | 0.00 | 341.58 | 0.00 | 1534.97 | 166.35 | 0.00 | 0.00 |
| HA NK | | | | | | | | |

TABLE 143

| | | | IL-2 + αCD3/28 | | IL-2 + αPD-1 | |
|---|---|---|---|---|---|---|
| | IL-2 treated | | treated | | treated | |
| CD8 (1:5) | Mean | SD | Mean | SD | Mean | SD |
| P113 HA | 68.2 | 78.0 | 1023.8 | 96.8 | 0.0 | 0.0 |
| H130 RS | | | 1676.1 | 78.6 | | |

TABLE 144

| | H131 JC | H132 TC | H133 WO |
|---|---|---|---|
| NK | 9.55% | 11.2% | 12.3% |
| CD4 | 34.4% | 30.1% | 35.3% |
| CD8 | 23.0% | 26.3% | 14.3% |
| CD8/CD3 | 40.0% | 47.0% | 29.0% |
| CD19 | 16.0% | 9.35% | 10.3% |
| CD14 | 19.6% | 19.0% | 29.7% |

TABLE 145

| | | | | | IL-2 + | | IL-2 + | | IL-2 + | | IL-2 + | |
|---|---|---|---|---|---|---|---|---|---|---|---|---|
| | | | IL-2 | | aCD16 | | aCD3/28 | | sAJ2 | | aPD-1 | |
| | untreated | | treated | | treated | | treated | | treated | | treated | |
| | Mean | SD | Mean | SD | Mean | SD | Mean | SD | Mean | SD | Mean | SD |
| H131 JC | 5.48 | 0.37 | 29.22 | 1.57 | 24.04 | 0.00 | 31.61 | 0.93 | 39.17 | 1.74 | 34.00 | 7.49 |
| H132 TC | 5.48 | 0.39 | 36.04 | 3.17 | 24.06 | 0.00 | 36.25 | 1.71 | 61.49 | 2.06 | 41.63 | 18.32 |
| H133 WO | 10.99 | 3.13 | 54.49 | 15.29 | 17.38 | 0.15 | 136.26 | 17.63 | 69.32 | 3.16 | 42.30 | 1.71 |

TABLE 146

| | | | | | IL-2 + | | IL-2 + | | IL-2 + | |
|---|---|---|---|---|---|---|---|---|---|---|
| | | | IL-2 | | αCD16 | | αCD3/28 | | sAJ2 | |
| PBMC | untreated | | treated | | treated | | treated | | treated | |
| 1:10 | Mean | SD | Mean | SD | Mean | SD | Mean | SD | Mean | SD |
| H131 JC | 65.94 | 76.67 | 138.23 | 27.32 | 324.55 | 128.67 | 455.41 | 237.94 | 1885.41 | 19.34 |
| H132 TC | 26.69 | 0.00 | 10.48 | 0.00 | 177.49 | 0.00 | 361.94 | 0.00 | 1497.60 | 29.01 |
| H133 WO | 125.77 | 81.96 | 267.22 | 148.05 | 59.09 | 45.83 | 389.36 | 17.63 | 1700.78 | 161.63 |

TABLE 147

| CD8 | IL-2 | IL-2 + αCD3/28 | IL-2 + SAJ2 | IL-2 + αPD-1 |
|---|---|---|---|---|
| straight | treated | treated | treated | treated |
| H131 JC | 64.48 | 596.72 | | 44.13 |
| H132 TC | 4.18 | 407.50 | 111.98 | 46.39 |
| H133 WO | 72.78 | 549.98 | 119.52 | 86.35 |

TABLE 148

| | H136 JC | H137 TC |
|---|---|---|
| NK | 8.42% | 11.1% |
| CD4 | 36.3% | 31.2% |
| CD8 | 18.0% | 17.5% |
| CD8/CD3 | 33% | 36% |
| CD19 | 14.0% | 11.6% |
| CD14 | 14.3% | 16.4% |

TABLE 149

| | H140 JC | H141 RS |
|---|---|---|
| NK | 24.9% | 37.1% |
| CD4 | 33.7% | 28.4% |
| CD8 | 16.2% | 10.7% |
| CD8/CD3 | 32% | 27% |
| CD19 | 9.5% | 2.9% |
| CD14 | 26.9% | 15.9% |

TABLE 150

| | PBMC H140 JC | | PBMC H141 RS | | PBMC H142 JS | |
|---|---|---|---|---|---|---|
| LU 30/106 | LU | LU | LU | LU | LU | LU |
| cells | AVERAGE | SD | AVERAGE | SD | AVERAGE | SD |
| unt | 4.41 | 0.65 | 0.59 | 0.07 | 3.91 | 0.23 |
| IL2 | 18.03 | 0.50 | 2.14 | 0.27 | 15.16 | 1.72 |
| IL2 antiCD16 | 10.67 | 0.22 | 1.33 | 0.19 | 11.75 | 0.95 |
| IL2 aCD3/28 | 18.04 | 1.78 | 1.51 | 0.03 | 18.31 | 1.61 |
| IL2 sAJ2 | 18.40 | 0.61 | 1.15 | 0.41 | 24.88 | 0.42 |
| IL2 aPD1 | 10.06 | 0.26 | 2.46 | 0.18 | 9.46 | 0.69 |
| aPD1 | 1.70 | 0.09 | 0.37 | 0.16 | 1.61 | 0.06 |

| | NK H140 JC | | NK H141 RS | | NK H142 JS | |
|---|---|---|---|---|---|---|
| LU 30/106 | LU | LU | LU | LU | LU | LU |
| cells | AVERAGE | SD | AVERAGE | SD | AVERAGE | SD |
| IL2 | 167.50 | 5.45 | 14.79 | 0.67 | 58.61 | 1.14 |
| IL2 aCD16 | 28.65 | 24.70 | 10.77 | 1.71 | 39.08 | 0.79 |
| IL2 sAJ2 | 180.88 | 22.95 | 11.53 | 0.45 | 73.43 | 1.33 |
| IL2 aPD1 | 102.23 | 1.84 | 10.61 | 2.65 | 43.21 | 2.34 |

TABLE 151
Exp203 H143 JC H144 TC H145 WO

| | H143 JC | H144 TC | H145 WO | H146 JC | H147 TC | H148 WO |
|---|---|---|---|---|---|---|
| NK | 13.0% | 10.6% | 15.1% | 10.8% | 8.52% | 11.6% |
| CD4 | 35.7% | 37.9% | 33.9% | 37.2% | 39.2% | 35.6% |
| CD8 | 20.8% | 28.1% | 14.4% | 20.7% | 25.5% | 16.1% |
| CD8/CD3 | 36.8% | 42% | 30% | 35.7% | 39% | 31% |
| CD19 | 11.0% | 10.3% | 8.34% | 11.9% | 10.4% | 10.4% |
| CD14 | 19.8% | 10.9% | 28.0% | 15.9% | 9.64% | 17.7% |

TABLE 152

| | | | | | IL-2 + | | IL-2 + | | IL-2 + | | IL-2 + | |
|---|---|---|---|---|---|---|---|---|---|---|---|---|
| | | | IL-2 | | aCD16 | | aCD3/28 | | sAJ2 | | aPD-1 | |
| | untreated | | treated | | treated | | treated | | treated | | treated | |
| PBMC | Mean | SD | Mean | SD | Mean | SD | Mean | SD | Mean | SD | Mean | SD |
| H143 JC | 5.93 | 0.86 | 20.96 | 0.63 | 14.60 | 0.96 | 26.49 | 0.17 | 49.89 | 0.53 | 13.21 | 0.59 |
| H144 TC | 3.38 | 0.14 | 16.77 | 1.83 | 10.96 | 0.48 | 39.49 | 0.17 | 28.99 | 0.83 | 20.27 | 2.11 |
| H145 WO | 3.33 | 0.83 | 11.70 | 0.07 | 17.97 | 1.08 | 25.96 | 2.75 | 38.69 | 6.93 | 13.04 | 3.04 |
| H146 JC | 3.55 | 0.36 | 21.79 | 1.73 | 12.47 | 0.27 | 34.18 | 0.54 | 24.33 | 3.31 | 18.32 | 1.97 |
| H147 TC | 2.62 | 0.23 | 12.29 | 0.57 | 8.03 | 0.15 | 20.94 | 1.53 | 13.65 | 3.32 | 14.32 | 0.04 |
| H148 WO | 3.78 | 1.64 | 8.22 | 1.63 | 6.52 | 0.78 | 26.54 | 1.38 | 31.91 | 0.52 | 14.34 | 1.70 |

TABLE 153

| | | | | | IL-2 + | | IL-2 + | | IL-2 + | |
|---|---|---|---|---|---|---|---|---|---|---|
| | | | IL-2 | | aCD16 | | sAJ2 | | aPD-1 | |
| | untreated | | treated | | treated | | treated | | treated | |
| NK | Mean | SD | Mean | SD | Mean | SD | Mean | SD | Mean | SD |
| H143 JC | 27.05 | 2.64 | 117.46 | 3.21 | 61.55 | 3.96 | 217.99 | 1.92 | 141.99 | 16.43 |
| H144 TC | 23.03 | 2.92 | 205.18 | 3.39 | 11.23 | 4.01 | 272.31 | 38.27 | 159.41 | 18.76 |
| H145 WO | 14.24 | 3.32 | 171.54 | 12.72 | 24.68 | 1.39 | 387.04 | 32.02 | 197.50 | 12.61 |

TABLE 154

| | | | | | IL-2 + | | IL-2 + | | IL-2 + | |
|---|---|---|---|---|---|---|---|---|---|---|
| | | | IL-2 | | αCD16 | | αCD3/28 | | sAJ2 | |
| 1:10 | untreated | | treated | | treated | | treated | | treated | |
| PBMCs | Mean | SD | Mean | SD | Mean | SD | Mean | SD | Mean | SD |
| H143 JC | 0.00 | 1.27 | 13.50 | 0.00 | 867.94 | 0.00 | 403.73 | 26.71 | 2123.79 | 25.90 |
| H145 WO | 4.50 | 1.70 | 0.00 | 1.33 | 574.87 | 16.19 | 783.80 | 4.05 | 2325.27 | 9.71 |
| H146 JC | 0.00 | 5.52 | 6.30 | 4.24 | 622.38 | 46.14 | 197.66 | 10.52 | 2103.75 | 212.90 |
| H147 TC | 1.20 | 0.42 | 58.00 | 5.67 | | | 257.19 | 28.33 | 1977.25 | 76.09 |
| H148 WO | 6.90 | 5.94 | 7.50 | 9.34 | 329.31 | 7.29 | 563.43 | 84.19 | 2368.20 | 96.33 |

TABLE 155

| | | | | | IL-2 + | | IL-2 + | |
|---|---|---|---|---|---|---|---|---|
| | | | IL-2 | | αCD3/28 | | αPD-1 | |
| | untreated | | treated | | treated | | treated | |
| 1:5 | Mean | SD | Mean | SD | Mean | SD | Mean | SD |
| H143 JC CD8 | 0.0 | 1.1 | 46.1 | 7.2 | 545.2 | 6.8 | 41.8 | 2.7 |
| H144 TC CD8 | 0.0 | 2.3 | 6.2 | 9.9 | 550.9 | 6.4 | 5.4 | 10.2 |
| H145 WO CD8 | 4.6 | 0.0 | 8.1 | 5.7 | 565.1 | 92.5 | 17.7 | 7.2 |

TABLE 156

| Straight | untreated | IL-2 + sAJ2 treated |
|---|---|---|
| H143 JC NK | 36.04 | 1721.83 |
| H144 TC NK | 24.15 | 419.61 |
| H145 WO NK | 4.69 | 801.94 |

TABLE 157

| | GCSF | IL-17A | IL-6 | RANTES | TNFa |
|---|---|---|---|---|---|
| Analyte Sample | pg/ml | pg/ml | pg/ml | pg/ml | pg/ml |
| H143 JC NK IL2 | 2.94 | 2.34 | 42.5 | 859.54 | 22.64 |
| H144 TC NK IL2 | 2.37 | 2.38 | 21.79 | 1944 | 19.74 |
| H145 WO NK IL2 | 2.51 | 2.3 | 3.5 | 5684 | 7.42 |
| H144 JC NK il2acd16 | 2.94 | 2.3 | 12.63 | 1477 | 43.56 |
| H145 TC NK il2acd16 | 3.53 | 2.54 | 8.19 | 2788 | 52.61 |
| H146 WO NK il2acd16 | 4.46 | 2.38 | 11.4 | 9431 | 49.12 |
| H143 JC CD8 IL2aC328 | 6.98 | 2.87 | 228.57 | 1004 | 633.99 |
| H144 TC CD8 IL2aC328 | 4.95 | 3.13 | 19.36 | 3485 | 752.71 |
| H145 WO CD8 IL2aC328 | 191.23 | 4.21 | 2322 | 19395 | 1687 |

TABLE 158

| Analyte Sample | GCSF | IFNg | IL-17A | IL-6 | RANTES | TNFa |
|---|---|---|---|---|---|---|
| H153 RS NK IL2 | 2.8 | 58.75 | 2.3 | 2.44 | 478.7 | 115.89 |
| H153 RS CD8 IL2aCd328 | 7.69 | 1145 | 13.53 | 7.51 | 1288 | 4790 |
| H153 RS NK il2acd16 | 3.23 | 153.64 | 2.46 | 2.31 | 1911 | 273.39 |

TABLE 159

| | H153 RS |
|---|---|
| NK | 12.9% |
| CD4 | 33.8% |
| CD8 | 16.4% |
| CD8/CD3 | 32.8% |
| CD19 | 5.49% |
| CD14 | 9.93% |
| CD15 | 31.0% |

TABLE 160

| | | | | | IL-2 + | | IL-2 + | | IL-2 + | | IL-2 + | |
|---|---|---|---|---|---|---|---|---|---|---|---|---|
| | | | IL-2 | | aCD16 | | aCD3/28 | | sAJ2 | | aPD-1 | |
| | untreated | | treated | | treated | | treated | | treated | | treated | |
| Lu70/10⁶ | Mean | SD | Mean | SD | Mean | SD | Mean | SD | Mean | SD | Mean | SD |
| P153 | 0.26 | 0.35 | 31.23 | 4.41 | 32.24 | 1.52 | 40.07 | 0.08 | 28.31 | 2.68 | 31.66 | 0.41 |
| PBMC | | | | | | | | | | | | |
| P153 NK | 13.04 | 2.08 | 94.51 | 0.28 | 51.95 | 2.15 | | | 87.45 | 20.86 | 96.21 | 22.85 |

TABLE 161

| | H160 JC | H161 TC | H162 WO |
|---|---|---|---|
| NK | 11.9% | 10.7% | 14.1% |
| CD4 | 31.2% | 38.4% | 27.3% |
| CD8 | 21.2% | 24.7% | 8.67% |
| CD19 | 20.3% | 10.4% | 6.86% |
| CD14 | 10.7% | 16.3% | 8.82% |
| Total | 95.3% | 100.5% | 65.75% |
| CD8/CD4 | 40% | 39% | 24% |

TABLE 162

| | H165 RS |
|---|---|
| NK | 16.6% |
| CD4 | 39.3% |
| CD8 | 12.8% |
| CD19 | 17.8% |
| CD14 | 11.3% |
| total | 97.8% |
| CD8/CD4 + CD8 | 24% |

TABLE 163

| | | | | | IL-2 + | | IL-2 + | | IL-2 + | | IL-2 + | |
|---|---|---|---|---|---|---|---|---|---|---|---|---|
| | | | IL-2 | | αCD16 | | αCD3/28 | | sAJ2 | | αPD-1 | |
| | untreated | | treated | | treated | | treated | | treated | | treated | |
| PBMC | Mean | SD | Mean | SD | Mean | SD | Mean | SD | Mean | SD | Mean | SD |
| H160 JC | 4.11 | 1.81 | 3.93 | 0.20 | 6.38 | 0.54 | 1.51 | 0.38 | 1.57 | 0.78 | 1.91 | 0.15 |
| H161 TC | 1.08 | 0.04 | 3.71 | 0.09 | 1.17 | 0.28 | 4.89 | 0.17 | 9.99 | 0.94 | 3.06 | 0.43 |
| H162 WO | 5.61 | 0.38 | 32.75 | 2.61 | 15.18 | 0.75 | 38.14 | 1.39 | 44.52 | 0.03 | 31.01 | 0.00 |

TABLE 164

| | | | | | IL-2 + | | IL-2 + | | IL-2 + | | IL-2 + | |
|---|---|---|---|---|---|---|---|---|---|---|---|---|
| | | | IL-2 | | aCD16 | | aCD3/28 | | sAJ2 | | aPD-1 | |
| | untreated | | treated | | treated | | treated | | treated | | treated | |
| | Mean | SD | Mean | SD | Mean | SD | Mean | SD | Mean | SD | Mean | SD |
| H165 | 15.94 | 0.65 | 80.29 | 3.57 | 17.74 | 0.69 | 70.31 | 1.82 | 67.28 | 0.50 | 45.09 | 1.95 |
| RS | | | | | | | | | | | | |

TABLE 165

| | | | | | IL-2 + | | IL-2 + | | IL-2 + | | IL-2 + | |
|---|---|---|---|---|---|---|---|---|---|---|---|---|
| | | | IL-2 | | αCD16 | | αCD3/28 | | sAJ2 | | αPD-1 | |
| PBMC | untreated | | treated | | treated | | treated | | treated | | treated | |
| 1:10 | Mean | SD | Mean | SD | Mean | SD | Mean | SD | Mean | SD | Mean | SD |
| H160 JC | 25.17 | 5.31 | 50.53 | 2.66 | 336.54 | 24.57 | 322.46 | 8.63 | 2751.93 | 205.90 | 106.89 | 1.33 |
| H161 TC | 5.44 | 10.63 | 20.94 | 3.32 | 43.96 | 9.30 | 43.02 | 30.55 | 719.31 | 15.94 | 32.21 | 24.57 |
| H162 WO | 3.10 | 5.98 | 22.35 | 0.00 | 98.90 | 24.57 | 158.55 | 2.66 | 2790.44 | 65.09 | 22.82 | 1.99 |

TABLE 166

| | | | | | IL-2 + | | IL-2 + | | IL-2 + | | IL-2 + | |
|---|---|---|---|---|---|---|---|---|---|---|---|---|
| | | | IL-2 | | αCD16 | | αCD3/28 | | sAJ2 | | αPD-1 | |
| PBMC | untreated | | treated | | treated | | treated | | treated | | treated | |
| 1:10 | Mean | SD | Mean | SD | Mean | SD | Mean | SD | Mean | SD | Mean | SD |
| H165 | 18.30 | 7.72 | 53.31 | 8.36 | 33.76 | 1.29 | 202.24 | 7.40 | 2352.36 | 134.92 | 27.85 | 0.64 |
| RS | | | | | | | | | | | | |

TABLE 167

| | | | | | IL-2 + | | IL-2 + | | IL-2 + | |
|---|---|---|---|---|---|---|---|---|---|---|
| | | | IL-2 | | αCD3/28 | | sAJ2 | | αPD-1 | |
| CD8 | untreated | | treated | | treated | | treated | | treated | |
| (1:5) | Mean | SD | Mean | SD | Mean | SD | Mean | SD | Mean | SD |
| H160 JC | 11.41 | 7.74 | 28.37 | 2.32 | 1104.77 | 36.35 | 1105.86 | 64.20 | 34.93 | 28.62 |
| H161 TC | 26.73 | 24.75 | 9.77 | 3.87 | 453.90 | 30.17 | 71.03 | 3.87 | 22.35 | 4.64 |
| H162 WO | 19.62 | 14.70 | 76.50 | 87.41 | 1750.71 | 69.62 | | | | |

TABLE 168

| | untreated | | IL-2 + αCD3/28 treated | | IL-2 + sAJ2 treated | |
|---|---|---|---|---|---|---|
| | Mean | SD | Mean | SD | Mean | SD |
| H165 RS CD8 1:5 | 10.11 | 1.29 | 2677.98 | 86.53 | 56.72 | 7.40 |

TABLE 169

| | H169 JC | H170 TC |
|---|---|---|
| NK | 14.0% | 14.9% |
| CD4 | 29.0% | 26.2% |
| CD8 | 20.8% | 25.9% |
| CD19 | 21.0% | 15.5% |
| CD14 | 14.6% | 11.2% |
| total | 99.4% | 93.7% |
| CD8/CD4 + CD8 | 42% | 50% |

TABLE 170

| | | | | | IL-2 + anti- | | IL-2 + anti- | | IL-2 + | | IL-2 + anti- | |
|---|---|---|---|---|---|---|---|---|---|---|---|---|
| | | | IL-2 | | CD16 | | CD3/28 | | sAJ2 | | PD-1 | |
| | untreated | | treated | | treated | | treated | | treated | | treated | |
| | Mean | SD | Mean | SD | Mean | SD | Mean | SD | Mean | SD | Mean | SD |
| H169 JC | 6.28 | 1.26 | 34.90 | 0.35 | 28.56 | 0.16 | 49.93 | 6.26 | 40.26 | 3.44 | 35.59 | 0.82 |
| PBMC | | | | | | | | | | | | |
| H170 TC | 6.59 | 0.11 | 24.21 | 0.41 | 24.67 | 0.45 | 74.93 | 4.57 | 45.96 | 0.29 | 26.74 | 0.19 |
| PBMC | | | | | | | | | | | | |

TABLE 171

| | | | IL-2 + anti- | IL-2 + anti- | IL-2 + | IL-2 + anti- |
|---|---|---|---|---|---|---|
| | | IL-2 | CD16 | CD3/28 | sAJ2 | PD-1 |
| | untreated | treated | treated | treated | treated | treated |
| H169 JC | 0.00 | 30.70 | 240.91 | 1104.50 | Overflow | 0.00 |
| PBMC | | | | | | |
| (1:10) | | | | | | |
| H170 TC | 0.00 | 35.89 | 132.53 | 298.98 | Overflow | 0.00 |
| PBMC | | | | | | |
| (1:10) | | | | | | |

TABLE 172

| | | IL-2 + | IL-2 + | IL-2 + |
|---|---|---|---|---|
| | IL-2 | anti-CD3/28 | sAJ2 | anti-PD-1 |
| | treated | treated | treated | treated |
| H169 JC | 316.25 | 1374.80 | 1675.70 | 119.51 |
| CD8 (1:5) | | | | |
| H170 TC | 19.64 | 162.13 | 328.33 | 12.20 |
| CD8 (1:5) | | | | |

TABLE 173

| | H171 JC | H172 TC |
|---|---|---|
| NK | 11.5% | 8.37% |
| CD4 | 36.7% | 37.7% |
| CD8 | 24.9% | 25.7% |
| CD19 | 17.7% | 12.8% |
| CD14 | 9.47% | 7.54% |
| total | 100.2% | 92.11% |
| CD8/CD4 + CD8 | 40% | 40% |

TABLE 174

| | | | | | IL-2 + anti- | | IL-2 + anti- | | IL-2 + | | IL-2 + anti- | |
|---|---|---|---|---|---|---|---|---|---|---|---|---|
| | | | IL-2 | | CD16 | | CD3/28 | | sAJ2 | | PD-1 | |
| | untreated | | treated | | treated | | treated | | treated | | treated | |
| | Mean | SD | Mean | SD | Mean | SD | Mean | SD | Mean | SD | Mean | SD |
| H171 JC | 8.65 | 0.93 | 16.43 | 0.48 | 16.93 | 1.85 | 27.33 | 1.20 | 22.48 | 0.21 | 30.50 | 0.65 |
| PBMC | | | | | | | | | | | | |
| H172 TC | 8.00 | 1.42 | 33.37 | 10.1 | 12.23 | 1.65 | 34.69 | 0.75 | 45.96 | 0.29 | 15.93 | 0.33 |
| PBMC | | | | | | | | | | | | |

TABLE 175

| | | | | | IL-2 + | | IL-2 + | | IL-2 + anti- | |
|---|---|---|---|---|---|---|---|---|---|---|
| | | | IL-2 | | anti-CD16 | | sAJ2 | | PD-1 | |
| | untreated | | treated | | treated | | treated | | treated | |
| | Mean | SD | Mean | SD | Mean | SD | Mean | SD | Mean | SD |
| H171 JC | 10.58 | 0.05 | 57.06 | 1.73 | 18.63 | 0.50 | 104.9 | 6.40 | 73.86 | 0.20 |
| NK | | | | | | | | | | |
| H172 TC | 15.31 | 4.84 | 69.13 | 15.3 | 24.91 | 1.23 | 121.8 | 1.38 | 92.64 | 7.83 |
| NK | | | | | | | | | | |

TABLE 176

| | | | IL-2 + anti- | IL-2 + anti- | IL-2 + | IL-2 + anti- |
|---|---|---|---|---|---|---|
| | | IL-2 | CD16 | CD3/28 | sAJ2 | PD-1 |
| | untreated | treated | treated | treated | treated | treated |
| H171 JC | 0.00 | 107.35 | 1501.09 | 766.87 | overflow | 43.38 |
| PBMC | | | | | | |
| (1:10) | | | | | | |
| H172 TC | 0.00 | 51.25 | 221.62 | 217.76 | overflow | 23.13 |
| PBMC | | | | | | |
| (1:10) | | | | | | |

TABLE 177

| | | | IL-2 + | IL-2 + | IL-2 + |
|---|---|---|---|---|---|
| | | IL-2 | anti-CD3/28 | sAJ2 | anti-PD1 |
| | untreated | treated | treated | treated | treated |
| H171 JC | 4.51 | 0.00 | 1694.44 | 159.43 | 5.67 |
| CD8 (1:5) | | | | | |
| H172 TC | 0.00 | 3.47 | 874.21 | 72.75 | 5.15 |
| CD8 (1:5) | | | | | |

TABLE 178

| | | | IL-2 + | IL-2 + | IL-2 + |
|---|---|---|---|---|---|
| | | IL-2 | anti-CD16 | sAJ2 | anti-PD1 |
| | untreated | treated | treated | treated | treated |
| H171 JC | 0.00 | 157.01 | 158.45 | overflow | 7.70 |
| NK (1:5) | | | | | |
| H172 TC | 0.00 | 27.63 | 116.99 | overflow | 32.29 |
| NK (1:5) | | | | | |

TABLE 179

| | H174 JC | H175 TC |
|---|---|---|
| NK | 14.2% | 9.15% |
| CD4 | 46.4% | 47.7% |
| CD8 | 26.4% | 29.6% |
| CD19 | 14.2% | 10.2% |
| CD14 | 7.63% | 8.08% |
| total | 108.83% | 104.73% |
| CD8/CD4 + CD8 | 36% | 38% |

TABLE 180

| | | | | | IL-2 + anti- | | IL-2 + anti- | | IL-2 + | | IL-2 + anti- | |
|---|---|---|---|---|---|---|---|---|---|---|---|---|
| | | | IL-2 | | CD16 | | CD3/28 | | sAJ2 | | PD-1 | |
| | untreated | | treated | | treated | | treated | | treated | | treated | |
| | Mean | SD | Mean | SD | Mean | SD | Mean | SD | Mean | SD | Mean | SD |
| H171 JC | 6.79 | 0.57 | 42.30 | 0.22 | 41.29 | | 75.62 | | 33.95 | 1.90 | 32.67 | |
| PBMC | | | | | | | | | | | | |
| H172 TC | 5.38 | 1.98 | 31.50 | 1.98 | 20.57 | 5.31 | 49.12 | | 34.49 | 0.67 | 33.09 | 0.70 |
| PBMC | | | | | | | | | | | | |

TABLE 181

| | | | | | IL-2 + | | IL-2 + | | IL-2 + anti- | |
|---|---|---|---|---|---|---|---|---|---|---|
| | | | IL-2 | | anti-CD16 | | sAJ2 | | PD-1 | |
| | untreated | | treated | | treated | | treated | | treated | |
| | Mean | SD | Mean | SD | Mean | SD | Mean | SD | Mean | SD |
| H174 JC | 58.77 | 10.7 | 142.1 | 5.10 | 47.85 | 1.02 | 163.7 | 0.38 | 177.3 | 22.7 |
| NK | | | | | | | | | | |
| H175 TC | 32.81 | 0.44 | 154.9 | 6.70 | 61.45 | 6.90 | 219.6 | 2.67 | 130.5 | 17.0 |
| NK | | | | | | | | | | |

TABLE 182

| | | | IL-2 + anti- | IL-2 + anti- | IL-2 + | IL-2 + anti- |
|---|---|---|---|---|---|---|
| | | IL-2 | CD16 | CD3/28 | sAJ2 | PD-1 |
| | untreated | treated | treated | treated | treated | treated |
| H174 JC | 0.00 | 33.73 | 615.22 | 499.78 | overflow | 52.05 |
| PBMC | | | | | | |
| (1:10) | | | | | | |
| H175 TC | 0.00 | 39.96 | 423.51 | 193.10 | 1362.74 | 16.28 |
| PBMC | | | | | | |
| (1:10) | | | | | | |

TABLE 183

| | | IL-2 + | IL-2 + | IL-2 + |
|---|---|---|---|---|
| | IL-2 | anti-CD3/28 | sAJ2 | anti-PD1 |
| | treated | treated | treated | treated |
| H174 JC | 0.00 | 379.14 | 143.63 | 0.00 |
| CD8 (1:5) | | | | |
| H175 TC | 0.00 | 108.00 | 66.13 | 0.00 |
| CD8 (1:5) | | | | |

TABLE 184

| | | | IL-2 + | IL-2 + | IL-2 + |
|---|---|---|---|---|---|
| | | IL-2 | anti-CD16 | sAJ2 | anti-PD1 |
| | untreated | treated | treated | treated | treated |
| H174 JC | 0.00 | 20.15 | 125.81 | 2005.61 | 180.39 |
| NK (1:5) | | | | | |
| H175 TC | 2.20 | 608.98 | 596.84 | 1838.02 | 37.47 |
| NK (1:5) | | | | | |

TABLE 185

| | | | | | | | IL- | | | |
|---|---|---|---|---|---|---|---|---|---|---|
| | ITAC | GM-CSF | Franktalkine | IFNg | IL-10 | MIP-3a | 12p70 | IL-13 | IL-17A | IL-1b |
| Analyte Sample | pg/ml | pg/ml | pg/ml | pg/ml | pg/ml | pg/ml | pg/ml | pg/ml | pg/ml | pg/ml |
| JC sera Apr. 5, 2020 | 18.73 | 91.74 | 39.54 | 11.6 | 8.12 | 11.35 | 2.32 | 4.83 | 6.07 | 0.87 |
| (NAC infusion) | | | | | | | | | | |
| JC sera Apr. 16, 2020 | 11.26 | 54.8 | 33.33 | 7.11 | 4.47 | 5.58 | 2.1 | 4.44 | 3.15 | 0.65 |
| (NAC infusion | | | | | | | | | | |
| JC sera Apr. 23, 2020 | 10.71 | 84.17 | 34.75 | 6.15 | 6 | 6.78 | 2 | 4.17 | 2.78 | 0.77 |
| (NAC infusion | | | | | | | | | | |
| JC sera Apr. 30, 2020 | 14.07 | 59.71 | 35.49 | 9.14 | 5.71 | 8.17 | 2.32 | 4.26 | 5.22 | 1.03 |
| (NAC infusion | | | | | | | | | | |
| JC sera May 7, 2020 | 13.07 | 58.44 | 33.33 | 4.81 | 6.63 | 7.8 | 1.6 | 3.99 | 2.78 | 0.72 |
| (NAC infusion | | | | | | | | | | |
| JC sera May 14, 2020 | 12.13 | 53.64 | 31.36 | 4.14 | 4.47 | 6.78 | 1.81 | 4.08 | 2.24 | 0.8 |
| (NAC infusion | | | | | | | | | | |
| JC sera May 21, 2020 | 15.13 | 48.19 | 32.65 | 5.58 | 3.7 | 6.78 | 1.72 | 3.99 | 2.85 | 0.8 |
| (NAC infusion | | | | | | | | | | |
| JC sera May 28, 2020 | 14.77 | 75.6 | 37.04 | 8.94 | 6.63 | 8.17 | 2.16 | 4.44 | 5.49 | 0.87 |
| (NAC infusion | | | | | | | | | | |
| JC sera Jun. 22, 2020 | 13.07 | 50.3 | 35.49 | 10.42 | 8.54 | 10.65 | 2.32 | 5.03 | 6.86 | 0.91 |
| (NAC infusion | | | | | | | | | | |
| JC sera Jun. 24, 2020 | 10.44 | 111.4 | 35.11 | 4.58 | 23.69 | 8.55 | 1.64 | 3.95 | 2.46 | 0.77 |
| (NAC infusion | | | | | | | | | | |
| H184 JC sera | 62.12 | 70.88 | 53.3 | 13.16 | 34.61 | 14.62 | 6.24 | 6.66 | 13.92 | 39.92 |
| Jul. 16, 2020 | | | | | | | | | | |
| H185 TC sera | 14.07 | 80.63 | 25.04 | 11.6 | 17.05 | 5.86 | 3.15 | 0.91 | 15.48 | 2.44 |
| Jul. 15, 2020 | | | | | | | | | | |
| H183 NC sera | 21.95 | 49.23 | 60.11 | 0.86 | 2.08 | 1.41 | 0.49 | 2.02 | 0.71 | 0.54 |
| Jul. 15, 2020 | | | | | | | | | | |
| Ave JC for NAC | 13.34 | 68.8 | 34.8 | 7.25 | 7.8 | 8.1 | 2 | 4.32 | 4 | 0.82 |
| infusion | | | | | | | | | | |
| SD | 2.5 | 21.1 | 2.3 | 2.63 | 5.8 | 1.8 | 0.29 | 0.37 | 1.72 | 10.11 |

TABLE 186

| | | | | | | | | | MIP- | MIP- | |
|---|---|---|---|---|---|---|---|---|---|---|---|
| | IL-2 | IL-21 | IL-4 | IL-23 | IL-5 | IL-6 | IL-7 | IL-8 | 1a | 1b | TNFa |
| Analyte Sample | pg/ml | pg/ml | pg/ml | pg/ml | pg/ml | pg/ml | pg/ml | pg/ml | pg/ml | pg/ml | pg/ml |
| JC sera Apr. 5, 2020 | 0.14 | 0.82 | 18.46 | 144.83 | 2.64 | 2.74 | 5.61 | 5.32 | 11.35 | 24.47 | 9.39 |
| JC sera Apr. 16, 2020 | 0.32 | 0.62 | 11.22 | 116.26 | 2.21 | 1.55 | 4.9 | 3.55 | 10.8 | 18.56 | 7.45 |
| JC sera Apr. 23, 2020 | 5.42 | 0.68 | 15.94 | 132.6 | 2.21 | 1.55 | 6.39 | 3.72 | 11.24 | 16.32 | 7.45 |
| JC sera Apr. 30, 2020 | 0.06 | 0.86 | 14.43 | 138.55 | 2 | 2.34 | 6.25 | 4.28 | 11.35 | 17.78 | 8.46 |
| JC sera May 7, 2020 | 0.21 | 0.62 | 7.5 | 116.26 | 1.77 | 1.37 | 4.06 | 3.39 | 10.39 | 16.67 | 7.45 |
| JC sera May 14, 2020 | 8.16 | 0.56 | 13.73 | 106.21 | 1.72 | 1.07 | 6.39 | 5.32 | 12.15 | 19.37 | 7.77 |
| JC sera May 12, 2020 | 0 | 0.62 | 10.14 | 92.37 | 2.05 | 1.76 | 5.13 | 4.18 | 11.8 | 25.53 | 7.14 |
| JC sera May 28, 2020 | 0.01 | 0.75 | 11.8 | 144.83 | 2.21 | 2.03 | 5.99 | 4.47 | 11.46 | 25.53 | 8.64 |
| JC sera Jun. 22, 2020 | 0.04 | 0.86 | 15.17 | 158.41 | 2.44 | 3.33 | 7.25 | 5.21 | 12.15 | 24.21 | 9.2 |
| JC sera Jun. 24, 2020 | 0.17 | 0.62 | 10.14 | 106.21 | 2.1 | 1.31 | 5.13 | 4.47 | 12.15 | 27.78 | 8.82 |
| H184 JC sera | 0.01 | 3.74 | 45.29 | 251.93 | 3.66 | 15.17 | 7.72 | 837746 | 35.79 | 50.64 | 16.57 |
| Jul. 16, 2020 | | | | | | | | | | | |
| H185 TC sera | 0.01 | 0.49 | 28.05 | 96.82 | 2.99 | 1.55 | 6.12 | 2.4 | 8.53 | 7.22 | 5.09 |
| Jul. 15, 2020 | | | | | | | | | | | |
| H183 NC sera | | 0.3 | 8.29 | 12.09 | 1.26 | 0.23 | 4.26 | 1.31 | 8.03 | 9.23 | 3.05 |
| Jul. 15, 2020 | | | | | | | | | | | |
| Ave JC for NAC | 1.45 | 0.7 | 12.8 | 125.6 | 2.13 | 1.9 | 5.7 | 4.4 | 11.5 | 21.6 | 8.2 |
| infusion | | | | | | | | | | | |
| SD | 2.9 | 0.11 | 3.3 | 21.2 | 0.28 | 0.71 | 0.93 | 0.72 | 0.59 | 4.28 | 0.82 |

TABLE 187
Before NAC

| | ITAC | | Franktalkine | | IL-23 | | IL-4 | | GM-CSF | | MIP-1β | |
|---|---|---|---|---|---|---|---|---|---|---|---|---|
| | JC | TC | JC | TC | JC | TC | JC | TC | JC | TC | JC | TC |
| Mean | 70.61 | 73.56 | 234.9 | 275.3 | 157.5 | 75.93 | 68.09 | 37.07 | 63.6 | 72.79 | 33.01 | 21.78 |
| SD | 50.99 | 33.24 | 23.52 | 31.79 | 25.45 | 22.19 | 15.13 | 8.249 | 15.56 | 8.275 | 5.853 | 5.678 |

After NAC

| | | | | | GM- | |
|---|---|---|---|---|---|---|
| | ITAC | Franktalkine | IL-23 | IL-4 | CSF | MIP-1b |
| Ave JC 4/5 to 6/24 | 13.34 | 34.8 | 125.6 | 12.8 | 68.8 | 21.6 |
| SD | 2.5 | 2.3 | 21.2 | 3.3 | 21.1 | 4.28 |
| Fold change | 0.19 | 0.15 | 0.80 | 0.19 | 1.1 | 0.65 |

TABLE 188

| | IFNγ | | IL-10 | | MIP-3α | | IL-13 | | IL-7 | | IL-8 | | MIP-1α | |
|---|---|---|---|---|---|---|---|---|---|---|---|---|---|---|
| | JC | TC | JC | TC | JC | TC | JC | TC | JC | TC | JC | TC | JC | TC |
| Mean | 5.094 | 3.455 | 14.43 | 3.563 | 9.378 | 3.075 | 6.091 | 3.893 | 8.125 | 8.013 | 5.441 | 5.825 | 15.56 | 15.06 |
| SD | 1.273 | 0.8174 | 8.433 | 2.251 | 2.18 | 0.8399 | 0.8124 | 1.067 | 0.8436 | 1.218 | 1.743 | 3.724 | 2.001 | 4.987 |

| | IFNg | IL-10 | MIP-3a | IL-13 | IL-7 | IL-8 | MIP-1a |
|---|---|---|---|---|---|---|---|
| Ave JC 4/5 to | 7.25 | 7.8 | 8.1 | 4.32 | 5.7 | 4.4 | 11.5 |
| 6/24 | | | | | | | |
| SD | 2.63 | 5.8 | 1.8 | 0.37 | 0.93 | 0.72 | 0.59 |
| Fold change | 1.4 | 0.54 | 0.86 | 0.71 | 0.70 | 0.81 | 0.74 |

TABLE 189

| | IL- | | | | | | | | | | | | | | | |
|---|---|---|---|---|---|---|---|---|---|---|---|---|---|---|---|---|
| | 12p70 | | IL-1β | | IL-2 | | IL-21 | | IL-5 | | IL-6 | | TNF-α | | IL-17A | |
| | JC | TC | JC | TC | JC | TC | JC | TC | JC | TC | JC | TC | JC | TC | JC | TC |
| Mean | 3.678 | 1.643 | 1.17 | 1.168 | 6.063 | 0.4775 | 2.089 | 1.723 | 3.606 | 2.878 | 1.923 | 1.168 | 6.443 | 3.235 | 2.309 | 1.648 |
| SD | 0.9621 | 0.5848 | 0.2351 | 0.1443 | 10.42 | 0.1996 | 0.6836 | 0.733 | 2.45 | 0.3902 | 1.469 | 0.405 | 1.071 | 0.3371 | 0.9033 | 0.6232 |

| | IL-12p70 | IL-1b | IL-2 | IL-21 | IL-5 | IL-6 | TNFa | IL-17A |
|---|---|---|---|---|---|---|---|---|
| Ave JC 4/5 to | 2 | 0.82 | 1.45 | 0.7 | 2.13 | 1.9 | 8.2 | 4 |
| 6/24 | | | | | | | | |
| SD | 0.29 | 0.11 | 2.9 | 0.11 | 0.28 | 0.71 | 0.82 | 1.72 |
| Fold | 0.54 | 0.7 | 0.24 | 0.33 | 0.59 | 1 | 1.27 | 1.74 |
| change | | | | | | | | |

TABLE 190

| | H184 JC | H185 TC | H183 NC |
|---|---|---|---|
| NK | 12.8% | 4.45% | 14.5% |
| CD4 | 37.4% | 38.5% | 34.4% |
| CD8 | 24.4% | 23.9% | 27.1% |
| CD19 | 15.7% | 11.6% | 10.3% |
| CD14 | 20.5% | 15.1% | 16.9% |
| total | 110.8% | 93.55% | 103.2% |

TABLE 191

| | | | | | IL-2 + anti- | | IL-2 + anti- | | IL-2 + | | IL-2 + anti- | |
|---|---|---|---|---|---|---|---|---|---|---|---|---|
| | | | IL-2 | | CD16 | | CD3/28 | | sAJ2 | | PD-1 | |
| | untreated | | treated | | treated | | treated | | treated | | treated | |
| | Mean | SD | Mean | SD | Mean | SD | Mean | SD | Mean | SD | Mean | SD |
| H184 JC | 7.89 | 6.74 | 38.83 | 1.75 | 21.00 | 1.45 | 134.78 | 21.09 | 48.01 | 0.53 | 42.13 | 0.03 |
| PBMC | | | | | | | | | | | | |
| H185 TC | 21.48 | | 22.48 | 0.75 | 29.09 | 1.76 | 59.45 | 10.73 | 28.30 | 0.54 | 28.16 | 1.74 |
| PBMC | | | | | | | | | | | | |

TABLE 192

| | | | | | IL-2 + anti- | | IL-2 + anti- | | IL-2 + | | IL-2 + anti- | |
|---|---|---|---|---|---|---|---|---|---|---|---|---|
| | | | IL-2 | | CD16 | | CD3/28 | | sAJ2 | | PD-1 | |
| | untreated | | treated | | treated | | treated | | treated | | treated | |
| | Mean | SD | Mean | SD | Mean | SD | Mean | SD | Mean | SD | Mean | SD |
| H184 JC | 0.00 | 29.8 | 0.00 | 46.5 | 43.97 | 32.97 | 689.17 | 5.87 | 1872.6 | 40.7 | 0.00 | 48.8 |
| PBMC | | | | | | | | | | | | |
| (1:10) | | | | | | | | | | | | |
| H185 TC | 0.00 | 8.58 | 0.00 | 34.8 | 0.00 | 19.42 | 183.87 | 1.36 | 119.03 | 14.5 | 0.00 | 10.4 |
| PBMC | | | | | | | | | | | | |
| (1:10) | | | | | | | | | | | | |

TABLE 193

| | | | | | IL-2 + anti- | | IL-2 + | | IL-2 + anti- | |
|---|---|---|---|---|---|---|---|---|---|---|
| | | | IL-2 | | CD3/28 | | sAJ2 | | PD-1 | |
| | untreated | | treated | | treated | | treated | | treated | |
| | Mean | SD | Mean | SD | Mean | SD | Mean | SD | Mean | SD |
| H184 JC | 0.00 | 23.0 | 0.00 | 37.0 | 1660.8 | 23.94 | 0.00 | 24.4 | 0.00 | 17.6 |
| CD8 (1:5) | | | | | | | | | | |
| H185 TC | 0.00 | 14.0 | 0.00 | 17.6 | 133.41 | 12.20 | 0.00 | 28.9 | 0.00 | 51.0 |
| CD8 (1:5) | | | | | | | | | | |

TABLE 194

| | H186 JC | H187 TC | H190 RS |
|---|---|---|---|
| NK | 18.1% | 14.7% | 21.2% |
| CD4 | 36.4% | 29.8% | 32.2% |
| CD8 | 27.9% | 31.4% | 20.2% |
| CD19 | 10.8% | 16.8% | 8.01% |
| CD14 | 19.9% | 18.8% | 18.9% |
| total | 113.1% | 111.5% | 100.51% |

TABLE 195

| | | | | | IL-2 + anti- | | IL-2 + anti- | | IL-2 + | | IL-2 + anti- | |
|---|---|---|---|---|---|---|---|---|---|---|---|---|
| | | | IL-2 | | CD16 | | CD3/28 | | sAJ2 | | PD-1 | |
| | untreated | | treated | | treated | | treated | | treated | | treated | |
| | Mean | SD | Mean | SD | Mean | SD | Mean | SD | Mean | SD | Mean | SD |
| H186 JC | 6.41 | 0.10 | 31.20 | 0.82 | 7.91 | 1.09 | 52.78 | 4.83 | 33.68 | 9.50 | 40.19 | 0.86 |
| PBMC | | | | | | | | | | | | |
| H187 TC | 0.00 | — | 6.37 | — | 4.84 | 0.73 | 18.19 | 0.23 | 9.49 | 0.69 | 7.49 | 0.34 |
| PBMC | | | | | | | | | | | | |
| H190 RS | 6.18 | 0.52 | 38.68 | — | 9.13 | 0.13 | 22.41 | 0.28 | 23.11 | 0.54 | 25.52 | 0.01 |
| PBMC | | | | | | | | | | | | |

TABLE 196

| | | | | | IL-2 + anti- | | IL-2 + anti- | | IL-2 + | | IL-2 + anti- | |
|---|---|---|---|---|---|---|---|---|---|---|---|---|
| | | | IL-2 | | CD16 | | CD3/28 | | sAJ2 | | PD-1 | |
| | untreated | | treated | | treated | | treated | | treated | | treated | |
| | Mean | SD | Mean | SD | Mean | SD | Mean | SD | Mean | SD | Mean | SD |
| H186 JC | 0.00 | 0.00 | 0.00 | 17.0 | 18.59 | 11.81 | 1688.0 | 19.37 | 1904.5 | 64.7 | 0.00 | 17.9 |
| PBMC | | | | | | | | | | | | |
| (1:10) | | | | | | | | | | | | |
| H187 TC | 0.00 | 4.25 | 0.00 | 0.00 | 0.00 | 13.23 | 16.59 | 31.66 | 98.12 | 38.2 | 0.00 | 26.9 |
| PBMC | | | | | | | | | | | | |
| (1:10) | | | | | | | | | | | | |
| H190 RS | 0.00 | 13.7 | 0.00 | 28.4 | 0.00 | 11.34 | ND | ND | 1086.2 | 30.2 | 0.00 | 27.4 |
| PBMC | | | | | | | | | | | | |
| (1:10) | | | | | | | | | | | | |

TABLE 197

| | | | | | | IL-2 + |
|---|---|---|---|---|---|---|
| | | | IL-2 + anti- | IL-2 + anti- | IL-2 + | anti- |
| | | IL-2 | CD16 | CD3/28 | sAJ2 | PD-1 |
| | untreated | treated | treated | treated | treated | treated |
| H186 JC | 0.00 | 73.73 | 876.36 | 2005.12 | 1802.62 | 310.98 |
| PBMC | | | | | | |
| straight | | | | | | |
| H187 TC | 0.00 | 0.00 | 64.37 | 961.23 | 1045.44 | 0.00 |
| PBMC | | | | | | |
| straight | | | | | | |
| H190 RS | 0.00 | 90.44 | 178.65 | ND | overflow | 188.68 |
| PBMC | | | | | | |
| straight | | | | | | |

TABLE 198

| | | | | | IL-2 + | | IL-2 + | | IL-2 + anti- | |
|---|---|---|---|---|---|---|---|---|---|---|
| | | | IL-2 | | anti-CD3/28 | | sAJ2 | | PD-1 | |
| | untreated | | treated | | treated | | treated | | treated | |
| | Mean | SD | Mean | SD | Mean | SD | Mean | SD | Mean | SD |
| H186 JC | 0.00 | 2.71 | 0.00 | 38.7 | 866.47 | 27.80 | 156.78 | 19.7 | 0.00 | 14.9 |
| CD8 | | | | | | | | | | |
| (1:5) | | | | | | | | | | |
| H187 TC | 2.37 | 10.2 | 0.00 | 35.3 | 129.45 | 111.89 | 0.00 | 61.0 | 0.00 | 39.3 |
| CD8 | | | | | | | | | | |
| (1:5) | | | | | | | | | | |
| H190 RS | 0.00 | 15.6 | 0.00 | 50.2 | 1171.9 | 44.76 | 1588.2 | 14.9 | 15.32 | 6.78 |
| CD8 | | | | | | | | | | |
| (1:5) | | | | | | | | | | |

TABLE 199

| | | | IL-2 + | IL-2 + |
|---|---|---|---|---|
| | | IL-2 | anti-CD3/28 | sAJ2 |
| | untreated | treated | treated | treated |
| H186 JC CD8 | 0 | 0 | 1830 | 806 |
| straight | | | | |
| H187 TC CD8 | 0 | 0 | 763 | 90 |
| straight | | | | |
| H190 RS CD8 | 0 | 0 | 2490 | 2802 |
| straight | | | | |

TABLE 200

| | H194 JC | H196 TC | H195 RS | H197 YC | H198 MK |
|---|---|---|---|---|---|
| NK | 16.3% | 10.7% | 12.9% | 14.3% | 18.7% |
| CD4 | 35.2% | 37.8% | 36.8% | 34.4% | 30.2% |
| CD8 | 26.1% | 21.6% | 18.4% | 21.2% | 25.5% |
| CD19 | 12.0% | 10.7% | 14.7% | 13.3% | 11.1% |
| CD14 | 14.9% | 20.4% | 21.4% | 15.2% | 13.4% |
| total | 104.9% | 101.2% | 104.2% | 98.4% | 98.9% |

TABLE 201

| | | | | | IL-2 + | | IL-2 + | | IL-2 + | | IL-2 + | |
|---|---|---|---|---|---|---|---|---|---|---|---|---|
| | | | IL-2 | | αCD16 | | αCD3/28 | | sAJ2 | | αPD-1 | |
| | untreated | | treated | | treated | | treated | | treated | | treated | |
| PBMC | Mean | SD | Mean | SD | Mean | SD | Mean | SD | Mean | SD | Mean | SD |
| H194 JC | 2.33 | 0.23 | 9.69 | 0.43 | 6.59 | 0.38 | 42.65 | 1.96 | 20.12 | 2.02 | 10.23 | 0.27 |
| H196 TC | 1.09 | 0.20 | 7.23 | 0.86 | 1.16 | 0.25 | 2.90 | 0.37 | 12.10 | 0.11 | 1.97 | 0.40 |
| H195 RS | 1.61 | 0.44 | 8.03 | 0.35 | 4.82 | 0.18 | 6.45 | 0.86 | 5.40 | 0.33 | 7.86 | 0.61 |
| H197 YC | 0.74 | 0.46 | 13.85 | 0.01 | 11.46 | 0.52 | 17.42 | 0.43 | 12.09 | 1.97 | 12.35 | 0.33 |
| H198 MK | 1.88 | 0.27 | 8.32 | 0.18 | 2.53 | 0.35 | 16.72 | 1.28 | 13.95 | 1.16 | 8.82 | 0.16 |

TABLE 202

| | | | | | IL-2 + anti- | | IL-2 + anti- | | IL-2 + | | IL-2 + anti- | |
|---|---|---|---|---|---|---|---|---|---|---|---|---|
| | | | | | CD16 | | CD3/28 | | sAJ2 | | PD-1 | |
| PBMC | untreated | | IL-2 treated | | treated | | treated | | treated | | treated | |
| (1:10) | Mean | SD | Mean | SD | Mean | SD | Mean | SD | Mean | SD | Mean | SD |
| H194 | 0.00 | 1.70 | 0.00 | 0.97 | 0.00 | 9.72 | 307.73 | 14.33 | 136.16 | 6.31 | 0.00 | 18.94 |
| JC | | | | | | | | | | | | |
| H196 | 0.00 | 22.10 | 0.00 | 6.07 | 0.00 | 6.80 | 37.57 | 7.29 | 588.19 | 10.20 | 0.00 | 13.60 |
| TC | | | | | | | | | | | | |
| H195 | 0.00 | 11.42 | 0.00 | 10.44 | 10.00 | 14.33 | 0.00 | 2.67 | 9.75 | 6.31 | 0.00 | 5.59 |
| RS | | | | | | | | | | | | |
| H197 | 0.00 | 12.63 | 0.00 | 13.12 | 34.31 | 19.67 | 82.40 | 1.21 | 340.88 | 25.75 | 0.00 | 0.97 |
| YC | | | | | | | | | | | | |
| H198 | 0.00 | 0.97 | 171.36 | 16.76 | 13.87 | 14.09 | 133.75 | 13.12 | overflow | | 28.47 | 1.21 |
| MK | | | | | | | | | | | | |

TABLE 203

| | | | IL-2 + | IL-2 + | | IL-2 + |
|---|---|---|---|---|---|---|
| | | | anti- | anti- | IL-2 + | anti- |
| PBMC | | IL-2 | CD16 | CD3/28 | sAJ2 | PD-1 |
| straight | untreated | treated | treated | treated | treated | treated |
| H194 JC | 0.00 | 0.00 | 243.80 | 1546.18 | 896.14 | 0.00 |
| H196 TC | 0.00 | 0.00 | 0.00 | 601.78 | 1855.00 | 0.00 |
| H195 RS | 0.00 | 0.00 | 0.00 | 407.46 | 531.22 | 0.00 |
| H197 YC | 0.00 | 0.00 | 235.12 | 572.86 | 1287.09 | 0.00 |
| H198 MK | 0.00 | 870.70 | 207.36 | 866.07 | overflow | 510.98 |

TABLE 204

| | | | IL-2 | | IL-2 + αCD16 | | IL-2 + sAJ2 | | IL-2 + αPD-1 | |
|---|---|---|---|---|---|---|---|---|---|---|
| | untreated | | treated | | treated | | treated | | treated | |
| NK | Mean | SD | Mean | SD | Mean | SD | Mean | SD | Mean | SD |
| H196 TC | 4.27 | 0.65 | 19.53 | 2.29 | 4.66 | 0.21 | 26.65 | 0.64 | 20.00 | 3.20 |
| H195 RS | 4.80 | 1.13 | 17.21 | 0.45 | 5.24 | 1.67 | 17.91 | 0.12 | 18.42 | 0.82 |

TABLE 205

| | | | | | IL-2 + | | IL-2 + | | IL-2 + anti- | |
|---|---|---|---|---|---|---|---|---|---|---|
| | | | IL-2 | | anti-CD16 | | sAJ2 | | PD-1 | |
| NK | untreated | | treated | | treated | | treated | | treated | |
| (1:5) | Mean | SD | Mean | SD | Mean | SD | Mean | SD | Mean | SD |
| H196 TC | 26.58 | 11.17 | 0.00 | 18.94 | 0.00 | 8.99 | 0.00 | 3.89 | 0.00 | 1.94 |
| H195 RS | 0.00 | 8.26 | 0.00 | 18.22 | 0.00 | 10.69 | 10.95 | 6.56 | 0.00 | 7.04 |

TABLE 206

| | | | IL-2 | | IL-2 + anti- | | IL-2 + sAJ2 | | IL-2 + anti-PD-1 | |
|---|---|---|---|---|---|---|---|---|---|---|
| CD8 | untreated | | treated | | CD3/28 treated | | treated | | treated | |
| (1:5) | Mean | SD | Mean | SD | Mean | SD | Mean | SD | Mean | SD |
| H194 JC | 0.00 | 4.50 | 0.00 | 3.68 | 213.73 | 9.00 | 0.00 | 3.27 | 0.00 | 23.72 |
| H196 TC | 0.00 | 27.40 | 0.00 | 2.86 | 170.06 | 24.94 | 0.00 | 2.04 | 0.00 | 23.31 |
| H195 RS | | | | | | | | | | |

TABLE 207

| | | | IL-2 + | IL-2 + | IL-2 + anti- |
|---|---|---|---|---|---|
| CD8 | | IL-2 | anti-CD3/28 | sAJ2 | PD-1 |
| straight | untreated | treated | treated | treated | treated |
| H194 JC | 0.00 | 0.00 | 1829.83 | 805.57 | 0.00 |
| H196 TC | 10.53 | 20.12 | 763.37 | 90.13 | 131.37 |
| H195 RS | 0.00 | 0.00 | 2489.65 | 2802.29 | 0.00 |

TABLE 208
Cell count (×10⁶)

| | H194 JC | H196 TC-1 | H196 TC-2 | H195 RS-1 | H195 RS-2 | H195 RS-3 |
|---|---|---|---|---|---|---|
| Day 0 | | | | | | |
| Day 4 | 0.172 | 0.212 | 0.760 | 0.804 | 0.560 | 0.533 |
| Put | 0.172 (no | 0.172 ( | 0.588 | 0.172 | 0.588 | 1.197 |
| back | change) | | | | | |
| Day 10 | 1.36 (7.9 | 2.1 (12.2 | 3.5 (5.95 | 1.3 (7.6 | 4.9(8.3 fold | 11(9.2 fold |
| | fold | fold | fold | fold | increase) | increase) |
| | increase) | increase) | increase) | increase) | | |
| Put | 1.36 | 1.36 | 4.3 | 1.36 | 4.3 | freeze |
| back | | | | | | |
| Day 12 | 1.008 (.258 | 1.272 (0.94 | 2.6 (0.6 | 3.7(2.72 | 3.8(0.88 | — |
| | fold | fold | fold | fold | fold | |
| | decrease) | decrease) | decrease) | increase) | decrease) | |
| Put | 1.008 | 1.000 | freeze | 1.000 | freeze | — |
| back | | | | | | |
| Day 15 | 0.860 (0.15 | 1.26 (1.26 | — | 1.06(no | — | |
| | fold | fold | | change) | | |
| | decrease) | increase) | | | | |
| Put | 0.500 | 0.500 | — | 0.500 | — | — |
| back | | | | | | |
| Day 18 | 0.510 (no | 0.710 (1.42 | — | 1.200(2.4 | — | — |
| | change) | fold | | fold | | |
| | | increase) | | increase) | | |
| Put | 0.510 | 0.510 | 0.210 | 0.510 | 0.210 | 0.48 |
| back | | | | | | |
| Day20 | 0.340(0.67 | 0.840(1.65 | 0.664 (1.3 | 1.16 (2.3 | 0.520(2.5 | 2.00 (4.2 |
| | fold | fold | fold | fold | fold | fold |
| | decrease) | increase) | increase) | increase) | increase) | increase) |
| Put | 0.340 | 0.340 | 1.64 | 0.340 | 1.64 | 1.7 |
| back | | | | | | |

Cell count, resuspend into 0.5 million per ml with IL-2, anit-CD3/28 and differentiation supplement every time when splitting the cultures.

TABLE 209

| 1:2 dilution | | |
|---|---|---|
| H194 JC | H196 TC | H195 RS |

| | Mean | SD | Mean | SD | Mean | SD |
|---|---|---|---|---|---|---|
| Day 4 | 32.13 | 6.73 | 58.26 | 12.81 | 13.58 | 5.62 |
| Day 7 | 16.08 | 12.01 | 21.33 | 8.19 | 12.47 | 10.79 |
| Day 12 | 18.56 | 10.72 | 12.55 | 9.79 | 10.75 | 7.60 |
| Day 15 | 14.02 | 9.44 | 19.07 | 5.48 | 11.66 | 4.93 |

TABLE 210

| Straight | H194 JC | H196 TC | H195 RS |
|---|---|---|---|
| Day 4 | | 84.69 | 89.01 |
| Day 7 | 27.66 | 29.77 | 16.67 |
| Day 12 | 19.86 | 12.15 | 18.19 |
| Day 15 | 5.68 | 20.20 | 18.53 |

TABLE 211

| IL-10 (pg/ml Per million cells) | | | |
|---|---|---|---|
| | H194 JC | H196 TC | H195 RS |
| 1:2 diution | | | |
| Day 4 | 186.80 | 274.83 | 16.89 |
| Day 7 | 11.82 | 10.16 | 9.59 |
| Day 12 | 18.41 | 9.86 | 2.91 |
| Day 15 | 16.30 | 15.14 | 11.00 |
| Straight | | | |
| Day 4 | | 399.50 | 110.71 |
| Day 7 | 20.34 | 14.18 | 12.82 |
| Day 12 | 19.70 | 9.55 | 4.92 |
| Day 15 | 6.60 | 16.03 | 17.48 |

TABLE 212

| | 1:2 dilution | | | | | |
|---|---|---|---|---|---|---|
| | H194 JC | | H196 TC | | H195 RS | |
| | Mean | SD | Mean | SD | Mean | SD |
| Day 4 | | | 396.45 | 1.03 | | |
| Day 12 | 78.62 | 63.43 | 79.87 | 46.66 | 92.57 | 36.65 |
| Day 15 | 99.12 | 32.97 | 86.32 | 61.96 | 112.03 | 78.89 |

TABLE 213

| straight | H194 JC | H196 TC | H195 RS |
|---|---|---|---|
| Day 12 | 112.24 | 38.76 | 158.24 |
| Day 15 | 131.80 | 165.52 | 161.57 |

TABLE 214

| IL-6 (pg/ml Per million cells) | | | |
|---|---|---|---|
| | H194 JC | H196 TC | H195 RS |
| 1:2 diution | | | |
| Day 4 | | 1870.06 | |
| Day 12 | 78.00 | 62.79 | 25.02 |
| Day 15 | 115.26 | 68.51 | 105.69 |
| straight | | | |
| Day 12 | 111.35 | 30.47 | 42.77 |
| Day 15 | 153.26 | 131.37 | 152.42 |

TABLE 215

| | 1:2 dilution | | | | | |
|---|---|---|---|---|---|---|
| | H194 JC | | H196 TC | | H195 RS | |
| | Mean | SD | Mean | SD | Mean | SD |
| Day 12 | 102.48 | 54.63 | 57.64 | 69.27 | 604.71 | 55.61 |
| Day 15 | 554.00 | 43.42 | 649.55 | 40.98 | 1417.38 | 47.81 |

TABLE 216

| straight | H194 JC | H196 TC | H195 RS |
|---|---|---|---|
| Day 12 | 196.31 | 79.72 | 791.66 |
| Day 15 | 670.25 | 1006.90 | 1863.72 |

TABLE 217

| IFN-γ (pg/ml Per million cells) | | | |
|---|---|---|---|
| | H194 JC | H196 TC | H195 RS |
| 1:2 diution | | | |
| Day 12 | 101.67 | 45.32 | 163.43 |
| Day 15 | 644.19 | 515.52 | 1337.15 |
| straight | | | |
| Day 12 | 194.75 | 62.67 | 213.96 |
| Day 15 | 779.36 | 799.13 | 1758.23 |

TABLE 218

| | | | | | IL-2 + anti- | | IL-2 + anti- | | IL-2 + | | IL-2 + anti- | |
|---|---|---|---|---|---|---|---|---|---|---|---|---|
| | | | IL-2 | | CD16 | | CD3/28 | | sAJ2 | | PD-1 | |
| | untreated | | treated | | treated | | treated | | treated | | treated | |
| | Mean | SD | Mean | SD | Mean | SD | Mean | SD | Mean | SD | Mean | SD |
| H202 JC | 9.13 | 0.04 | 43.67 | 1.25 | 23.82 | 0.64 | 52.14 | 2.49 | 68.77 | 5.02 | 39.13 | 0.74 |
| PBMC | | | | | | | | | | | | |
| H203 TC | 5.96 | 1.82 | 26.92 | 0.65 | 30.40 | 0.98 | 72.63 | 7.60 | 48.09 | 2.21 | 51.73 | 0.11 |
| PBMC | | | | | | | | | | | | |
| H204 RS | 7.28 | 2.56 | 35.63 | 0.51 | 10.43 | 0.13 | 32.61 | 0.77 | 20.87 | 0.16 | 14.32 | 0.55 |
| PBMC | | | | | | | | | | | | |

TABLE 219

| | | | | | IL-2 + anti- | | IL-2 + anti- | | IL-2 + | | IL-2 + anti- | |
|---|---|---|---|---|---|---|---|---|---|---|---|---|
| | | | IL-2 | | CD16 | | CD3/28 | | sAJ2 | | PD-1 | |
| | untreated | | treated | | treated | | treated | | treated | | treated | |
| | Mean | SD | Mean | SD | Mean | SD | Mean | SD | Mean | SD | Mean | SD |
| H202 JC | 0.00 | 0.00 | 0.00 | 17.0 | 0.00 | 11.81 | 211.04 | 19.37 | overflow | 64.7 | 0.00 | 17.9 |
| PBMC | | | | | | | | | | | | |
| (1:10) | | | | | | | | | | | | |
| H203 TC | 0.00 | 13.7 | 0.00 | 28.3 | 0.00 | 11.34 | 19.69 | 30.72 | 577.71 | 30.2 | 0.00 | 27.4 |
| PBMC | | | | | | | | | | | | |
| (1:10) | | | | | | | | | | | | |
| H204 RS | 0.00 | 4.25 | 0.00 | 0.00 | 26.88 | 13.23 | 217.40 | 31.66 | 1465.6 | 38.3 | 0.00 | 26.9 |
| PBMC | | | | | | | | | | | | |
| (1:10) | | | | | | | | | | | | |

TABLE 220

| | | | IL-2 + | IL-2 + | | IL-2 + |
|---|---|---|---|---|---|---|
| | | | anti- | anti- | IL-2 + | anti- |
| | | IL-2 | CD16 | CD3/28 | sAJ2 | PD-1 |
| | untreated | treated | treated | treated | treated | treated |
| H202 JC | 0.00 | 247.26 | 305.89 | 1279.24 | 1286.98 | 69.19 |
| PBMC | | | | | | |
| straight | | | | | | |
| H203 TC | 0.00 | 63.65 | 753.30 | 1327.90 | 1669.68 | 4.48 |
| PBMC | | | | | | |
| straight | | | | | | |
| H204 RS | 0.00 | 33.24 | 138.32 | 814.13 | 1587.28 | 246.71 |
| PBMC | | | | | | |
| straight | | | | | | |

TABLE 221

| | | | | | IL-2 + anti- | | IL-2 + | | IL-2 + anti- | |
|---|---|---|---|---|---|---|---|---|---|---|
| | | | IL-2 | | CD3/28 | | sAJ2 | | PD-1 | |
| | untreated | | treated | | treated | | treated | | treated | |
| | Mean | SD | Mean | SD | Mean | SD | Mean | SD | Mean | SD |
| H202 JC | 0.00 | 2.71 | 0.00 | 38.7 | 494.47 | 27.80 | 29.37 | 19.7 | 0.00 | 14.9 |
| CD8 (1:5) | | | | | | | | | | |
| H203 TC | 0.00 | 15.6 | 0.00 | 50.2 | 174.26 | 44.76 | 0.00 | 14.9 | 0.00 | 6.78 |
| CD8 (1:5) | | | | | | | | | | |

TABLE 222

| | | | IL-2 + | IL-2 + | IL-2 + |
|---|---|---|---|---|---|
| | | IL-2 | anti-CD3/28 | sAJ2 | anti-PD-1 |
| | untreated | treated | treated | treated | treated |
| H203 JC CD8 | 0.00 | 0.00 | 1201.16 | 62.42 | 0.00 |
| straight | | | | | |
| H204 TC CD8 | 0.00 | 0.00 | 850.07 | 0.00 | 0.00 |
| straight | | | | | |

TABLE 223

| | H206 JC | H209 TS |
|---|---|---|
| NK | 13.1% | 12.9% |
| CD4 | 34.8% | 37.6% |
| CD8 | 22.7% | 19.5% |
| CD19 | 18.1% | 8.69% |
| CD14 | 14.0% | 30.5% |
| total | 102.7% | 109.19% |

TABLE 224

| | | | | | IL-2 + anti- | | IL-2 + anti- | | IL-2 + | | IL-2 + anti- | |
|---|---|---|---|---|---|---|---|---|---|---|---|---|
| | | | IL-2 | | CD16 | | CD3/28 | | sAJ2 | | PD-1 | |
| | untreated | | treated | | treated | | treated | | treated | | treated | |
| | Mean | SD | Mean | SD | Mean | SD | Mean | SD | Mean | SD | Mean | SD |
| H206 JC | 0.00 | 4.34 | 9.66 | 8.68 | 246.17 | 12.79 | 466.94 | 0.46 | 0.00 | 2.65 | 76.15 | 5.25 |
| PBMC | | | | | | | | | | | | |
| (1:10) | | | | | | | | | | | | |
| H209 TC | 0.00 | 0.23 | 266.43 | 9.93 | 786.32 | 118.50 | 526.40 | 21.18 | 2619.6 | 89.4 | 362.40 | 5.94 |
| PBMC | | | | | | | | | | | | |
| (1:10) | | | | | | | | | | | | |

TABLE 225

| | | | IL-2 + | IL-2 + | | IL-2 + |
|---|---|---|---|---|---|---|
| | | | anti- | anti- | IL-2 + | anti- |
| | un- | IL-2 | CD16 | CD3/28 | sAJ2 | PD-1 |
| | treated | treated | treated | treated | treated | treated |
| H206 JC | 19.07 | 48.12 | 2836.87 | 2906.89 | 2218.26 | 733.57 |
| PBMC | | | | | | |
| straight | | | | | | |
| H209 TS | 26.03 | 2371.71 | 3027.74 | 2311.29 | 2520.37 | 2089.74 |
| PBMC | | | | | | |
| straight | | | | | | |

TABLE 226

| | | | | | IL-2 + anti- | | | | IL-2 | | IL-2 + anti- | |
|---|---|---|---|---|---|---|---|---|---|---|---|---|
| | | | IL-2 | | CD3/28 | | Untreated | | treated | | CD3/28 | |
| | Untreated | | treated | | treated with | | without | | without | | treated | |
| | with PTX | | with PTX | | PTX | | PTX | | PTX | | without PTX | |
| | Mean | SD | Mean | SD | Mean | SD | Mean | SD | Mean | SD | Mean | SD |
| H206 | 3.32 | 3.18 | 3.32 | 18.8 | 417.54 | 46.79 | 0.00 | 34.72 | 0.00 | 31.8 | 819.41 | 196 |
| JC | | | | | | | | | | | | |
| CD8 | | | | | | | | | | | | |
| (1:5) | | | | | | | | | | | | |
| H209 | 0.00 | 13.0 | 0.86 | 23.4 | 58.76 | 23.73 | 5.57 | 2.31 | 0.00 | 25.1 | 94.36 | 32.4 |
| TS | | | | | | | | | | | | |
| CD8 | | | | | | | | | | | | |
| (1:5) | | | | | | | | | | | | |

TABLE 227

| | | | IL-2 + | | | IL-2 + anti- |
|---|---|---|---|---|---|---|
| | Un- | IL-2 | anti- | Un- | IL-2 | CD3/28 |
| | treated | treated | CD3/28 | treated | treated | treated |
| | with | with | treated | without | without | without |
| | PTX | PTX | with PTX | PTX | PTX | PTX |
| H206 JC | 0.00 | 0.00 | 1927.65 | 5.98 | 19.89 | 2750.56 |
| CD8 | | | | | | |
| straight | | | | | | |
| H209 TS | 12.93 | 17.02 | 551.34 | 59.99 | 57.53 | 328.83 |
| CD8 | | | | | | |
| straight | | | | | | |

TABLE 228

| | H211 JC | H212 TC |
|---|---|---|
| NK | 13.4% | 13.0% |
| CD4 | 37.2% | 31.7% |
| CD8 | 20.9% | 29.5% |
| CD19 | 13.9% | 8.98% |
| CD14 | 14.3% | 13.0% |
| total | 99.7% | 96.2% |

TABLE 229

| | | | | | IL-2 + anti- | | IL-2 + anti- | | | | IL-2 + anti- | |
|---|---|---|---|---|---|---|---|---|---|---|---|---|
| | | | IL-2 | | CD16 | | CD3/28 | | IL-2 + sAJ2 | | PD-1 | |
| | untreated | | treated | | treated | | treated | | treated | | treated | |
| | Mean | SD | Mean | SD | Mean | SD | Mean | SD | Mean | SD | Mean | SD |
| H211 | 0.12 | 0.03 | 9.20 | 0.65 | 8.54 | 0.73 | 16.87 | 0.09 | 13.62 | 0.47 | 9.17 | 1.02 |
| JC | | | | | | | | | | | | |
| PBMC | | | | | | | | | | | | |
| H212 | 0.06 | 0.04 | 6.96 | 1.04 | 5.48 | 1.03 | 12.50 | 0.41 | 12.82 | 1.05 | 8.10 | 0.24 |
| TC | | | | | | | | | | | | |
| PBMC | | | | | | | | | | | | |

TABLE 230

| | | | | | | | | | IL-2 + anti- | |
|---|---|---|---|---|---|---|---|---|---|---|
| | | | IL-2 | | IL-2 + anti-CD16 | | IL-2 + sAJ2 | | PD-1 | |
| | untreated | | treated | | treated | | treated | | treated | |
| | Mean | SD | Mean | SD | Mean | SD | Mean | SD | Mean | SD |
| H211 | 23.03 | 0.59 | 81.51 | 0.02 | 39.87 | 0.02 | 123.20 | 0.03 | 92.50 | 0.02 |
| JC NK | | | | | | | | | | |
| H211 | 0.01 | 0.00 | 46.58 | 0.09 | 0.18 | 0.11 | 50.57 | 0.19 | 48.93 | 0.02 |
| JC NK | | | | | | | | | | |

TABLE 231

| | | | | | IL-2 + anti- | | IL-2 + anti- | | | | IL-2 + anti- | |
|---|---|---|---|---|---|---|---|---|---|---|---|---|
| | | | IL-2 | | CD16 | | CD3/28 | | IL-2 + sAJ2 | | PD-1 | |
| | untreated | | treated | | treated | | treated | | treated | | treated | |
| | Mean | SD | Mean | SD | Mean | SD | Mean | SD | Mean | SD | Mean | SD |
| H211 | 0.00 | 15.1 | 0.00 | 1.67 | 66.47 | 3.58 | 118.66 | 8.12 | 321.18 | 1.19 | 0.00 | 2.15 |
| JC | | | | | | | | | | | | |
| PBMC | | | | | | | | | | | | |
| (1:10) | | | | | | | | | | | | |
| H212 | 0.00 | 20.8 | 0.00 | 30.3 | 0.00 | 18.63 | 98.05 | 9.55 | 148.73 | 0.96 | 0.00 | 8.84 |
| TC | | | | | | | | | | | | |
| PBMC | | | | | | | | | | | | |
| (1:10) | | | | | | | | | | | | |

TABLE 232

| | | | IL-2 + | IL-2 + | | IL-2 + |
|---|---|---|---|---|---|---|
| | | | anti- | anti- | IL-2 + | anti- |
| | | IL-2 | CD16 | CD3/28 | sAJ2 | PD-1 |
| | untreated | treated | treated | treated | treated | treated |
| H211 JC | 1.26 | 20.16 | 1009.54 | 1206.15 | 1661.94 | 40.77 |
| PBMC | | | | | | |
| straight | | | | | | |
| H212 TC | 2.32 | 35.04 | 49.91 | 1197.22 | 1431.49 | 28.87 |
| PBMC | | | | | | |
| straight | | | | | | |

TABLE 233

| | | | | | IL-2 + | | | | IL-2 + anti- | |
|---|---|---|---|---|---|---|---|---|---|---|
| | | | IL-2 | | anti-CD16 | | IL-2 + sAJ2 | | PD-1 | |
| | untreated | | treated | | treated | | treated | | treated | |
| | Mean | SD | Mean | SD | Mean | SD | Mean | SD | Mean | SD |
| H211 JC | 0.00 | 4.06 | 6.33 | 4.06 | 85.55 | 12.90 | 36.91 | 22.5 | 0.00 | 23.4 |
| NK (1:5) | | | | | | | | | | |
| H212 TC | 0.00 | 5.73 | 0.00 | 6.21 | 0.00 | 20.07 | 0.00 | 4.54 | 0.00 | 28.7 |
| NK (1:5) | | | | | | | | | | |

TABLE 234

| | | | IL-2 + | IL-2 + | IL-2 + |
|---|---|---|---|---|---|
| | | IL-2 | anti-CD16 | sAJ2 | anti-PD-1 |
| | untreated | treated | treated | treated | treated |
| H211 JC NK | 40.56 | 102.81 | 546.10 | 189.26 | 80.08 |
| straight | | | | | |
| H212 TC NK | 21.44 | 29.51 | 41.20 | 68.18 | 51.82 |
| straight | | | | | |

TABLE 235

| | | | | | IL-2 + anti- | | | | IL-2 + anti- | |
|---|---|---|---|---|---|---|---|---|---|---|
| | | | IL-2 | | CD3/28 | | IL-2 + sAJ2 | | PD-1 | |
| | untreated | | treated | | treated | | treated | | treated | |
| | Mean | SD | Mean | SD | Mean | SD | Mean | SD | Mean | SD |
| H211 JC | 0.00 | 12.4 | 0.00 | 27.7 | 59.37 | 16.48 | 6.50 | 32.0 | 0.00 | 8.12 |
| CD8 (1:5) | | | | | | | | | | |
| H212 TC | 0.00 | 6.21 | 0.00 | 12.2 | 0.00 | 11.94 | 0.00 | 10.8 | 0.00 | 0.72 |
| CD8 (1:5) | | | | | | | | | | |

TABLE 236

| | | | IL-2 + | IL-2 + | IL-2 + |
|---|---|---|---|---|---|
| | | IL-2 | anti-CD3/28 | sAJ2 | anti-PD-1 |
| | untreated | treated | treated | treated | treated |
| H211 JC CD8 | 33.76 | 44.81 | 437.58 | 57.13 | 47.78 |
| straight | | | | | |
| H212 TC CD8 | 41.20 | 31.21 | 125.33 | 42.26 | 39.07 |
| straight | | | | | |

TABLE 237

| | H211 JC | H212 TC |
|---|---|---|
| NK | 14.1% | 13.1% |
| CD4 | 34.6% | 32.0% |
| CD8 | 16.6% | 27.4% |
| CD19 | 11.9% | 9.67% |
| CD14 | 6.28% | 9.96% |
| total | 83.5% | 92.1% |

TABLE 238

| | | | | | IL-2 + anti- | | IL-2 + anti- | | IL-2 + | | IL-2 + anti- | |
|---|---|---|---|---|---|---|---|---|---|---|---|---|
| | | | IL-2 | | CD16 | | CD3/28 | | sAJ2 | | PD-1 | |
| | untreated | | treated | | treated | | treated | | treated | | treated | |
| | Mean | SD | Mean | SD | Mean | SD | Mean | SD | Mean | SD | Mean | SD |
| H213 | 8.35 | 0.40 | 21.64 | 0.28 | 13.36 | — | 41.49 | — | 40.12 | — | 20.18 | 0.23 |
| JC | | | | | | | | | | | | |
| PBMC | | | | | | | | | | | | |
| H214 | 1.40 | 0.29 | 22.56 | 0.73 | 5.81 | 1.14 | 17.26 | 0.25 | 27.17 | 0.26 | 19.40 | 0.69 |
| TC | | | | | | | | | | | | |
| PBMC | | | | | | | | | | | | |

TABLE 239

| | | | | | | | | | IL-2 + anti- | |
|---|---|---|---|---|---|---|---|---|---|---|
| | | | | | IL-2 + anti- | | IL-2 + sAJ2 | | PD-1 | |
| | untreated | | IL-2 treated | | CD16 treated | | treated | | treated | |
| | Mean | SD | Mean | SD | Mean | SD | Mean | SD | Mean | SD |
| H213 | 23.85 | 0.07 | 157.04 | 12.9 | 41.03 | 0.10 | 128.00 | 8.06 | 137.40 | 8.26 |
| JC NK | | | | | | | | | | |
| H214 | 0.31 | 0.03 | 80.80 | 1.62 | 16.46 | 0.09 | 82.93 | 4.35 | 74.72 | 3.28 |
| TC NK | | | | | | | | | | |

TABLE 240

| | | | | | IL-2 + anti- | | IL-2 + anti- | | IL-2 + | | IL-2 + anti- | |
|---|---|---|---|---|---|---|---|---|---|---|---|---|
| | | | IL-2 | | CD16 | | CD3/28 | | sAJ2 | | PD-1 | |
| | untreated | | treated | | treated | | treated | | treated | | treated | |
| | Mean | SD | Mean | SD | Mean | SD | Mean | SD | Mean | SD | Mean | SD |
| H213 | 0.00 | 2.13 | 0.00 | 3.20 | 10.99 | 14.51 | 185.85 | 4.48 | 1815.4 | 104 | 0.00 | 3.63 |
| JC | | | | | | | | | | | | |
| PBMC | | | | | | | | | | | | |
| (1:10) | | | | | | | | | | | | |
| H214 | 0.00 | 27.3 | 28.19 | 10.2 | 0.00 | 4.05 | 125.50 | 5.76 | 2001.7 | 263 | 0.00 | 16.2 |
| TC | | | | | | | | | | | | |
| PBMC | | | | | | | | | | | | |
| (1:10) | | | | | | | | | | | | |

TABLE 241

| | | | IL-2 + | IL-2 + | | IL- |
|---|---|---|---|---|---|---|
| | | | anti- | anti- | IL-2 + | 2 + anti- |
| | | IL-2 | CD16 | CD3/28 | sAJ2 | PD-1 |
| | untreated | treated | treated | treated | treated | treated |
| H213 JC | 10.08 | 0.00 | 258.77 | 923.01 | 946.11 | 0.00 |
| PBMC | | | | | | |
| straight | | | | | | |
| H214 TC | 0.00 | 23.66 | 46.29 | 950.44 | 1139.61 | 23.06 |
| PBMC | | | | | | |
| straight | | | | | | |

TABLE 242

| | | | | | IL-2 + | | | | IL-2 + anti- | |
|---|---|---|---|---|---|---|---|---|---|---|
| | | | IL-2 | | anti-CD16 | | IL-2 + sAJ2 | | PD-1 | |
| | untreated | | treated | | treated | | treated | | treated | |
| | Mean | SD | Mean | SD | Mean | SD | Mean | SD | Mean | SD |
| H213 JC | 0.00 | 0.21 | 29.09 | 32.0 | 100.60 | 23.47 | 1692.7 | 133 | 0.00 | 0.43 |
| NK (1:5) | | | | | | | | | | |
| H214 | 0.00 | 3.41 | 0.00 | 6.83 | 10.23 | 10.03 | 1372.8 | 9.19 | 12.19 | 5.12 |
| TC NK | | | | | | | | | | |
| (1:5) | | | | | | | | | | |

TABLE 243

| | | | IL-2 + | IL-2 + | IL-2 + |
|---|---|---|---|---|---|
| | | IL-2 | anti-CD16 | sAJ2 | anti-PD-1 |
| | untreated | treated | treated | treated | treated |
| H213 JC NK | 0.00 | 26.07 | 251.55 | 863.80 | 36.33 |
| straight | | | | | |
| H214 TC NK | 0.00 | 22.45 | 87.63 | 1219.03 | 0.73 |
| straight | | | | | |

TABLE 244

| | | | | | IL-2 + | | | | IL-2 + anti- | |
|---|---|---|---|---|---|---|---|---|---|---|
| | | | IL-2 | | anti-CD3/28 | | IL-2 + sAJ2 | | PD-1 | |
| | untreated | | treated | | treated | | treated | | treated | |
| | Mean | SD | Mean | SD | Mean | SD | Mean | SD | Mean | SD |
| H213 JC | 3.16 | 10.9 | 19.87 | 9.15 | 253.25 | 13.17 | 25.82 | 1.62 | 14.75 | 2.51 |
| CD8 (1:5) | | | | | | | | | | |
| H214 TC | 4.31 | 1.33 | 2.53 | 0.59 | 344.06 | 1.10 | 25.82 | 2.81 | 8.69 | 2.51 |
| CD8 (1:5) | | | | | | | | | | |

TABLE 245

| | | | IL-2 + | IL-2 + | IL-2 + |
|---|---|---|---|---|---|
| | | IL-2 | anti-CD3/28 | sAJ2 | anti-PD-1 |
| | untreated | treated | treated | treated | treated |
| H213 JC CD8 | 13.81 | 25.92 | 906.22 | 85.02 | 13.39 |
| straight | | | | | |
| H214 TC CD8 | 7.13 | 15.27 | 969.35 | 115.71 | 10.26 |
| straight | | | | | |

TABLE 246
After NAC treatment in ALS patient

| | |
|---|---|
| JC sera 04052020 | (NAC infusion) |
| JC sera 04162020 | (NAC infusion |
| JC sera 04232020 | (NAC infusion |
| JC sera 04302020 | (NAC infusion |
| JC sera 05072020 | (NAC infusion |
| JC sera 05142020 | (NAC infusion |
| JC sera 05212020 | (NAC infusion |
| JC sera 05282020 | (NAC infusion |
| JC sera 06222020 | (NAC infusion |
| JC sera 06242020 | (NAC infusion |

Total of 10 data points

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the present invention described herein. Such equivalents are intended to be encompassed by the following claims.

1. A method of treating a neurodegenerative disease in a subject in need thereof, or a method of inhibiting degeneration and/or death of a nerve cell in a subject, the method comprising administering to the subject at least one agent that decreases the level of one or more pro-inflammatory cytokines, chemokines, and/or growth factors.

2. The method of claim 1, wherein the neurodegenerative disease is Amyotrophic Lateral Sclerosis (ALS).

3. The method of claim 1, wherein the at least one agent comprises
(a) at least one of N-Acetyl-Cysteine (NAC), an anti-IL-6 antibody, an anti-TNF-α antibody, an anti-Rantes antibody, and an anti-IFN-g antibody;
(b) NAC;
(c) anti-IL-6 antibody and an anti-TNF-α antibody;
(d) anti-IFN-g antibody: or
(e) NAC, an anti-TNF-α antibody, and an anti-IFN-g antibody.

4-7. (canceled)

8. The method of claim 1, wherein the one or more pro-inflammatory cytokines, chemokines, and/or growth factors are selected from
(a) Rantes, EGF, FGF2, Eotaxin, TGF-α, FIT3L, GM-CSF, FRACTALKINE, IFNa2, IFN-g, MCP3, IL-12, MDC, PDGF-AA, PDGF-AB, PDGF-BB, IL-13, IL-15, sCD40L, IL-1Ra, IL-1a, IL-9, IL-1b, IL-3, IL-4, IL-7, IL-8, IP-10, MCP1, TNF-β, VEGF, IL-10, TNF-α, IL-17A, IL-1β, IL-2, IL-21, IL-4, IL-23, IL-5, IL-6, MIP-3α, MIP-1α, and MIP-1β;
(b) IL-10, IL-12, IFN-g, TNF-α, IL-13, IL-17A, IL-2, IL-21, IL-4, IL-23, IL-5, IL-6, and MIP-3α, or
(c) IL-4, IL-10, IL-12, IL-2, IL-13, IL-6, TNF-α, and IFN-g.

9-10. (canceled)

11. The method of claim 1, wherein the at least one agent
(a) decreases inflammation in the subject,
(b) inhibits the degeneration and/or death of a nerve cell; and/or
(c) decreases the likelihood of pulmonary embolism and/or cardiac failure.

12-13. (canceled)

14. The method of claim 1, further comprising administering to the subject at least one additional therapy that treats a neurodegenerative disease or at least one additional therapy that inhibits degeneration and/or death of a nerve cell.

15. The method of claim 14, wherein the at least one additional therapy is administered to the subject before, after, or concurrently with the agent that decreases the level of one or more pro-inflammatory cytokines, chemokines, and/or growth factors.

16. The method of claim 14, wherein the at least one additional therapy is edavarone and/or riluzole.

17-31. (canceled)

32. A method of determining whether a subject afflicted with a neurodegenerative disease would likely respond to treatment with at least one agent that decreases the level of one or more pro-inflammatory cytokines, chemokines, and/or growth factors, the method comprising:
a) determining the amount of at least one biomarker in a subject sample;
b) determining the amount of the same biomarker(s) in a control; and
c) comparing the amount of the biomarker(s) in a) and b);
wherein the at least one biomarker is selected from Rantes, IL-10, IL-12, IFN-g, TNF-α, IL-13, IL-17A, IL-2, IL-21, IL-4, IL-23, IL-5, IL-6, and MIP-3α; and
wherein a significant increase in the amount of the biomarker(s) in the subject sample relative to the control indicates that the subject would benefit from treatment with an agent that decreases the level of one or more pro-inflammatory cytokines, chemokines, and/or growth factors.

33. The method of claim 32, wherein the neurodegenerative disease is Amyotrophic Lateral Sclerosis (ALS).

34. The method of claim 32, wherein
(a) the amount of the biomarker is the amount of protein; and/or
(b) the sample comprises serum.

35. The method of claim 32, wherein the control is determined from a subject not afflicted with the degenerative disease.

36. The method of claim 32, further comprising prescribing at least one agent that decreases the level of one or more pro-inflammatory cytokines, chemokines, and/or growth factors, if the amount of the biomarker(s) in the subject sample is increased relative to the control.

37. The method of claim 32, wherein the at least one agent comprises
(a) at least one of N-Acetyl-Cysteine (NAC), an anti-IL-6 antibody, an anti-TNF-α antibody, an anti-Rantes antibody, and an anti-IFN-g antibody;
(b) NAC;
(c) an anti-IL-6 antibody and an anti-TNF-α antibody;
(d) an anti-IFN-g antibody; or
(e) NAC, an anti-TNF-α antibody, and an anti-IFN-g antibody.

38. The method of claim 36, wherein the at least one agent comprises
(a) at least one of N-Acetyl-Cysteine (NAC), an anti-IL-6 antibody, an anti-TNF-α antibody, an anti-Rantes antibody, and an anti-IFN-g antibody;
(b) NAC;
(c) an anti-IL-6 antibody and an anti-TNF-α antibody;
(d) an anti-IFN-g antibody; or
(e) NAC, an anti-TNF-α antibody, and an anti-IFN-g antibody.

39-41. (canceled)

42. The method of claim 32, wherein the one or more pro-inflammatory cytokines, chemokines, and/or growth factors are selected from
a) Rantes, EGF, FGF2, Eotaxin, TGF-α, FIT3L, GM-CSF, FRACTALKINE, IFNa2, IFN-g, MCP3, IL-12, MDC, PDGF-AA, PDGF-AB, PDGF-BB, IL-13, IL-15, sCD40L, IL-1Ra, IL-1a, IL-9, IL-1b, IL-3, IL-4, IL-7, IL-8, IP-10, MCP1, TNF-β, VEGF, IL-10, TNF-α, IL-17A, IL-β, IL-2, IL-21, IL-4, IL-23, IL-5, IL-6, MIP-3α, MIP-1α, and MIP-1β;
(b) Rantes, IL-10, IL-12, IFN-g, TNF-α, IL-13, IL-17A, IL-2, IL-21, IL-4, IL-23, IL-5, IL-6, and MIP-3α; or
(c) Rantes, IL-4, IL-10, IL-12, IL-2, IL-13, IL-6, TNF-α, and IFN-g.

43. The method of claim 36, wherein the one or more pro-inflammatory cytokines, chemokines, and/or growth factors are selected from
(a) Rantes, EGF, FGF2, Eotaxin, TGF-α, FIT3L, GM-CSF, FRACTALKINE, IFNa2, IFN-g, MCP3, IL-12, MDC, PDGF-AA, PDGF-AB, PDGF-BB, IL-13, IL-15, sCD40L, IL-1Ra, IL-1a, IL-9, IL-1b, IL-3, IL-4, IL-7, IL-8, IP-10, MCP1, TNF-β, VEGF, IL-10, TNF-α, IL-17A, IL-1β, IL-2, IL-21, IL-4, IL-23, IL-5, IL-6, MIP-3α, MIP-1α, and MIP-1β;
(b) Rantes, IL-10, IL-12, IFN-g, TNF-α, IL-13, IL-17A, IL-2, IL-21, IL-4, IL-23, IL-5, IL-6, and MIP-3α; or
(c) Rantes, IL-4, IL-10, IL-12, IL-2, IL-13, IL-6, TNF-α, and IFN-g.

44. (canceled)

45. The method of claim 32, wherein the at least one agent decreases (a) inflammation in the subject; and/or (b) the likelihood of pulmonary embolism and/or cardiac failure.

46. (canceled)

47. The method of claim 32, wherein the subject is a mammal or a human.

48. The method of claim 1, wherein the subject is a mammal or a human.
